(19) United States
(12) Patent Application Publication
(10) Pub. No.: US 2025/0263405 A1
(43) Pub. Date: Aug. 21, 2025

(54) HISTONE DEACETYLASE 6 INHIBITOR COMPOUNDS AND USES THEREOF

(71) Applicant: VALO HEALTH, INC., Boston, MA (US)

(72) Inventor: Brian RAIMUNDO, Boston, MA (US)

(21) Appl. No.: 18/706,756

(22) PCT Filed: Nov. 4, 2022
(86) PCT No.: PCT/US2022/048911

§ 371 Date: May 2, 2024

Related U.S. Application Data
(60) Provisional application No. 63/275,765, filed on Nov. 4, 2021.

(51) Int. Cl. C07D 413/14; A61K 31/454; A61K 31/553; C07D 413/04; C07D 413/12; C07D 417/14; C07D 471/04; C07D 471/08; C07D 487/04; C07D 498/04

(52) U.S. Cl. CPC C07D 413/14; A61K 31/454; A61K 31/553; C07D 413/04; C07D 413/12; C07D 417/14; C07D 471/04; C07D 471/08; C07D 487/04; C07D 498/04

(57) ABSTRACT

The present disclosure provides compounds, pharmaceutically acceptable compositions thereof, and methods of using the same, useful in the inhibition of HDAC6.

### TECHNICAL FIELD OF INVENTION

The present disclosure relates to compounds and methods useful for inhibition of Histone Deacetylase 6 (HDAC6). The disclosure also provides pharmaceutically acceptable compositions comprising compounds of the present disclosure and methods of using said compositions in the treatment of various diseases, disorders, and conditions as described herein.

### BACKGROUND OF THE INVENTION

There remains a need for compounds that inhibit HDAC6 useful as therapeutic agents.

Eleven members of the HDAC6 family have been identified in humans, which share a conserved catalytic domain and are grouped into two classes: class I (HDACs 1, 2, 3, and 8); class IIa (HDACs 4, 5, 7, and 9); and class IIb (HDACs 6 and 10). HDAC11 shares homologies with both classes, but is also distinct from the other ten subtypes.

HDAC6 is unique in its structural and physiological functions. For example, besides histone modification, largely due to its cytoplasmic localization, HDAC6 also targets several non-histone proteins, including Hsp90, α-tubulin, cortactin, HSF1, among others. Accordingly, targeting HDAC6 has been of interest in various disease pathways. However, despite extensive efforts, few HDAC6-selective inhibitors have been identified.

### SUMMARY OF THE INVENTION

Most HDAC inhibitors share a common pharmacophore that consists of a zinc-binding group (ZBG) that coordinates with the zinc ion located in the catalytic cavity, a linker that occupies the hydrophobic tunnel, and a capping group that interacts with the rim of the pocket. See Sravani Pulya, S., et al. “HDAC6 as privileged target in drug discovery: A perspective.” Pharmacological Research, 2020, in press. The majority of reported HDAC6 inhibitor compounds use a hydroxamic acid moiety as the zinc-binding group. See, e.g., US 2015/0239869, WO 2015/054474, WO 2017/075192, WO 2018/089651, WO 2014/181137, WO 2016/067038, WO 2017/206032, WO 2016/168598, WO 2016/168660, WO 2017/218950, US 2016/0221973, US 2016/0222022, US 2016/0221997, WO 2014/178606, WO 2015/087151, WO 2015/102426, WO 2015/137750, WO 2018/189340, and WO 2018/130155. Other zinc-binding groups have also been described, for example, mercaptoacetamides (see, e.g., Lv, W. et al. ACS Med. Chem. Lett. 2017, 8(5), 510-515), and di/trifluoromethyloxadiazoles (see, e.g., WO 2017/222950, WO 2017/222951, WO 2017/222952, WO 2016/031815, WO 2017/014170, WO 2017/014321, WO 2017/033946, WO 2019/027054, WO 2019/166824, WO 2019/110663, WO 2017/018803, WO 2017/018805, WO 2017/023133, and WO 2017/065473).

Additionally or alternatively, structures of the linker and capping groups reported vary. In some aspects, compounds with the zinc binding group bound to an aromatic ring are reported (see, e.g., US 2015/0239869, WO 2015/054474, WO 2017/075192, WO 2018/089651, WO 2014/181137, WO 2016/067038, WO 2017/206032, WO 2016/168598, WO 2016/168660, WO 2017/218950, US 2016/0221973, US 2016/0222022, US 2016/0221997, WO 2014/178606, WO 2015/087151, WO 2015/102426, WO 2015/137750, WO 2018/189340, and WO 2018/130155; and WO 2017/222950, WO 2017/222951, WO 2017/222952, WO 2016/031815, WO 2017/014170, WO 2017/014321, WO 2017/033946, WO 2019/027054, WO 2019/166824, WO 2019/110663, WO 2017/018803, WO 2017/018805, WO 2017/023133, and WO 2017/065473). Such reported aromatic rings include both monocyclic (see, e.g., US 2015/0239869, WO 2015/054474, WO 2017/075192, WO 2018/089651, WO 2014/181137, WO 2016/067038, WO 2017/206032, WO 2014/178606, WO 2015/087151, WO 2015/102426, WO 2015/137750, WO 2018/189340, and WO 2018/130155) and bicyclic rings (see, e.g., WO 2016/168598, WO 2016/168660, WO 2017/218950, US 2016/0221973, US 2016/0222022, US 2016/0221997). In other aspects, compounds with the zinc binding group bound to an alkyl group are reported (see, e.g., WO 2014/072714, WO 2016/067040, WO 2016/190630, WO 2019/139921, and Lv, W. et al. ACS Med. Chem. Lett. 2017, 8(5), 510-515).

In some aspects, the present disclosure provides the recognition that a particular zinc-binding group, di/trifluoromethyloxadiazoles, bound to an aromatic portion of a particular linker/capping group comprising a 6,7-bicyclic ring provides improved HDAC6 inhibitors. In some embodiments, provided compounds exhibit improved HDAC6 potency, selectivity, and/or pharmacological properties (e.g., enhanced lipophilicity, stability (e.g., lower rate of metabolism), and/or reduced toxicity), and/or brain penetration properties).

Accordingly, in some embodiments, the present disclosure provides the recognition that there remains a need to find inhibitors of HDAC6 useful as therapeutic agents. It has now been found that compounds of the present disclosure, and pharmaceutically acceptable salts and compositions thereof, are effective as inhibitors of HDAC6. Such compounds have general Formula I:

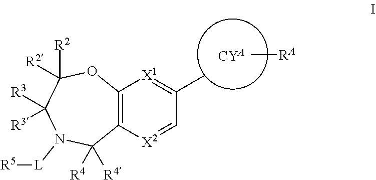

or a pharmaceutically acceptable salt thereof, wherein:
each of X¹ and X² is independently CR¹ or N;
each R¹ is independently hydrogen, halogen, —CN, or an optionally substituted C₁₋₆ aliphatic;
Cy^{A} is 1,2,4-oxadiazolyl or 1,3,4-oxadiazolyl;
R^{A} is methyl, optionally substituted with 1-3 fluoro;
each of R², R^{2′}, R³, R^{3′}, R⁴, R^{4′}, and R⁵ is independently hydrogen, halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur; or
R³ and R^{3′}, together with their intervening atoms, form an optionally substituted 3- to 6-membered spirocyclic ring having 0-3 heteroatoms independently selected from oxygen, nitrogen, or sulfur; or
R³ and R^{3′} form ═O;
L is a covalent bond, or an optionally substituted C₁₋₃ hydrocarbon chain, wherein 1-3 methylene units are optionally and independently replaced with —O—, —C(O)—, —S(O)₂—, or —NR—; and
each R is independently hydrogen or optionally substituted C₁₋₆ aliphatic.

Compounds described herein, and pharmaceutically acceptable compositions thereof, are useful for treating a variety of diseases, disorders, or conditions associated with HDAC6. Such diseases, disorders, or conditions include those described herein.

### DETAILED DESCRIPTION OF CERTAIN EMBODIMENTS

### 1. Definitions

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “partially unsaturated”, as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

The term “lower alkyl”, as used herein, refers to a C₁₋₄ straight or branched alkyl group. Exemplary lower alkyl groups are methyl, ethyl, propyl, isopropyl, butyl, isobutyl, and tert-butyl.

The term “halogen” means F, Cl, Br, or I.

The term “aryl”, as used herein, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl” is a group in which an aromatic ring is fused to one or more non-aromatic rings, such as indanyl, phthalimidyl, naphthimidyl, phenanthridinyl, or tetrahydronaphthyl, and the like.

The term “heteroaryl”, as used herein, does not differ significantly from the common meaning of the term in the art, and refers to a cyclic aromatic radical having from five to twelve ring atoms of which one ring atom is selected from S, O and N; zero, one, two, three, four, or five ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

The term “heteroaryl” as used herein, refers to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” as used herein, refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, tetrahydroquinolinyl, and tetrahydroisoquinolinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula I and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

As used herein, the terms “heterocycle”, “heterocyclyl”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, tetrahydroquinolinyl, or tetrahydroisoquinolinyl where the radical or point of attachment is on the heterocyclyl ring. A heterocyclyl group may be mono- or bicyclic.

As described herein, compounds may contain “optionally substituted” moieties. In general, the term “substituted”, whether preceded by the term “optionally” or not, means that one or more hydrogens of the designated moiety of compounds are replaced with a suitable substituent. “Substituted” applies to one or more hydrogens that are either explicit or implicit from the structure (e.g.,

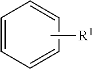

refers to at least

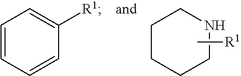

refers to at least

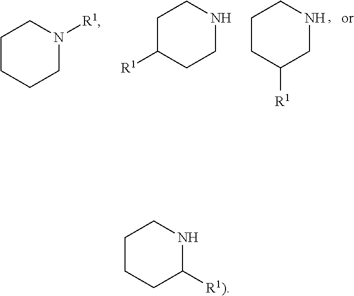

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5- to 6 membered heteroaryl ring), or a 5- to 6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR*, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula I, and subgenera thereof, include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted -OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein the term “biological sample” includes, without limitation, cell cultures or extracts thereof; biopsied material obtained from an animal (e.g., mammal) or extracts thereof, and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds described herein that may be combined with the carrier materials to produce a composition in a single dosage form will vary depending upon the host treated, the particular mode of administration, etc.

The expression “unit dosage form” as used herein refers to a physically discrete unit of a provided compound and/or compositions thereof appropriate for the subject to be treated. It will be understood, however, that the total daily usage of the active agent (i.e., compounds and compositions described herein) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

### 2. Description of Exemplary Embodiments

In some embodiments, the present disclosure provides a compound of Formula I.

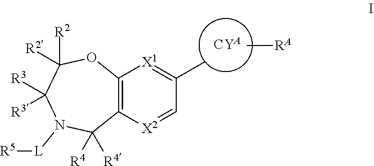

or a pharmaceutically acceptable salt thereof, wherein each of Cy^{A}, R^{A}, X¹, X², R², R^{2′}, R³, R^{3′}, R⁴, R^{4′}, R⁵, and L is as defined and described above and herein.

As defined generally above, each of X¹ and X² is independently CR¹ or N. In some embodiments, X¹ is CR¹. In some embodiments, X¹ is CH. In some embodiments, X¹ is CF. In some embodiments, X¹ is N. In some embodiments, X² is CR¹. In some embodiments, X² is CH. In some embodiments, X² is CF. In some embodiments, X² is N. In some embodiments, X¹ and X² are CR¹. In some embodiments, X¹ and X² are CH. In some embodiments, X¹ and X² are N. In some embodiments, X¹ is N and X² is CR¹. In some embodiments, X¹ is CR¹ and X² is N. In some embodiments, X¹ is CH and X² is CF. In some embodiments, X¹ is CF and X² is CH. In some embodiments, X¹ is CH and X² is CH or CF.

As defined generally above, each R¹ is independently hydrogen, halogen, —CN, or an optionally substituted C₁₋₆ aliphatic. In some embodiments, R¹ is hydrogen. In some embodiments, R¹ is halogen. In some embodiments, R¹ is fluoro or chloro. In some embodiments, R¹ is fluoro. In some embodiments, R¹ is —CN. In some embodiments, R¹ is an optionally substituted C₁₋₆ aliphatic. In some embodiments, R¹ is methyl, ethyl, or propyl. In some embodiments, R¹ is hydrogen or fluoro.

As defined generally above, Cy^{A} is 1,2,4-oxadiazolyl or 1,3,4-oxadiazolyl. In some embodiments, Cy^{A} is 1,2,4-oxadiazolyl. In some embodiments, Cy^{A} is 1,3,4-oxadiazolyl. In some embodiments, Cy^{A} is

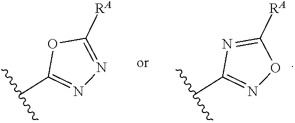

In some embodiments, Cy^{A} is

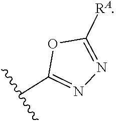

In some embodiments, Cy^{A} is

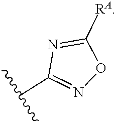

As defined generally above, R^{A} is methyl, optionally substituted with 1-3 fluoro. In some embodiments, R^{A} is methyl. In some embodiments, R^{A} is methyl substituted with 1-3 fluoro. In some embodiments, R^{A} is —CF₂ or CF₃. In some embodiments, R^{A} is —CF₂. In some embodiments, R^{A} is —CF₃.

As defined generally above, each of R² and R^{2′} is independently hydrogen, halogen, —CN, —OR, —N(R)₂, —S(O)₂R, or an optionally substituted group selected from the group consisting of C₁₋aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur.

In some embodiments, R² is hydrogen. In some embodiments, R^{2′} is hydrogen. In some embodiments, R² and R^{2′} are hydrogen.

In some embodiments, R^{2′} is hydrogen, and R² is halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur. In some embodiments, R^{2′} is hydrogen, and R² is halogen. In some embodiments, R^{2′} is hydrogen, and R² is —CN. In some embodiments, R^{2′} is hydrogen, and R² is —OR. In some embodiments, R^{2′} is hydrogen, and R² is —N(R)₂. In some embodiments, R^{2′} is hydrogen, and R² is —S(O)₂R.

In some embodiments, R^{2′} is hydrogen, and R² is an optionally substituted group selected from the group consisting of C₁₋aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur. In some embodiments, R^{2′} is hydrogen, and R² is an optionally substituted group selected from the group consisting of C₁₋₆ aliphatic, a 3- to 7-membered saturated or partially unsaturated monocyclic carbocycyl, or phenyl. In some embodiments, R^{2′} is hydrogen, and R² is an optionally substituted group selected from the group consisting of C₁₋aliphatic or phenyl. In some embodiments, R^{2′} is hydrogen, and R² is C₁₋aliphatic or phenyl. In some embodiments, R^{2′} is hydrogen, and R² is methyl or phenyl.

In some embodiments, R^{2′} is hydrogen, and R² is an optionally substituted C₁₋aliphatic. In some embodiments, R^{2′} is hydrogen, and R² is C₁₋₆ aliphatic. In some embodiments, R^{2′} is hydrogen, and R² is methyl. In some embodiments, R^{2′} is hydrogen, and R² is ethyl. In some embodiments, R^{2′} is hydrogen, and R² is n-propyl. In some embodiments, R^{2′} is hydrogen, and R² is isopropyl. In some embodiments, R^{2′} is hydrogen, and R² is C₁₋aliphatic, optionally substituted with —(CH₂)₀₋₄OR^{◯}, wherein R^{◯} is hydrogen, or C₁₋₆ aliphatic. In some embodiments, R^{2′} is hydrogen, and R² is a 3- to 7-membered saturated or partially unsaturated monocyclic carbocycyl. In some embodiments, R^{2′} is hydrogen, and R² is cyclopropyl. In some embodiments, R^{2′} is hydrogen, and R² is a 3- to 7-membered saturated or partially unsaturated monocyclic heterocyclyl having 1-2 heteroatoms independently selected from oxygen, nitrogen, or sulfur. In some embodiments, R^{2′} is hydrogen, and R² is optionally substituted phenyl. In some embodiments, R^{2′} is hydrogen, and R² is phenyl. In some embodiments, R^{2′} is hydrogen, and R² is a 5- to 6-membered monocyclic heteroaryl having 1-3 heteroatoms independently selected from oxygen, nitrogen, or sulfur. In some embodiments, R^{2′} is hydrogen, and R² is an 8- to 10-membered bicyclic aryl. In some embodiments, R^{2′} is hydrogen, and R² is a 7- to 12-member saturated or partially unsaturated bicyclic heterocyclyl having 1-4 heteroatoms independently selected from oxygen, nitrogen, or sulfur. In some embodiments, R^{2′} is hydrogen, and R² is an 8- to 12-membered bicyclic heteroaryl having 1-4 heteroatoms independently selected from oxygen, nitrogen or sulfur.

In some embodiments, R^{2′} is hydrogen, and R² is hydrogen, methyl, isopropyl, phenyl, cyclopropyl, or

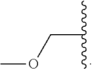 [SMILES: *COC]

In some embodiments, R^{2′} is hydrogen, and R² is hydrogen, methyl, or isopropyl.

As defined generally above, each of R³ and R^{3′} is independently hydrogen, halogen, —CN, —OR, —N(R)₂, —S(O)₂R or an optionally substituted group selected from the group consisting of C₁₋aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur; or R³ and R^{3′}, together with their intervening atoms, form an optionally substituted 3- to 6-membered spirocyclic ring having 0-3 heteroatoms independently selected from oxygen, nitrogen, or sulfur; or R³ and R^{3′} form ═O. In some embodiments, each of R³ and R^{3′} is independently hydrogen, halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur.

In some embodiments, R³ is hydrogen. In some embodiments, R^{3′} is hydrogen. In some embodiments, R³ and R^{3′} are hydrogen.

In some embodiments, R^{3′} is hydrogen, and R³ is halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur. In some embodiments, R^{3′} is hydrogen, and R³ is halogen. In some embodiments, R^{3′} is hydrogen, and R³ is —CN. In some embodiments, R^{3′} is hydrogen, and R³ is —OR. In some embodiments, R^{3′} is hydrogen, and R³ is —N(R)₂. In some embodiments, R^{3′} is hydrogen, and R³ is —S(O)₂R.

In some embodiments, R^{3′} is hydrogen, and R³ is an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, and an 8- to 12-membered bicyclic heteroaryl having 1-4 heteroatoms independently selected from oxygen, nitrogen or sulfur. In some embodiments, R^{3′} is hydrogen, and R³ is an optionally substituted group selected from the group consisting of C₁₋₆ aliphatic, a 3- to 7-membered saturated or partially unsaturated monocyclic carbocycyl, or phenyl. In some embodiments, R^{3′} is hydrogen, and R³ is an optionally substituted group selected from the group consisting of C₁₋₆ aliphatic or phenyl. In some embodiments, R^{3′} is hydrogen, and R³ is C₁₋₆ aliphatic or phenyl. In some embodiments, R^{3′} is hydrogen, and R³ is methyl or phenyl.

In some embodiments, R^{3′} is hydrogen, and R³ is an optionally substituted C₁₋₆ aliphatic. In some embodiments, R^{3′} is hydrogen, and R³ is C₁₋₆ aliphatic. In some embodiments, R^{3′} is hydrogen, and R³ is methyl. In some embodiments, R^{3′} is hydrogen, and R³ is ethyl. In some embodiments, R^{3′} is hydrogen, and R³ is n-propyl. In some embodiments, R^{3′} is hydrogen, and R³ is isopropyl. In some embodiments, R^{3′} is hydrogen, and R³ is C₁₋₆ aliphatic, optionally substituted with —(CH₂)₀₋₄OR^{◯}, wherein R^{◯} is hydrogen, or C₁₋₆ aliphatic. In some embodiments, R^{3′} is hydrogen, and R³ is a 3- to 7-membered saturated or partially unsaturated monocyclic carbocycyl. In some embodiments, R^{3′} is hydrogen, and R³ is cyclopropyl. In some embodiments, R^{3′} is hydrogen, and R³ is a 3- to 7-membered saturated or partially unsaturated monocyclic heterocyclyl having 1-2 heteroatoms independently selected from oxygen, nitrogen, or sulfur. In some embodiments, R^{3′} is hydrogen, and R³ is optionally substituted phenyl. In some embodiments, R^{3′} is hydrogen, and R³ is phenyl. In some embodiments, R^{3′} is hydrogen, and R³ is a 5- to 6-membered monocyclic heteroaryl having 1-3 heteroatoms independently selected from oxygen, nitrogen, or sulfur. In some embodiments, R^{3′} is hydrogen, and R³ is an 8- to 10-membered bicyclic aryl. In some embodiments, R^{3′} is hydrogen, and R³ is a 7- to 12-member saturated or partially unsaturated bicyclic heterocyclyl having 1-4 heteroatoms independently selected from oxygen, nitrogen, or sulfur. In some embodiments, R^{3′} is hydrogen, and R³ is an 8- to 12-membered bicyclic heteroaryl having 1-4 heteroatoms independently selected from oxygen, nitrogen or sulfur.

In some embodiments, R^{3′} is hydrogen, and R³ is hydrogen, methyl, isopropyl, phenyl, cyclopropyl, or

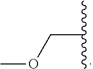 [SMILES: *COC]

In some embodiments, R^{3′} is hydrogen, and R³ is hydrogen, methyl, phenyl, cyclopropyl, or

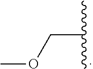 [SMILES: *COC]

In some embodiments, R³ and R^{3′}, together with their intervening atoms, form an optionally substituted 3- to 6-membered spirocyclic ring having 0-3 heteroatoms independently selected from oxygen, nitrogen, or sulfur. In some embodiments, R³ and R^{3′}, together with their intervening atoms, form an optionally substituted 3-membered spirocyclic ring having 0-3 heteroatoms independently selected from oxygen, nitrogen, or sulfur. In some embodiments, R³ and R^{3′}, together with their intervening atoms, form a 3-membered spirocarbocyclic ring. In some embodiments, R³ and R^{3′} form═O.

As defined generally above, each of R⁴ and R^{4′} is independently hydrogen, halogen, —CN, —OR, —N(R)₂, —S(O)₂R, or an optionally substituted group selected from the group consisting of C₁₋aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur.

In some embodiments, R⁴ is hydrogen. In some embodiments, R^{4′} is hydrogen. In some embodiments, R⁴ and R^{4′} are hydrogen.

In some embodiments, R^{4′} is hydrogen, and R⁴ is halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur. In some embodiments, R^{4′} is hydrogen, and R⁴ is halogen. In some embodiments, R^{4′} is hydrogen, and R⁴ is —CN. In some embodiments, R^{4′} is hydrogen, and R⁴ is —OR. In some embodiments, R^{4′} is hydrogen, and R⁴ is —N(R)₂. In some embodiments, R^{4′} is hydrogen, and R⁴ is —S(O)₂R.

In some embodiments, R^{4′} is hydrogen, and R⁴ is an optionally substituted group selected from the group consisting of C₁₋aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, and an 8- to 12-membered bicyclic heteroaryl having 1-4 heteroatoms independently selected from oxygen, nitrogen or sulfur. In some embodiments, R^{4′} is hydrogen, and R⁴ is an optionally substituted group selected from the group consisting of C₁₋aliphatic, a 3- to 7-membered saturated or partially unsaturated monocyclic carbocycyl, or phenyl. In some embodiments, R^{4′} is hydrogen, and R⁴ is an optionally substituted group selected from the group consisting of C₁₋aliphatic or phenyl. In some embodiments, R^{4′} is hydrogen, and R⁴ is C₁₋₆ aliphatic or phenyl. In some embodiments, R^{4′} is hydrogen, and R⁴ is methyl or phenyl.

In some embodiments, R^{4′} is hydrogen, and R⁴ is an optionally substituted C₁₋₆ aliphatic. In some embodiments, R^{4′} is hydrogen, and R⁴ is C₁₋₆ aliphatic. In some embodiments, R^{4′} is hydrogen, and R⁴ is methyl. In some embodiments, R^{4′} is hydrogen, and R⁴ is ethyl. In some embodiments, R^{4′} is hydrogen, and R⁴ is n-propyl. In some embodiments, R^{4′} is hydrogen, and R⁴ is isopropyl. In some embodiments, R^{4′} is hydrogen, and R⁴ is C₁₋₆ aliphatic, optionally substituted with —(CH₂)₀₋₄OR^{◯}, wherein R^{◯} is hydrogen, or C₁₋₆ aliphatic. In some embodiments, R^{4′} is hydrogen, and R⁴ is a 3- to 7-membered saturated or partially unsaturated monocyclic carbocycyl. In some embodiments, R^{4′} is hydrogen, and R⁴ is cyclopropyl. In some embodiments, R^{4′} is hydrogen, and R⁴ is a 3- to 7-membered saturated or partially unsaturated monocyclic heterocyclyl having 1-2 heteroatoms independently selected from oxygen, nitrogen, or sulfur. In some embodiments, R^{4′} is hydrogen, and R⁴ is optionally substituted phenyl. In some embodiments, R^{4′} is hydrogen, and R⁴ is phenyl. In some embodiments, R^{4′} is hydrogen, and R⁴ is a 5- to 6-membered monocyclic heteroaryl having 1-3 heteroatoms independently selected from oxygen, nitrogen, or sulfur. In some embodiments, R^{4′} is hydrogen, and R⁴ is an 8- to 10-membered bicyclic aryl. In some embodiments, R^{4′} is hydrogen, and R⁴ is a 7- to 12-member saturated or partially unsaturated bicyclic heterocyclyl having 1-4 heteroatoms independently selected from oxygen, nitrogen, or sulfur. In some embodiments, R^{4′} is hydrogen, and R⁴ is an 8- to 12-membered bicyclic heteroaryl having 1-4 heteroatoms independently selected from oxygen, nitrogen or sulfur.

In some embodiments, R^{4′} is hydrogen, and R⁴ is hydrogen, methyl, isopropyl, phenyl, cyclopropyl, or

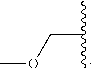 [SMILES: *COC]

As defined above and described herein, L is a covalent bond or an optionally substituted C₁₋₃ hydrocarbon chain, wherein 1-3 methylene units are optionally and independently replaced with —O—, —C(O)—, —S(O)₂—, or —NR—. In some embodiments, L is an optionally substituted C₁₋₃ hydrocarbon chain, wherein 1-3 methylene units are optionally and independently replaced with —O—, —C(O)—, or —S(O)₂—. In some embodiments, L is an optionally substituted C₁₋₃ hydrocarbon chain, wherein 1 methylene unit is replaced with —C(O)—. In some embodiments, L is a covalent bond, —C(O)—, —C(O)CH₂—*, —C(O)CH₂CH₂—*, —C(O)O—*, —C(O)CH₂O—*, —CH₂—, —S(O)₂—, or —C(O)CH₂C(O)—, wherein * represents the point of attachment to R⁵. In some embodiments, L is —C(O)—, —C(O)CH₂—*, —C(O)CH₂CH₂—*, —C(O)O—*, —C(O)CH₂O—*, —CH₂—, —S(O)₂—, or —C(O)CH₂C(O)—, wherein * represents the point of attachment to R⁵. In some embodiments, L is —C(O)—, —CH₂—, or —S(O)₂—, wherein * represents the point of attachment to R⁵. In some embodiments, L is —C(O)—. In some embodiments, L is —C(O)CH₂—*, wherein * represents the point of attachment to R⁵. In some embodiments, L is —C(O)CH₂CH₂—*, wherein * represents the point of attachment to R⁵. In some embodiments, L is —C(O)O—*, wherein * represents the point of attachment to R⁵. In some embodiments, L is —C(O)CH₂O—*, wherein * represents the point of attachment to R⁵. In some embodiments, L is —CH₂—. In some embodiments, L is —S(O)₂—. In some embodiments, L is a covalent bond. In some embodiments, L is —C(O)CH₂C(O)—.

As defined generally above, R⁵ is hydrogen, halogen, —CN, —OR, —N(R)₂, —S(O)₂R, or an optionally substituted group selected from the group consisting of C₁₋aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur. In some embodiments, R⁵ is hydrogen. In some embodiments, R⁵ is halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, a saturated or partially unsaturated 7- to 12-member bicyclic heterocyclyl having 1-4 heteroatoms independently selected from oxygen, nitrogen, or sulfur, an 8- to 10-membered bicyclic aryl, and an 8- to 12-membered bicyclic heteroaryl having 1-4 heteroatoms independently selected from oxygen, nitrogen or sulfur.

In some embodiments, R⁵ is —CN. In some embodiments, R⁵ is —OR. In some embodiments, R⁵ is —OH. In some embodiments, R⁵ is —OMe. In some embodiments, R⁵ is —N(R)₂. In some embodiments, R⁵ is —NH₂. In some embodiments, R⁵ is —N(CH₃)₂. In some embodiments, R⁵ is —NHCH₃. In some embodiments, R⁵ is —S(O)₂R. In some embodiments, R⁵ is —S(O)₂CH₃. In some embodiments, R⁵ is optionally substituted C₁₋₆ aliphatic. In some embodiments, R⁵ is C₁₋₆ aliphatic, optionally substituted with halogen. In some embodiments, R⁵ is C₁₋₆ aliphatic, optionally substituted with fluoro. In some embodiments, R⁵ is —CF₂CH₃. In some embodiments, R⁵ is —CF₃. In some embodiments, R⁵ is methyl, ethyl, n-propyl, isopropyl, n-butyl, s-butyl, or t-butyl. In some embodiments, R⁵ is methyl, isopropyl, or t-butyl. In some embodiments, R⁵ is C₁₋₆ aliphatic optionally substituted with halogen (e.g., fluoro), —(CH₂)₀₋₄OR^{◯}, or —(CH₂)₀₋₄N(R^{◯})₂, wherein R^{◯} is hydrogen or C₁₋₆ aliphatic. In some embodiments, R⁵ is C₁₋₃ aliphatic optionally substituted with halogen (e.g., fluoro), —(CH₂)₀₋₄OR^{◯}, or —(CH₂)₀₋₄N(R^{◯})₂, wherein R^{◯} is hydrogen or C₁₋₆ aliphatic. In some embodiments, R⁵ is C₁₋₃ aliphatic optionally substituted with fluoro, —OH, or —NH₂. In some embodiments, R⁵ is —C(CH₃)OH. In some embodiments, R⁵ is —C(CH₃)NH₂. In some embodiments, R⁵ is —C(CH₃)F.

In some embodiments, R⁵ is an optionally substituted group selected from the group consisting of a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, and a 5- to 6-membered monocyclic heteroaryl having 1-3 heteroatoms independently selected from oxygen, nitrogen, or sulfur. In some embodiments, R⁵ is an optionally substituted 3- to 7-membered saturated or partially unsaturated monocyclic carbocycyl. In some embodiments, R⁵ is an optionally substituted cyclopropyl. In some embodiments, R⁵ is cyclopropyl optionally substituted with —(CH₂)₀₋₄OR^{◯}, wherein R^{◯} is hydrogen or C₁₋₆ aliphatic. In some embodiments, R⁵ is

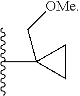 [SMILES: *C1(COC)CC1]

In some embodiments, R⁵ is

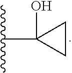 [SMILES: *C1(O)CC1]

In some embodiments, R⁵ is cyclopropyl, optionally substituted with —CN. In some embodiments, R⁵ is

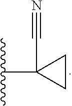 [SMILES: *C1(C#N)CC1]

In some embodiments, R⁵ is an optionally substituted cyclobutyl.

In some embodiments, R⁵ is an optionally substituted 3- to 7-membered saturated or partially unsaturated monocyclic heterocyclyl having 1-2 heteroatoms independently selected from oxygen, nitrogen, or sulfur. In some embodiments, R⁵ is an optionally substituted 4- to 6-membered saturated or partially unsaturated monocyclic heterocyclyl having 1-2 heteroatoms independently selected from oxygen, nitrogen, or sulfur.

In some embodiments, R⁵ is an optionally substituted 4- to 6-membered saturated or partially unsaturated monocyclic heterocyclyl having 1 oxygen heteroatom. In some embodiments, R⁵ is optionally substituted oxetanyl. In some embodiments, R⁵ is oxetanyl, optionally substituted with —(CH₂)₀₋₄R^{◯} or —(CH₂)₀₋₄OR^{◯}, wherein R^{◯} is hydrogen or C₁₋aliphatic. In some embodiments, R⁵ is optionally substituted tetrahydrofuranyl. In some embodiments, R⁵ is optionally substituted

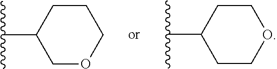 [SMILES: *C1CCCOC1 ; *C1CCOCC1]

In some embodiments, R⁵ is tetrahydrofuranyl optionally substituted with —(CH₂)₀₋₄R^{◯}, wherein R^{◯} is hydrogen or C₁₋aliphatic. In some embodiments, R⁵ is tetrahydrofuranyl. In some embodiments, R⁵ is optionally substituted tetrahydropyranyl. In some embodiments, R⁵ is tetrahydropyranyl optionally substituted with —(CH₂)₀₋₄R^{◯}, wherein R^{◯} is hydrogen or C₁₋aliphatic. In some embodiments, R⁵ is tetrahydropyranyl. In some embodiments, R⁵ is optionally substituted

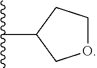 [SMILES: *C1CCOC1]

In some embodiments, R⁵ is an optionally substituted 4- to 6-membered saturated or partially unsaturated monocyclic heterocyclyl having 1 nitrogen heteroatom. In some embodiments, R⁵ is optionally substituted azetidinyl. In some embodiments, R⁵ is

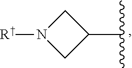

wherein R^{†} is C₁₋₄ aliphatic (e.g., methyl). In some embodiments, R⁵ is azetidinyl, optionally substituted with —(CH₂)₀₋₄R^{◯} or —(CH₂)₀₋₄OR^{◯}, wherein R^{◯} is hydrogen or C₁₋₆ aliphatic. In some embodiments, R⁵ is azetidinyl, substituted with —CH₃. In some embodiments, R⁵ is optionally substituted azetidinonyl. In some embodiments, R⁵ is

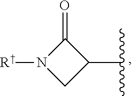

wherein R^{†} is C₁₋₄ aliphatic (e.g., methyl). In some embodiments, R⁵ is azetidinonyl, optionally substituted with —(CH₂)₀₋₄R^{◯} or —(CH₂)₀₋₄OR^{◯}, wherein R^{◯} is hydrogen or C₁₋₆ aliphatic. In some embodiments, R⁵ is azetidinonyl, substituted with —CH₃.

In some embodiments, R⁵ is optionally substituted pyrrolidinyl. In some embodiments, R⁵ is pyrrolidinyl, wherein one or more carbon atoms are optionally substituted with one or more ═O or —(CH₂)₀₋₄R^{◯}, and one or more nitrogen atoms are optionally substituted with —R^{†}, wherein each —R^{†} is independently C₁₋₄ aliphatic (e.g., methyl). In some embodiments, R⁵ is optionally substituted pyrrolidonyl. In some embodiments, R⁵ is pyrrolidonyl, wherein one or more carbon atoms are optionally substituted with one or more-(CH₂)₀₋₄R^{◯}, and one or more nitrogen atoms are optionally substituted with —R^{†}, wherein each —R^{†} is independently C₁₋₄ aliphatic (e.g. methyl). In some embodiments R⁵ is:

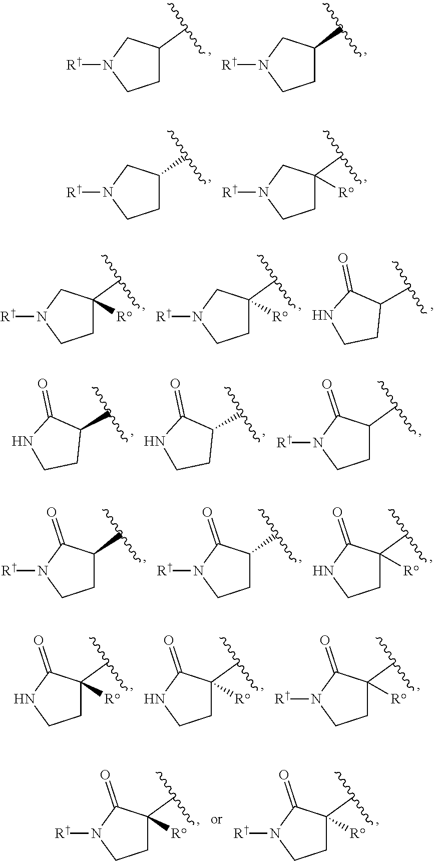

wherein R^{†} is C₁₋₄ aliphatic (e.g., methyl), and R^{◯} is C₁₋₆ aliphatic (e.g., methyl).

In some embodiments, R⁵ is optionally substituted piperidinyl. In some embodiments, R⁵ is piperidinyl, wherein one or more carbon atoms are optionally substituted with one or more halogen (e.g., fluoro), —CN, —(CH₂)₀₋₄R^{◯}, or —(CH₂)₀₋₄OR^{◯}; and one or more nitrogen atoms are optionally substituted with —R^{†}— or —C(O)R^{†}. In some embodiments, R⁵ is piperidinyl, wherein one or more carbon atoms are optionally substituted with one or more ═O, halogen (e.g., fluoro), —CN, —(CH₂)₀₋₄R^{◯}, or —(CH₂)₀₋₄OR^{◯}, wherein each R^{◯} is independently C₁₋₆ aliphatic optionally substituted with halogen (e.g., fluoro); and one or more nitrogen atoms are optionally substituted with —R^{†}— or —C(O)R^{†}, wherein each —R is independently C₁₋₆ aliphatic. In some embodiments, R⁵ is piperidinyl optionally substituted with one or more methyl, ethyl, isopropyl, —C(O)CH₃, —CN, fluoro, —CH₂CH₂OCH₃, —CHF₂, or ═O. In some embodiments, R⁵ is optionally substituted piperidinyl. In some embodiments, R⁵ is piperidonyl, wherein one or more carbon atoms are optionally substituted with one or more halogen (e.g., fluoro), —CN, —(CH₂)₀₋₄R^{◯}, or —(CH₂)₀₋₄OR^{◯}; and one or more nitrogen atoms are optionally substituted with —R^{†}— or —C(O)R^{†}. In some embodiments, R⁵ is piperidonyl, wherein one or more carbon atoms are optionally substituted with one or more halogen (e.g., fluoro), —CN, —(CH₂)₀₋₄R^{◯}, or —(CH₂)₀₋₄OR^{◯}, wherein each R^{◯} is independently C₁₋₆ aliphatic optionally substituted with halogen (e.g., fluoro); and one or more nitrogen atoms are optionally substituted with —R^{†}— or —C(O)R^{†}, wherein each —R^{†} is independently C₁₋₆ aliphatic. In some embodiments, R⁵ is piperidonyl optionally substituted with one or more methyl, ethyl, isopropyl, —C(O)CH₃, —CN, fluoro, —CH₂CH₂OCH₃, or —CHF₂. In some embodiments R⁵ is:

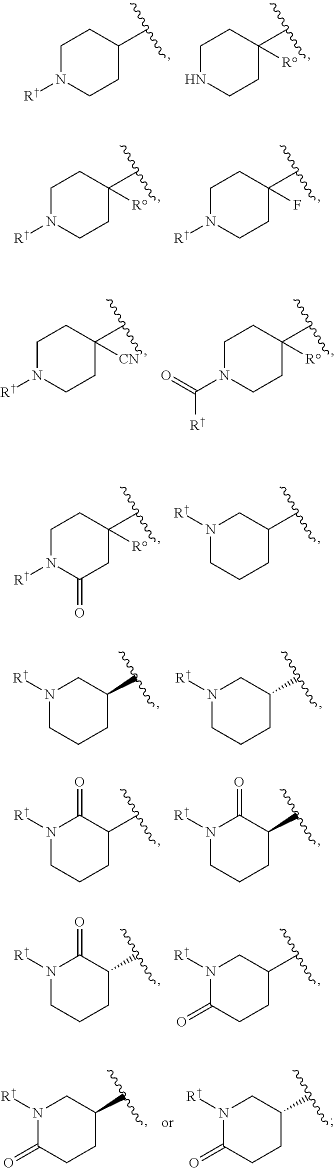

wherein R^{†} is C₁₋₄ aliphatic (e.g., methyl, ethyl, isopropyl), and R^{◯} is C₁₋₆ aliphatic (e.g., methyl, ethyl, isopropyl) optionally substituted with halogen (e.g., fluoro).

In some embodiments, R⁵ is

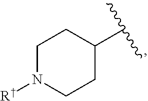

wherein R^{†} is C₁₋₄ aliphatic (e.g., methyl). In some embodiments, R⁵ is

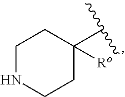

wherein R^{◯} is C₁₋₆ aliphatic (e.g., methyl). In some embodiments, R⁵ is

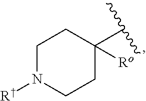

wherein R^{†} is C₁₋₄ aliphatic (e.g., methyl, ethyl, isopropyl), and R^{◯} is halogen (e.g., fluoro), —CN or C₁₋₆ aliphatic (e.g., methyl) optionally substituted with halogen (e.g., fluoro). In some embodiments, R⁵ is

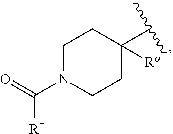

wherein R^{†} is C₁₋₄ aliphatic (e.g., methyl), and R^{◯} is C₁₋₆ aliphatic (e.g., methyl). In some embodiments, R⁵ is

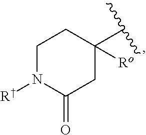

R^{†} is C₁₋₄ aliphatic (e.g., methyl), and R^{◯} is C₁₋aliphatic (e.g., methyl). In some embodiments, R⁵ is

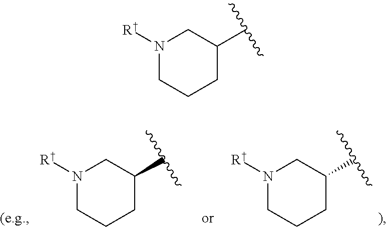

wherein R^{†} is C₁₋₄ aliphatic (e.g., methyl). In some embodiments, R⁵ is

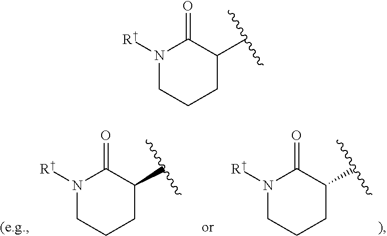

wherein R^{†} is C₁₋₄ aliphatic (e.g., methyl). In some embodiments, R⁵ is

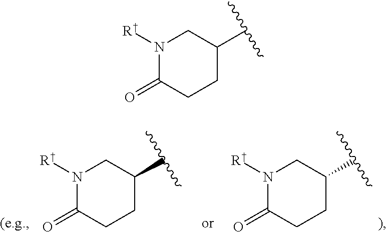

wherein R^{†} is C₁₋₄ aliphatic (e.g., methyl).

In some embodiments, R⁵ is an optionally substituted 4- to 6-membered saturated or partially unsaturated monocyclic heterocyclyl having 1 sulfur heteroatom. In some embodiments, R⁵ is an optionally substituted 4-membered saturated or partially unsaturated monocyclic heterocyclyl having 1 sulfur heteroatom. In some embodiments, R⁵ is dioxidothietanyl.

In some embodiments, R⁵ is optionally substituted phenyl. In some embodiments, R⁵ is phenyl optionally substituted with —(CH₂)₀₋₄OR^{◯}, wherein R^{◯} is hydrogen or C₁₋₆ aliphatic. In some embodiments, R⁵ is phenyl optionally substituted with —(CH₂)₀₋₄C(O)NR^{◯}₂, where each R^{◯} is independently hydrogen or C₁₋₆ aliphatic (e.g., methyl). In some embodiments, R⁵ is phenyl optionally substituted with —C(O)NHCH₃. In some embodiments, R⁵ is phenyl optionally substituted with —(CH₂)₀₋₄OR^{◯}, wherein R^{◯} is hydrogen or C₁₋₆ aliphatic (e.g., methyl). In some embodiments, R⁵ is phenyl optionally substituted with —OMe.

In some embodiments, R⁵ is optionally substituted 5- to 6-membered monocyclic heteroaryl having 1-3 heteroatoms independently selected from oxygen, nitrogen, or sulfur. In some embodiments, R⁵ is optionally substituted 5-membered monocyclic heteroaryl having 1-3 heteroatoms independently selected from oxygen, nitrogen, or sulfur. In some embodiments, R⁵ is an optionally substituted 5-membered monocyclic heteroaryl having one oxygen heteroatom. In some embodiments, R⁵ is optionally substituted furanyl. In some embodiments, R⁵ is an optionally substituted 5-membered monocyclic heteroaryl having one oxygen and one nitrogen heteroatom. In some embodiments, R⁵ is optionally substituted isoxazolyl. In some embodiments, R⁵ is isoxazolyl, optionally substituted with —(CH₂)₀₋₄R^{◯}, wherein R^{◯} is hydrogen or C₁₋₆ aliphatic (e.g., methyl). In some embodiments, R⁵ is an optionally substituted 5-membered monocyclic heteroaryl having one sulfur and one nitrogen heteroatom. In some embodiments, R⁵ is an optionally substituted thiazolyl. In some embodiments, R⁵ is an optionally substituted 5-membered monocyclic heteroaryl having one sulfur and two nitrogen heteroatoms. In some embodiments, R⁵ is an optionally substituted thiadiazolyl.

In some embodiments, R⁵ is an optionally substituted 6-membered monocyclic heteroaryl having 1-3 heteroatoms independently selected from oxygen, nitrogen, or sulfur. In some embodiments, R⁵ is optionally substituted 6-membered monocyclic heteroaryl having 1 nitrogen heteroatom. In some embodiments, R⁵ is optionally substituted pyridinyl. In some embodiments, R⁵ is pyridinyl, optionally substituted with —(CH₂)₀₋₄R^{◯}, wherein R^{◯} is a 5- to 6-membered saturated, partially unsaturated, or aryl ring having 0-4 heteroatoms independently selected from nitrogen, oxygen, or sulfur. In some embodiments, R⁵ is pyridinyl, optionally substituted with —(CH₂)₀₋₄R^{◯}, wherein R^{◯} is a 5- to 6-membered saturated or partially unsaturated ring having 1-3 heteroatoms independently selected from nitrogen, oxygen, or sulfur. In some embodiments, R⁵ is pyridinyl optionally substituted with morpholine. In some embodiments, R⁵ is pyridinyl.

In some embodiments, R⁵ is an optionally substituted 8- to 10-membered bicyclic aryl. In some embodiments, R⁵ is an optionally substituted 10-membered bicyclic aryl. In some embodiments, R⁵ is an optionally substituted 10-membered bicyclic aryl, wherein an aromatic ring is fused to one or more non-aromatic rings. In some embodiments, R⁵ is a 10-membered bicyclic aryl, optionally substituted with ═O. In some embodiments, R⁵ is an optionally substituted benzo[b][1,4]oxazinonyl.

In some embodiments, R⁵ is an optionally substituted 7-12-membered saturated or partially unsaturated bicyclic heterocyclyl having 1-4 heteroatoms independently selected from oxygen, nitrogen, or sulfur. In some embodiments, R⁵ is an optionally substituted 8-membered saturated or partially unsaturated bicyclic heterocyclyl having one nitrogen. In some embodiments, R⁵ is

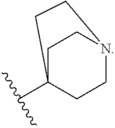 [SMILES: *C12CCN(CC1)CC2]

In some embodiments, In some embodiments, R⁵ is an optionally substituted 8-membered saturated or partially unsaturated bicyclic heterocyclyl having 1-4 nitrogen heteroatoms. In some embodiments, In some embodiments, R⁵ is an optionally substituted triazolopyridinyl.

In some embodiments, R⁵ is an optionally substituted 8- to 12-membered bicyclic heteroaryl having 1-4 heteroatoms independently selected from oxygen, nitrogen or sulfur. In some embodiments, R⁵ is an optionally substituted 8-membered bicyclic heteroaryl having 1-4 heteroatoms independently selected from oxygen, nitrogen or sulfur. In some embodiment, R⁵ is an optionally substituted 8- to 12-membered bicyclic heteroaryl having two nitrogen heteroatoms. In some embodiment, R⁵ is an optionally substituted 9-membered bicyclic heteroaryl having two nitrogen heteroatoms. In some embodiments, R⁵ is an optionally substituted benzoimidazolyl. In some embodiments, R⁵ is an optionally substituted pyrazolopyridinyl. In some embodiment, R⁵ is an optionally substituted 8- to 12-membered bicyclic heteroaryl having two nitrogen heteroatoms and one oxygen heteroatom. In some embodiment, R⁵ is an optionally substituted 9-membered bicyclic heteroaryl having two nitrogen heteroatoms and one oxygen heteroatom. In some embodiments, R⁵ is an optionally substituted pyrrolooxazolyl. In some embodiment, R⁵ is an optionally substituted 8- to 12-membered bicyclic heteroaryl having three nitrogen heteroatoms. In some embodiment, R⁵ is an optionally substituted 9-membered bicyclic heteroaryl having three nitrogen heteroatoms. In some embodiments, R⁵ is an optionally substituted pyrazolopyrimidinyl.

In some embodiments, R⁵ is hydrogen, —CN, —CH₃, —CF₃, —OH, —OMe, —C(CH₃)₂OH, C(CH₃)₂NH₂, —C(CH₃)₂F, —NH₂, —NHCH₃, —N(CH₃)₂, isopropyl, t-butyl, —S(O)₂CH₃,

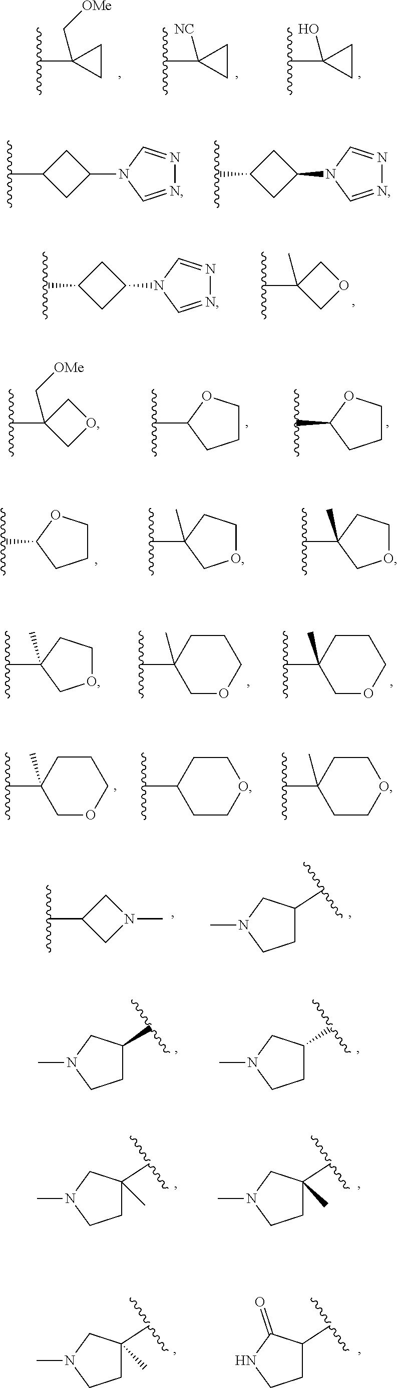

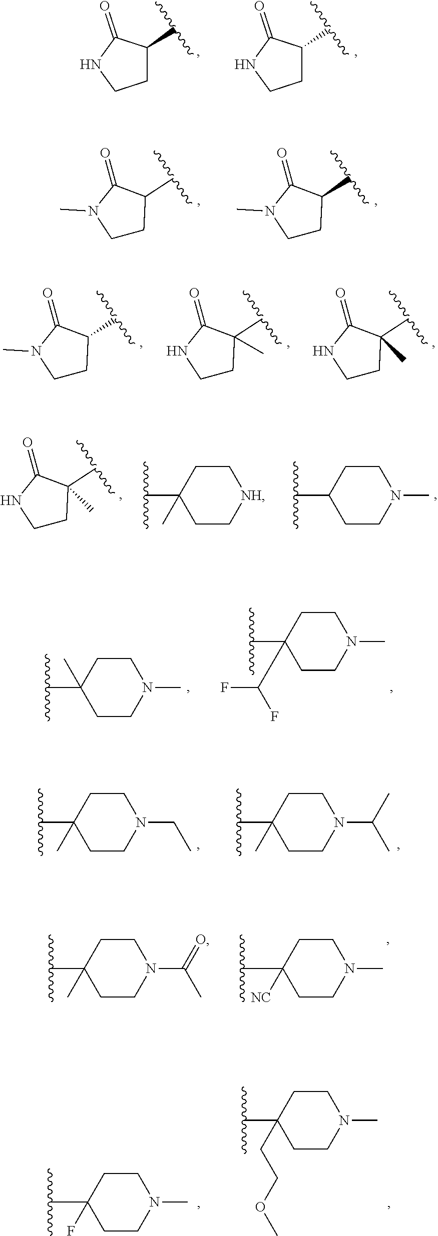

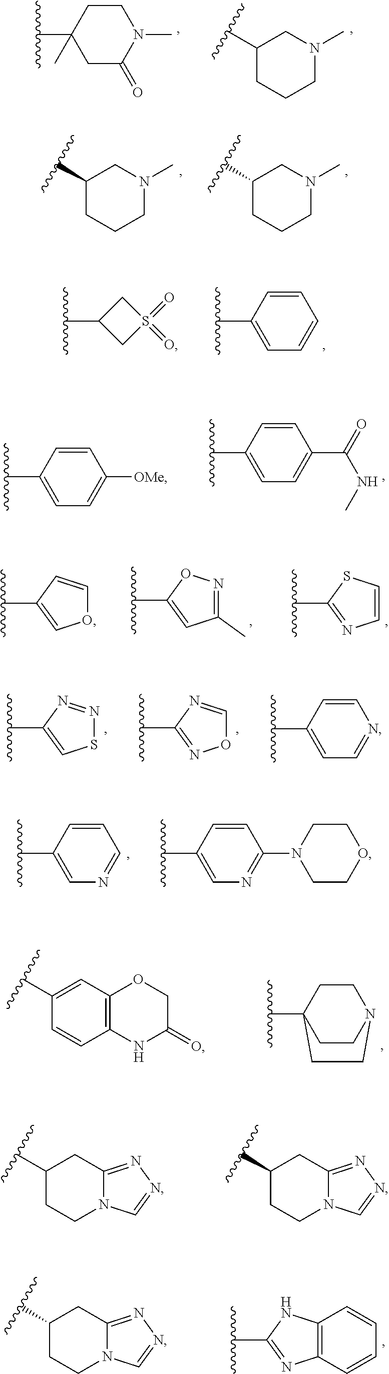

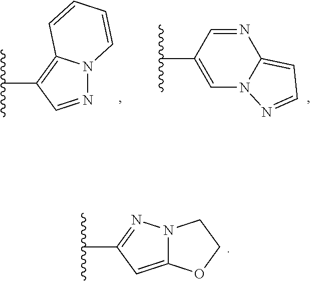 [SMILES: *c1cnn2ccccc12 ; *c1cnc2ccnn2c1 ; *c1cc2n(n1)CCO2]

In some embodiments, -L-R⁵ is:

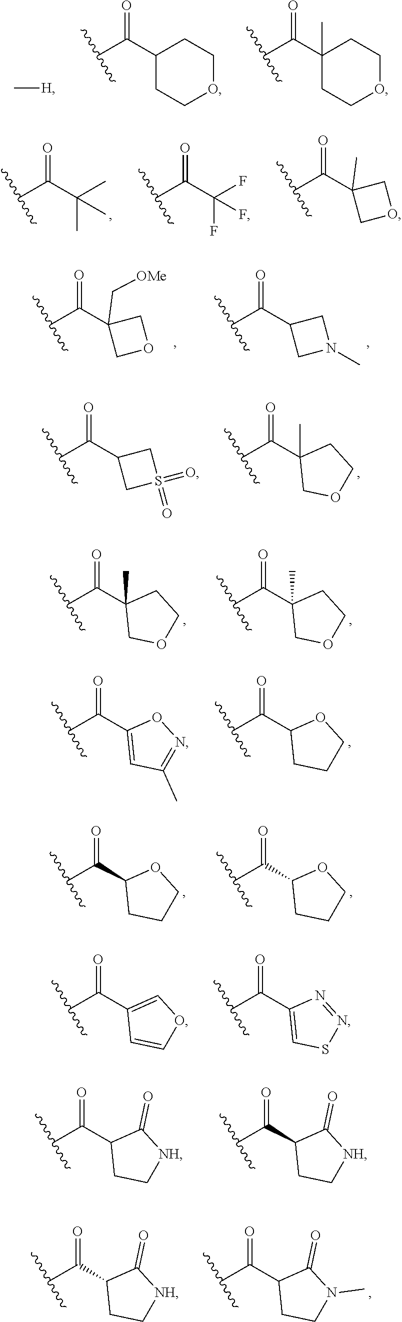

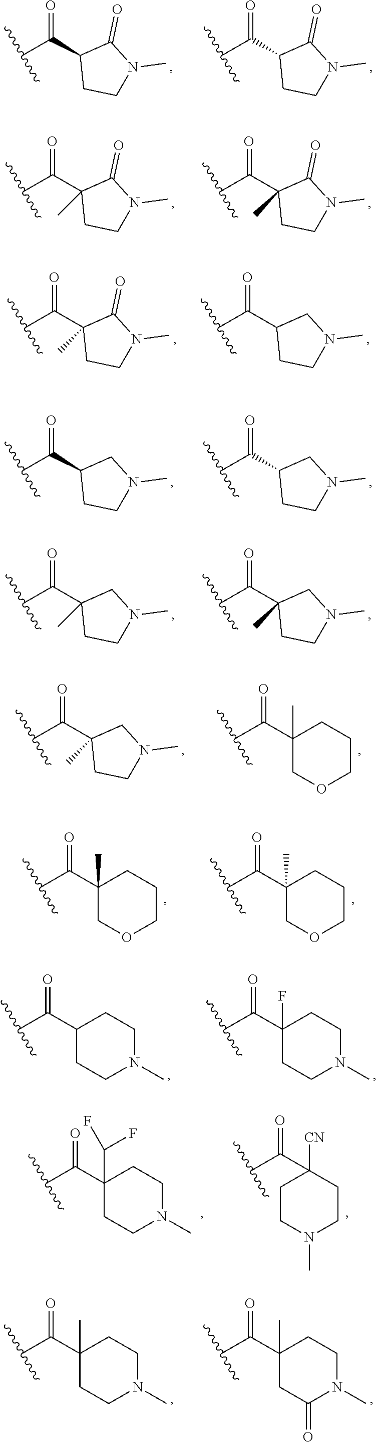

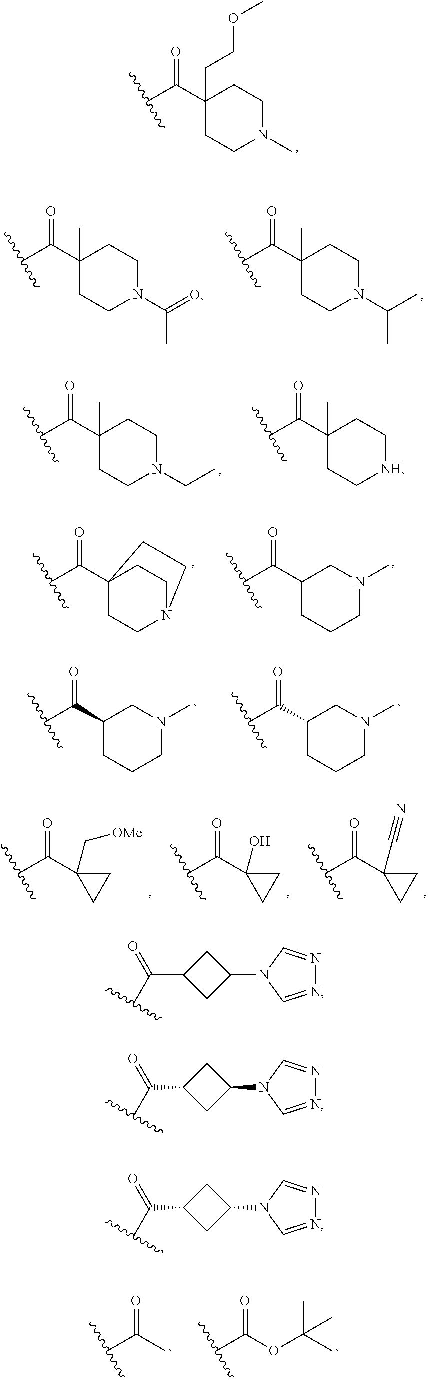

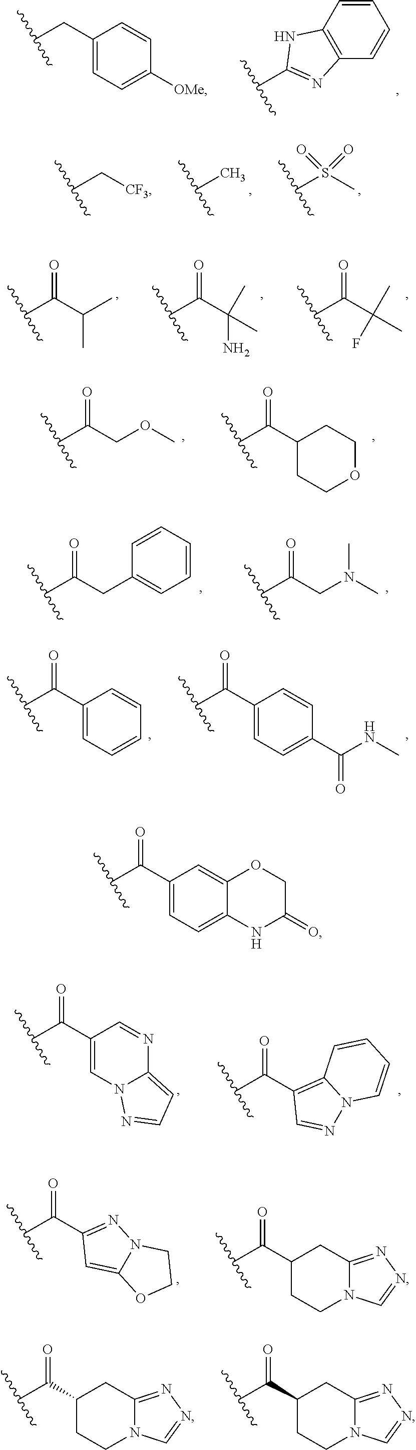

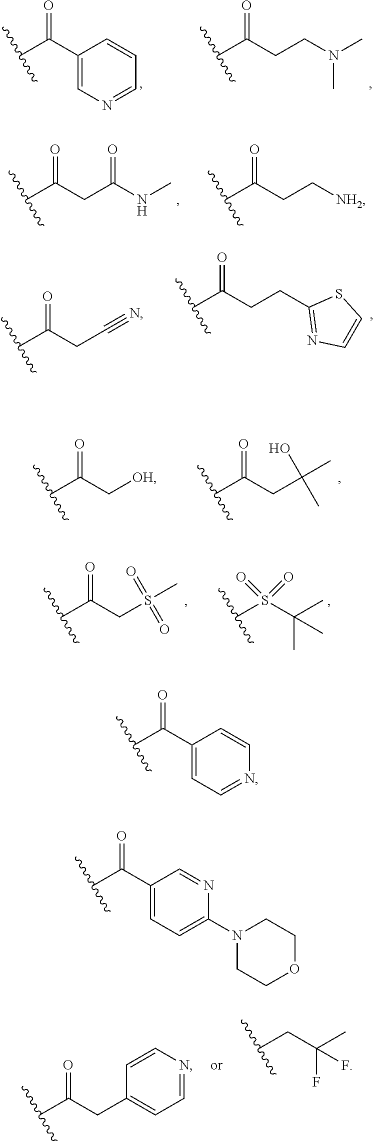

In some embodiments, a provided compound is of Formula II:

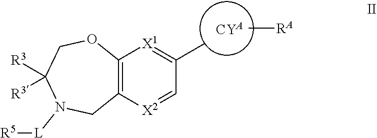

or a pharmaceutically acceptable salt thereof,
wherein each of Cy^{A}, R^{A}, X¹, X², R³, R^{3′}, L, and R⁵ is defined and described in classes and subclasses wherein, both singly and in combination.

It will be understood that, unless otherwise specified or prohibited by the foregoing definition of Formula II, embodiments of variables Cy^{A}, R^{A}, X, X², R³, R^{3′}, L, and R⁵ as defined above and described in classes and subclasses herein, also apply to compounds of Formula II, both singly and in combination.

In some embodiments, a provided compound is of Formula II-a or II-b:

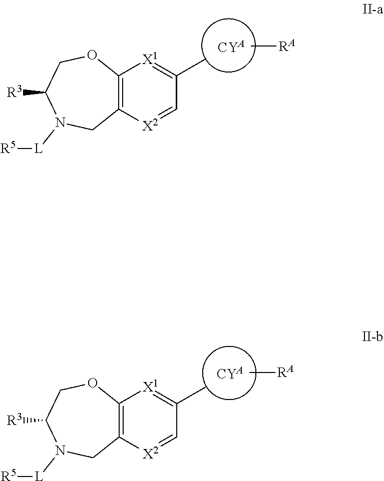

or a pharmaceutically acceptable salt thereof,
wherein each of Cy^{A}, R^{A}, X¹, X², R³, L, and R⁵ is defined and described in classes and subclasses wherein, both singly and in combination.

It will be understood that, unless otherwise specified or prohibited by the foregoing definition of Formula II-a or II-b, embodiments of variables Cy^{A}, R^{A}, X¹, X², R³, L, and R⁵ as defined above and described in classes and subclasses herein, also apply to compounds of Formula II-a or II-b, both singly and in combination.

In some embodiments, a provided compound is of Formula III-a or III-b:

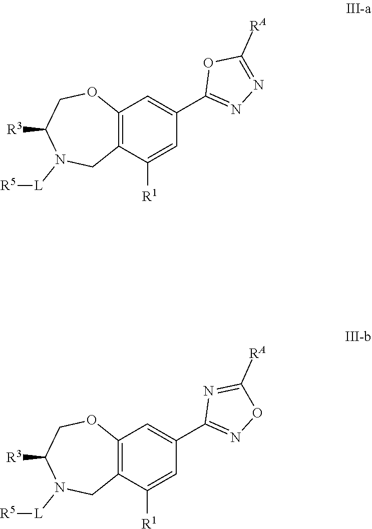

or a pharmaceutically acceptable salt thereof,
wherein each of R^{A}, R¹, R³, L, and R⁵ is defined and described in classes and subclasses wherein, both singly and in combination.

It will be understood that, unless otherwise specified or prohibited by the foregoing definition of Formula III-a or III-b, embodiments of variables R^{A}, R¹, R³, L, and R⁵ as defined above and described in classes and subclasses herein, also apply to compounds of Formula III-a or III-b, both singly and in combination.

In some embodiments, a provided compound is of Formula IV-a, IV-b, IV-c, or IV-d:

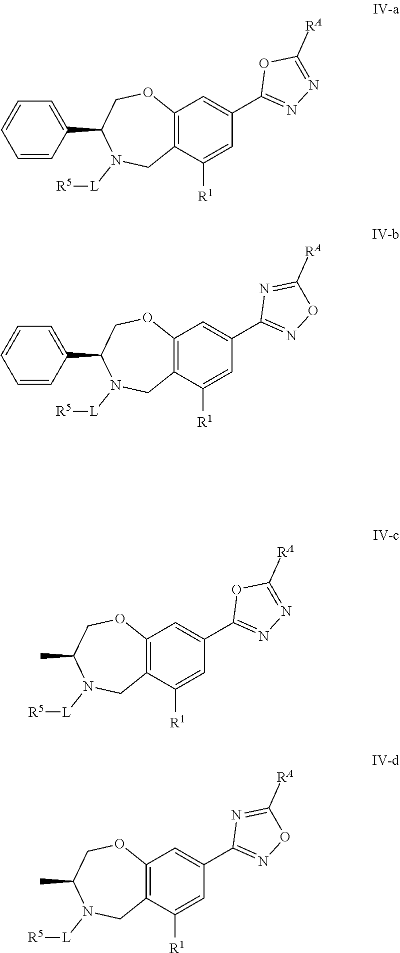

or a pharmaceutically acceptable salt thereof,
wherein each of R^{A}, R¹, L, and R⁵ is defined and described in classes and subclasses wherein, both singly and in combination.

It will be understood that, unless otherwise specified or prohibited by the foregoing definition of Formula IV-a, IV-b, IV-c, or IV-d, embodiments of variables R^{A}, R¹, L, and R⁵ as defined above and described in classes and subclasses herein, also apply to compounds of Formula IV-a, IV-b, IV-c, or IV-d, both singly and in combination.

In some embodiments, a provided compound is of Formula V:

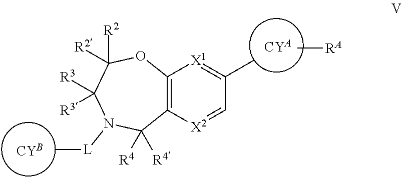

or a pharmaceutically acceptable salt thereof,
wherein:
Cy^{B} is an optionally substituted 3- to 7-membered saturated or partially unsaturated monocyclic heterocyclyl having 1-2 heteroatoms independently selected from oxygen, nitrogen, or sulfur, 5- to 6-membered monocyclic heteroaryl having 1-3 heteroatoms independently selected from oxygen, nitrogen, or sulfur, a 7- to 12-member saturated or partially unsaturated bicyclic heterocyclyl having 1-4 heteroatoms independently selected from oxygen, nitrogen, or sulfur, or an 8- to 12-membered bicyclic heteroaryl having 1-4 heteroatoms independently selected from oxygen, nitrogen or sulfur; and
   wherein Cy^{B} comprises at least one oxygen or nitrogen heteroatom; and
each of R^{A}, Cy^{A}, X¹, X², R², R^{2′}, R³, R^{3′}, R⁴, R^{4′}, L, and R⁵ is defined and described in classes and subclasses wherein, both singly and in combination.

It will be understood that, unless otherwise specified or prohibited by the foregoing definition of Formula V, embodiments of variables R^{A}, Cy^{A}, X, X², R², R^{2′}, R³, R^{3′}, R⁴, R^{4′}, L, and R⁵ as defined above and described in classes and subclasses herein, also apply to compounds of Formula V, both singly and in combination.

As generally defined above, Cy^{B} is an optionally substituted 3- to 7-membered saturated or partially unsaturated monocyclic heterocyclyl having 1-2 heteroatoms independently selected from oxygen, nitrogen, or sulfur, 5- to 6-membered monocyclic heteroaryl having 1-3 heteroatoms independently selected from oxygen, nitrogen, or sulfur, a 7- to 12-member saturated or partially unsaturated bicyclic heterocyclyl having 1-4 heteroatoms independently selected from oxygen, nitrogen, or sulfur, or an 8- to 12-membered bicyclic heteroaryl having 1-4 heteroatoms independently selected from oxygen, nitrogen or sulfur, and wherein Cy^{B} comprises at least one oxygen or nitrogen heteroatom. In some embodiments, Cy^{B} is an optionally substituted 3- to 7-membered saturated or partially unsaturated monocyclic heterocyclyl having 1-2 heteroatoms independently selected from oxygen, nitrogen, or sulfur, 5- to 6-membered monocyclic heteroaryl having 1-3 heteroatoms independently selected from oxygen, nitrogen, or sulfur, a 7- to 12-member saturated or partially unsaturated bicyclic heterocyclyl having 1-4 heteroatoms independently selected from oxygen, nitrogen, or sulfur, or an 8- to 12-membered bicyclic heteroaryl having 1-4 heteroatoms independently selected from oxygen, nitrogen or sulfur, and wherein Cy^{B} comprises at least one oxygen or nitrogen heteroatom.

In some embodiments, Cy^{B} is an optionally substituted 3- to 7-membered saturated or partially unsaturated monocyclic heterocyclyl having 1-2 heteroatoms independently selected from oxygen, nitrogen, or sulfur, and wherein Cy^{B} comprises at least one oxygen or nitrogen heteroatom. In some embodiments, Cy^{B} is an optionally substituted 4- to 6-membered saturated or partially unsaturated monocyclic heterocyclyl having 1-2 heteroatoms independently selected from oxygen, nitrogen, or sulfur, and wherein Cy^{B} comprises at least one oxygen or nitrogen heteroatom.

In some embodiments, Cy^{B} is an optionally substituted 4- to 6-membered saturated or partially unsaturated monocyclic heterocyclyl having 1 oxygen heteroatom. In some embodiments, Cy^{B} is optionally substituted oxetanyl. In some embodiments, Cy^{B} is oxetanyl, optionally substituted with —(CH₂)₀₋₄R^{◯} or —(CH₂)₀₋₄OR^{◯}, wherein R^{◯} is hydrogen or C₁₋aliphatic. In some embodiments, Cy^{B} is optionally substituted tetrahydrofuranyl. In some embodiments, Cy^{B} is tetrahydrofuranyl optionally substituted with —(CH₂)₀₋₄R^{◯}, wherein R^{◯} is hydrogen or C₁₋₆ aliphatic. In some embodiments, Cy^{B} is tetrahydrofuranyl. In some embodiments, Cy^{B} is optionally substituted

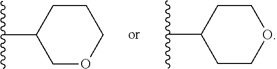 [SMILES: *C1CCCOC1 ; *C1CCOCC1]

In some embodiments, Cy^{B} is optionally substituted tetrahydropyranyl. In some embodiments, Cy^{B} is tetrahydropyranyl optionally substituted with —(CH₂)₀₋₄R^{◯}, wherein R^{◯} is hydrogen or C₁₋aliphatic. In some embodiments, Cy^{B} is tetrahydropyranyl. In some embodiments, Cy^{B} is optionally substituted

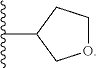 [SMILES: *C1CCOC1]

In some embodiments, Cy^{B} is an optionally substituted 4- to 6-membered saturated or partially unsaturated monocyclic heterocyclyl having 1 nitrogen heteroatom. In some embodiments, Cy^{B} is optionally substituted azetidinyl. In some embodiments, Cy^{B} is

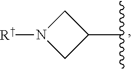

wherein R^{†} is C₁₋₄ aliphatic (e.g., methyl). In some embodiments, Cy^{B} is azetidinyl, optionally substituted with —(CH₂)₀₋₄R^{◯} or —(CH₂)₀₋₄OR^{◯}, wherein R^{◯} is hydrogen or C₁₋aliphatic. In some embodiments, Cy^{B} is azetidinyl, substituted with —CH₃. In some embodiments, Cy^{B} is optionally substituted azetidinonyl. In some embodiments, R⁵ is

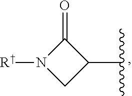

wherein R^{†} is C₁₋₄ aliphatic (e.g., methyl). In some embodiments, Cy^{B} is azetidinonyl, optionally substituted with —(CH₂)₀₋₄R^{◯} or —(CH₂)₀₋₄OR^{◯}, wherein R^{◯} is hydrogen or C₁₋aliphatic. In some embodiments, Cy^{B} is azetidinonyl, substituted with —CH₃.

In some embodiments, Cy^{B} is optionally substituted pyrrolidinyl. In some embodiments, Cy^{B} is pyrrolidinyl, wherein one or more carbon atoms are optionally substituted with one or more ═O or —(CH₂)₀₋₄R^{◯}, and one or more nitrogen atoms are optionally substituted with —R^{†}, wherein each —R^{†} is independently C₁₋₄ aliphatic (e.g., methyl). In some embodiments, Cy^{B} is optionally substituted pyrrolidonyl. In some embodiments, Cy^{B} is pyrrolidonyl, wherein one or more carbon atoms are optionally substituted with one or more-(CH₂)₀₋₄R^{◯}, and one or more nitrogen atoms are optionally substituted with —R^{†}, wherein each —R^{†} is independently C₁₋₄ aliphatic (e.g., methyl). In some embodiments, Cy^{B} is:

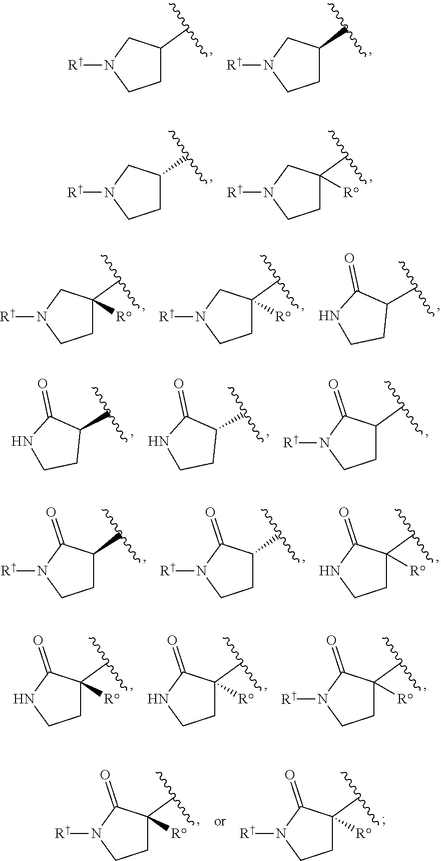

wherein R is C₁₋₄ aliphatic (e.g., methyl), and R^{◯} is C₁₋aliphatic (e.g., methyl).

In some embodiments, Cy^{B} is optionally substituted piperidinyl. In some embodiments, Cy^{B} is piperidinyl, wherein one or more carbon atoms are optionally substituted with one or more halogen (e.g., fluoro), —CN, —(CH₂)₀₋₄R^{◯}, or —(CH₂)₀₋₄OR^{◯}; and one or more nitrogen atoms are optionally substituted with —R^{†}— or —C(O)R^{†}. In some embodiments, Cy^{B} is piperidinyl, wherein one or more carbon atoms are optionally substituted with one or more ═O, halogen (e.g., fluoro), —CN, —(CH₂)₀₋₄R^{◯}, or —(CH₂)₀₋₄OR^{◯}, wherein each R^{◯} is independently C₁₋₆ aliphatic optionally substituted with halogen (e.g., fluoro); and one or more nitrogen atoms are optionally substituted with —R^{†}— or —C(O)R^{†}, wherein each —R is independently C₁₋₆ aliphatic. In some embodiments, Cy^{B} is piperidinyl optionally substituted with one or more methyl, ethyl, isopropyl, —C(O)CH₃, —CN, fluoro, —CH₂CH₂OCH₃, —CHF₂, or ═O. In some embodiments, Cy^{B} is optionally substituted piperidinyl. In some embodiments, Cy^{B} is piperidonyl, wherein one or more carbon atoms are optionally substituted with one or more halogen (e.g., fluoro), —CN, —(CH₂)₀₋₄R^{◯}, or —(CH₂)₀₋₄OR^{◯}; and one or more nitrogen atoms are optionally substituted with —R^{†}— or —C(O)R^{†}. In some embodiments, Cy^{B} is piperidonyl, wherein one or more carbon atoms are optionally substituted with one or more halogen (e.g., fluoro), —CN, —(CH₂)₀₋₄R^{◯}, or —(CH₂)₀₋₄OR^{◯}, wherein each R^{◯} is independently C₁₋aliphatic optionally substituted with halogen (e.g., fluoro); and one or more nitrogen atoms are optionally substituted with —R^{†}— or —C(O)R^{†}, wherein each —R is independently C₁₋₆ aliphatic. In some embodiments, Cy^{B} is piperidonyl optionally substituted with one or more methyl, ethyl, isopropyl, —C(O)CH₃, —CN, fluoro, —CH₂CH₂OCH₃, or —CHF₂. In some embodiments, Cy^{B} is:

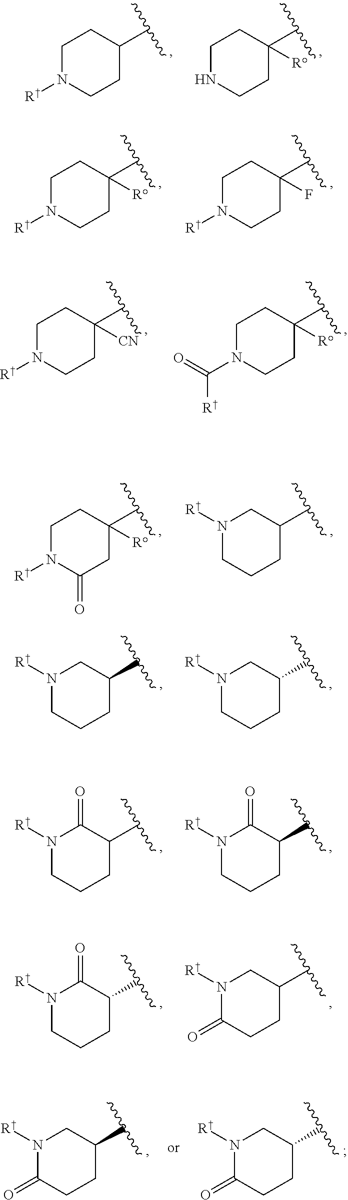

wherein R^{†} is C₁₋₄ aliphatic (e.g., methyl, ethyl, isopropyl), and R^{◯} is C₁₋₆ aliphatic (e.g., methyl) optionally substituted with halogen (e.g., fluoro).

In some embodiments, Cy^{B} is:

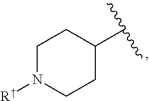

wherein R^{†} is C₁₋₄ aliphatic (e.g., methyl). In some embodiments, Cy^{B} is

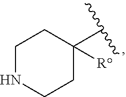

wherein R^{◯} is C₁₋₆ aliphatic (e.g., methyl). In some embodiments, Cy^{B} is

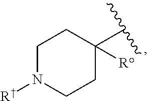

wherein R^{†} is C₁₋₄ aliphatic (e.g., methyl, ethyl, isopropyl), and R^{◯} is halogen (e.g., fluoro), —CN or C₁₋₆ aliphatic (e.g., methyl) optionally substituted with halogen (e.g., fluoro). In some embodiments, Cy^{B} is

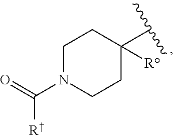

wherein R^{†} is C₁₋₄ aliphatic (e.g., methyl), and R^{◯} is C₁₋₆ aliphatic (e.g., methyl). In some embodiments, Cy^{B} is

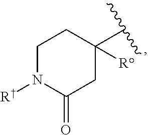

R^{†} is C₁₋₄ aliphatic (e.g., methyl), and R^{◯} is C₁₋₆ aliphatic (e.g., methyl). In some embodiments, Cy^{B} is

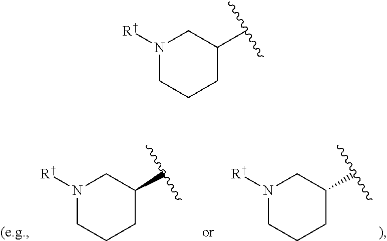

wherein R^{†} is C₁₋₄ aliphatic (e.g., methyl). In some embodiments, Cy^{B} is

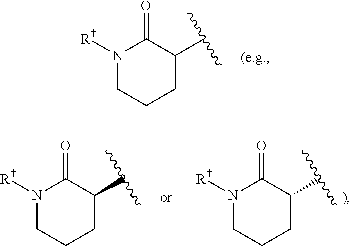

wherein R^{†} is C₁₋₄ aliphatic (e.g., methyl). In some embodiments, Cy^{B} is

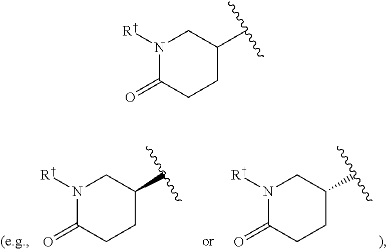

wherein R^{†} is C₁₋₄ aliphatic (e.g., methyl).

In some embodiments, Cy^{B} is optionally substituted 5- to 6-membered monocyclic heteroaryl having 1-3 heteroatoms independently selected from oxygen, nitrogen, or sulfur, and wherein Cy^{B} comprises at least one oxygen or nitrogen heteroatom. In some embodiments, Cy^{B} is optionally substituted 5-membered monocyclic heteroaryl having 1-3 heteroatoms independently selected from oxygen, nitrogen, or sulfur, and wherein Cy^{B} comprises at least one oxygen or nitrogen heteroatom. In some embodiments, Cy^{B} is an optionally substituted 5-membered monocyclic heteroaryl having one oxygen heteroatom. In some embodiments, Cy^{B} is optionally substituted furanyl. In some embodiments, Cy^{B} is an optionally substituted 5-membered monocyclic heteroaryl having one oxygen and one nitrogen heteroatom. In some embodiments, Cy^{B} is optionally substituted isoxazolyl. In some embodiments, Cy^{B} is isoxazolyl, optionally substituted with —(CH₂)₀₋₄R^{◯}, wherein R^{◯} is hydrogen or C₁₋₆ aliphatic (e.g., methyl). In some embodiments, Cy^{B} is an optionally substituted 5-membered monocyclic heteroaryl having one sulfur and one nitrogen heteroatom. In some embodiments, Cy^{B} is an optionally substituted thiazolyl. In some embodiments, Cy^{B} is an optionally substituted 5-membered monocyclic heteroaryl having one sulfur and two nitrogen heteroatoms. In some embodiments, Cy^{B} is an optionally substituted thiadiazolyl.

In some embodiments, Cy^{B} is an optionally substituted 6-membered monocyclic heteroaryl having 1-3 heteroatoms independently selected from oxygen, nitrogen, or sulfur, and wherein Cy^{B} comprises at least one oxygen or nitrogen heteroatom. In some embodiments, Cy^{B} is optionally substituted 6-membered monocyclic heteroaryl having 1 nitrogen heteroatom. In some embodiments, Cy^{B} is optionally substituted pyridinyl. In some embodiments, Cy^{B} is pyridinyl, optionally substituted with —(CH₂)₀₋₄R^{◯}, wherein R^{◯} is a 5- to 6-membered saturated, partially unsaturated, or aryl ring having 0-4 heteroatoms independently selected from nitrogen, oxygen, or sulfur. In some embodiments, Cy^{B} is pyridinyl, optionally substituted with —(CH₂)₀₋₄R^{◯}, wherein R^{◯} is a 5- to 6-membered saturated or partially unsaturated ring having 1-3 heteroatoms independently selected from nitrogen, oxygen, or sulfur. In some embodiments, Cy^{B} is pyridinyl optionally substituted with morpholine. In some embodiments, Cy^{B} is pyridinyl.

In some embodiments, Cy^{B} is an optionally substituted 7-12-membered saturated or partially unsaturated bicyclic heterocyclyl having 1-4 heteroatoms independently selected from oxygen, nitrogen, or sulfur, and wherein Cy^{B} comprises at least one oxygen or nitrogen heteroatom. In some embodiments, Cy^{B} is an optionally substituted 8-membered saturated or partially unsaturated bicyclic heterocyclyl having one nitrogen. In some embodiments, Cy^{B} is

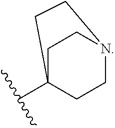 [SMILES: *C12CCN(CC1)CC2]

In some embodiments, In some embodiments, Cy^{B} is an optionally substituted 8-membered saturated or partially unsaturated bicyclic heterocyclyl having 1-4 nitrogen heteroatoms. In some embodiments, Cy^{B} is an optionally substituted triazolopyridinyl.

In some embodiments, Cy^{B} is an optionally substituted 8- to 12-membered bicyclic heteroaryl having 1-4 heteroatoms independently selected from oxygen, nitrogen or sulfur, and wherein Cy^{B} comprises at least one oxygen or nitrogen heteroatom. In some embodiments, Cy^{B} is an optionally substituted 9-membered bicyclic heteroaryl having 1-4 heteroatoms independently selected from oxygen, nitrogen or sulfur, and wherein Cy^{B} comprises at least one oxygen or nitrogen heteroatom. In some embodiment, Cy^{B} is an optionally substituted 8- to 12-membered bicyclic heteroaryl having two nitrogen heteroatoms. In some embodiment, Cy^{B} is an optionally substituted 9-membered bicyclic heteroaryl having two nitrogen heteroatoms. In some embodiments, Cy^{B} is an optionally substituted benzoimidazolyl. In some embodiments, Cy^{B} is an optionally substituted pyrazolopyridinyl. In some embodiment, Cy^{B} is an optionally substituted 8- to 12-membered bicyclic heteroaryl having two nitrogen heteroatoms and one oxygen heteroatom. In some embodiment, Cy^{B} is an optionally substituted 9-membered bicyclic heteroaryl having two nitrogen heteroatoms and one oxygen heteroatom. In some embodiments, Cy^{B} is an optionally substituted pyrrolooxazolyl. In some embodiment, Cy^{B} is an optionally substituted 8- to 12-membered bicyclic heteroaryl having three nitrogen heteroatoms. In some embodiment, Cy^{B} is an optionally substituted 9-membered bicyclic heteroaryl having three nitrogen heteroatoms. In some embodiments, Cy^{B} is an optionally substituted pyrazolopyrimidinyl.

In some embodiments, Cy^{B} is

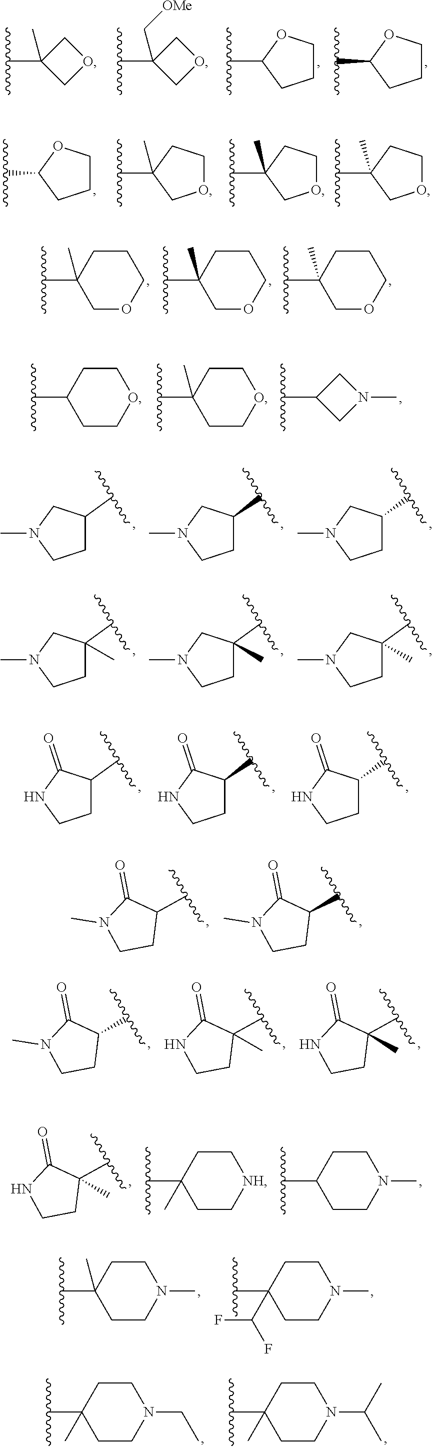

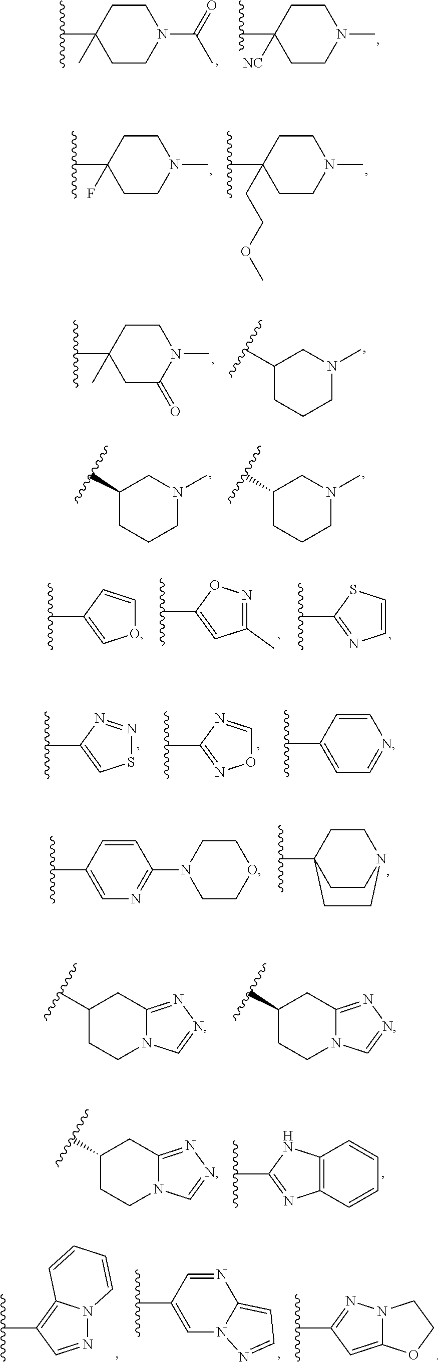

In some embodiments, L-Cy^{B} is:

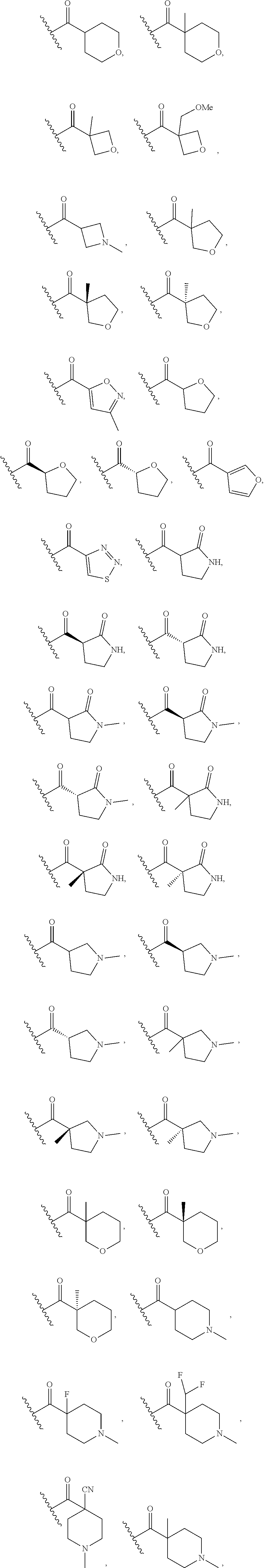

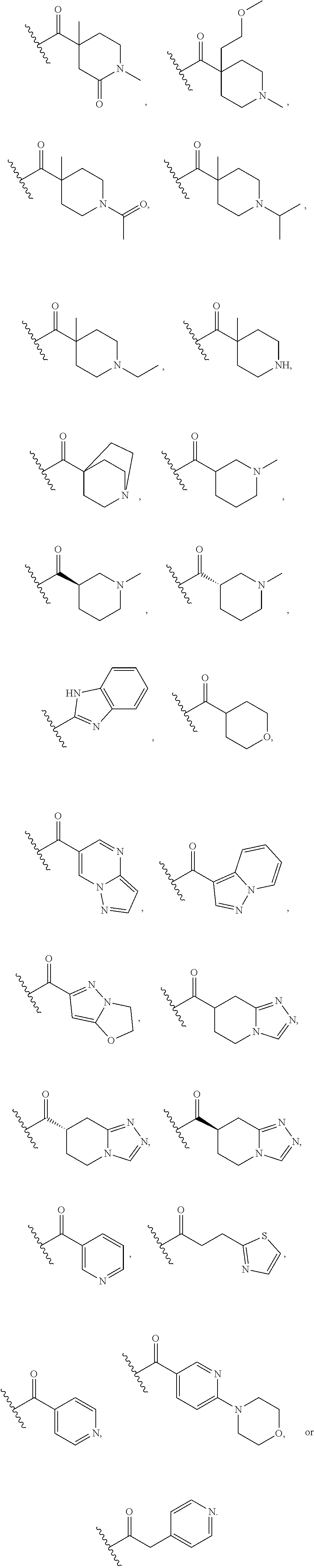

In some embodiments, a provided compound is of Formula VI:

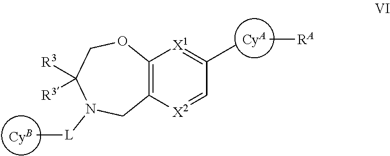

or a pharmaceutically acceptable salt thereof,
wherein each of R^{A}, Cy^{A}, X¹, X², R³, R^{3′}, L, and Cy^{B} defined and described in classes and subclasses wherein, both singly and in combination

It will be understood that, unless otherwise specified or prohibited by the foregoing definition of Formula VI, embodiments of variables R^{A}, Cy^{A}, X¹, X², R³, R^{3′}, L, and Cy as defined above and described in classes and subclasses herein, also apply to compounds of Formula VI, both singly and in combination.

In some embodiments, a provided compound is of Formula VI-a or VI-b:

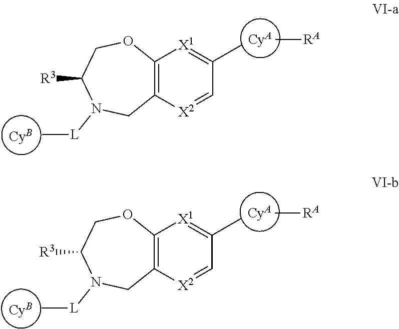

or a pharmaceutically acceptable salt thereof,
wherein each of R^{A}, Cy^{A}, X¹, X², R³, L, and Cy^{B} is defined and described in classes and subclasses wherein, both singly and in combination

It will be understood that, unless otherwise specified or prohibited by the foregoing definition of Formula VI-a or VI-b, embodiments of variables R^{A}, Cy^{A}, X¹, X², R³, L, and Cy^{B} as defined above and described in classes and subclasses herein, also apply to compounds of Formula VI-a or VI-b, both singly and in combination.

In some embodiments, a provided compound is of Formula VII-a or VII-b:

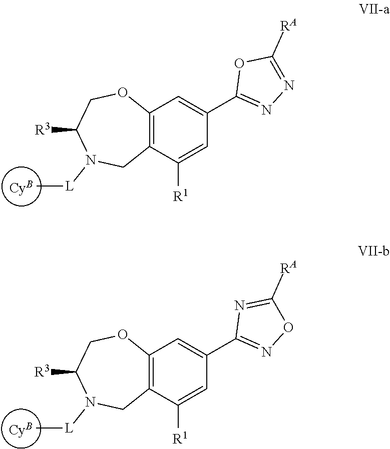

or a pharmaceutically acceptable salt thereof,
wherein each of R^{A}, R¹, R³, L, and Cy^{B} is defined and described in classes and subclasses wherein, both singly and in combination

It will be understood that, unless otherwise specified or prohibited by the foregoing definition of Formula VII-a or VII-b, embodiments of variables R^{A}, R¹, R³, L, and Cy^{B} as defined above and described in classes and subclasses herein, also apply to compounds of Formula VII-a or VII-b, both singly and in combination.

In some embodiments, a provided compound is of Formula VIII-a, VIII-b, VIII-c, or VIII-d:

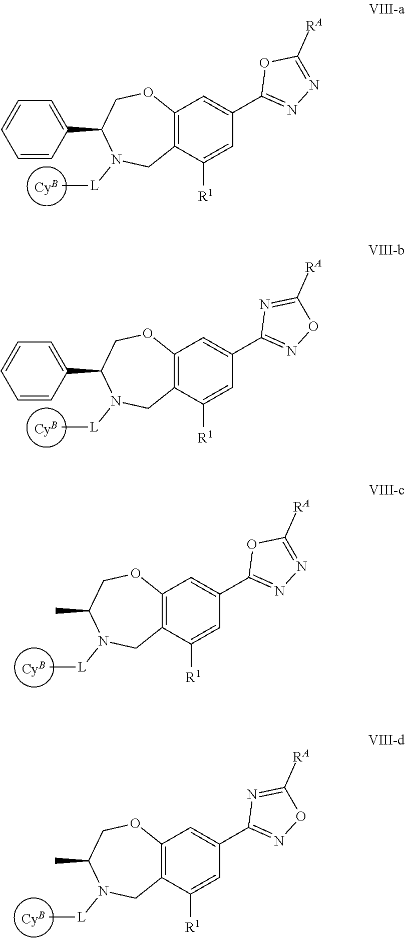

or a pharmaceutically acceptable salt thereof,
wherein each of R^{A}, R¹, L, and Cy^{B} is defined and described in classes and subclasses wherein, both singly and in combination

It will be understood that, unless otherwise specified or prohibited by the foregoing definition of Formula VIII-a, VIII-b, VIII-c, or VIII-d, embodiments of variables R^{A}, R¹, R³, L, and Cy^{B}, as defined above and described in classes and subclasses herein, also apply to compounds of Formula VIII-a, VIII-b, VIII-c, or VIII-d, both singly and in combination.

In some embodiments, a provided compound is selected from the group consisting of:

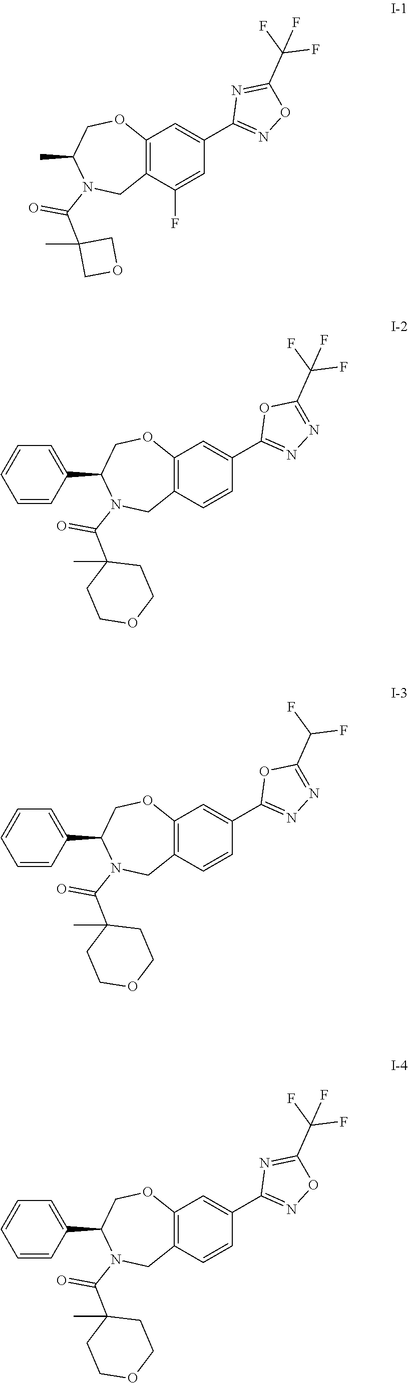 [SMILES: C[C@H]1COc2cc(-c3noc(C(F)(F)F)n3)cc(F)c2CN1C(=O)C1(C)COC1 ; CC1(C(=O)N2Cc3ccc(-c4nnc(C(F)(F)F)o4)cc3OC[C@@H]2c2ccccc2)CCOCC1 ; CC1(C(=O)N2Cc3ccc(-c4nnc(C(F)F)o4)cc3OC[C@@H]2c2ccccc2)CCOCC1 ; CC1(C(=O)N2Cc3ccc(-c4noc(C(F)(F)F)n4)cc3OC[C@@H]2c2ccccc2)CCOCC1]

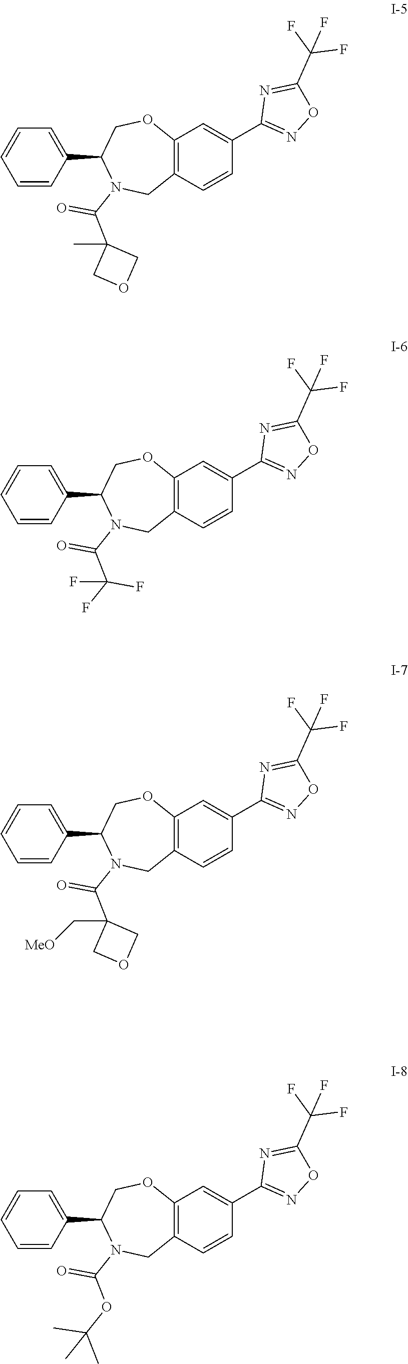 [SMILES: CC1(C(=O)N2Cc3ccc(-c4noc(C(F)(F)F)n4)cc3OC[C@@H]2c2ccccc2)COC1 ; O=C(N1Cc2ccc(-c3noc(C(F)(F)F)n3)cc2OC[C@@H]1c1ccccc1)C(F)(F)F ; COCC1(C(=O)N2Cc3ccc(-c4noc(C(F)(F)F)n4)cc3OC[C@@H]2c2ccccc2)COC1 ; CC(C)(C)OC(=O)N1Cc2ccc(-c3noc(C(F)(F)F)n3)cc2OC[C@@H]1c1ccccc1]

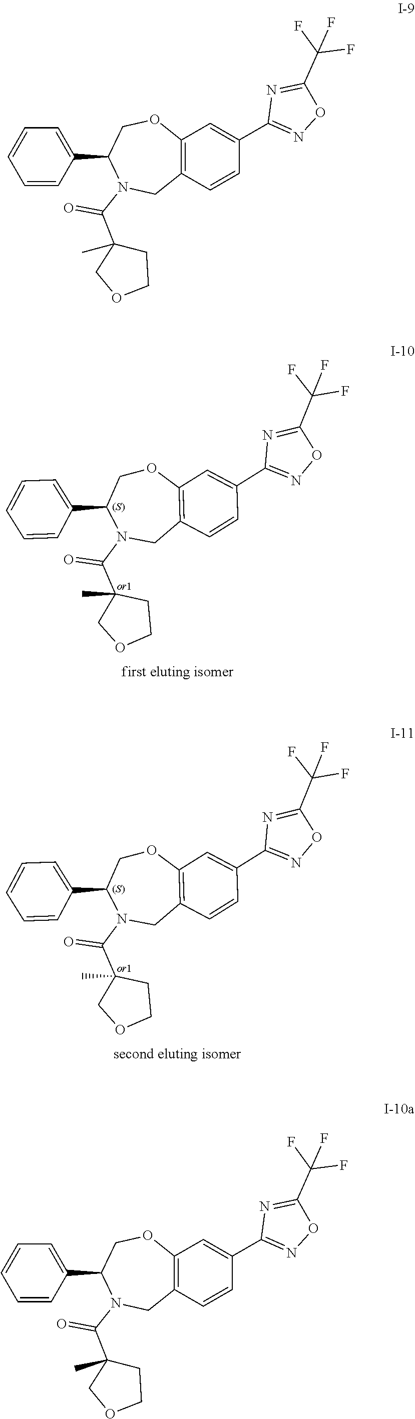

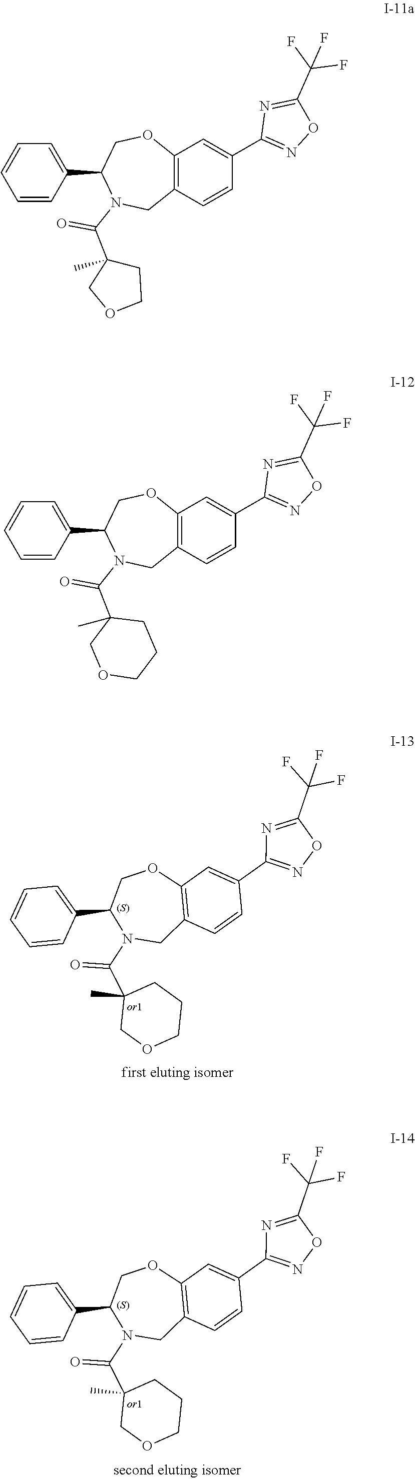

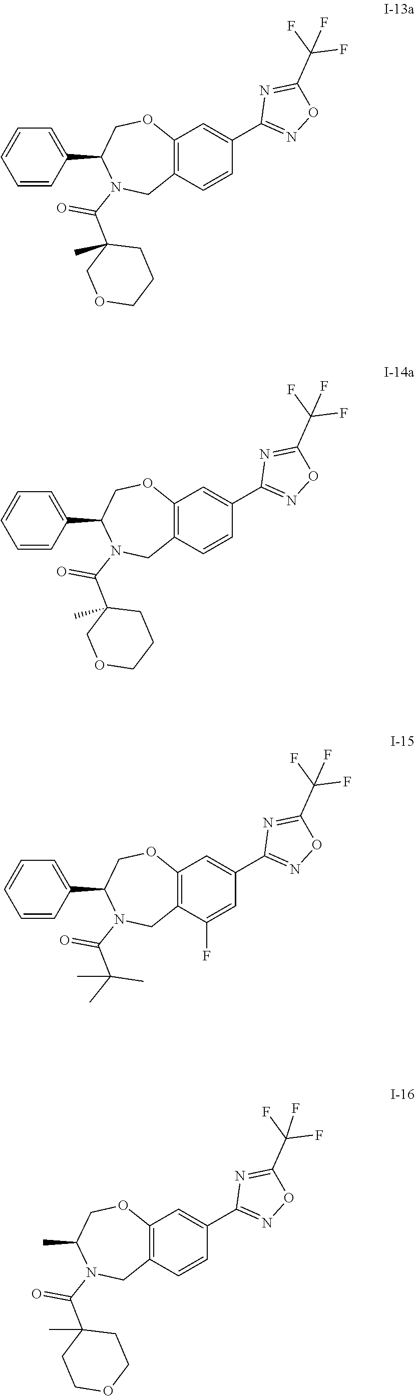 [SMILES: C[C@@]1(C(=O)N2Cc3ccc(-c4noc(C(F)(F)F)n4)cc3OC[C@@H]2c2ccccc2)CCCOC1 ; C[C@]1(C(=O)N2Cc3ccc(-c4noc(C(F)(F)F)n4)cc3OC[C@@H]2c2ccccc2)CCCOC1 ; CC(C)(C)C(=O)N1Cc2c(F)cc(-c3noc(C(F)(F)F)n3)cc2OC[C@@H]1c1ccccc1 ; C[C@H]1COc2cc(-c3noc(C(F)(F)F)n3)ccc2CN1C(=O)C1(C)CCOCC1]

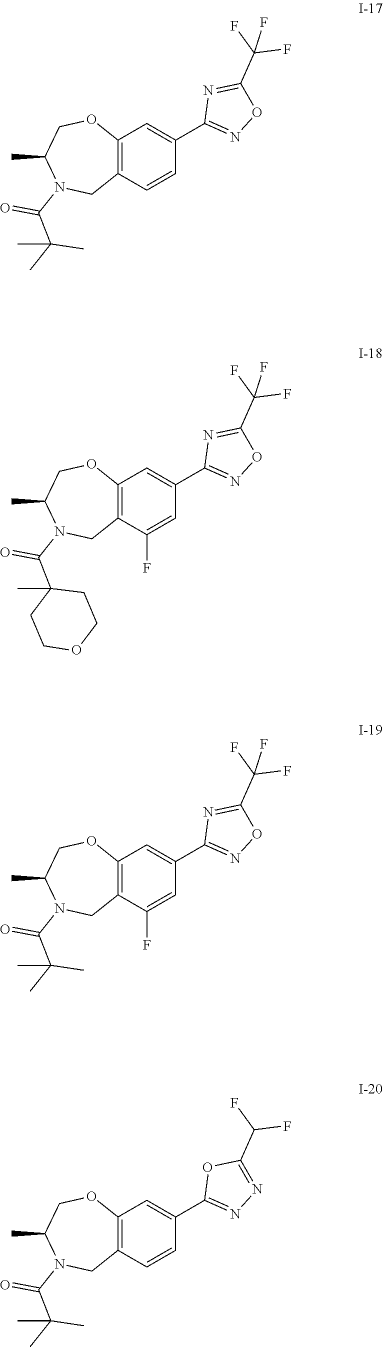 [SMILES: C[C@H]1COc2cc(-c3noc(C(F)(F)F)n3)ccc2CN1C(=O)C(C)(C)C ; C[C@H]1COc2cc(-c3noc(C(F)(F)F)n3)cc(F)c2CN1C(=O)C1(C)CCOCC1 ; C[C@H]1COc2cc(-c3noc(C(F)(F)F)n3)cc(F)c2CN1C(=O)C(C)(C)C ; C[C@H]1COc2cc(-c3nnc(C(F)F)o3)ccc2CN1C(=O)C(C)(C)C]

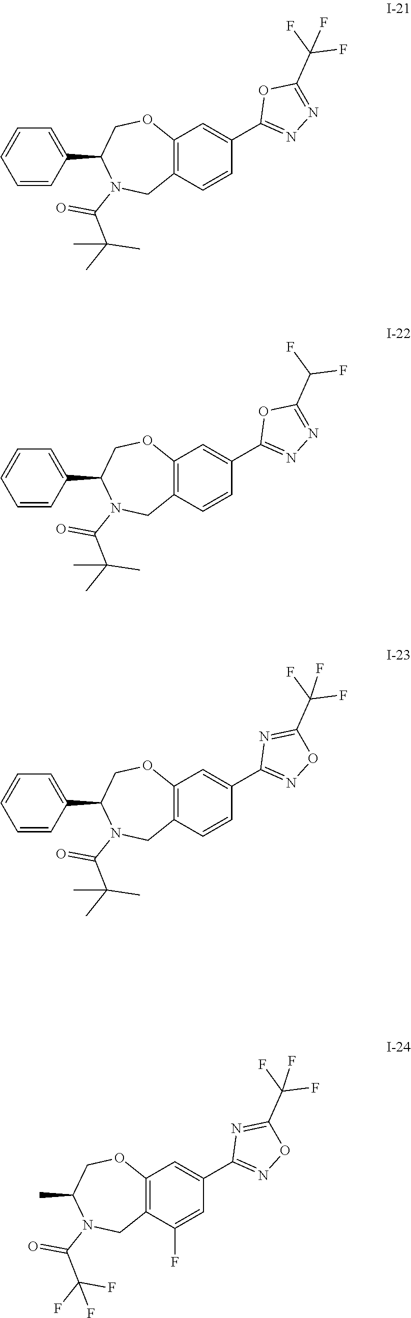 [SMILES: CC(C)(C)C(=O)N1Cc2ccc(-c3nnc(C(F)(F)F)o3)cc2OC[C@@H]1c1ccccc1 ; CC(C)(C)C(=O)N1Cc2ccc(-c3nnc(C(F)F)o3)cc2OC[C@@H]1c1ccccc1 ; CC(C)(C)C(=O)N1Cc2ccc(-c3noc(C(F)(F)F)n3)cc2OC[C@@H]1c1ccccc1 ; C[C@H]1COc2cc(-c3noc(C(F)(F)F)n3)cc(F)c2CN1C(=O)C(F)(F)F]

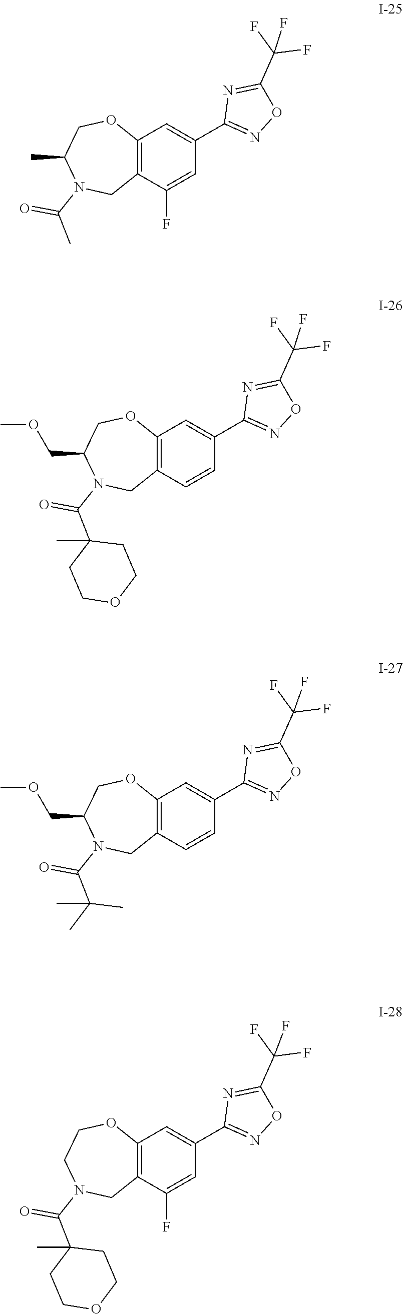 [SMILES: CC(=O)N1Cc2c(F)cc(-c3noc(C(F)(F)F)n3)cc2OC[C@@H]1C ; COC[C@H]1COc2cc(-c3noc(C(F)(F)F)n3)ccc2CN1C(=O)C1(C)CCOCC1 ; COC[C@H]1COc2cc(-c3noc(C(F)(F)F)n3)ccc2CN1C(=O)C(C)(C)C ; CC1(C(=O)N2CCOc3cc(-c4noc(C(F)(F)F)n4)cc(F)c3C2)CCOCC1]

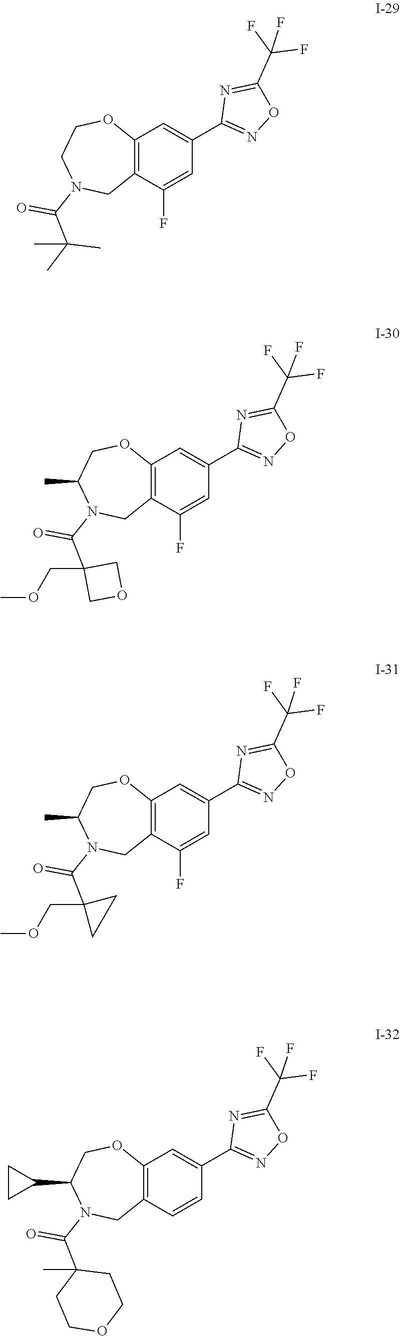 [SMILES: CC(C)(C)C(=O)N1CCOc2cc(-c3noc(C(F)(F)F)n3)cc(F)c2C1 ; COCC1(C(=O)N2Cc3c(F)cc(-c4noc(C(F)(F)F)n4)cc3OC[C@@H]2C)COC1 ; COCC1(C(=O)N2Cc3c(F)cc(-c4noc(C(F)(F)F)n4)cc3OC[C@@H]2C)CC1 ; CC1(C(=O)N2Cc3ccc(-c4noc(C(F)(F)F)n4)cc3OC[C@@H]2C2CC2)CCOCC1]

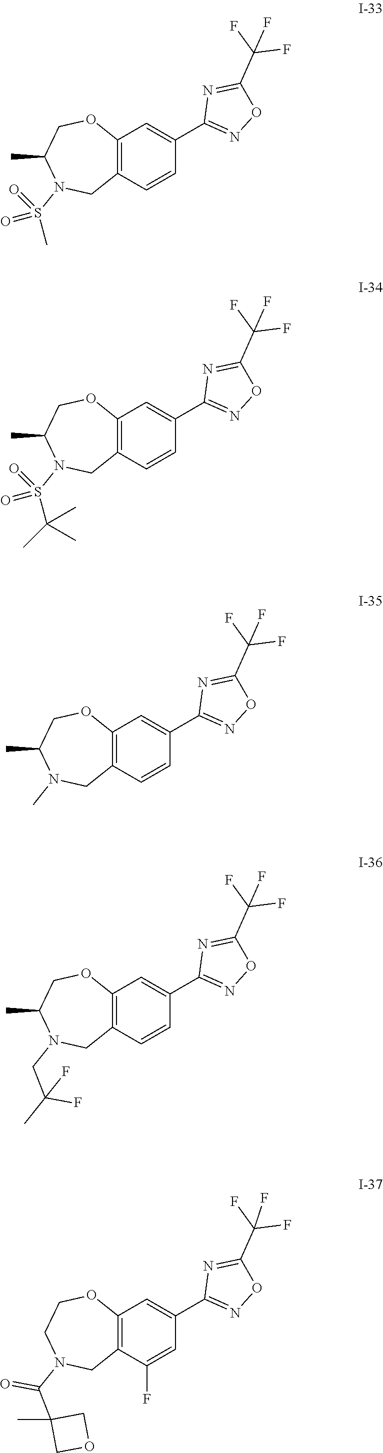 [SMILES: C[C@H]1COc2cc(-c3noc(C(F)(F)F)n3)ccc2CN1S(C)(=O)=O ; C[C@H]1COc2cc(-c3noc(C(F)(F)F)n3)ccc2CN1S(=O)(=O)C(C)(C)C ; C[C@H]1COc2cc(-c3noc(C(F)(F)F)n3)ccc2CN1C ; C[C@H]1COc2cc(-c3noc(C(F)(F)F)n3)ccc2CN1CC(C)(F)F ; CC1(C(=O)N2CCOc3cc(-c4noc(C(F)(F)F)n4)cc(F)c3C2)COC1]

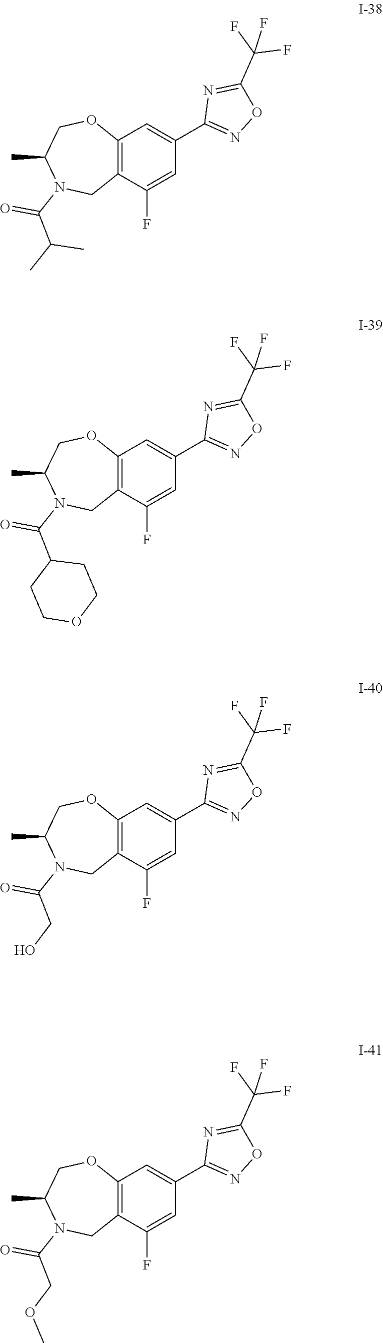 [SMILES: CC(C)C(=O)N1Cc2c(F)cc(-c3noc(C(F)(F)F)n3)cc2OC[C@@H]1C ; C[C@H]1COc2cc(-c3noc(C(F)(F)F)n3)cc(F)c2CN1C(=O)C1CCOCC1 ; C[C@H]1COc2cc(-c3noc(C(F)(F)F)n3)cc(F)c2CN1C(=O)CO ; COCC(=O)N1Cc2c(F)cc(-c3noc(C(F)(F)F)n3)cc2OC[C@@H]1C]

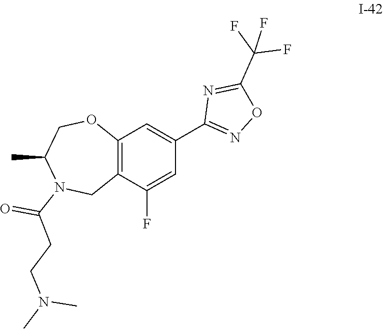 [SMILES: C[C@H]1COc2cc(-c3noc(C(F)(F)F)n3)cc(F)c2CN1C(=O)CCN(C)C]

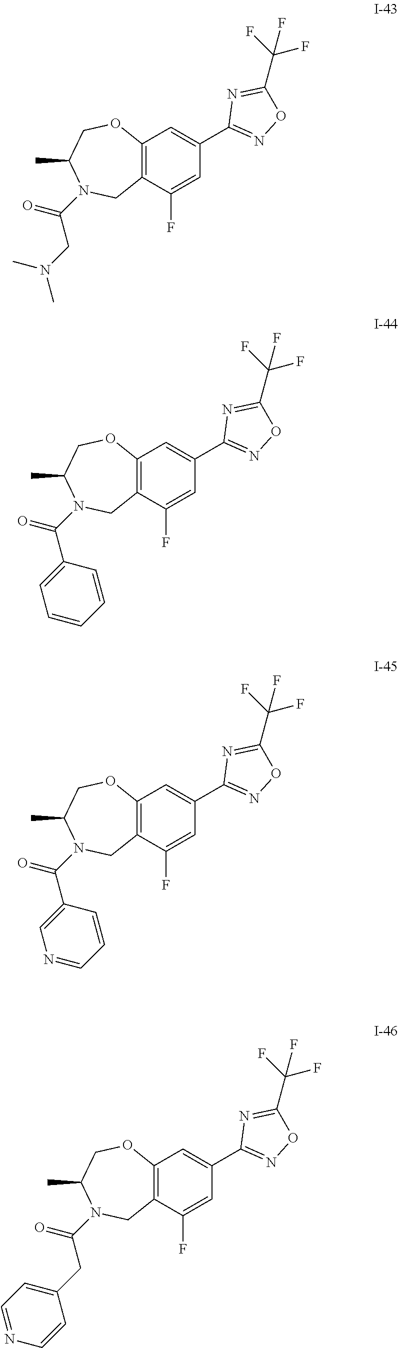 [SMILES: C[C@H]1COc2cc(-c3noc(C(F)(F)F)n3)cc(F)c2CN1C(=O)CN(C)C ; C[C@H]1COc2cc(-c3noc(C(F)(F)F)n3)cc(F)c2CN1C(=O)c1ccccc1 ; C[C@H]1COc2cc(-c3noc(C(F)(F)F)n3)cc(F)c2CN1C(=O)c1cccnc1 ; C[C@H]1COc2cc(-c3noc(C(F)(F)F)n3)cc(F)c2CN1C(=O)Cc1ccncc1]

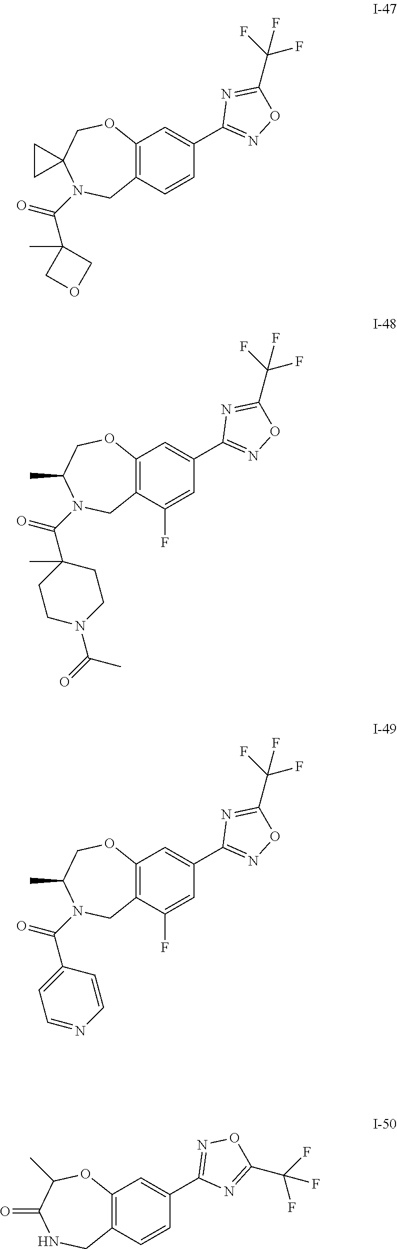 [SMILES: CC1(C(=O)N2Cc3ccc(-c4noc(C(F)(F)F)n4)cc3OCC23CC3)COC1 ; CC(=O)N1CCC(C)(C(=O)N2Cc3c(F)cc(-c4noc(C(F)(F)F)n4)cc3OC[C@@H]2C)CC1 ; C[C@H]1COc2cc(-c3noc(C(F)(F)F)n3)cc(F)c2CN1C(=O)c1ccncc1 ; CC1Oc2cc(-c3noc(C(F)(F)F)n3)ccc2CNC1=O]

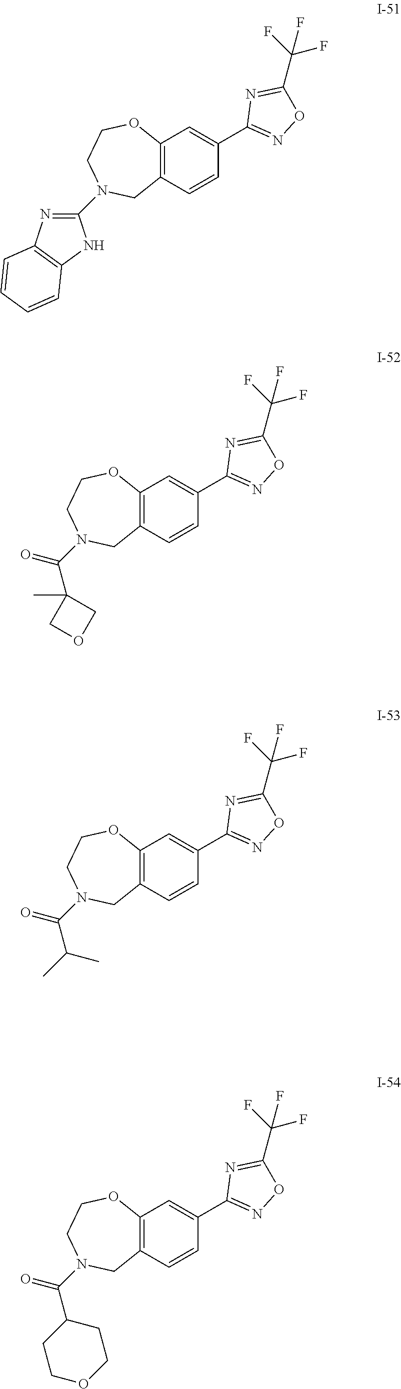 [SMILES: FC(F)(F)c1nc(-c2ccc3c(c2)OCCN(c2nc4ccccc4[nH]2)C3)no1 ; CC1(C(=O)N2CCOc3cc(-c4noc(C(F)(F)F)n4)ccc3C2)COC1 ; CC(C)C(=O)N1CCOc2cc(-c3noc(C(F)(F)F)n3)ccc2C1 ; O=C(C1CCOCC1)N1CCOc2cc(-c3noc(C(F)(F)F)n3)ccc2C1]

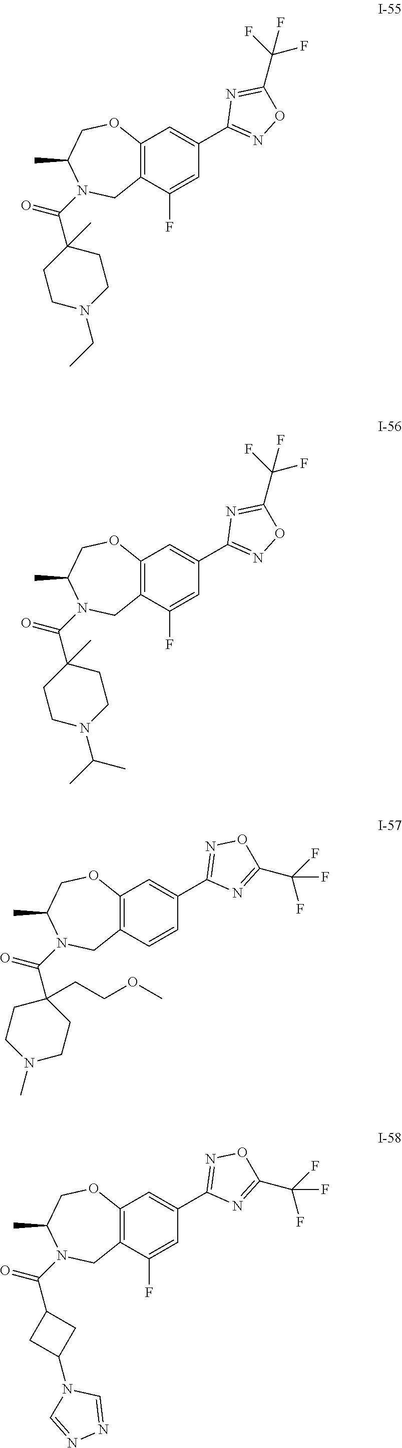 [SMILES: CCN1CCC(C)(C(=O)N2Cc3c(F)cc(-c4noc(C(F)(F)F)n4)cc3OC[C@@H]2C)CC1 ; CC(C)N1CCC(C)(C(=O)N2Cc3c(F)cc(-c4noc(C(F)(F)F)n4)cc3OC[C@@H]2C)CC1 ; COCCC1(C(=O)N2Cc3ccc(-c4noc(C(F)(F)F)n4)cc3OC[C@@H]2C)CCN(C)CC1 ; C[C@H]1COc2cc(-c3noc(C(F)(F)F)n3)cc(F)c2CN1C(=O)C1CC(n2cnnc2)C1]

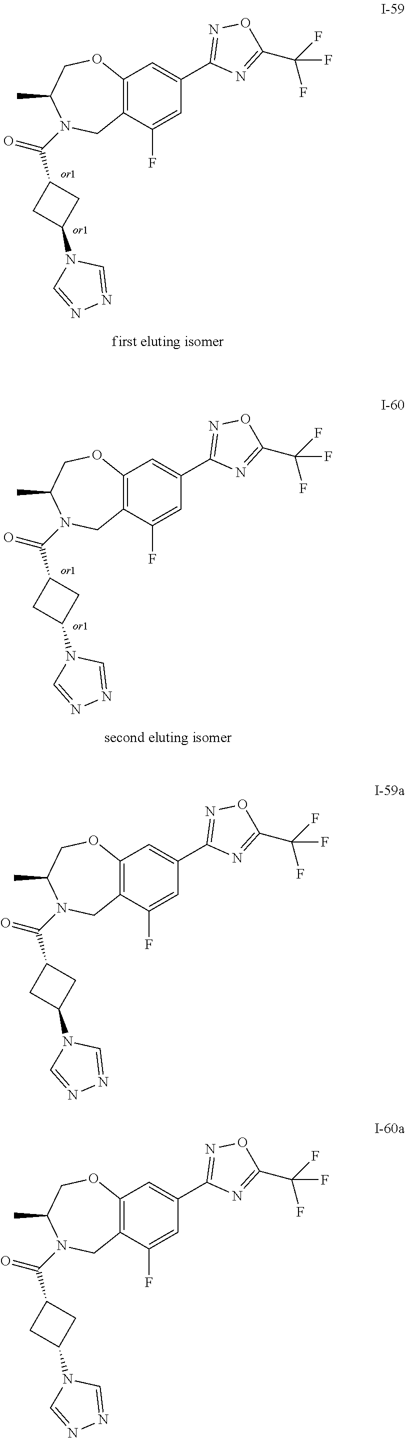 [SMILES: C[C@H]1COc2cc(-c3noc(C(F)(F)F)n3)cc(F)c2CN1C(=O)[C@H]1C[C@H](n2cnnc2)C1 ; C[C@H]1COc2cc(-c3noc(C(F)(F)F)n3)cc(F)c2CN1C(=O)[C@H]1C[C@@H](n2cnnc2)C1 ; C[C@H]1COc2cc(-c3noc(C(F)(F)F)n3)cc(F)c2CN1C(=O)[C@H]1C[C@H](n2cnnc2)C1 ; C[C@H]1COc2cc(-c3noc(C(F)(F)F)n3)cc(F)c2CN1C(=O)[C@H]1C[C@@H](n2cnnc2)C1]

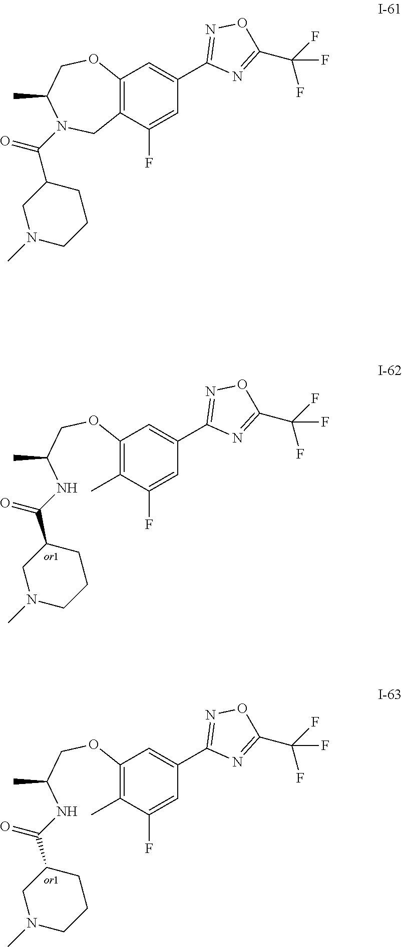 [SMILES: C[C@H]1COc2cc(-c3noc(C(F)(F)F)n3)cc(F)c2CN1C(=O)C1CCCN(C)C1 ; Cc1c(F)cc(-c2noc(C(F)(F)F)n2)cc1OC[C@H](C)NC(=O)[C@H]1CCCN(C)C1 ; Cc1c(F)cc(-c2noc(C(F)(F)F)n2)cc1OC[C@H](C)NC(=O)[C@@H]1CCCN(C)C1]

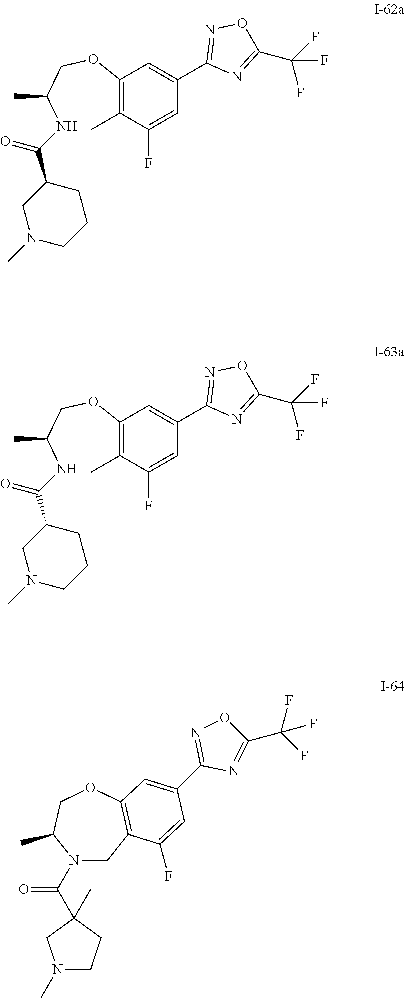 [SMILES: Cc1c(F)cc(-c2noc(C(F)(F)F)n2)cc1OC[C@H](C)NC(=O)[C@H]1CCCN(C)C1 ; Cc1c(F)cc(-c2noc(C(F)(F)F)n2)cc1OC[C@H](C)NC(=O)[C@@H]1CCCN(C)C1 ; C[C@H]1COc2cc(-c3noc(C(F)(F)F)n3)cc(F)c2CN1C(=O)C1(C)CCN(C)C1]

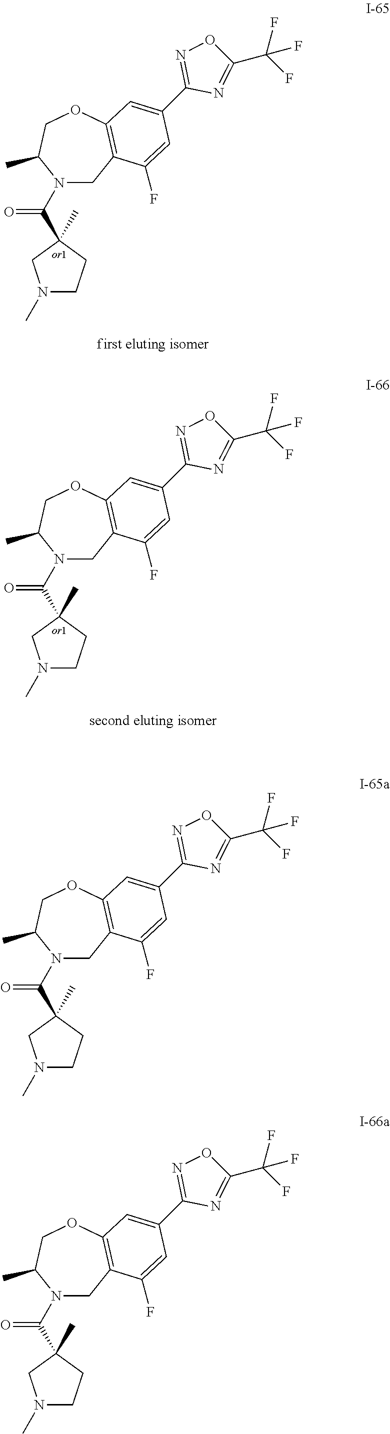 [SMILES: C[C@H]1COc2cc(-c3noc(C(F)(F)F)n3)cc(F)c2CN1C(=O)[C@@]1(C)CCN(C)C1 ; C[C@H]1COc2cc(-c3noc(C(F)(F)F)n3)cc(F)c2CN1C(=O)[C@]1(C)CCN(C)C1 ; C[C@H]1COc2cc(-c3noc(C(F)(F)F)n3)cc(F)c2CN1C(=O)[C@@]1(C)CCN(C)C1 ; C[C@H]1COc2cc(-c3noc(C(F)(F)F)n3)cc(F)c2CN1C(=O)[C@]1(C)CCN(C)C1]

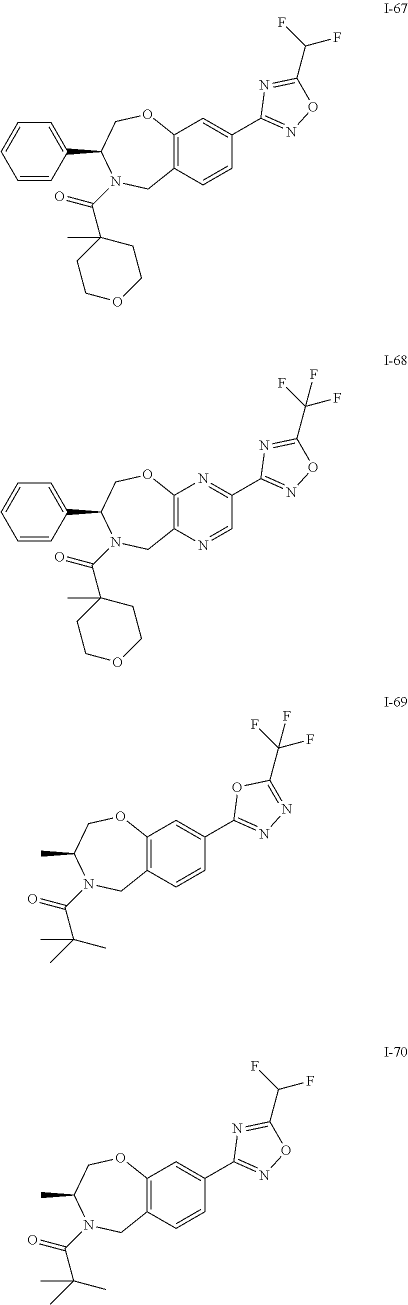 [SMILES: CC1(C(=O)N2Cc3ccc(-c4noc(C(F)F)n4)cc3OC[C@@H]2c2ccccc2)CCOCC1 ; CC1(C(=O)N2Cc3ncc(-c4noc(C(F)(F)F)n4)nc3OC[C@@H]2c2ccccc2)CCOCC1 ; C[C@H]1COc2cc(-c3nnc(C(F)(F)F)o3)ccc2CN1C(=O)C(C)(C)C ; C[C@H]1COc2cc(-c3noc(C(F)F)n3)ccc2CN1C(=O)C(C)(C)C]

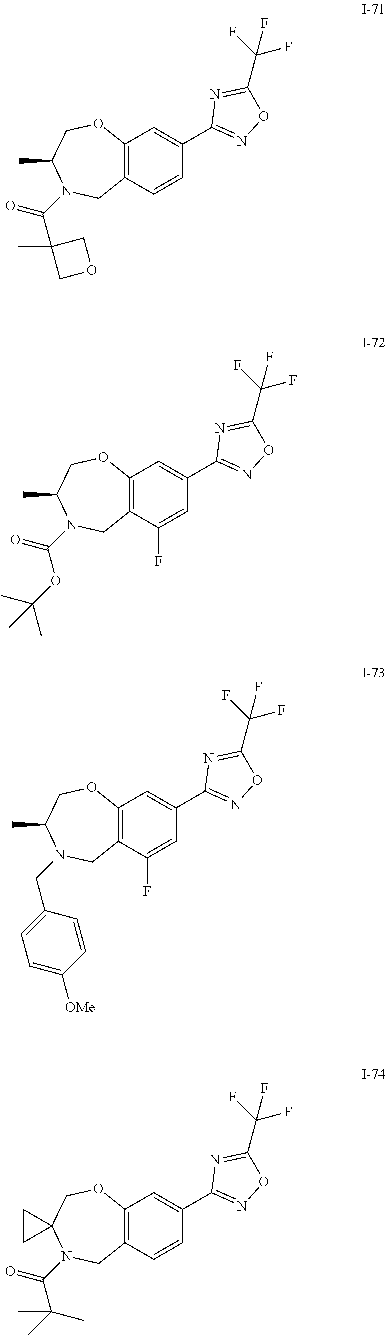 [SMILES: C[C@H]1COc2cc(-c3noc(C(F)(F)F)n3)ccc2CN1C(=O)C1(C)COC1 ; C[C@H]1COc2cc(-c3noc(C(F)(F)F)n3)cc(F)c2CN1C(=O)OC(C)(C)C ; COc1ccc(CN2Cc3c(F)cc(-c4noc(C(F)(F)F)n4)cc3OC[C@@H]2C)cc1 ; CC(C)(C)C(=O)N1Cc2ccc(-c3noc(C(F)(F)F)n3)cc2OCC12CC2]

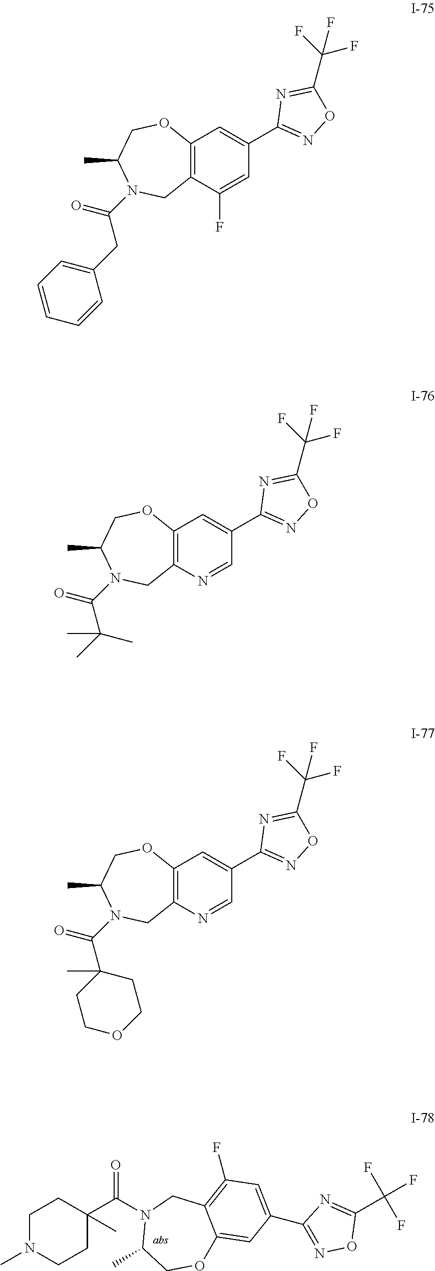 [SMILES: C[C@H]1COc2cc(-c3noc(C(F)(F)F)n3)cc(F)c2CN1C(=O)Cc1ccccc1 ; C[C@H]1COc2cc(-c3noc(C(F)(F)F)n3)cnc2CN1C(=O)C(C)(C)C ; C[C@H]1COc2cc(-c3noc(C(F)(F)F)n3)cnc2CN1C(=O)C1(C)CCOCC1 ; C[C@H]1COc2cc(-c3noc(C(F)(F)F)n3)cc(F)c2CN1C(=O)C1(C)CCN(C)CC1]

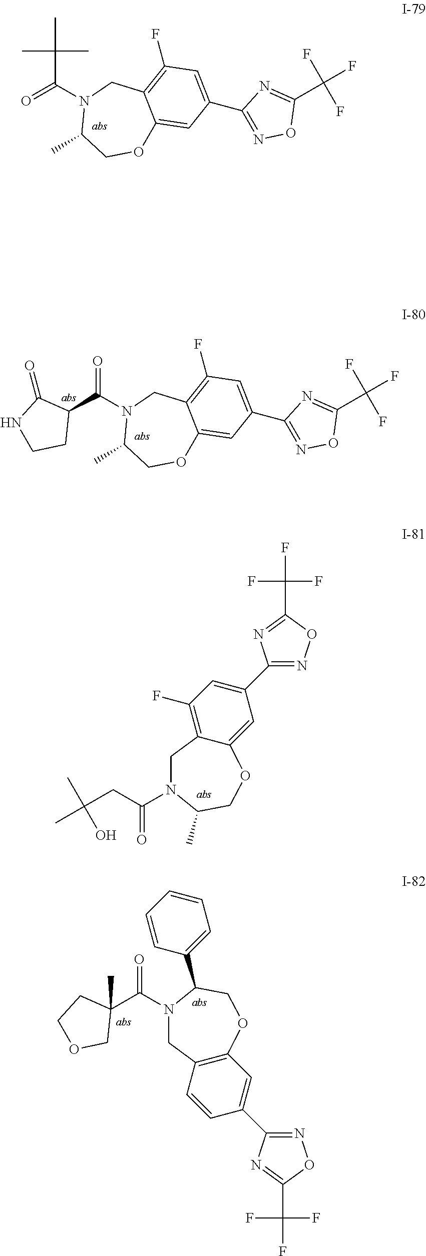 [SMILES: C[C@H]1COc2cc(-c3noc(C(F)(F)F)n3)cc(F)c2CN1C(=O)C(C)(C)C ; C[C@H]1COc2cc(-c3noc(C(F)(F)F)n3)cc(F)c2CN1C(=O)[C@H]1CCNC1=O ; C[C@H]1COc2cc(-c3noc(C(F)(F)F)n3)cc(F)c2CN1C(=O)CC(C)(C)O ; C[C@]1(C(=O)N2Cc3ccc(-c4noc(C(F)(F)F)n4)cc3OC[C@@H]2c2ccccc2)CCOC1]

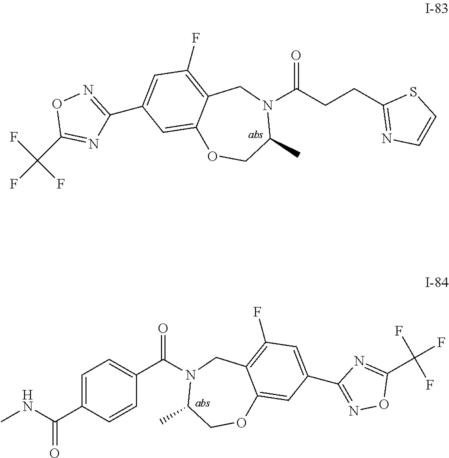 [SMILES: C[C@H]1COc2cc(-c3noc(C(F)(F)F)n3)cc(F)c2CN1C(=O)CCc1nccs1 ; CNC(=O)c1ccc(C(=O)N2Cc3c(F)cc(-c4noc(C(F)(F)F)n4)cc3OC[C@@H]2C)cc1]

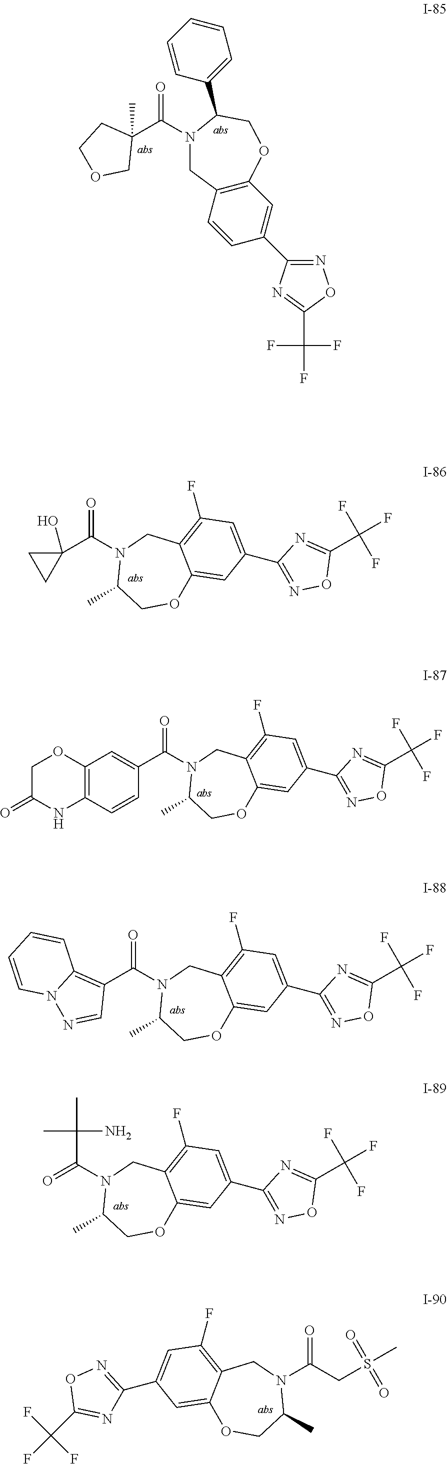 [SMILES: C[C@@]1(C(=O)N2Cc3ccc(-c4noc(C(F)(F)F)n4)cc3OC[C@@H]2c2ccccc2)CCOC1 ; C[C@H]1COc2cc(-c3noc(C(F)(F)F)n3)cc(F)c2CN1C(=O)C1(O)CC1 ; C[C@H]1COc2cc(-c3noc(C(F)(F)F)n3)cc(F)c2CN1C(=O)c1ccc2c(c1)OCC(=O)N2 ; C[C@H]1COc2cc(-c3noc(C(F)(F)F)n3)cc(F)c2CN1C(=O)c1cnn2ccccc12 ; C[C@H]1COc2cc(-c3noc(C(F)(F)F)n3)cc(F)c2CN1C(=O)C(C)(C)N ; C[C@H]1COc2cc(-c3noc(C(F)(F)F)n3)cc(F)c2CN1C(=O)CS(C)(=O)=O]

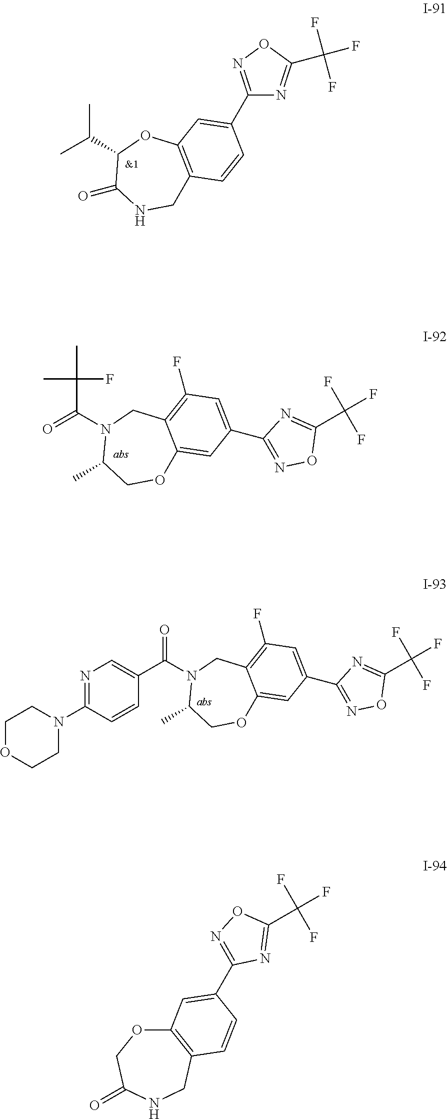 [SMILES: CC(C)[C@@H]1Oc2cc(-c3noc(C(F)(F)F)n3)ccc2CNC1=O ; C[C@H]1COc2cc(-c3noc(C(F)(F)F)n3)cc(F)c2CN1C(=O)C(C)(C)F ; C[C@H]1COc2cc(-c3noc(C(F)(F)F)n3)cc(F)c2CN1C(=O)c1ccc(N2CCOCC2)nc1 ; O=C1COc2cc(-c3noc(C(F)(F)F)n3)ccc2CN1]

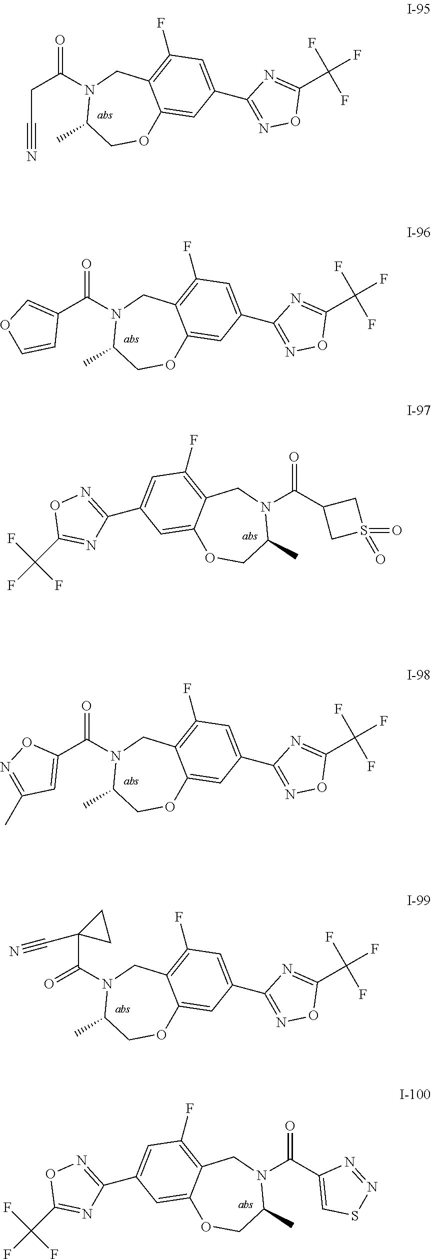 [SMILES: C[C@H]1COc2cc(-c3noc(C(F)(F)F)n3)cc(F)c2CN1C(=O)CC#N ; C[C@H]1COc2cc(-c3noc(C(F)(F)F)n3)cc(F)c2CN1C(=O)c1ccoc1 ; C[C@H]1COc2cc(-c3noc(C(F)(F)F)n3)cc(F)c2CN1C(=O)C1CS(=O)(=O)C1 ; Cc1cc(C(=O)N2Cc3c(F)cc(-c4noc(C(F)(F)F)n4)cc3OC[C@@H]2C)on1 ; C[C@H]1COc2cc(-c3noc(C(F)(F)F)n3)cc(F)c2CN1C(=O)C1(C#N)CC1 ; C[C@H]1COc2cc(-c3noc(C(F)(F)F)n3)cc(F)c2CN1C(=O)c1csnn1]

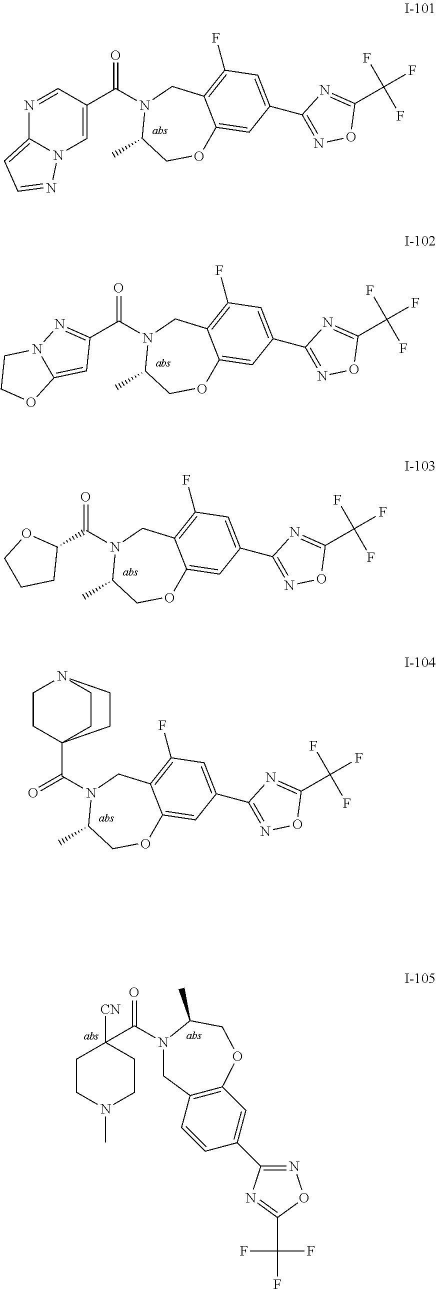 [SMILES: C[C@H]1COc2cc(-c3noc(C(F)(F)F)n3)cc(F)c2CN1C(=O)c1cnc2ccnn2c1 ; C[C@H]1COc2cc(-c3noc(C(F)(F)F)n3)cc(F)c2CN1C(=O)c1cc2n(n1)CCO2 ; C[C@H]1COc2cc(-c3noc(C(F)(F)F)n3)cc(F)c2CN1C(=O)[C@@H]1CCCO1 ; C[C@H]1COc2cc(-c3noc(C(F)(F)F)n3)cc(F)c2CN1C(=O)C12CCN(CC1)CC2 ; C[C@H]1COc2cc(-c3noc(C(F)(F)F)n3)ccc2CN1C(=O)C1(C#N)CCN(C)CC1]

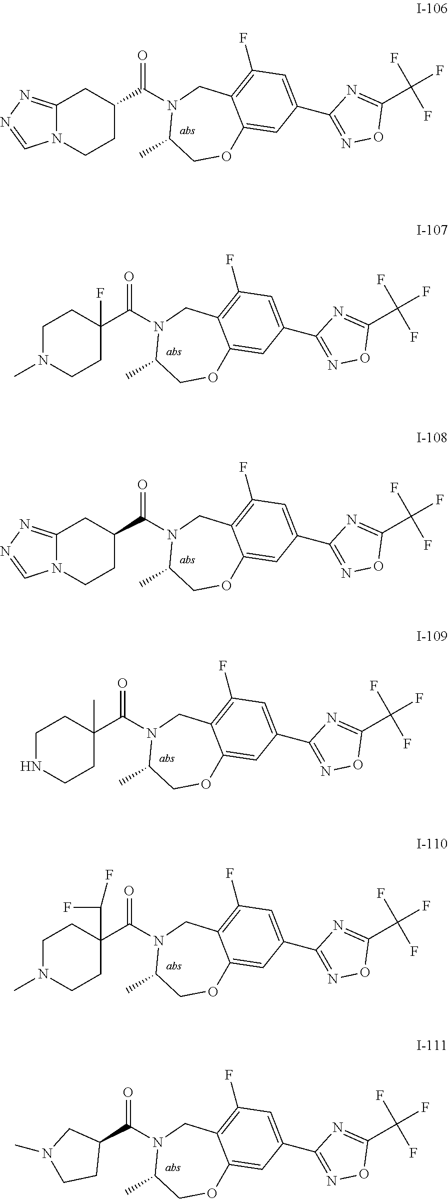 [SMILES: C[C@H]1COc2cc(-c3noc(C(F)(F)F)n3)cc(F)c2CN1C(=O)[C@@H]1CCn2cnnc2C1 ; C[C@H]1COc2cc(-c3noc(C(F)(F)F)n3)cc(F)c2CN1C(=O)C1(F)CCN(C)CC1 ; C[C@H]1COc2cc(-c3noc(C(F)(F)F)n3)cc(F)c2CN1C(=O)[C@H]1CCn2cnnc2C1 ; C[C@H]1COc2cc(-c3noc(C(F)(F)F)n3)cc(F)c2CN1C(=O)C1(C)CCNCC1 ; C[C@H]1COc2cc(-c3noc(C(F)(F)F)n3)cc(F)c2CN1C(=O)C1(C(F)F)CCN(C)CC1 ; C[C@H]1COc2cc(-c3noc(C(F)(F)F)n3)cc(F)c2CN1C(=O)[C@H]1CCN(C)C1]

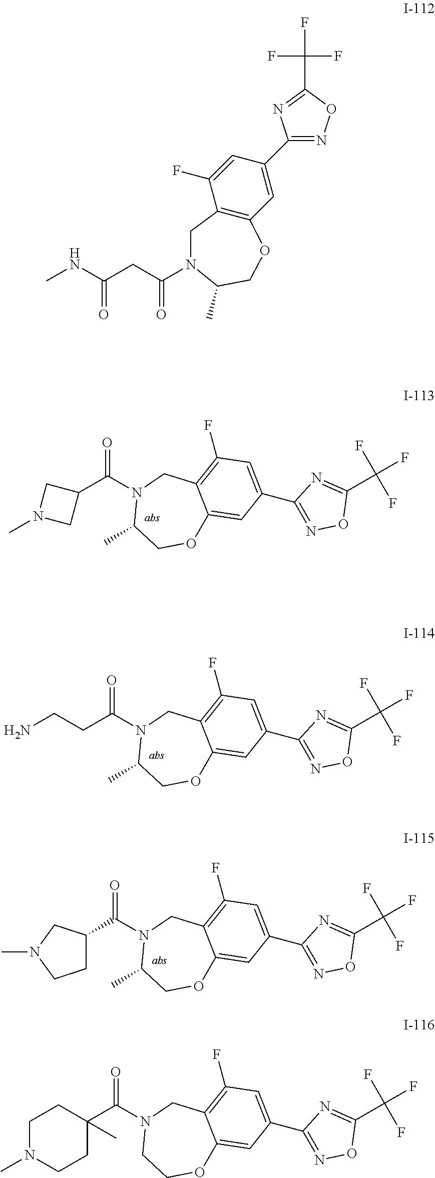 [SMILES: CNC(=O)CC(=O)N1Cc2c(F)cc(-c3noc(C(F)(F)F)n3)cc2OC[C@@H]1C ; C[C@H]1COc2cc(-c3noc(C(F)(F)F)n3)cc(F)c2CN1C(=O)C1CN(C)C1 ; C[C@H]1COc2cc(-c3noc(C(F)(F)F)n3)cc(F)c2CN1C(=O)CCN ; C[C@H]1COc2cc(-c3noc(C(F)(F)F)n3)cc(F)c2CN1C(=O)[C@@H]1CCN(C)C1 ; CN1CCC(C)(C(=O)N2CCOc3cc(-c4noc(C(F)(F)F)n4)cc(F)c3C2)CC1]

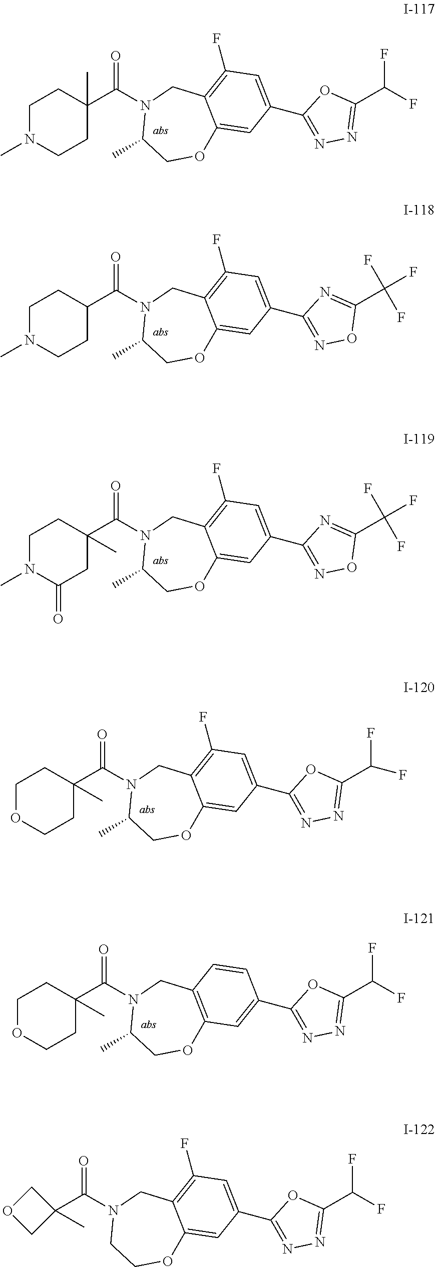 [SMILES: C[C@H]1COc2cc(-c3nnc(C(F)F)o3)cc(F)c2CN1C(=O)C1(C)CCN(C)CC1 ; C[C@H]1COc2cc(-c3noc(C(F)(F)F)n3)cc(F)c2CN1C(=O)C1CCN(C)CC1 ; C[C@H]1COc2cc(-c3noc(C(F)(F)F)n3)cc(F)c2CN1C(=O)C1(C)CCN(C)C(=O)C1 ; C[C@H]1COc2cc(-c3nnc(C(F)F)o3)cc(F)c2CN1C(=O)C1(C)CCOCC1 ; C[C@H]1COc2cc(-c3nnc(C(F)F)o3)ccc2CN1C(=O)C1(C)CCOCC1 ; CC1(C(=O)N2CCOc3cc(-c4nnc(C(F)F)o4)cc(F)c3C2)COC1]

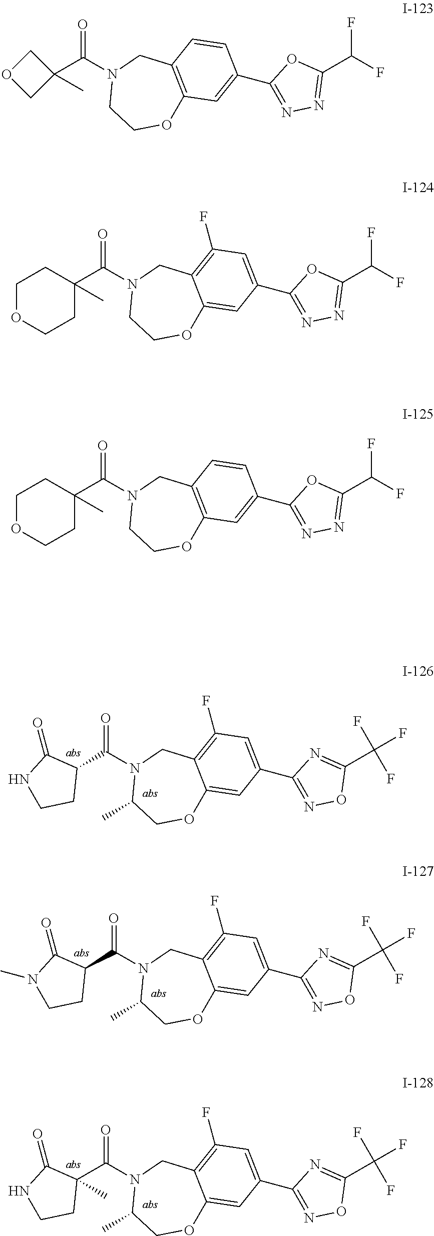 [SMILES: CC1(C(=O)N2CCOc3cc(-c4nnc(C(F)F)o4)ccc3C2)COC1 ; CC1(C(=O)N2CCOc3cc(-c4nnc(C(F)F)o4)cc(F)c3C2)CCOCC1 ; CC1(C(=O)N2CCOc3cc(-c4nnc(C(F)F)o4)ccc3C2)CCOCC1 ; C[C@H]1COc2cc(-c3noc(C(F)(F)F)n3)cc(F)c2CN1C(=O)[C@@H]1CCNC1=O ; C[C@H]1COc2cc(-c3noc(C(F)(F)F)n3)cc(F)c2CN1C(=O)[C@H]1CCN(C)C1=O ; C[C@H]1COc2cc(-c3noc(C(F)(F)F)n3)cc(F)c2CN1C(=O)[C@@]1(C)CCNC1=O]

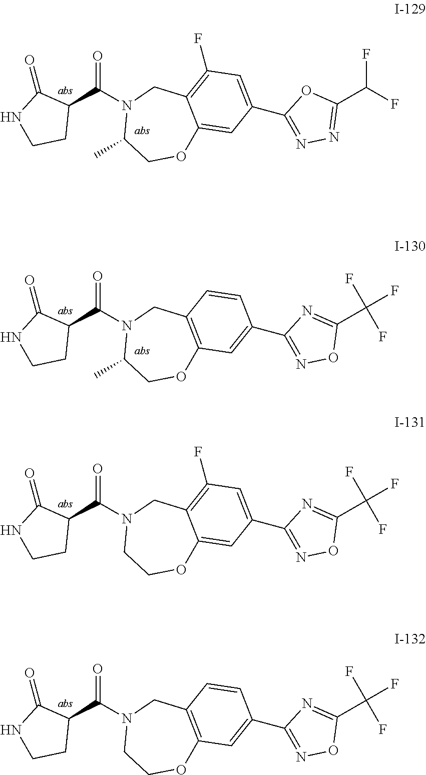 [SMILES: C[C@H]1COc2cc(-c3nnc(C(F)F)o3)cc(F)c2CN1C(=O)[C@H]1CCNC1=O ; C[C@H]1COc2cc(-c3noc(C(F)(F)F)n3)ccc2CN1C(=O)[C@H]1CCNC1=O ; O=C1NCC[C@@H]1C(=O)N1CCOc2cc(-c3noc(C(F)(F)F)n3)cc(F)c2C1 ; O=C1NCC[C@@H]1C(=O)N1CCOc2cc(-c3noc(C(F)(F)F)n3)ccc2C1]

or a pharmaceutically acceptable salt thereof.

### 3. Description of Exemplary Enumerated Embodiments

1. A compound of formula I:

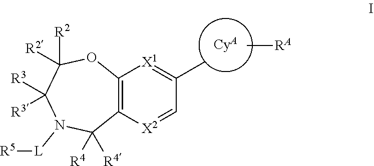

or a pharmaceutically acceptable salt thereof, wherein:
each of X¹ and X² is independently CR¹ or N;
each R¹ is independently hydrogen, halogen, —CN, or an optionally substituted C₁₋₆ aliphatic;
Cy^{A} is 1,2,4-oxadiazolyl or 1,3,4-oxadiazolyl;
R^{A} is methyl, optionally substituted with 1-3 fluoro;
each of R², R^{2′}, R³, R^{3′}, R⁴, R^{4′}, and R⁵ is independently hydrogen, halogen, —CN, —OR, —N(R)₂, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, and an 8- to 12-membered bicyclic heteroaryl having 1-4 heteroatoms independently selected from oxygen, nitrogen or sulfur; or
R³ and R^{3′}, together with their intervening atoms, form an optionally substituted 3- to 6-membered spirocyclic ring having 0-3 heteroatoms independently selected from oxygen, nitrogen, or sulfur;
L is an optionally substituted C₁₋₃ hydrocarbon chain, wherein 1-3 methylene units are optionally and independently replaced with —O—, —C(O)—, —S(O)₂—, or —NR—; and
each R is independently hydrogen or optionally substituted C₁₋₆ aliphatic.

2. The compound of embodiment 1, wherein the compound is of formula II:

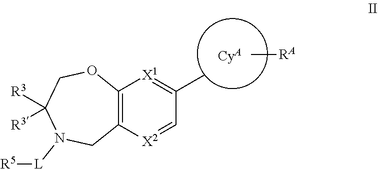

or a pharmaceutically acceptable salt thereof, wherein each of X¹, X², Cy^{A}, R^{a}, R³, R^{3′}, L, and R⁵ is as described above and within classes and subclasses herein.

3. The compound of embodiment 1 or 2, wherein X¹ and X² are CR¹.

4. The compound of embodiment 1 or 2, wherein X¹ and X² are N.

5. The compound of embodiment 1 or 2, wherein X¹ is N and X² is CR¹.

6. The compound of embodiment 1 or 2, wherein X¹ is CR¹ and X² is N.

7. The compound of embodiment 1 or 2, wherein the compound is of formula III-a or III-b:

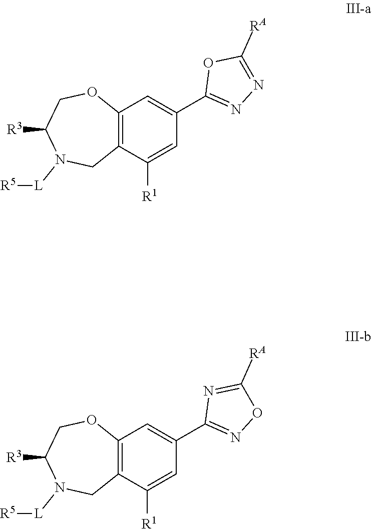

or a pharmaceutically acceptable salt thereof, wherein each of R^{A}, R¹, R³, L, and R⁵ is as described above and within classes and subclasses herein.

8. The compound of any one of embodiments 1-7, wherein R^{A} is —CF₂ or —CF₃.

9. The compound of any one of embodiments 1-8, wherein R^{A} is —CF₃.

10. The compound of any one of embodiments 1-9, wherein R³ is hydrogen, methyl, phenyl, cyclopropyl, or

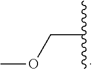 [SMILES: *COC]

11. The compound of any one of embodiments 1-10, wherein L is —C(O)—, —C(O)CH₂—*, —C(O)CH₂CH₂—*, —C(O)O—*, —C(O)CH₂O—*, —CH₂—, or —S(O)₂—, wherein * represents the point of attachment to R⁵.

12. The compound of any one of embodiments 1-11, wherein L is —C(O)—.

13. The compound of any one of embodiments 1-12, wherein R⁵ is hydrogen, —CH₃, —CF₃, —OH, —N(CH₃)₂, isopropyl t-butyl,

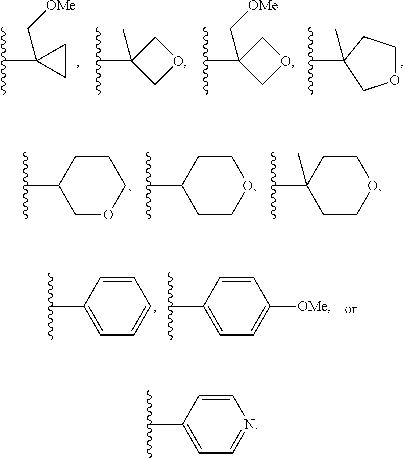

14. The compound of embodiment 1, wherein the compound is selected from the group consisting of:

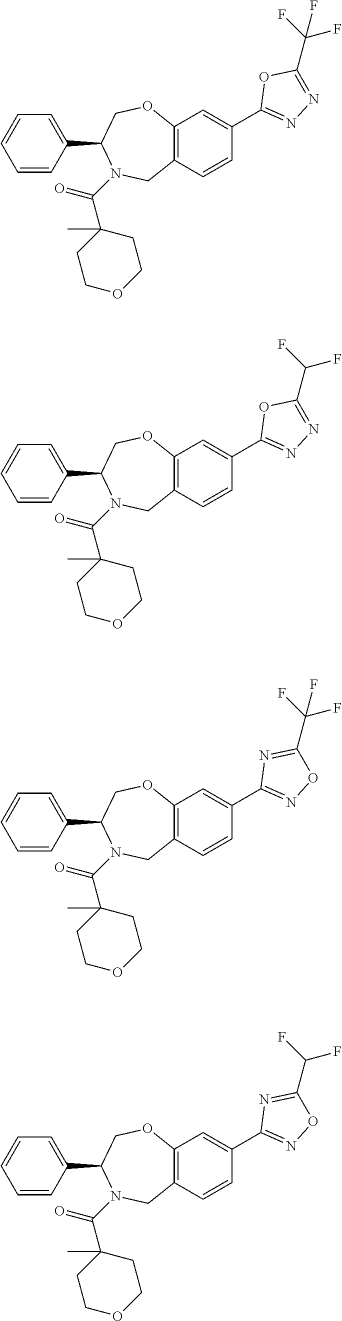 [SMILES: CC1(C(=O)N2Cc3ccc(-c4nnc(C(F)(F)F)o4)cc3OC[C@@H]2c2ccccc2)CCOCC1 ; CC1(C(=O)N2Cc3ccc(-c4nnc(C(F)F)o4)cc3OC[C@@H]2c2ccccc2)CCOCC1 ; CC1(C(=O)N2Cc3ccc(-c4noc(C(F)(F)F)n4)cc3OC[C@@H]2c2ccccc2)CCOCC1 ; CC1(C(=O)N2Cc3ccc(-c4noc(C(F)F)n4)cc3OC[C@@H]2c2ccccc2)CCOCC1]

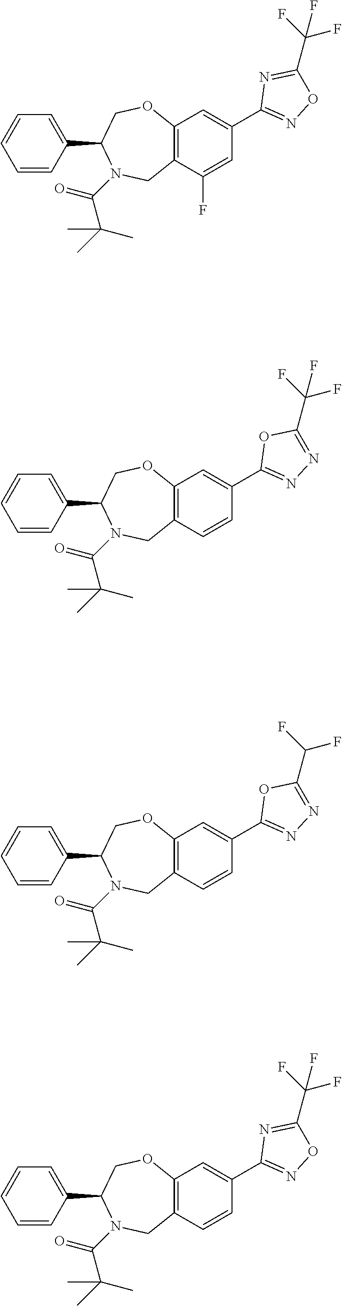 [SMILES: CC(C)(C)C(=O)N1Cc2c(F)cc(-c3noc(C(F)(F)F)n3)cc2OC[C@@H]1c1ccccc1 ; CC(C)(C)C(=O)N1Cc2ccc(-c3nnc(C(F)(F)F)o3)cc2OC[C@@H]1c1ccccc1 ; CC(C)(C)C(=O)N1Cc2ccc(-c3nnc(C(F)F)o3)cc2OC[C@@H]1c1ccccc1 ; CC(C)(C)C(=O)N1Cc2ccc(-c3noc(C(F)(F)F)n3)cc2OC[C@@H]1c1ccccc1]

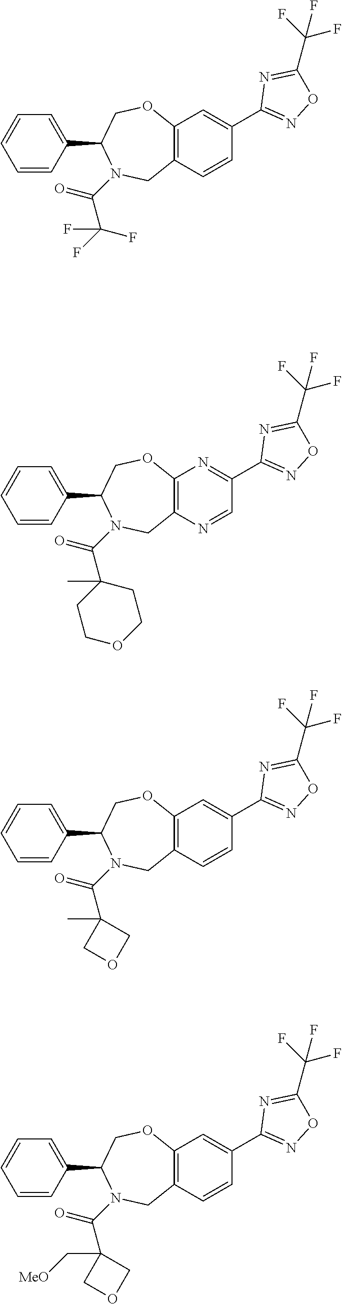 [SMILES: O=C(N1Cc2ccc(-c3noc(C(F)(F)F)n3)cc2OC[C@@H]1c1ccccc1)C(F)(F)F ; CC1(C(=O)N2Cc3ncc(-c4noc(C(F)(F)F)n4)nc3OC[C@@H]2c2ccccc2)CCOCC1 ; CC1(C(=O)N2Cc3ccc(-c4noc(C(F)(F)F)n4)cc3OC[C@@H]2c2ccccc2)COC1 ; COCC1(C(=O)N2Cc3ccc(-c4noc(C(F)(F)F)n4)cc3OC[C@@H]2c2ccccc2)COC1]

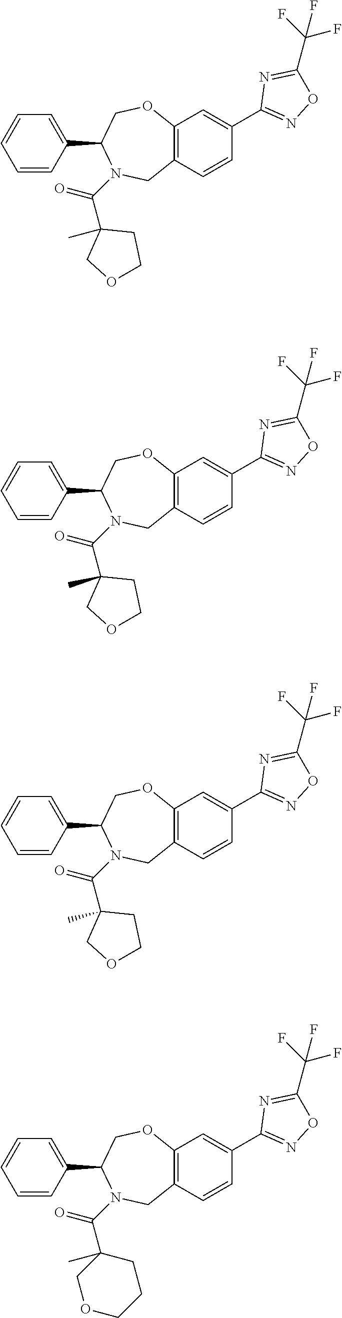 [SMILES: CC1(C(=O)N2Cc3ccc(-c4noc(C(F)(F)F)n4)cc3OC[C@@H]2c2ccccc2)CCOC1 ; C[C@@]1(C(=O)N2Cc3ccc(-c4noc(C(F)(F)F)n4)cc3OC[C@@H]2c2ccccc2)CCOC1 ; C[C@]1(C(=O)N2Cc3ccc(-c4noc(C(F)(F)F)n4)cc3OC[C@@H]2c2ccccc2)CCOC1 ; CC1(C(=O)N2Cc3ccc(-c4noc(C(F)(F)F)n4)cc3OC[C@@H]2c2ccccc2)CCCOC1]

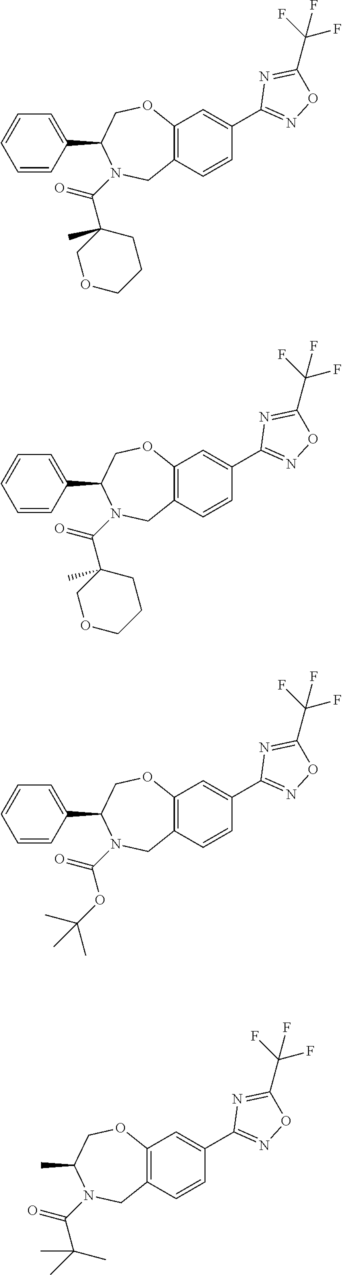 [SMILES: C[C@@]1(C(=O)N2Cc3ccc(-c4noc(C(F)(F)F)n4)cc3OC[C@@H]2c2ccccc2)CCCOC1 ; C[C@]1(C(=O)N2Cc3ccc(-c4noc(C(F)(F)F)n4)cc3OC[C@@H]2c2ccccc2)CCCOC1 ; CC(C)(C)OC(=O)N1Cc2ccc(-c3noc(C(F)(F)F)n3)cc2OC[C@@H]1c1ccccc1 ; C[C@H]1COc2cc(-c3noc(C(F)(F)F)n3)ccc2CN1C(=O)C(C)(C)C]

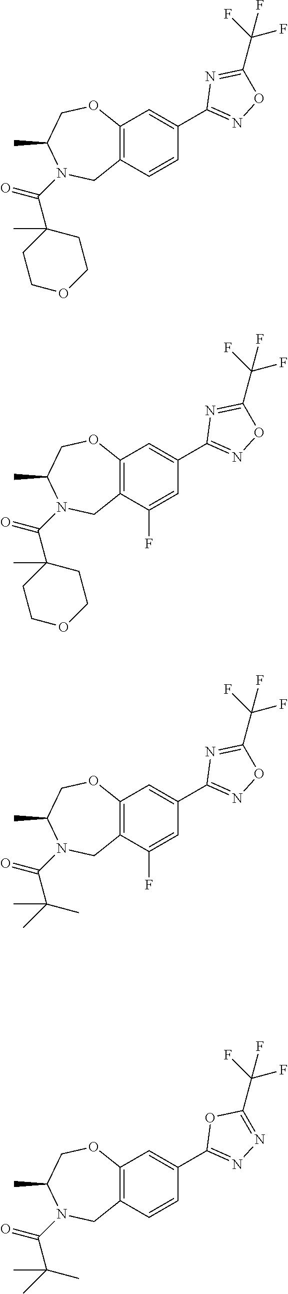 [SMILES: C[C@H]1COc2cc(-c3noc(C(F)(F)F)n3)ccc2CN1C(=O)C1(C)CCOCC1 ; C[C@H]1COc2cc(-c3noc(C(F)(F)F)n3)cc(F)c2CN1C(=O)C1(C)CCOCC1 ; C[C@H]1COc2cc(-c3noc(C(F)(F)F)n3)cc(F)c2CN1C(=O)C(C)(C)C ; C[C@H]1COc2cc(-c3nnc(C(F)(F)F)o3)ccc2CN1C(=O)C(C)(C)C]

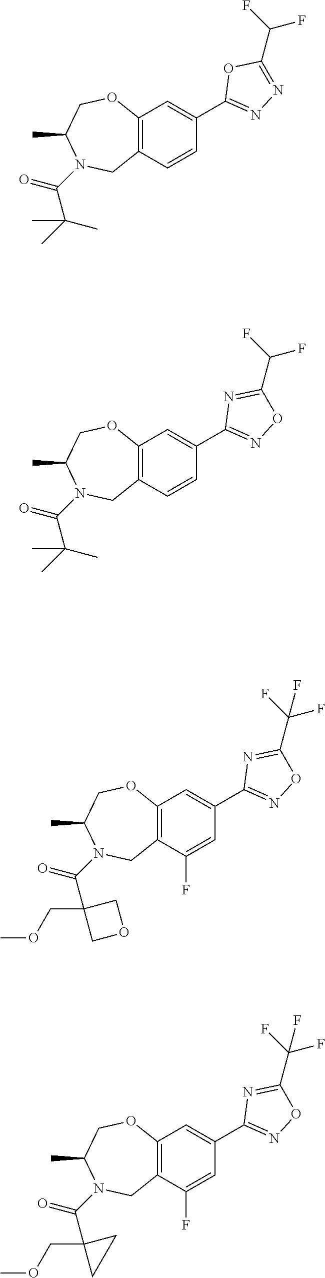 [SMILES: C[C@H]1COc2cc(-c3nnc(C(F)F)o3)ccc2CN1C(=O)C(C)(C)C ; C[C@H]1COc2cc(-c3noc(C(F)F)n3)ccc2CN1C(=O)C(C)(C)C ; COCC1(C(=O)N2Cc3c(F)cc(-c4noc(C(F)(F)F)n4)cc3OC[C@@H]2C)COC1 ; COCC1(C(=O)N2Cc3c(F)cc(-c4noc(C(F)(F)F)n4)cc3OC[C@@H]2C)CC1]

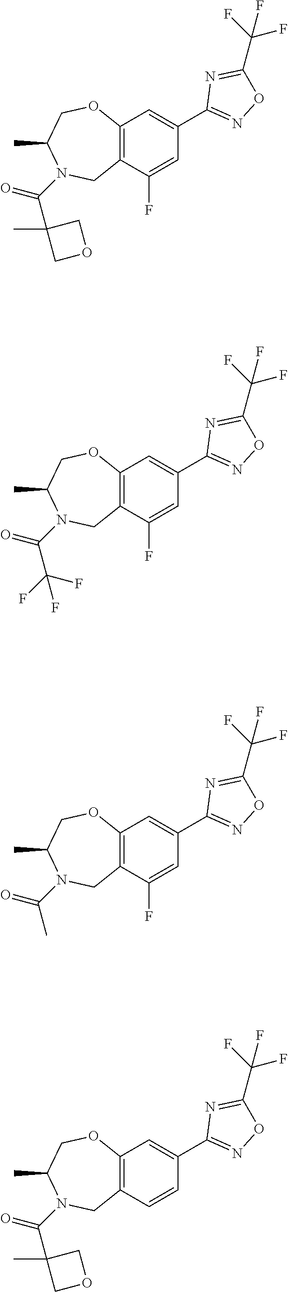 [SMILES: C[C@H]1COc2cc(-c3noc(C(F)(F)F)n3)cc(F)c2CN1C(=O)C1(C)COC1 ; C[C@H]1COc2cc(-c3noc(C(F)(F)F)n3)cc(F)c2CN1C(=O)C(F)(F)F ; CC(=O)N1Cc2c(F)cc(-c3noc(C(F)(F)F)n3)cc2OC[C@@H]1C ; C[C@H]1COc2cc(-c3noc(C(F)(F)F)n3)ccc2CN1C(=O)C1(C)COC1]

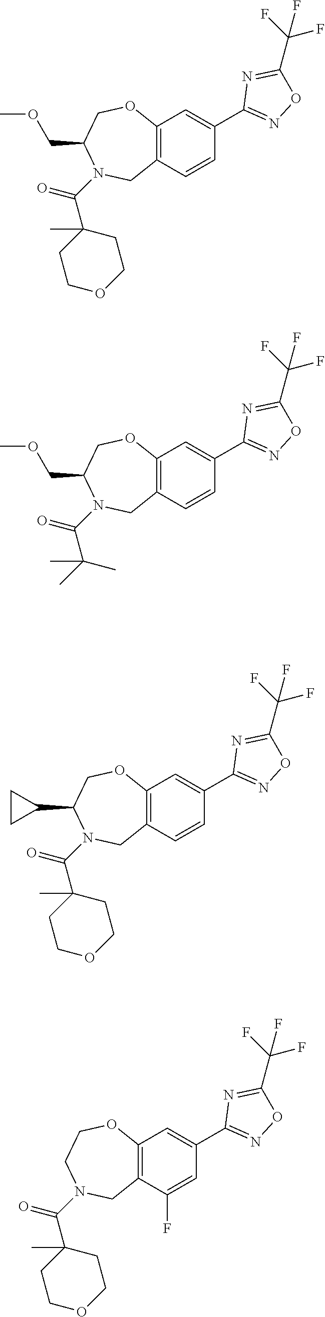 [SMILES: COC[C@H]1COc2cc(-c3noc(C(F)(F)F)n3)ccc2CN1C(=O)C1(C)CCOCC1 ; COC[C@H]1COc2cc(-c3noc(C(F)(F)F)n3)ccc2CN1C(=O)C(C)(C)C ; CC1(C(=O)N2Cc3ccc(-c4noc(C(F)(F)F)n4)cc3OC[C@@H]2C2CC2)CCOCC1 ; CC1(C(=O)N2CCOc3cc(-c4noc(C(F)(F)F)n4)cc(F)c3C2)CCOCC1]

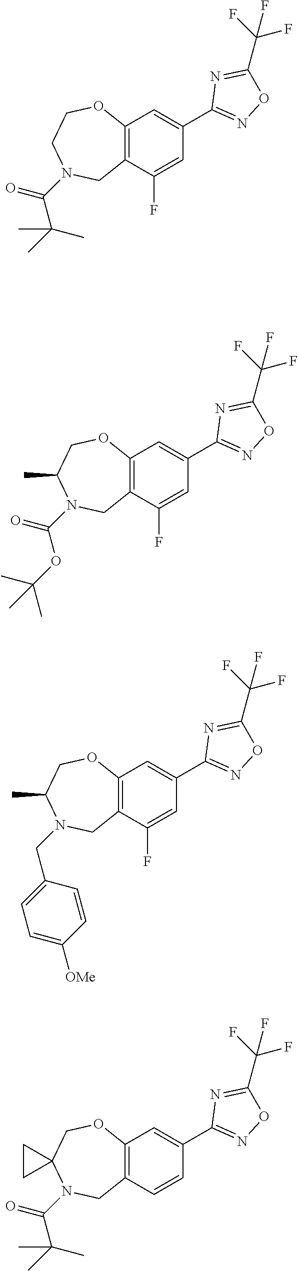 [SMILES: CC(C)(C)C(=O)N1CCOc2cc(-c3noc(C(F)(F)F)n3)cc(F)c2C1 ; C[C@H]1COc2cc(-c3noc(C(F)(F)F)n3)cc(F)c2CN1C(=O)OC(C)(C)C ; COc1ccc(CN2Cc3c(F)cc(-c4noc(C(F)(F)F)n4)cc3OC[C@@H]2C)cc1 ; CC(C)(C)C(=O)N1Cc2ccc(-c3noc(C(F)(F)F)n3)cc2OCC12CC2]

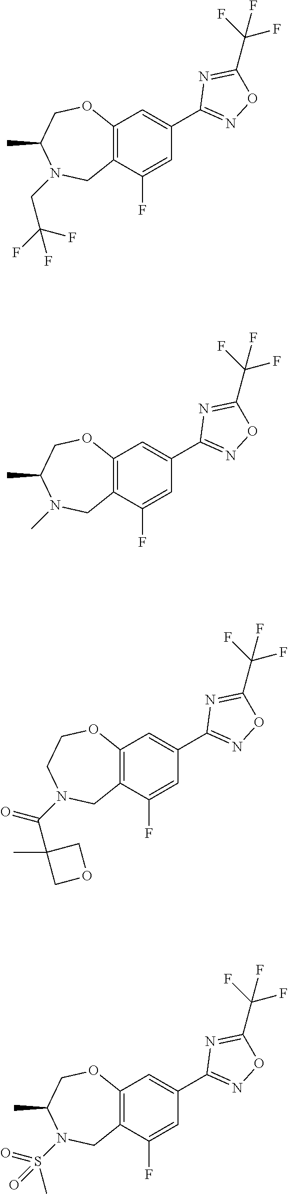 [SMILES: C[C@H]1COc2cc(-c3noc(C(F)(F)F)n3)cc(F)c2CN1CC(F)(F)F ; C[C@H]1COc2cc(-c3noc(C(F)(F)F)n3)cc(F)c2CN1C ; CC1(C(=O)N2CCOc3cc(-c4noc(C(F)(F)F)n4)cc(F)c3C2)COC1 ; C[C@H]1COc2cc(-c3noc(C(F)(F)F)n3)cc(F)c2CN1S(C)(=O)=O]

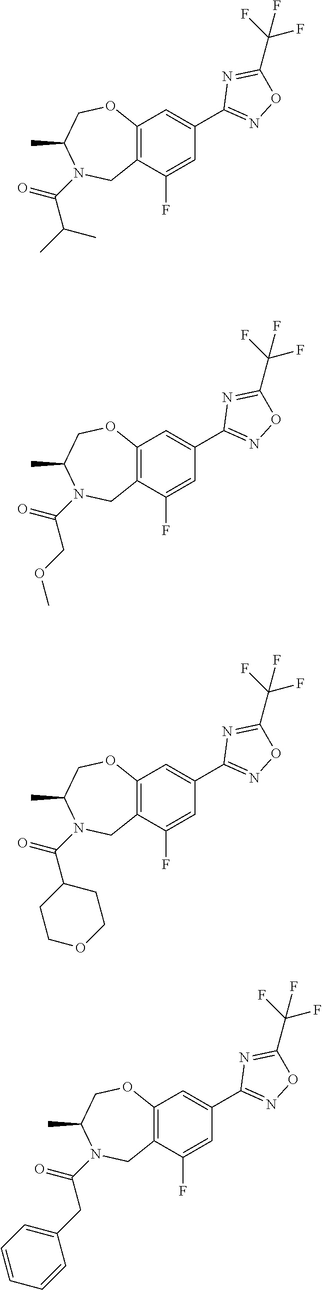 [SMILES: CC(C)C(=O)N1Cc2c(F)cc(-c3noc(C(F)(F)F)n3)cc2OC[C@@H]1C ; COCC(=O)N1Cc2c(F)cc(-c3noc(C(F)(F)F)n3)cc2OC[C@@H]1C ; C[C@H]1COc2cc(-c3noc(C(F)(F)F)n3)cc(F)c2CN1C(=O)C1CCOCC1 ; C[C@H]1COc2cc(-c3noc(C(F)(F)F)n3)cc(F)c2CN1C(=O)Cc1ccccc1]

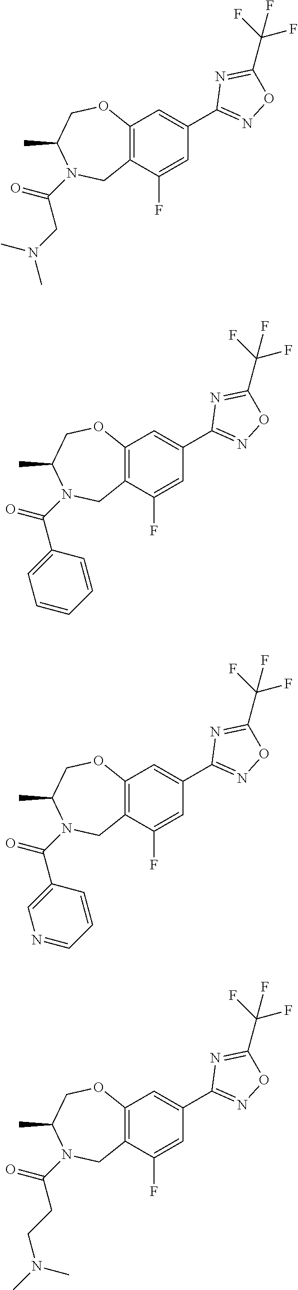 [SMILES: C[C@H]1COc2cc(-c3noc(C(F)(F)F)n3)cc(F)c2CN1C(=O)CN(C)C ; C[C@H]1COc2cc(-c3noc(C(F)(F)F)n3)cc(F)c2CN1C(=O)c1ccccc1 ; C[C@H]1COc2cc(-c3noc(C(F)(F)F)n3)cc(F)c2CN1C(=O)c1cccnc1 ; C[C@H]1COc2cc(-c3noc(C(F)(F)F)n3)cc(F)c2CN1C(=O)CCN(C)C]

or a pharmaceutically acceptable salt thereof.

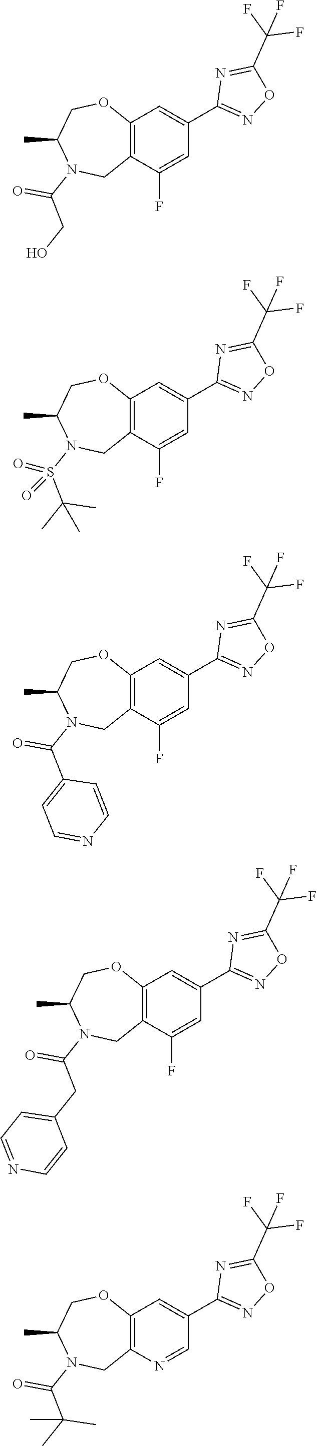 [SMILES: C[C@H]1COc2cc(-c3noc(C(F)(F)F)n3)cc(F)c2CN1C(=O)CO ; C[C@H]1COc2cc(-c3noc(C(F)(F)F)n3)cc(F)c2CN1S(=O)(=O)C(C)(C)C ; C[C@H]1COc2cc(-c3noc(C(F)(F)F)n3)cc(F)c2CN1C(=O)c1ccncc1 ; C[C@H]1COc2cc(-c3noc(C(F)(F)F)n3)cc(F)c2CN1C(=O)Cc1ccncc1 ; C[C@H]1COc2cc(-c3noc(C(F)(F)F)n3)cnc2CN1C(=O)C(C)(C)C]

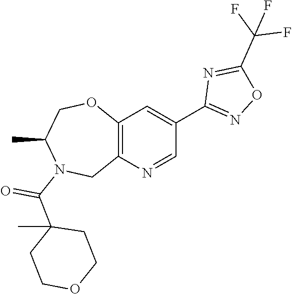 [SMILES: C[C@H]1COc2cc(-c3noc(C(F)(F)F)n3)cnc2CN1C(=O)C1(C)CCOCC1]

15. A pharmaceutical composition comprising a compound of any one of embodiments 1-14, or a pharmaceutically acceptable salt thereof, and a pharmaceutically acceptable carrier, adjuvant, or vehicle.

16. A method of inhibiting activity of HDAC6, or a mutant thereof, in a biological sample or in a patient, comprising a step of contacting the biological sample or administering to a patient a compound according to any one of embodiments 1-14, or a pharmaceutically acceptable salt thereof, or a pharmaceutical composition according to embodiment 15.

17. A method of treating a disease or disorder associated with HDAC6, or a mutant thereof, the method comprising a step of administering to a patient in need thereof a compound according to any one of embodiments 1-14, or a pharmaceutically acceptable salt thereof, or a pharmaceutical composition according to embodiment 15.

### 4. Uses, Formulation, and Administration

### Pharmaceutically Acceptable Compositions

According to another embodiment, the present disclosure provides a composition comprising a compound described herein or a pharmaceutically acceptable salt thereof and a pharmaceutically acceptable carrier, adjuvant, or vehicle. In certain embodiments, the amount of compound in compositions described herein is such that it is effective to measurably inhibit activity of a HDAC6, or a mutant thereof, in a biological sample or in a patient. In certain embodiments, a composition described herein is formulated for administration to a patient in need of such composition. In some embodiments, a composition described herein is formulated for oral administration to a patient.

Compounds and compositions, according to method of the present disclosure, are administered using any amount and any route of administration effective for treating or lessening the severity of a disorder provided herein (i.e., a HDAC6-mediated disease or disorder). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers, lubricating agents, or diluents may be used.

Solid dosage forms for oral administration include capsules, tablets, pills, powders, and granules. In such solid dosage forms, the active compound is mixed with at least one inert, pharmaceutically acceptable excipient or carrier and/or a) fillers or extenders, b) binders, c) humectants, d) disintegrating agents, e) solution retarding agents, f) absorption accelerators, g) wetting agents, h) absorbents, and/or i) lubricants, and mixtures thereof. In the case of capsules, tablets and pills, the dosage form may also comprise buffering agents.

### Uses of Compounds and Pharmaceutically Acceptable Compositions

Another aspect of the disclosure relates to a method of treating a disease associated with modulation of HDAC6, or a mutant thereof, in a subject in need thereof. The method involves administering to a patient in need of treatment for diseases or disorders associated with HDAC6 modulation an effective amount of a provided compound. In some embodiments, the disease can be, but is not limited to, cancer, neurodegenerative disease, neurodevelopmental disease, inflammatory or autoimmune disease, infection, metabolic disease, hematologic disease, or cardiovascular disease. In some embodiments, a provided compound is for use in medicine.

Another aspect of the disclosure is directed to a method of inhibiting activity of HDAC6, or a mutant thereof. The method involves administering to a patient in need thereof an effective amount of a provided compound.

The present disclosure relates to compositions capable of modulating the activity of (e.g., inhibiting) HDAC6, or a mutant thereof. The present disclosure also relates to the therapeutic use of such compounds. Exemplary therapeutic uses are disclosed in PCT Publication Numbers WO2016/126724 and WO2016/126725.

### HDAC6

Among the eleven zinc-dependent HDACs (comprising Classes I, IIa, IIb, and IV), HDAC6 is a member of Class IIB that is localized largely to the cytoplasm. HDAC6 is unique in that it contains tandem catalytic domains, which provide capacity to deacetylate a variety of non-histone proteins, e.g., α-tubulin, HSP90, peroxiredoxin, cortactin, surviving, β-catenin. See Brindisi, M. et al. J. Med. Chem. 2019: published online. 2019 Aug. 15; Shen, S. et al. Expert Opinion on Therapeutic Patents 2020, 30(2), 121-136. In some embodiments, compounds disclosed herein disrupt or inhibit the interaction between HDAC6 and a non-histone substrate (e.g., α-tubulin, HSP90, peroxiredoxin, cortactin, survivin, β-catenin). In some embodiments, compounds disclosed herein inhibit deacetylation of a non-histone substrate (e.g., α-tubulin, HSP90, peroxiredoxin, cortactin, survivin, β-catenin) by HDAC6, or a mutant thereof. In some embodiments, compounds disclosed herein bind to HDAC6, or a mutant thereof, and inhibit deacetylation of a non-histone substrate (e.g., α-tubulin, HSP90, peroxiredoxin, cortactin, survivin, β-catenin).

In some embodiments, the present disclosure provides methods of inhibiting, reducing, or lessening deacetylation of a non-histone substrate (e.g., α-tubulin, HSP90, peroxiredoxin, cortactin, survivin, β-catenin) comprising contacting a cell with a provided compound (e.g., a compound of formula I). In some embodiments, compounds disclosed herein disrupt or inhibit the interaction between HDAC6 and a non-histone substrate. In some embodiments, compounds disclosed herein disrupt or inhibit the interaction between HDAC6 and α-tubulin. In some embodiments, compounds disclosed herein disrupt or inhibit the interaction between HDAC6 and HSP90. In some embodiments, compounds disclosed herein disrupt or inhibit the interaction between HDAC6 and peroxiredoxin. In some embodiments, compounds disclosed herein disrupt or inhibit the interaction between HDAC6 and cortactin. In some embodiments, compounds disclosed herein disrupt or inhibit the interaction between HDAC6 and survivin. In some embodiments, compounds disclosed herein disrupt or inhibit the interaction between HDAC6 and β-catenin.

In some embodiments, compounds disclosed herein are inhibitors of HDAC6, or mutants thereof, and are therefore useful for treating one or more disorders associated with activity of HDAC6. In some embodiments, the present disclosure provides methods for treating an HDAC6-mediated disease, disorder, or condition comprising a step of administering to a patient in need thereof a compound disclosed herein, or a pharmaceutically acceptable composition thereof. As used herein, the term “HDAC6-mediated” diseases, disorders, or conditions refers to any diseases, disorders, or conditions in which HDAC6, or a mutant thereof, is known to play a role.

### Cancer

The interaction of HDAC6 with histone and nonhistone substrates is involved in gene transcription, DNA damage repair, and cell movement; and once the expression level of HDAC6 changes or its activity increases, it can lead to oncogenic cell transformation and tumor cell proliferation, invasion, metastasis, and mitosis. See Li, T., et al. “Histone deacetylase 6 in cancer.” Journal of Hematology and Oncology. 2018, 11, 111. The up-regulation of HDAC6 in diverse tumors and cell lines also suggests an important role in cancer, e.g., HDAC6 is considered to be required for the efficient activation of the oncogenic Ras signaling pathway. See Aldana-Masangkay, G., et al. “The Role of HDAC6 in Cancer” Journal of Biomedicine and Biotechnology 2011, Article ID 875824. In some embodiments, the present disclosure provides methods of treating cancer comprising a step of administering to a patient in need thereof a therapeutically effective compound of the present disclosure, or a pharmaceutically acceptable composition thereof. In some embodiments, a cancer is multiple myeloma, colon cancer, lymphoma (e.g., histiocytic lymphoma, cutaneous T-cell lymphomas, and relapsed or refractory peripheral T-cell lymphomas), or glioblastoma.

In some aspects, inhibitors of HDAC6 may be useful in treating cancer (e.g., multiple myeloma) alone, or in combination with a proteasome inhibitor (e.g., bortezomib) and/or a glucocorticoid (e.g., dexamethasone). See Vogl et al. “Ricolinostat, the First Selective Histone Deacetylase 6 Inhibitor, in Combination with Bortezomib and Dexamethasone for Relapsed or Refractory Multiple Myeloma.” Clin Cancer Res. 2017, 23(13), 3307-3315. In some embodiments, provided compounds may be useful in treating multiple myeloma. In some embodiments, the present disclosure provides methods of treating multiple myeloma comprising a step of administering to a patient in need thereof a therapeutically effective amount of a compound of the present disclosure, or a pharmaceutically acceptable composition thereof. In some embodiments, provided methods of treating a cancer (e.g., multiple myeloma) further comprises administering to a patient in need thereof a proteasome inhibitor (e.g., bortezomib, carfilzomib, or ixazomib). In some embodiments, administration of a proteasome inhibitor (e.g., bortezomib, carfilzomib, or ixazomib) occurs prior to or concurrently with administration of a provided compound. In some embodiments, provided methods of treating a cancer (e.g., multiple myeloma) further comprises a step of administering to a patient in need thereof a corticosteroid (e.g., a glucocorticoid such as dexamethasone). In some embodiments, administration of a corticosteroid (e.g., a glucocorticoid such as dexamethasone) occurs prior to or concurrently with administration of a provided compound. In some embodiments, a patient is receiving or has received a proteasome inhibitor (e.g., bortezomib, carfilzomib, or ixazomib) or a corticosteroid (e.g., a glucocorticoid such as dexamethasone). In some embodiments, a patient is receiving or has received a proteasome inhibitor (e.g., bortezomib, carfilzomib, or ixazomib) and a corticosteroid (e.g., a glucocorticoid such as dexamethasone).

In some aspects, inhibitors of HDAC6 may be useful in treating cancer (e.g., colon cancer or lymphoma, such as histiocytic lymphoma, cutaneous T-cell lymphomas, and relapsed or refractory peripheral T-cell lymphomas). See Relitti, N. et al. “Novel quinolone-based potent and selected HDAC6 inhibitors: Synthesis, molecular modeling studies and biological investigation.” European Journal of medicinal Chemistry 2020, in press. In some embodiments, the present disclosure provides methods of treating colon cancer or lymphoma (e.g., histiocytic lymphoma, cutaneous T-cell lymphomas, and relapsed or refractory peripheral T-cell lymophomas) comprising a step of administering to a patient in need thereof a therapeutically effective amount of a compound of the present disclosure, or a pharmaceutically acceptable composition thereof.

In some aspects, inhibitors of HDAC6 may be useful in treating ARID1A-mutated cancers (e.g., ovarian, endometrial, hepatocellular, bladder, colorectal, gastric, and non-small cell lung cancers) alone, or in combination with an immune checkpoint blockade (e.g., anti-PD-L1 immune checkpoint blockade). HDAC6 inhibitors may suppress ARID1A-mutated tumors (e.g., ovarian, endometrial, hepatocellular, bladder, colorectal, gastric, and non-small cell lung cancers) by both targeting cancer cells and restoring antitumor immunity. See Fukumoto, T., et al. “HDAC6 inhibition synergizes with anti-PD-L1 therapy in ARID1A-inactivated ovarian cancer.” Cancer Res. 2019, 79(21), 5482-5489; and Hung, et al. “ARID1A mutations and expression loss in non-small cell lung carcinomas: clinicopathologic and molecular analysis” Modern Pathology 2020, 33, 2256-2268. In some embodiments, the present disclosure provides methods of treating ARID1A-mutated cancers (e.g., ovarian, endometrial, hepatocellular, bladder, colorectal, gastric, and non-small cell lung cancers) comprising a step of administering to a patient in need thereof a therapeutically effective amount of a compound of the present disclosure, or a pharmaceutically acceptable composition thereof. In some embodiments, provided methods of treating a ARID1A-mutated cancers (e.g., ovarian, endometrial, hepatocellular, bladder, colorectal, gastric, and non-small cell lung cancers) further comprises a step of administering to a patient in need thereof an immune checkpoint blockade (e.g., anti-PD-L1 immune checkpoint blockade). In some embodiments, administration of an immune checkpoint blockade (e.g., anti-PD-L1 immune checkpoint blockade) occurs prior to or concurrently with administration of a provided compound. In some embodiments, a patient is receiving or has previously received an immune checkpoint blockade (e.g., anti-PD-L1 immune checkpoint blockade).

In some aspects, inhibitors of HDAC6 may be useful in treating breast cancer alone, or in combination with an immune checkpoint blockade (e.g., anti-PD-L1 immune checkpoint blockade). See Banik, D. et al. “HDAC6 Plays a Noncanonical Role in the Regulation of Antitumor Immune Responses, Dissemination, and Invasiveness of Breast Cancer.” Cancer Res. 2020, 80, 3649-3662. In some embodiments, the present disclosure provides methods of treating breast cancer comprising a step of administering to a patient in need thereof a therapeutically effective amount of a compound of the present disclosure, or a pharmaceutically acceptable composition thereof. In some embodiments, provided methods of treating breast cancer further comprises a step of administering to a patient in need thereof an immune checkpoint blockade (e.g., anti-PD-L1 immune checkpoint blockade). In some embodiments, administration of an immune checkpoint blockade (e.g., anti-PD-L1 immune checkpoint blockade) occurs prior to or concurrently with administration of a provided compound. In some embodiments, a patient is receiving or has previously received an immune checkpoint blockade (e.g., anti-PD-L1 immune checkpoint blockade).

### Neurological Diseases, Disorders, or Conditions

In some embodiments, the present disclosure provides the recognition that provided compounds, which comprise a particular zinc-binding group (di/trifluoromethyloxadiazoles) bound to a particular linker/capping group comprising a 6,7-bicyclic ring, exhibit improved brain penetration properties, e.g., as compared to a reference compound. In some embodiments, a reference compound is a corresponding compound with a different zinc-binding group (e.g., a hydroxamic acid or mercaptoacetamide moiety).

Relatively high expression of HDAC6 has been observed in the brain. Koole, et al. “Clinical validation of the novel HDAC6 radiotracer [18F]EKZ-001 in the human brain.” Eur. J. Nucl. Med. Mol. Imaging 2020, published online. HDAC6 has also been found to target synaptic protein Bruchpilot and neurotransmitter release, and in pathological conditions, HDAC6 becomes abundant in the nucleus, with deleterious consequences for transcription regulation and synapses. See LoPresti, P. “HDAC6 in Diseases of Cognition and of Neurons.” Cells 2021, 10, 12.

Without wishing to be bound by a particular theory, due in part to their improved brain penetration properties, relatively high expression of HDAC6 observed in the brain, and/or HDAC6 function within neurons, compounds of the present disclosure are useful in treating diseases, disorders, or conditions associated with the central nervous system (e.g., brain and/or spinal cord) or the peripheral nervous system. In some embodiments, the present disclosure provides methods of treating a disease, disorder, or condition associated with the central nervous system comprising a step of administering to a patient in need thereof a therapeutically effective amount of a compound of the present disclosure, or a pharmaceutically acceptable composition thereof. In some embodiments, a disease, disorder, or condition associated with the central nervous system includes Amyotrophic Lateral Sclerosis (ALS), Alzheimer's disease, Parkinson's disease, Rett syndrome (RTT), Charcot-Marie-Tooth (CMT) disease, Fragile X Syndrome (FXS), Rubinstein-Taybi syndrome, depression, and schizophrenia.

In some aspects, inhibitors of HDAC6 may be useful in treating ALS. See Guo et al. “HDAC6 inhibition reverses axonal transport defects in motor neurons derived from FUS-ALS patients.” Nature Communications. 2017, 8:861, 1-15. In some embodiments, defects in axonal transport caused by mutant FUS may be rescued by HDAC6 inhibitors. See Guo et al. “HDAC6 inhibition reverses axonal transport defects in motor neurons derived from FUS-ALS patients.” Nature Communications. 2017, 8:861, 1-15. In some embodiments, the present disclosure provides methods of treating ALS comprising a step of administering to a patient in need thereof a therapeutically effective amount of a compound of the present disclosure, or a pharmaceutically acceptable composition thereof. In some embodiments, methods of treating ALS comprise lessening severity or progression of a symptom of ALS (e.g., loss of motor function). In some embodiments, the present disclosure provides methods of restoring axonal transport within patients suffering from ALS. In some embodiments, an ALS patient has an SOD1 mutation. In some embodiments, an ALS patient does not have an SOD1 mutation. In some embodiments, ALS is FUS-induced ALS.

In some embodiments, the present disclosure provides methods of treating a disease, disorder, or condition associated with the peripheral nervous system comprising a step of administering to a patient in need thereof a therapeutically effective amount of a compound of the present disclosure, or a pharmaceutically acceptable composition thereof. In some aspects, inhibitors of HDAC6 may be useful in treating peripheral nerve injury or peripheral inflammation. See Sakloth et al. Psychopharmacology (Berl). 2000, 237(7) 2139-2149. In some embodiments, the present disclosure provides methods of treating peripheral nerve injury or peripheral inflammation comprising a step of administering to a patient in need thereof a therapeutically effective amount of a compound of the present disclosure, or a pharmaceutically acceptable composition thereof. In some embodiments, the present disclosure provides methods of treating mechanical allodynia (e.g., associated with peripheral nerve injury or inflammation) comprising a step of administering to a patient in need thereof a therapeutically effective amount of a compound of the present disclosure, or a pharmaceutically acceptable composition thereof.

In some aspects, inhibitors of HDAC6 may be useful in treating peripheral neuropathies, including Charcot-Marie-Tooth (CMT) disease (e.g., CMT1A, CMT2A, CMT2D, CMT2F disease). See Ha et al., “A novel histone deacetylase 6 inhibitor improves myelination of Schwann cells in a model of Charcot-Marie-Tooth disease type 1A.” Br. J. Pharmacol. 2020, 177, 5096-5113; Picci et al. “HDAC6 inhibition promotes α-tubulin acetylation and ameliorates CMT2A peripheral neuropathy in mice.” 2020, 328, 113281, 1-14; Mo, et al. “Aberrant GlyRS-HDAC6 interaction linked to axonal transport deficits in Charcot-Marie-Tooth neuropathy.” Nature Communications 2018 9(1007), 1-11; Adalbert, R. et al. “Novel HDAC6 Inhibitors Increase Tubulin Acetylation and Rescue Axonal Transport of Mitochondria in a Model of Charcot-Marie-Tooth Type 2F.” ACS Chem. Neurosci. 2020, 11, 258-267; Rossaert, E., et al. “HDAC6 inhibitors: Translating genetic and molecular insights into a therapy for axonal CMT.” Brain Research 2020, 1733, 146692. In some embodiments, the present disclosure provides methods of treating CMT disease (e.g., CMT1A, CMT2A, CMT2D, CMT2F disease) comprising a step of administering to a patient in need thereof a therapeutically effective amount of a compound of the present disclosure, or a pharmaceutically acceptable composition thereof.

In some aspects, HDAC6 inhibitors may be useful in treating cancers of the brain. Overexpression of HDAC6 has been observed in glioblastoma. See Auzmendi-Iriarte, J. et al. “Characterization of a new small-molecule inhibitor of HDAC6 in glioblastoma” Cell Death & Disease 2020, 11, 417. In some embodiments, provided compounds may be useful in treating glioblastoma. In some embodiments, the present disclosure provides methods of treating glioblastoma comprising a step of administering to a patient in need thereof a therapeutically effective amount of a compound of the present disclosure, or a pharmaceutically acceptable composition thereof.

In some aspects, HDAC6 inhibitors may be useful in treating Fragile X Syndrome (FXS). See Kozikowski, A., et al. “Brain Penetrable Histone Deacetylase 6 Inhibitor SW-100 Ameliorates Memory and Learning Impairments in a Mouse Model of Fragile X Syndrome.” 2019, 10, 1679-1695. In some embodiments, provided compounds may be useful in treating FXS. In some embodiments, the present disclosure provides methods of treating FXS comprising a step of administering to a patient in need thereof a therapeutically effective amount of a compound of the present disclosure, or a pharmaceutically acceptable composition thereof.

### Chemotherapy-Induced Peripheral Neuropathy (CIPN)

CIPN is a nerve-damaging side effect common in cancer treatments, afflicting between about 30 and 40% of patients undergoing chemotherapy. CIPN involves various symptoms such as tingling, pain, and numbness in the hands and feet, which can impair activities of daily living and increase risk of falls and hospitalizations. Loprinzi, C. L. “Prevention and Management of Chemotherapy-Induced Peripheral Neuropathy in Survivors of Adult Cancers: ASCO Guideline Update” Journal of Clinical Oncology 2020, 38(28), 3325-3348. CIPN also often leads to patients reducing or discontinuing chemotherapy. CIPN frequently persists or even worsens after completion of chemotherapy. Symptoms of CIPN include pain, numbness, tingling, and temperature sensitivity.

In some embodiments, provided compounds may be useful in treating CIPN. In some embodiments, the present disclosure provides methods of treating CIPN comprising a step of administering to a patient in need thereof a therapeutically effective amount of a compound of the present disclosure, or a pharmaceutically acceptable composition thereof. In some embodiments, provided methods of treating CIPN further comprise a step of administering to a patient in need thereof a chemotherapy. In some embodiments, administration of a chemotherapy occurs prior to or concurrently with administration of a provided compound, or a pharmaceutically acceptable salt thereof. In some embodiments, a patient is receiving or has previously received a chemotherapy for treatment of cancer.

In some aspects, HDAC6 inhibitors may be useful in treating CIPN in patients that have received or are receiving taxol. See Lee et al. “Results of an abbreviated Phase Ib study of the HDAC6 inhibitor ricolinostat and paclitaxel in recurrent ovarian, fallopian tube, or primary peritoneal cancer” Gynecologic Oncology Reports 29 (2019) 118-122. In some embodiments, provided compounds may be useful in treating CIPN in patients that have received or are receiving taxol. In some embodiments, patients have received or are receiving taxol include those suffering from ovarian, fallopian tube, or primary peritoneal cancers.

In some aspects, HDAC6 inhibitors may be useful in treating CIPN in patients that have received or are receiving cisplatin. See Krukowski, et al. “HDAC6 inhibition effectively reverses chemotherapy-induced peripheral neuropathy.” Pain 2017. 158(6), 1126-1137. Without wishing to be bound to a particular theory, CIPN induced by cisplatin may be due to mitochondrial defects caused by cisplatin and/or a decrease of acetylation of α-tubulin, whereas HDAC6 inhibition may improve α-tubulin acetylation and/or mitochondrial health and transport and contribute to treating CIPN. See Krukowski, et al. “HDAC6 inhibition effectively reverses chemotherapy-induced peripheral neuropathy.” Pain 2017. 158(6), 1126-1137; Ma, J., et al. “Pharmacological inhibition of HDAC6 reverses cognitive impairment and tau pathology as a result of cisplatin treatment.” Acta Neuropathologica Communications 2018, 6, 103; Ma, J., et al. “Cell-specific role of histone deacetylase 6 in chemotherapy-induced mechanical allodynia and loss of intraepidermal nerve fibers.” Pain 2019, 160, 2877-2890. In some embodiments, provided compounds may be useful in treating CIPN in patients that have received or are receiving cisplatin. In some embodiments, patients have received or are receiving cisplatin include those suffering from testicular cancer, ovarian cancer, cervical cancer, breast cancer, bladder cancer, head and neck cancer, esophageal cancer, lung cancer, mesothelioma, brain cancer, and neuroblastoma.

### Preparation of Compounds

The compounds of the present disclosure can be prepared in a number of ways well known to those skilled in the art of organic synthesis. By way of example, compounds of the present disclosure can be synthesized using methods described PCT Publication Numbers WO2016/126724 and WO2016/126725. Further exemplification of certain compounds is provided in the ensuing examples, which may be adapted according to known methods and/or intermediates to prepare other compounds provided herein.

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

In some embodiments, the symbol “&” followed by a number appears adjacent to a stereocenter. In such cases, it is understood to include a mixture of both configurations (e.g., R- and S-) at that position. In some embodiments, the term “or” followed by a number appears adjacent to a stereocenter. In such cases, it is understood to denote either an “R—” or “S—” isomer, but the particular isomer was not determined.

In some embodiments, the numbering following the symbol “&” or term “or” refers to one stereocenter's relation to another stereocenter in that compound. For example, where two stereocenters in a compound are each denoted with the same number (e.g., two instances of “& 1”), it is understood that the configurations are relative to each other (e.g., if the structure is drawn as (S,S) and both stereocenters are denoted “&1”, it is understood to include a mixture of the (S,S) and (R,R) isomers, but not the (S,R) or (R,S) isomers). However, where each stereocenter is denoted with a different number (e.g., one instance of “&1” and one instance of “&2”), it is understood that that the configurations may be independent to each other (e.g., if the structure is drawn (S,S) and one stereocenter is denoted “&1” and one is denoted “&2,” it is understood to include a mixture of the (S,S), (S,R), (R,S), and (R,R) isomers).

### Example 1. Synthesis of Exemplary Compounds

### Example 1.1. Synthesis of (S)-(6-fluoro-3-methyl-8-(5-(trifluoromethyl)-1,2,4-oxadiazol-3-yl)-2,3-dihydrobenzo[f][1,4]oxazepin-4(5H)-yl)(3-methyloxetan-3-yl)methanone (I-1)

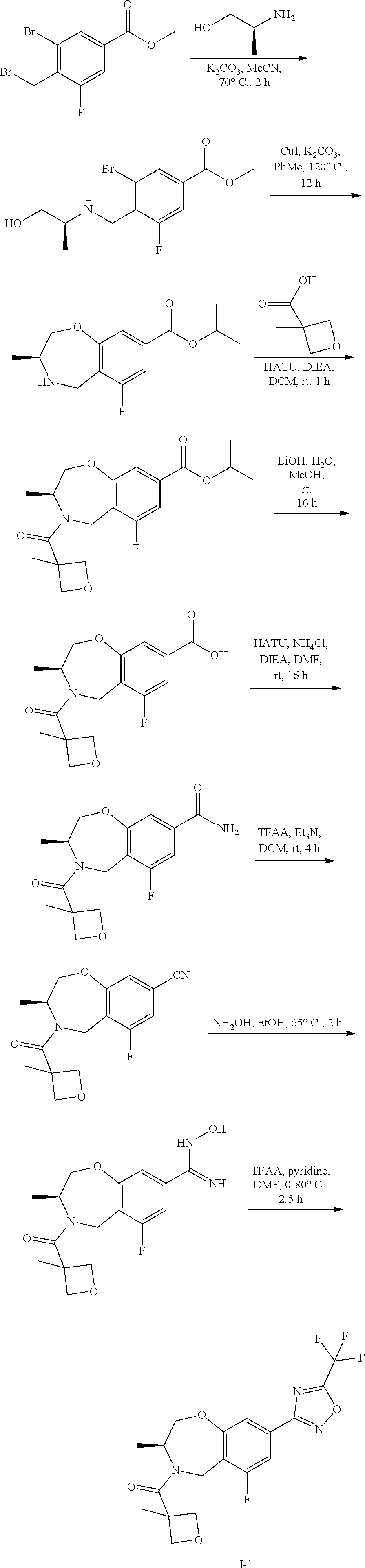

methyl (S)-3-bromo-5-fluoro-4-(((1-hydroxypropan-2-yl)amino)methyl)benzoate. To a solution of methyl 3-bromo-4-(bromomethyl)-5-fluorobenzoate (7.0 g, 21.5 mmol) and (2S)-2-aminopropan-1-ol (4.84 g, 64.5 mmol) in MeCN (100 mL) stirred under nitrogen atmosphere at room temperature was added K₂CO₃ (8.91 g, 64.5 mmol). The reaction mixture was stirred at room temperature for 12 h under nitrogen atmosphere. The mixture was quenched with water (50 mL) and extracted with DCM (3×50 mL). The combined organic layer was dried over Na₂SO₄ and concentrated under reduced pressure to get a residue which was purified by column chromatography on silica gel (80 g) eluting with MeOH in DCM (0-8%) to afford methyl 3-bromo-5-fluoro-4-({[(2S)-1-hydroxypropan-2-yl]amino}methyl)benzoate (6.1 g, 79%) as a white solid. MS (ESI): mass calcd. for C₁₂H₁₅BrFNO₃, 319.02, m/z found 320.0 [M+H]⁺.

isopropyl (S)-6-fluoro-3-methyl-2,3,4,5-tetrahydrobenzo[f][1,4]oxazepine-8-carboxylate. To a solution of methyl 3-bromo-5-fluoro-4-({[(2S)-1-hydroxypropan-2-yl]amino}methyl)benzoate (4.0 g, 12.5 mmol) and K₂CO₃ (5.2 g, 37.5 mmol) in IPA (40 mL) was added Copper(I) iodide (0.48 g, 2.5 mmol). The reaction mixture was stirred at 100° C. under nitrogen atmosphere for 16 h. The mixture was cooled to room temperature and concentrated under reduced pressure. The residue was diluted with water (30 mL) and extracted with DCM (3×30 mL). The combined organic layer was dried over Na₂SO₄ and concentrated under reduced pressure to get a residue which was purified by column chromatography on silica gel (40 g) eluting with EA in PE (0-30%) to afford methyl (3S)-6-fluoro-3-methyl-2,3,4,5-tetrahydro-1,4-benzoxazepine-8-carboxylate (2.2 g, 68%) as yellow oil. MS (ESI): mass calcd. for C₁₄H₁₈FNO₃, 267.13, m/z found 268.1 [M+H]⁺.

isopropyl (S)-6-fluoro-3-methyl-4-(3-methyloxetane-3-carbonyl)-2,3,4,5-tetrahydrobenzo[f][1,4]oxazepine-8-carboxylate. To a solution of isopropyl (3S)-6-fluoro-3-methyl-2,3,4,5-tetrahydro-1,4-benzoxazepine-8-carboxylate (600 mg, 2.24 mmol) and 3-methyloxetane-3-carboxylic acid (313 mg, 2.69 mmol) in DMF (20 mL) stirred under nitrogen atmosphere at room temperature was added DIEA (870 mg, 6.73 mmol), HATU (1.3 g, 3.37 mmol). The reaction mixture stirred at room temperature for 2 h under nitrogen atmosphere. The mixture was quenched with water (20 mL) and extracted with DCM (3×15 mL). The combined organic layer was dried over Na₂SO₄ and concentrated under reduced pressure to get a residue which was purified by column chromatography on silica gel (25 g) eluting with EA in PE (0-50%) to afford isopropyl (3S)-6-fluoro-3-methyl-4-[(3-methyloxetan-3-yl)carbonyl]-3,5-dihydro-2H-1,4-benzoxazepine-8-carboxylate (750 mg, 82%) as a yellow solid. MS (ESI): mass calcd. for C₁₉H₂₄FNO₅, 365.16, m/z found 366.1 [M+H]⁺.

(S)-6-fluoro-3-methyl-4-(3-methyloxetane-3-carbonyl)-2,3,4,5-tetrahydrobenzo[f][1,4]oxazepine-8-carboxylic acid. To a solution of isopropyl (3S)-6-fluoro-3-methyl-4-[(3-methyloxetan-3-yl)carbonyl]-3,5-dihydro-2H-1,4-benzoxazepine-8-carboxylate (750 mg, 2.05 mmol) in THE (2 mL) and H₂O (2 mL) was added Lithium hydroxide (148 mg, 6.16 mmol). The reaction mixture was stirred at room temperature for 2 h. The mixture was adjusted to pH=2-3 with 1M HCl and extracted with DCM (3×10 mL). The combined organic layer was dried over Na₂SO₄ and concentrated under reduced pressure to get (3S)-6-fluoro-3-methyl-4-[(3-methyloxetan-3-yl)carbonyl]-3,5-dihydro-2H-1,4-benzoxazepine-8-carboxylic acid (611 mg, 83%) as yellow oil. MS (ESI): mass calcd. for C₁₆H₁₈FNO₅, 323.12, m/z found 324.1 [M+H]⁺.

(S)-6-fluoro-3-methyl-4-(3-methyloxetane-3-carbonyl)-2,3,4,5-tetrahydrobenzo[f][1,4]oxazepine-8-carboxamide. To a solution of (3S)-6-fluoro-3-methyl-4-[(3-methyloxetan-3-yl)carbonyl]-3,5-dihydro-2H-1,4-benzoxazepine-8-carboxylic acid (611 mg, 1.89 mmol) and NH₄Cl (303 mg, 5.67 mmol) in DMF (20 mL) was added DIEA (733 mg, 5.67 mmol), HATU (1437 mg, 3.78 mmol). The reaction mixture was stirred at room temperature for 2 h. The mixture was quenched with water (20 mL) and extracted with EA (20 mL×3). The combined organic layer was washed with brine (20 mL×3) and concentrated to get the required product (3S)-6-fluoro-3-methyl-4-[(3-methyloxetan-3-yl)carbonyl]-3,5-dihydro-2H-1,4-benzoxazepine-8-carboxamide (800 mg, 79%) as yellow oil. MS (ESI): mass calcd. for C₁₆H₁₉FN₂O₄, 322.13, m/z found 323.1 [M+H]⁺.

(S)-6-fluoro-3-methyl-4-(3-methyloxetane-3-carbonyl)-2,3,4,5-tetrahydrobenzo[f][1,4]oxazepine-8-carbonitrile. To a solution of (3S)-6-fluoro-3-methyl-4-[(3-methyloxetan-3-yl)carbonyl]-3,5-dihydro-2H-1,4-benzoxazepine-8-carboxamide (800 mg, 2.48 mmol) and NEt₃ (753 mg, 7.45 mmol) in DCM (20 mL) stirred under nitrogen atmosphere at 0° C. was added TFAA (1.6 g, 7.45 mmol). The reaction mixture was stirred at room temperature for 2 h under nitrogen atmosphere. The mixture was quenched with water (10 mL) and extracted with DCM (3×10 mL). The combined organic layer was dried over Na₂SO₄ and concentrated under reduced pressure to get the required product (3S)-6-fluoro-3-methyl-4-[(3-methyloxetan-3-yl)carbonyl]-3,5-dihydro-2H-1,4-benzoxazepine-8-carbonitrile (347 mg, 41%) as yellow oil. MS (ESI): mass calcd. for C₁₆H₁₇FN₂O₃, 304.12, m/z found 305.1 [M+H]⁺.

(S)-6-fluoro-N-hydroxy-3-methyl-4-(3-methyloxetane-3-carbonyl)-2,3,4,5-tetrahydrobenzo[f][1,4]oxazepine-8-carboximidamide. To a solution of (3S)-6-fluoro-3-methyl-4-[(3-methyloxetan-3-yl)carbonyl]-3,5-dihydro-2H-1,4-benzoxazepine-8-carbonitrile (347 mg, 1.14 mmol) in EtOH (15 mL) was added NH₂OH/H₂O (3.8 g, 114 mmol, 50%) under nitrogen atmosphere at room temperature. The reaction mixture was stirred at 65° C. for 2 h under nitrogen atmosphere. The mixture was cooled to room temperature and concentrated under reduced pressure to get the required product (3S)-6-fluoro-N-hydroxy-3-methyl-4-[(3-methyloxetan-3-yl)carbonyl]-3,5-dihydro-2H-1,4-benzoxazepine-8-carboximidamide (327 mg, 76%) as a white solid. MS (ESI): mass calcd. for C₁₆H₂₀FN₃O₄, 337.14, m/z found 338.1 [M+H]⁺.

(S)-(6-fluoro-3-methyl-8-(5-(trifluoromethyl)-1,2,4-oxadiazol-3-yl)-2,3-dihydrobenzo[f][1,4]oxazepin-4(5H)-yl)(3-methyloxetan-3-yl)methanone. To a solution of (3S)-6-fluoro-N-hydroxy-3-methyl-4-[(3-methyloxetan-3-yl)carbonyl]-3,5-dihydro-2H-1,4-benzoxazepine-8-carboximidamide (327 mg, 0.97 mmol) and pyridine (230 mg, 2.91 mmol) in DMF (10 mL) was added TFAA (407 mg, 1.94 mmol) at 0° C. under nitrogen atmosphere. The reaction mixture was stirred at 80° C. for 2 h under nitrogen atmosphere. The mixture was cooled to room temperature and concentrated under reduced pressure to get a residue which was purified by column chromatography on silica gel (25 g) eluting with EA in PE (0-30%) to (3S)-6-fluoro-3-methyl-4-[(3-methyloxetan-3-yl)carbonyl]-8-[5-(trifluoromethyl)-1,2,4-oxadiazol-3-yl]-3,5-dihydro-2H-1,4-benzoxazepine (121.6 mg, 30%) as a white solid. MS (ESI): mass calcd. for C₁₈H₁₇F₄N₃O₄, 415.12, m/z found 416.1 [M+H]⁺. ¹H NMR (400 MHz, MeOD) δ 7.54-7.39 (m, 2H), 5.37-3.65 (m, 9H), 1.72-1.54 (m, 3H), 1.36-1.10 (m, 3H).

### Example 1.2. Synthesis of (S)-(4-methyltetrahydro-2H-pyran-4-yl)(3-phenyl-8-(5-(trifluoromethyl)-1,3,4-oxadiazol-2-yl)-2,3-dihydrobenzo[f][1,4]oxazepin-4(5H)-yl)methanone (I-2)

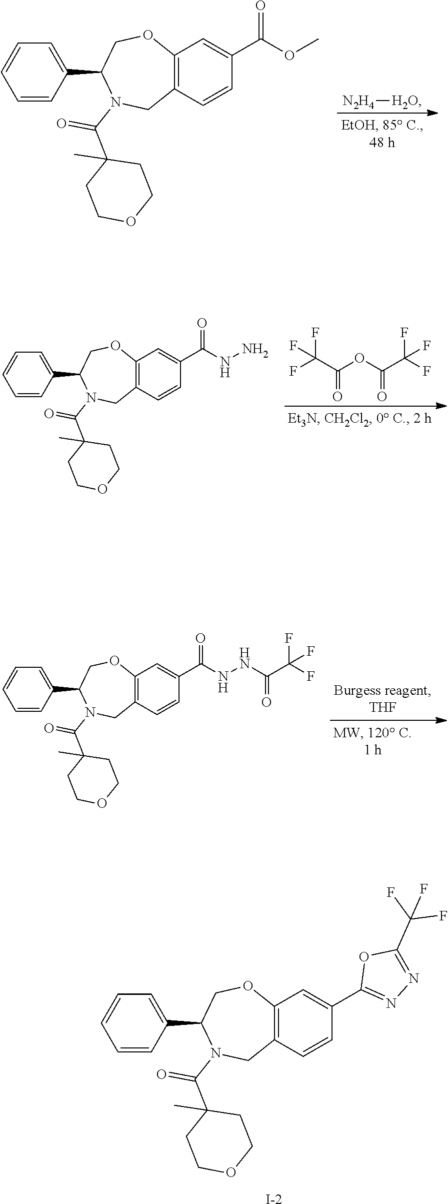

(S)-4-(4-methyltetrahydro-2H-pyran-4-carbonyl)-3-phenyl-2,3,4,5-tetrahydrobenzo[f][1,4]oxazepine-8-carbohydrazide. To a solution of methyl (S)-4-(4-methyltetrahydro-2H-pyran-4-carbonyl)-3-phenyl-2,3,4,5-tetrahydrobenzo[f][1,4]oxazepine-8-carboxylate (500 mg, 1.22 mmol) in EtOH (10 mL) stirred under nitrogen at 25° C. was added N₂H₄*H₂O (611.28 mg, 12.21 mmol). The reaction mixture was stirred at 85° C. for 24 hours. After cooling to room temperature, the mixture was concentrated under reduce pressure. The residue was purified by flash chromatograph on silica gel column DCM:MeOH=(10:1) to give (S)-4-(4-methyltetrahydro-2H-pyran-4-carbonyl)-3-phenyl-2,3,4,5-tetrahydrobenzo[f][1,4]oxazepine-8-carbohydrazide (500 mg, 99%) as a white solid. MS (ESI): mass calcd. for C₂₃H₂₇N₃O₄, 409.20, m/z found 410.3 [M+H]⁺.

(S)-4-(4-methyltetrahydro-2H-pyran-4-carbonyl)-3-phenyl-N′-(2,2,2-trifluoroacetyl)-2,3,4,5-tetrahydrobenzo[f][1,4]oxazepine-8-carbohydrazide. To a solution of (3S)-4-[(4-methyloxan-4-yl)carbonyl]-3-phenyl-3,5-dihydro-2H-1,4-benzoxazepine-8-carbohydrazide (250 mg, 0.61 mmol) and Et₃N (123.55 mg, 0.17 mL, 1.22 mmol) in DCM (10 mL) stirred under nitrogen at 0° C. was added a solution of 2,2,2-trifluoroacetyl 2,2,2-trifluoroacetate (128.22 mg, 0.61 mmol) in DCM (1 mL) slowly. The reaction mixture was stirred at 0° C. for 2 hours. The reaction was quenched with water (20 mL), followed by extraction with DCM (50 mL×3). The organic layer was washed with brine (30 mL), dried anhydrous Na₂SO₄, filtered and concentrated in vacuo. The residue was purified by flash chromatograph on silica gel column DCM:MeOH=(10/1) to give (S)-4-(4-methyltetrahydro-2H-pyran-4-carbonyl)-3-phenyl-N′-(2,2,2-trifluoroacetyl)-2,3,4,5-tetrahydrobenzo[f][1,4]oxazepine-8-carbohydrazide (220 mg, 71%) as a white solid. MS (ESI): mass calcd. for C₂₅H₂₆F₃N₃O₅, 505.18, m/z found 506.2 [M+H]⁺.

(S)-(4-methyltetrahydro-2H-pyran-4-yl)(3-phenyl-8-(5-(trifluoromethyl)-1,3,4-oxadiazol-2-yl)-2,3-dihydrobenzo[f][1,4]oxazepin-4(5H)-yl)methanone. The mixture of (S)-(4-methyltetrahydro-2H-pyran-4-yl)(3-phenyl-8-(5-(trifluoromethyl)-1,3,4-oxadiazol-2-yl)-2,3-dihydrobenzo[f][1,4]oxazepin-4(5H)-yl)methanone (150 mg, 0.29 mmol) and Burgess reagent (141.41 mg, 0.59 mmol) in THE (5 mL) was heated at 120° C. for 1 hours under microwaves. The mixture was cooled down to room temperature. Then saturated aqueous sodium bicarbonate solution was added to the reaction mixture, followed by extraction with DCM (50 mL×3). The organic layer was washed with brine and concentrated under reduced pressure. The residue was purified by prep-HPLC to give (S)-(4-methyltetrahydro-2H-pyran-4-yl)(3-phenyl-8-(5-(trifluoromethyl)-1,3,4-oxadiazol-2-yl)-2,3-dihydrobenzo[f][1,4]oxazepin-4(5H)-yl)methanone (115 mg, 80%) as a white solid. MS (ESI): mass calcd. for C₂₅H₂₄F₃N₃O₄, 487.17, m/z found 488.1 [M+H]⁺. ¹H NMR (400 MHz, DMSO-d₆) δ 7.67-7.30 (m, 8H), 5.95-5.91 (m, 1H), 5.19-5.15 (m, 1H), 4.81 (s, 1H), 4.59-4.53 (m, 2H), 3.60-3.55 (m, 1H), 3.42-3.37 (m, 1H), 3.31-3.10 (m, 2H), 2.01-1.98 (m, 1H), 1.86-1.76 (m, 2H), 1.40-1.38 (m, 2H), 1.25 (s, 3H).

### Example 1.3. Synthesis of (S)-(8-(5-(difluoromethyl)-1,3,4-oxadiazol-2-yl)-3-phenyl-2,3-dihydrobenzo[f][1,4]oxazepin-4(5H)-yl)(4-methyltetrahydro-2H-pyran-4-yl)methanone (I-3)

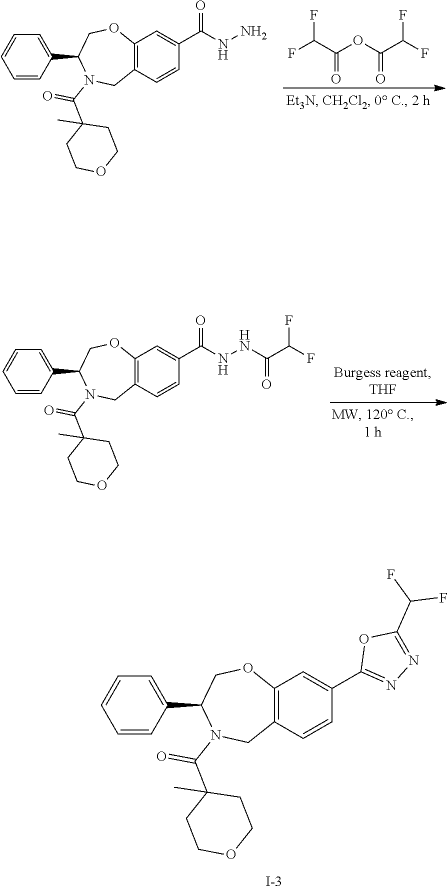

(S)—N′-(2,2-difluoroacetyl)-4-(4-methyltetrahydro-2H-pyran-4-carbonyl)-3-phenyl-2,3,4,5-tetrahydrobenzo[f][1,4]oxazepine-8-carbohydrazide. To a solution of (3S)-4-[(4-methyloxan-4-yl)carbonyl]-3-phenyl-3,5-dihydro-2H-1,4-benzoxazepine-8-carbohydrazide (200 mg, 0.49 mmol) in dry DCM (10 mL) stirred at 0° C. under N₂ was added triethylamine (98.78 mg, 0.98 mmol) and 2,2-difluoroacetyl 2,2-difluoroacetate (93.51 mg, 0.54 mmol). The reaction mixture was stirred at 0° C. for 2 hours. The reaction mixture was quenched with water (30 mL) and extracted with DCM (50 mL×3). The organic layer was washed with brine (30 mL), dried anhydrous Na₂SO₄, filtered and concentrated in vacuo. The residue was purified by flash chromatograph on silica gel column DCM:MeOH=(10/1) to give (S)—N′-(2,2-difluoroacetyl)-4-(4-methyltetrahydro-2H-pyran-4-carbonyl)-3-phenyl-2,3,4,5-tetrahydrobenzo[f][1,4]oxazepine-8-carbohydrazide (230 mg, 96%) as a white solid. MS (ESI): mass calcd. for C₂₅H₂₇F₂N₃O₅, 487.19, m/z found 488.2 [M+H]⁺.

(S)-(8-(5-(difluoromethyl)-1,3,4-oxadiazol-2-yl)-3-phenyl-2,3-dihydrobenzo[f][1,4]oxazepin-4(5H)-yl)(4-methyltetrahydro-2H-pyran-4-yl)methanone. The mixture of (3S)—N′-(2,2-difluoroacetyl)-4-[(4-methyloxan-4-yl)carbonyl]-3-phenyl-3,5-dihydro-2H-1,4-benzoxazepine-8-carbohydrazide (150 mg, 0.31 mmol) and Burgess reagent (146.65 mg, 0.62 mmol) in THE (4 mL) was heated at 120° C. for 1 hours under microwaves. The reaction mixture was cooled down to room temperature. Saturated aqueous sodium bicarbonate solution was added to the reaction mixture, followed by extraction with DCM (50 mL×3). The organic layer was concentrated under reduced pressure. The residue was purified by prep-HPLC to give (S)-(8-(5-(difluoromethyl)-1,3,4-oxadiazol-2-yl)-3-phenyl-2,3-dihydrobenzo[f][1,4]oxazepin-4(5H)-yl)(4-methyltetrahydro-2H-pyran-4-yl)methanone (125 mg, 86%) as a white solid. MS (ESI): mass calcd. for C₂₅H₂₅F₂N₃O₄, 469.18, m/z found 470.2 [M+H]⁺. ¹H NMR (400 MHz, DMSO-d₆) δ 7.66-7.63 (m, 1H), 7.54-7.30 (m, 7H), 5.95-5.90 (m, 1H), 5.19-5.14 (m, 1H), 4.84-4.77 (m, 1H), 4.59-4.50 (m, 2H), 3.60-3.55 (m, 1H), 3.40-3.37 (m, 1H), 3.19-3.15 (m, 1H), 2.02-1.98 (m, 1H), 1.89-1.70 (m, 2H), 1.41-1.38 (m, 2H), 1.25 (s, 3H).

### Example 1.4. Synthesis of (S)-(4-methyltetrahydro-2H-pyran-4-yl)(3-phenyl-8-(5-(trifluoromethyl)-1,2,4-oxadiazol-3-yl)-2,3-dihydrobenzo[f][1,4]oxazepin-4(5H)-yl)methanone (I-4)

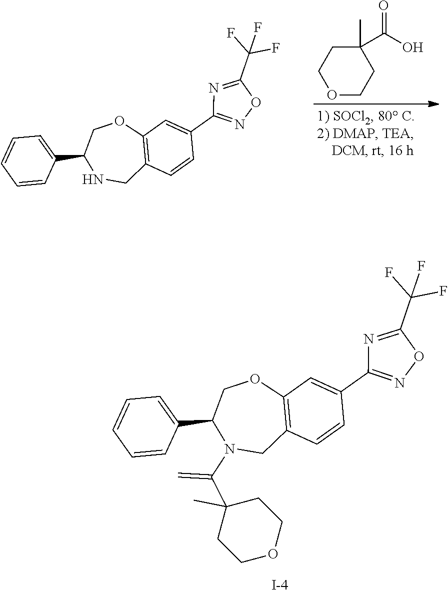

To a solution of 4-methyloxane-4-carboxylic acid (1297 mg, 8.89 mmol) in SOCl₂ (10 mL), the reaction mixture was stirred at 80° C. for 2 h, then was concentrated resulting in acyl chloride (1.5 g crude). To a solution of (3S)-3-phenyl-8-[5-(trifluoromethyl)-1,2,4-oxadiazol-3-yl]-2,3,4,5-tetrahydro-1,4-benzoxazepine (650 mg, 1.80 mmol) in DCM (10 mL), was added TEA (1817 mg, 17.99 mmol), 4-DMAP (22 mg, 0.18 mmol), acyl chloride (1.5 g crude). The reaction mixture was stirred at 25° C. for 16 h. The mixture was quenched by H₂O (20 mL), extracted with DCM (2*30 mL). The organic layer was washed with brine (20 mL), dried over anhydrous Na₂SO₄, concentrated under vacuum. The residue was applied onto silica gel column, this resulted product (500 mg), then purified by Pre-HPLC, (Instrument: Waters MS-triggered Prep-LC with QDA detector, Column: Xbridge 5 u C18 150×19 mm, A water (0.1% FA), B Acetonitrile 50-80% B in 8 min, hold at 100% B for 2 min, back to 50% B with 0.5 min, stop at 13 min, flow rate: 20 mL/min, wavelength: 214/254 nm, injection: 6) to afford the target compound (179.6 mg, 20%) as a white solid. MS (ESI): mass calcd. for C₂₅H₂₄F₃N₃O₄, 487.17, m/z found 488.1 [M+H]⁺. ¹H NMR (400 MHz, DMSO-d₆): δ 7.70-7.29 (m, 8H), 6.05-5.85 (m, 1H), 5.22-5.10 (m, 1H), 4.95-4.40 (m, 3H), 3.65-3.52 (m, 1H), 3.45-3.32 (m, 1H), 3.23-2.87 (m, 2H), 2.10-1.95 (m, 1H), 1.90-1.73 (m, 1H), 1.50-1.35 (m, 2H), 1.25 (s, 3H).

### Example 1.5. Synthesis of (3S)-4-[(3-methyloxetan-3-yl) carbonyl]-3-phenyl-8-[5-(trifluoromethyl)-1,2,4-oxadiazol-3-yl]-3,5-dihydro-2H-1,4-benzoxazepine (I-5)

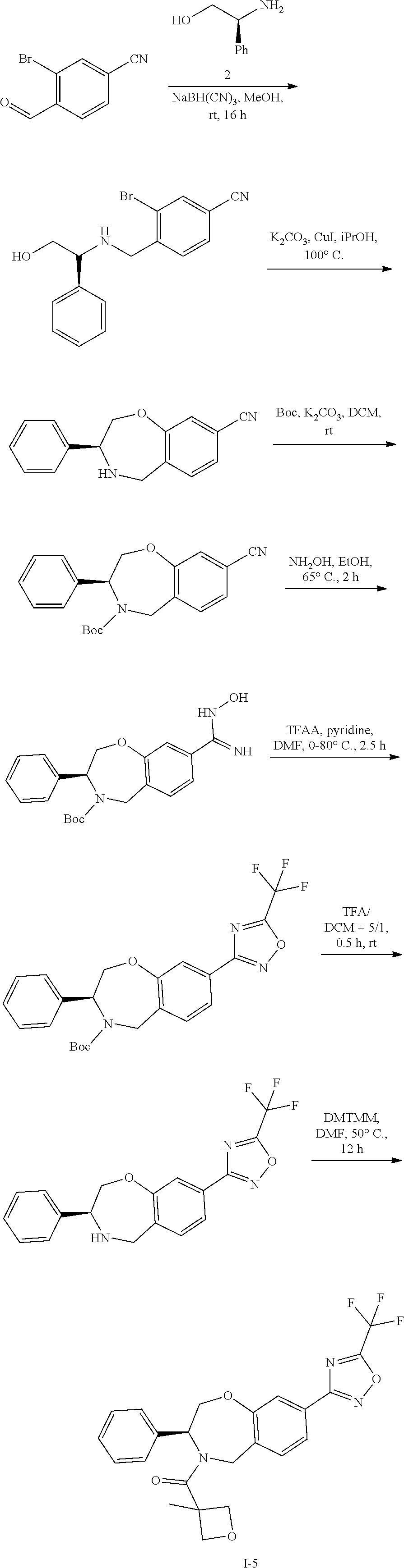

3-bromo-4-({[(1S)-2-hydroxy-1-phenylethyl]amino}methyl) benzonitrile. 3-bromo-4-formylbenzonitrile (20 g, 0.095 mol), (2S)-2-amino-2-phenylethanol (15.67 g, 0.114 mol) were added successively in MeOH (400 mL). The reaction mixture was stirred under nitrogen at 25° C. for 12 h and then cooled to 0° C. NaBH₃CN (17.95 g, 0.285 mol) was added. The reaction mixture was warm to room temperature and stirred for 1 h. The mixture was quenched by NH₄C₁ (aq, 100 mL) and extracted with EtOAc (150 mL×3). The organic layer was dried over anhydrous Na₂SO₄, filtered and concentrated under reduced pressure to give the crude product. The residue was purified by Flash Chromatography to give 3-bromo-4-({[(1S)-2-hydroxy-1-phenylethyl]amino}methyl) benzonitrile (26.2 g, 78%) as a yellow solid. MS (ESI): mass calcd. for C₁₆H₁₅BrN₂O, 330.04, m/z found 331.1 [M+H]⁺.

(3S)-3-phenyl-2,3,4,5-tetrahydro-1,4-benzoxazepine-8-carbonitrile. To a solution of 3-bromo-4-({[(1S)-2-hydroxy-1-phenylethyl]amino}methyl) benzonitrile (26.1 g, 0.078 mol) in i-PrOH (500 mL) was added K₂CO₃ (21.76 g, 0.157 mol) and Copper (I) iodide (7.5 g, 0.039 mol). The reaction mixture was stirred for 12 h under nitrogen at 100° C. The reaction mixture was cooled to room temperature. The mixture was filtered and concentrated under reduced pressure to give the crude product. The crude product was diluted with water and extracted with EtOAc (200 mL×3). The organic layer was dried over anhydrous Na₂SO₄, filtered and concentrated under reduced pressure. The residue was purified by flash chromatography to give (3S)-3-phenyl-2,3,4,5-tetrahydro-1,4-benzoxazepine-8-carbonitrile (7.0 g, 33%) as brown solid. MS (ESI): mass calcd. for C₁₆H₁₄N₂O, 250.11, m/z found 251.1 [M+H]⁺.

Tert-butyl (3S)-8-cyano-3-phenyl-3,5-dihydro-2H-1,4-benzoxazepin-4-yl formate. To a solution of (3S)-3-phenyl-2,3,4,5-tetrahydro-1,4-benzoxazepine-8-carbonitrile (1.0 g, 0.004 mmol) in DCM (30 mL) was added (Boc)₂O (1.05 g, 0.004 mmol) and triethylamine (1.21 g, 0.012 mmol). The mixture was stirred at 25° C. for 12 h. The mixture was diluted with water and extracted with EtOAc (50 mL×3). The organic layer was dried over anhydrous Na₂SO₄, filtered and concentrated under reduced pressure to give the crude product. The residue was purified by flash chromatography to give tert-butyl (3S)-8-cyano-3-phenyl-3,5-dihydro-2H-1,4-benzoxazepin-4-yl formate (0.977 g, 65%) as brown solid. MS (ESI): mass calcd. for C₂₁H₂₂N₂O₃, 350.16, m/z found 373.1 [M+Na]⁺.

Tert-butyl (3S)-8-(N-hydroxycarbamimidoyl)-3-phenyl-3,5-dihydro-2H-1,4-benzoxazepin-4-yl formate. A mixture of tert-butyl (3S)-8-cyano-3-phenyl-3,5-dihydro-2H-1,4-benzoxazepin-4-yl formate (970 mg, 2.76 mmol) and NH₂OH/H₂O (273 mg, 8.280 mmol) in EtOH (15 mL) was stirred at 65° C. for 2.5 h. The reaction mixture was cooled to room temperature. The mixture was diluted with water and extracted with EtOAc (30 mL×3). The organic layer was dried over anhydrous Na₂SO₄, filtered and concentrated under reduced pressure to give the product tert-butyl (3S)-8-(N-hydroxycarbamimidoyl)-3-phenyl-3,5-dihydro-2H-1,4-benzoxazepin-4-yl formate (1.21 g, 96%) as orange oil. MS (ESI): mass calcd. for C₂₁H₂₅N₃O₄, 383.18, m/z found 384.2 [M+H]⁺.

Tert-butyl (3S)-3-phenyl-8-[5-(trifluoromethyl)-1,2,4-oxadiazol-3-yl]-3,5-dihydro-2H-1,4-benzoxazepin-4-yl formate. To the mixture of tert-butyl (3S)-8-(N-hydroxycarbamimidoyl)-3-phenyl-3,5-dihydro-2H-1,4-benzoxazepin-4-yl formate (1 g, 0.0029 mol) and pyridine (0.69 g, 0.0087 mol) in DMF (20 mL) was added TFAA (0.91 g, 0.0043 mol). After stirring for 10 minutes at 0° C., the reaction mixture was stirred at 80° C. for additional 2.5 h. The reaction mixture was cooled to room temperature. The mixture was diluted with water and extracted with EtOAc (50 mL×3). The organic layer was dried over anhydrous Na₂SO₄, filtered and concentrated under reduced pressure to give the crude product. The residue was purified by Prep-HPLC to give tert-butyl (3S)-3-phenyl-8-[5-(trifluoromethyl)-1,2,4-oxadiazol-3-yl]-3,5-dihydro-2H-1,4-benzoxazepin-4-yl formate (0.457 g, 34%) as a white solid. MS (ESI): mass calcd. for C₂₃H₂₂F₃N₃O₄, 461.16, m/z found 484.1 [M+Na]⁺.

(3S)-3-phenyl-8-[5-(trifluoromethyl)-1,2,4-oxadiazol-3-yl]-2,3,4,5-tetrahydro-1,4-benzoxazepine. To a mixture of DCM (10 mL) and TFA (2 mL) was added tert-butyl (3S)-3-phenyl-8-[5-(trifluoromethyl)-1,2,4-oxadiazol-3-yl]-3,5-dihydro-2H-1,4-benzoxazepin-4-yl formate (450 mg, 0.973 mmol). The mixture was stirred at rt for 30 min. The mixture was quenched by Na₂CO₃ (aq, 20 mL) and concentrated under reduced pressure to get white solid. The solid was diluted with water and extracted with EtOAc (30 mL×3). The organic layer was dried over anhydrous Na₂SO₄, filtered and concentrated under reduced pressure to give the product (3S)-3-phenyl-8-[5-(trifluoromethyl)-1,2,4-oxadiazol-3-yl]-2,3,4,5-tetrahydro-1,4-benzoxazepine (320 mg, 81%) as orange solid. MS (ESI): mass calcd. for C₁₈H₁₄F₃N₃O₂, 361.10, m/z found 384.2 [M+Na]⁺.

(3S)-4-[(3-methyloxetan-3-yl) carbonyl]-3-phenyl-8-[5-(trifluoromethyl)-1,2,4-oxadiazol-3-yl]-3,5-dihydro-2H-1,4-benzoxazepine. 3-methyloxetane-3-carboxylic acid (57 mg, 0.498 mmol), DMTMM (229 mg, 0.830 mmol) were added successively in DMF (5 mL). The reaction mixture was stirred at rt for 30 min. (3S)-3-phenyl-8-[5-(trifluoromethyl)-1,2,4-oxadiazol-3-yl]-2,3,4,5-tetrahydro-1,4-benzoxazepine (150 mg, 0.415 mmol) was added. The reaction mixture was stirred at 50° C. for 12 h. The mixture was diluted with water and extracted with EtOAc (50 mL×3). The organic layer was dried over anhydrous Na₂SO₄, filtered and concentrated under reduced pressure to give the crude product. The residue was purified by Prep-HPLC to give (3S)-4-[(3-methyloxetan-3-yl) carbonyl]-3-phenyl-8-[5-(trifluoromethyl)-1,2,4-oxadiazol-3-yl]-3,5-dihydro-2H-1,4-benzoxazepine (50.0 mg, 26%) as a white solid. MS (ESI): mass calcd. for C₂₃H₂₀F₃N₃O₄, 459.14, m/z found 460.1 [M+H]⁺. ¹H NMR (400 MHz, DMSO) δ 7.68-7.63 (m, 1H), 7.58-7.44 (m, 3H), 7.41-7.29 (m, 4H), 5.85-5.81 (m, 1H), 5.25-5.19 (m, 1H), 4.86-4.79 (m, 1H), 4.74-4.72 (m, 1H), 4.70-4.62 (m, 1H), 4.57-4.50 (m, 1H), 4.45-4.39 (m, 1H), 4.32-4.25 (m, 1H), 4.20-4.11 (m, 1H), 1.56-1.49 (m, 1H).

### Example 1.6. Synthesis of (S)-2,2,2-trifluoro-1-(3-phenyl-8-(5-(trifluoromethyl)-1,2,4-oxadiazol-3-yl)-2,3-dihydrobenzo[f][1,4]oxazepin-4(5H)-yl)ethan-1-one (I-6)

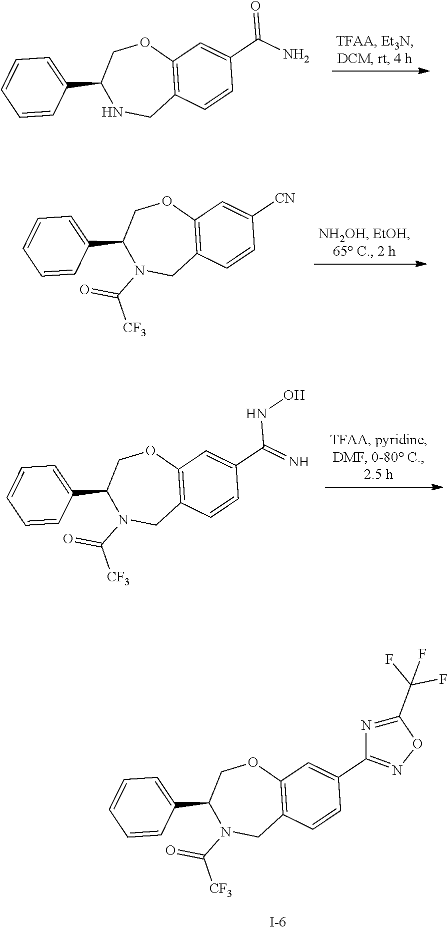

(S)-3-phenyl-4-(2,2,2-trifluoroacetyl)-2,3,4,5-tetrahydrobenzo[f][1,4]oxazepine-8-carbonitrile. To a solution of (3S)-3-phenyl-2,3,4,5-tetrahydro-1,4-benzoxazepine-8-carboxamide (300 mg, 1.12 mmol) in DCM (20 mL) were added TEA (677 mg, 6.71 mmol) and TFAA (704 mg, 3.35 mmol) at 0° C. The mixture was warmed to room temperature and stirred for 2 h. The reaction mixture was added to a saturated aqueous sodium hydrogen carbonate solution. The organic layer was washed with brine, dried over Na₂SO₄ and concentrated under reduced pressure. The residue was purified by silica gel column chromatography eluted with PE/EA (3:1) to afford (S)-3-phenyl-4-(2,2,2-trifluoroacetyl)-2,3,4,5-tetrahydrobenzo[f][1,4]oxazepine-8-carbonitrile (220 mg, 71%) as yellow oil. MS (ESI): mass calcd. for C₁₈H₁₃F₃N₂O₂, 346.09, m/z found 347.1 [M+H]⁺.

(S)—N-hydroxy-3-phenyl-4-(2,2,2-trifluoroacetyl)-2,3,4,5-tetrahydrobenzo[f][1,4]-oxazepane-8-carboximidamide. To a solution of (S)-3-phenyl-4-(2,2,2-trifluoroacetyl)-2,3,4,5-tetrahydrobenzo[f][1,4]oxazepine-8-carbonitrile (200 mg, 0.58 mmol) in EtOH (10 mL) was added NH₂OH/H₂O (57.07 mg, 1.73 mmol) and the mixture was stirred at 65° C. for 2 h under N₂. The organic solvent was removed under reduced pressure and the solid was collected by filtration, washed with EtOH (20 mL×3) and dried in vacuo to afford (S)—N-hydroxy-3-phenyl-4-(2,2,2-trifluoroacetyl)-2,3,4,5-tetrahydrobenzo[f][1,4]-oxazepane-8-carboximidamide (200 mg, 91%) as white solid. MS (ESI): mass calcd. for C₁₈H₁₆F₃N₃O₃, 379.11, m/z found 380.1 [M+H]⁺.

(S)-2,2,2-trifluoro-1-(3-phenyl-8-(5-(trifluoromethyl)-1,2,4-oxadiazol-3-yl)-2,3-dihydrobenzo[f][1,4]oxazepin-4(5H)-yl)ethan-1-one. To a solution of (S)—N-hydroxy-3-phenyl-4-(2,2,2-trifluoroacetyl)-2,3,4,5-tetrahydrobenzo[f][1,4]-oxazepane-8-carboximidamide (220 mg, 0.58 mmol) in DMF (5 mL) were added pyridine (150 mg, 1.74 mmol) and TFAA (198 mg, 0.87 mmol) at 0° C. and the mixture was stirred at 80° C. for 2 h. After completion, the mixture was extracted with EA (50 mL×2) and water. The organic layer was washed with brine, dried over Na₂SO₄, filtered and concentrated in vacuo. The residue was purified by silica gel column chromatography (PE/EA=10/1) to afford (S)-2,2,2-trifluoro-1-(3-phenyl-8-(5-(trifluoromethyl)-1,2,4-oxadiazol-3-yl)-2,3-dihydrobenzo[f][1,4]oxazepin-4(5H)-yl)ethan-1-one (200 mg, 67%) as white solid. MS (ESI): mass calcd. for C₂₀H₁₃F₆N₃O₃, 457.09, m/z found 458.0 [M+H]⁺. ¹H NMR (400 MHz, DMSO-d₆) δ 7.68-7.35 (m, 8H), 5.89-4.69 (m, 5H).

### Example 1.7. Synthesis of (3S)-4-{[3-(methoxymethyl) oxetan-3-yl]carbonyl}-3-phenyl-8-[5-(trifluoromethyl)-1, 2, 4-oxadiazol-3-yl]-3, 5-dihydro-2H-1, 4-benzoxazepine (I-7)

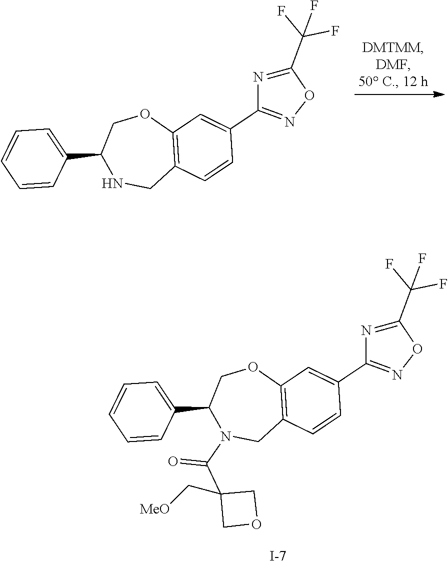

3-methyloxetane-3-carboxylic acid (72 mg, 0.498 mmol), DMTMM (229 mg, 0.830 mmol) were added successively in DMF (5 mL). The reaction mixture was stirred at rt for 30 min. Then (3S)-3-phenyl-8-[5-(trifluoromethyl)-1, 2, 4-oxadiazol-3-yl]-2, 3, 4, 5-tetrahydro-1, 4-benzoxazepine (150 mg, 0.415 mmol) was added. The reaction mixture was stirred for 12 h. The mixture was diluted with water and extracted with EtOAc (50 mL×3). The organic layer was dried over anhydrous Na₂SO₄, filtered and concentrated under reduced pressure to give the crude product. The residue was purified by Prep-HPLC to give (3S)-4-{[3-(methoxymethyl) oxetan-3-yl]carbonyl}-3-phenyl-8-[5-(trifluoromethyl)-1, 2, 4-oxadiazol-3-yl]-3, 5-dihydro-2H-1, 4-benzoxazepine (53 mg, 26%) as a white solid. MS (ESI): mass calcd. For C₂₄H₂₂F₃N₃O₅, 489.15, m/z found 490.2 [M+H]⁺. ¹H NMR (400 MHz, DMSO) δ 7.68-7.64 (m, 1H), 7.52-7.28 (m, 7H), 5.86-5.82 (m, 1H), 5.23-5.08 (m, 1H), 4.83-4.27 (m, 7H), 3.98-3.96 (m, 1H), 3.76-3.69 (m, 1H), 3.57-3.54 (m, 1H), 3.24-3.06 (m, 1H).

### Example 1.8. Synthesis of tert-butyl (S)-3-phenyl-8-(5-(trifluoromethyl)-1,2,4-oxadiazol-3-yl)-2,3-dihydrobenzo[f][1,4]oxazepine-4(5H)-carboxylate (I-8)

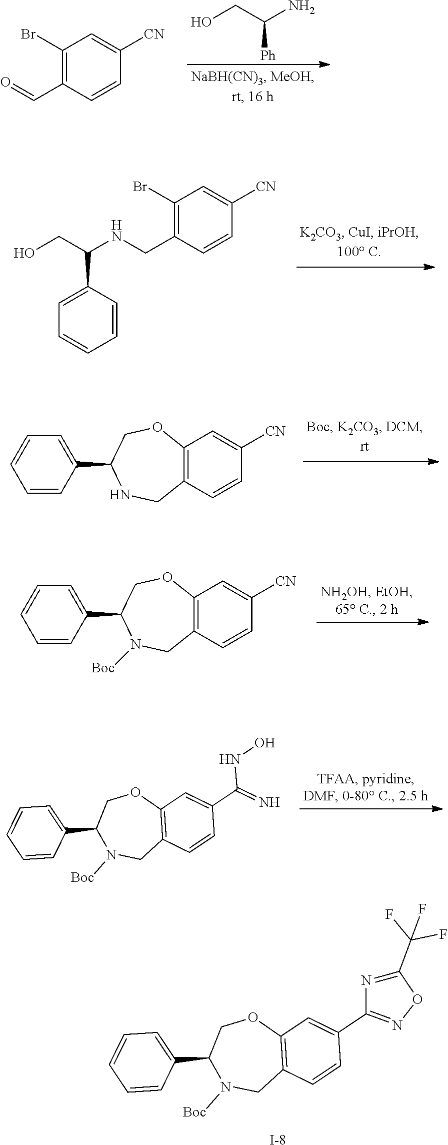

3-bromo-4-({[(1S)-2-hydroxy-1-phenylethyl]amino}methyl) benzonitrile. 3-bromo-4-formylbenzonitrile (20 g, 0.095 mol), (2S)-2-amino-2-phenylethanol (15.67 g, 0.114 mol) were added successively in MeOH (400 mL). The reaction mixture was stirred under nitrogen at 25° C. for 12 h and then cooled to 0° C. NaBH₃CN (17.95 g, 0.285 mol) was added. The reaction mixture was warm to room temperature and stirred for 1 h. The mixture was quenched by NH₄C₁ (aq, 100 mL) and extracted with EtOAc (150 mL×3). The organic layer was dried over anhydrous Na₂SO₄, filtered and concentrated under reduced pressure to give the crude product. The residue was purified by Flash Chromatography to give 3-bromo-4-({[(1S)-2-hydroxy-1-phenylethyl]amino}methyl) benzonitrile (26.2 g, 78%) as a yellow solid. MS (ESI): mass calcd. for C₁₆H₁₅BrN₂O, 330.04, m/z found 331.1 [M+H]⁺.

(3S)-3-phenyl-2,3,4,5-tetrahydro-1,4-benzoxazepine-8-carbonitrile. To a solution of 3-bromo-4-({[(1S)-2-hydroxy-1-phenylethyl]amino}methyl) benzonitrile (26.1 g, 0.078 mol) in i-PrOH (500 mL) was added K₂CO₃ (21.76 g, 0.157 mol) and Copper (I) iodide (7.5 g, 0.039 mol). The reaction mixture was stirred for 12 h under nitrogen at 100° C. The reaction mixture was cooled to room temperature. The mixture was filtered and concentrated under reduced pressure to give the crude product. The mixture was diluted with water and extracted with EtOAc (200 mL×3). The organic layer was dried over anhydrous Na₂SO₄, filtered and concentrated under reduced pressure to give the crude product. The residue was purified by flash chromatography to give (3S)-3-phenyl-2,3,4,5-tetrahydro-1,4-benzoxazepine-8-carbonitrile (7.0 g, 33%) as brown solid. MS (ESI): mass calcd. for C₁₆H₁₄N₂O, 250.11, m/z found 251.1 [M+H]⁺.

Tert-butyl (3S)-8-cyano-3-phenyl-3,5-dihydro-2H-1,4-benzoxazepin-4-yl formate. To a solution of (3S)-3-phenyl-2,3,4,5-tetrahydro-1,4-benzoxazepine-8-carbonitrile (1.0 g, 0.004 mmol) in DCM (30 mL) was added (Boc)₂O (1.05 g, 0.004 mmol) and triethylamine (1.21 g, 0.012 mmol). The mixture was stirred at 25° C. for 12 h. The mixture was diluted with water and extracted with EtOAc (50 mL×3). The organic layer was dried over anhydrous Na₂SO₄, filtered and concentrated under reduced pressure to give the crude product. The residue was purified by flash chromatography to give tert-butyl (S)-8-cyano-3-phenyl-2,3-dihydrobenzo[f][1,4]oxazepine-4(5H)-carboxylate (0.977 g, 65%) as brown solid. MS (ESI): mass calcd. for C₂₁H₂₂N₂O₃, 350.16, m/z found 373.1 [M+Na]⁺.

Tert-butyl (3S)-8-(N-hydroxycarbamimidoyl)-3-phenyl-3,5-dihydro-2H-1,4-benzoxazepin-4-yl formate. A mixture of tert-butyl (3S)-8-cyano-3-phenyl-3,5-dihydro-2H-1,4-benzoxazepin-4-yl formate (970 mg, 2.76 mmol) and NH₂OH/H₂O (273 mg, 8.280 mmol) in EtOH (15 mL) was stirred at 65° C. for 2.5 h. The reaction mixture was cooled to room temperature. The mixture was diluted with water and extracted with EtOAc (30 mL×3). The organic layer was dried over anhydrous Na₂SO₄, filtered and concentrated under reduced pressure to give the product tert-butyl (S)-8-(N-hydroxycarbamimidoyl)-3-phenyl-2,3-dihydrobenzo[f][1,4]oxazepine-4(5H)-carboxylate (1.21 g, 96%) as orange oil. MS (ESI): mass calcd. for C₂₁H₂₅N₃O₄, 383.18, m/z found 384.2 [M+H]⁺.

tert-butyl (S)-3-phenyl-8-(5-(trifluoromethyl)-1,2,4-oxadiazol-3-yl)-2,3-dihydrobenzo[f][1,4]oxazepine-4(5H)-carboxylate. To the mixture of tert-butyl (3S)-8-(N-hydroxycarbamimidoyl)-3-phenyl-3,5-dihydro-2H-1,4-benzoxazepin-4-yl formate (1 g, 0.0029 mol) and pyridine (0.69 g, 0.0087 mol) in DMF (20 mL) was added TFAA (0.91 g, 0.0043 mol). After stirring for 10 minutes at 0° C. the reaction mixture was stirred at 80° C. for additional 2.5 h. The reaction mixture was cooled to room temperature. The mixture was diluted with water and extracted with EtOAc (50 mL×3). The organic layer was dried over anhydrous Na₂SO₄, filtered and concentrated under reduced pressure to give the crude product. The residue was purified by Prep-HPLC to give tert-butyl (S)-3-phenyl-8-(5-(trifluoromethyl)-1,2,4-oxadiazol-3-yl)-2,3-dihydrobenzo[f][1,4]oxazepine-4(5H)-carboxylate (0.457 g, 34%) as a white solid. MS (ESI): mass calcd. for C₂₃H₂₂F₃N₃O₄, 461.16, m/z found 484.1 [M+Na]⁺. ¹H NMR (400 MHz, DMSO) δ 7.66-7.64 (m, 1H), 7.49-7.40 (m, 6H), 7.35-7.30 (m, 1H), 5.62-5.44 (m, 1H), 5.03-4.88 (m, 1H), 4.80-4.74 (m, 2H), 4.63-4.50 (m, 1H), 1.26-4.35 (s, 9H).

### Examples 1.9-1.11. Synthesis of (3S)-4-[(3-methyloxolan-3-yl) carbonyl]-3-phenyl-8-[5-(trifluoromethyl)-1,2,4-oxadiazol-3-yl]-3,5-dihydro-2H-1,4-benzoxazepine; racemate I-9) first and second eluting isomers I-10 and I-11)

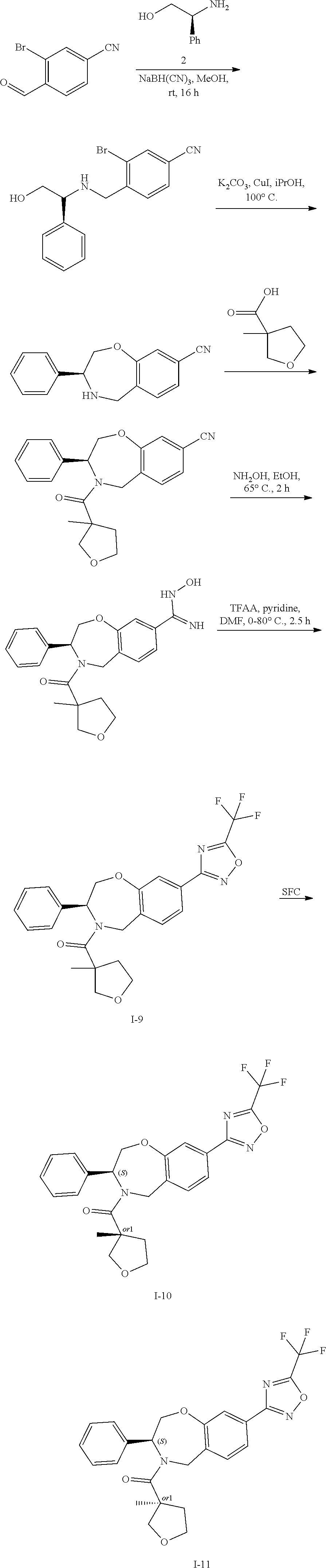

3-bromo-4-({[(1S)-2-hydroxy-1-phenylethyl]amino}methyl) benzonitrile. A solution of 3-bromo-4-formylbenzonitrile (10 g, 0.047 mol) and (2S)-2-amino-2-phenylethanol (7.84 g, 0.057 mol) in MeOH (200 ml) stirred at 25° C. for 12 h. NaBH₃CN (8.97 g, 0.142 mol) was added at 0° C. The reaction mixture was stirred at 25° C. for 0.5 h. The mixture was quenched by NH₄C₁ (aq, 100 mL) and extracted with EtOAc (150 mL×3). The organic layer was dried over anhydrous Na₂SO₄, filtered, and concentrated under reduced pressure. The residue was purified by Flash Chromatography to give 3-bromo-4-({[(1S)-2-hydroxy-1-phenylethyl]amino}methyl) benzonitrile (12.3 g, 70%) as an orange solid. MS (ESI): mass calcd. for C₁₆H₁₅BrN₂O, 330.04, m/z found 331.1 [M+H]⁺.

(3S)-3-phenyl-2,3,4,5-tetrahydro-1,4-benzoxazepine-8-carbonitrile. A mixture of 3-bromo-4-({[(1S)-2-hydroxy-1-phenylethyl]amino}methyl) benzonitrile (12.35 g, 0.037 mol), potassium carbonate (10.31 g, 0.074 mol) and copper(I) iodide (3.55 g, 0.018 mol) in i-PrOH (80 mL) was stirred at 100° C. for 16 h. The reaction mixture was cooled to room temperature. The mixture was filtered and concentrated under reduced pressure. The mixture was diluted with water and extracted with EtOAc (200 mL×3). The organic layer was dried over anhydrous Na₂SO₄, filtered and concentrated under reduced pressure. The residue was purified by flash chromatography to give (3S)-3-phenyl-2,3,4,5-tetrahydro-1,4-benzoxazepine-8-carbonitrile (2.4 g, 22%) as an orange oil. MS (ESI): mass calcd. for C₁₆H₁₄N₂O, 250.11, m/z found 251.1 [M+H]⁺.

(3S)—N-hydroxy-4-[(3-methyloxolan-3-yl) carbonyl]-3-phenyl-3,5-dihydro-2H-1,4-benzoxazepine-8-carboximidamide. A mixture of (3S)-4-[(3-methyloxolan-3-yl) carbonyl]-3-phenyl-3,5-dihydro-2H-1,4-benzoxazepine-8-carbonitrile (446 mg, 1.23 mmol) and NH₂OH/H₂O (122 mg, 3.69 mmol) in EtOH (10 ml) was stirred at 65° C. for 2 h. The reaction mixture was cooled to room temperature. The mixture was diluted with water (30 mL) and extracted with EtOAc (30 mL×3). The organic layer was dried over anhydrous Na₂SO₄, filtered and concentrated under reduced pressure to give the product (3S)—N-hydroxy-4-[(3-methyloxolan-3-yl) carbonyl]-3-phenyl-3,5-dihydro-2H-1,4-benzoxazepine-8-carboximidamide (510 mg, 89%) as yellow solid. MS (ESI): mass calcd. for C₂₂H₂₅N₃O₄, 395.18, m/z found 296.1 [M+H]⁺.

(3S)-4-[(3-methyloxolan-3-yl) carbonyl]-3-phenyl-8-[5-(trifluoromethyl)-1,2,4-oxadiazol-3-yl]-3,5-dihydro-2H-1,4-benzoxazepine. To a solution of (3S)—N-hydroxy-4-[(3-methyloxolan-3-yl) carbonyl]-3-phenyl-3,5-dihydro-2H-1,4-benzoxazepine-8-carboximidamide (488 mg, 1.234 mmol), pyridine (292 mg, 3.702 mmol) in DMF (15 ml) stirred under nitrogen at 0° C. was added TFAA (777 mg, 3.702 mmol). The reaction mixture was stirred at 80° C. for 2.5 h. The reaction mixture was cooled to room temperature. The mixture was diluted with water and extracted with EtOAc (50 mL×3). The organic layer was dried over anhydrous Na₂SO₄, filtered and concentrated under reduced pressure. The residue was purified by Prep-HPLC to give (3S)-4-[(3-methyloxolan-3-yl) carbonyl]-3-phenyl-8-[5-(trifluoromethyl)-1,2,4-oxadiazol-3-yl]-3,5-dihydro-2H-1,4-benzoxazepine (110 mg, 18%) as a white solid. MS (ESI): mass calcd. for C₂₄H₂₂F₃N₃O₄, 473.16, m/z found 474.1 [M+H]⁺.

(3S)-4-[(3-methyloxolan-3-yl) carbonyl]-3-phenyl-8-[5-(trifluoromethyl)-1,2,4-oxadiazol-3-yl]-3,5-dihydro-2H-1,4-benzoxazepine (110 mg) was separated by SFC to afford a first eluting isomer (36.5 mg, 35%) as a yellow solid and a second eluting isomer (29.5 mg, 24%) as a white solid.

(3S)-4-[(3-methyloxolan-3-yl) carbonyl]-3-phenyl-8-[5-(trifluoromethyl)-1,2,4-oxadiazol-3-yl]-3,5-dihydro-2H-1,4-benzoxazepine, first eluting isomer (I-10): MS (ESI): mass calcd. for C₂₄H₂₂F₃N₃O₄, 473.16, m/z found 474.1 [M+H]⁺. ¹H NMR (400 MHz, DMSO-d₆) δ 7.68-7.28 (m, 8H), 5.87-5.63 (m, 1H), 5.23-5.15 (m, 1H), 4.97-4.83 (m, 1H), 4.69-4.51 (m, 2H), 3.77-3.40 (m, 4H), 2.33-2.21 (m, 1H), 2.00-1.87 (m, 1H), 1.24-1.18 (m, 3H).

(3S)-4-[(3-methyloxolan-3-yl) carbonyl]-3-phenyl-8-[5-(trifluoromethyl)-1,2,4-oxadiazol-3-yl]-3,5-dihydro-2H-1,4-benzoxazepine, second eluting isomer (I-11): MS (ESI): mass calcd. for C₂₄H₂₂F₃N₃O₄, 473.16, m/z found 474.1 [M+H]⁺. ¹H NMR (400 MHz, DMSO-d₆) δ 7.68-7.28 (m, 8H), 5.84-5.68 (m, 1H), 5.22-4.84 (m, 2H), 4.72-4.50 (m, 2H), 3.86-3.49 (m, 4H), 1.99-1.64 (m, 2H), 1.38-1.27 (m, 3H).

Examples 1.12-1.14. Synthesis of (3S)-4-[(3-methyloxan-3-yl)carbonyl]-3-phenyl-8-[5-(trifluoromethyl)-1,2,4-oxadiazol-3-yl]-3,5-dihydro-2H-1,4-benzoxazepine; racemate (I-12) first and second eluting isomers (I-13 and I-14)

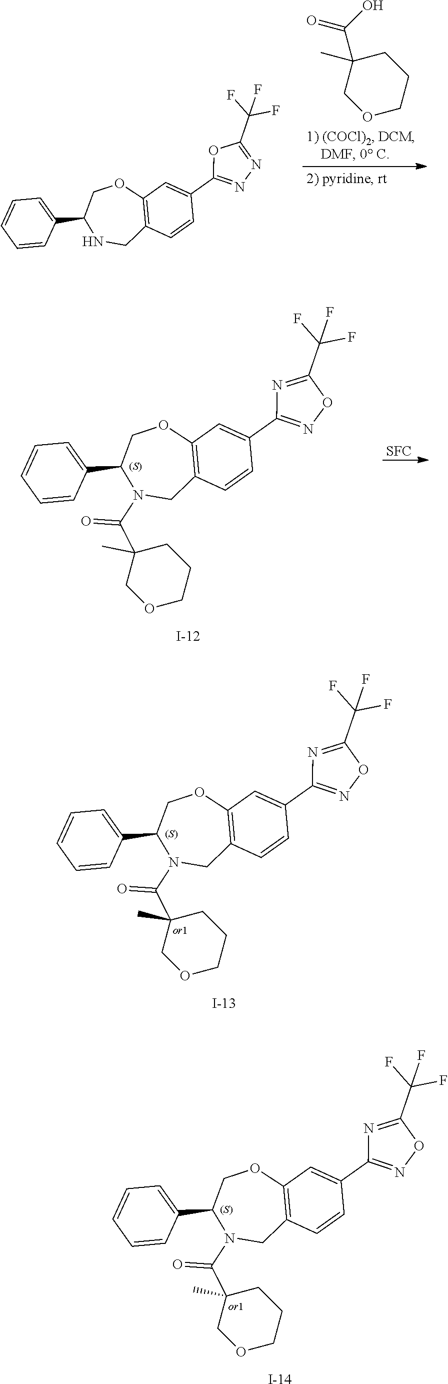

(3S)-4-[(3-methyloxan-3-yl)carbonyl]-3-phenyl-8-[5-(trifluoromethyl)-1,2,4-oxadiazol-3-yl]-3,5-dihydro-2H-1,4-benzoxazepine. To a solution of 3-methyloxane-3-carboxylic acid (200 mg, 1.3873 mmol) in dry DCM (5 mL) stirred under nitrogen at 0° C. was added (COCl)₂ (880 mg, 6.9365 mmol] and DMF (10 mg, 0.1387 mmol). The reaction mixture was stirred at 0° C. for 2 h. The reaction was concentrated under vacuum. To the residue under nitrogen at 0° C. was added a solution of isopropyl (3S)-3-phenyl-2,3,4,5-tetrahydro-1,4-benzoxazepine-8-carboxylate (751.9 mg, 2.08095 mmol) in dry pyridine. The reaction mixture was stirred at 0° C. for 4 h. The mixture was concentrated to afford the crude product. The crude product was purified by RP-C18 column eluted with H₂O (0.5% FA or NH₄OH)/CH₃CN (100:0-50:50) to afford (3S)-4-[(3-methyloxan-3-yl)carbonyl]-3-phenyl-8-[5-(trifluoromethyl)-1,2,4-oxadiazol-3-yl]-3,5-dihydro-2H-1,4-benzoxazepine (130 mg, 19.24%) as a white solid. MS (ESI): mass calcd. for C₂₅H₂₄F₃N₃O₄, 487.17, m/z found 488.2 [M+H]⁺.

(3S)-4-[(3-methyloxan-3-yl)carbonyl]-3-phenyl-8-[5-(trifluoromethyl)-1,2,4-oxadiazol-3-yl]-3,5-dihydro-2H-1,4-benzoxazepine (130 mg, 0.2667 mmol) was separated by SFC to afford a first eluting isomer (40.2 mg, 30.92%) as a white solid and a second eluting isomer (47.5 mg, 36.53%) as a white solid.

(3S)-4-[(3-methyloxan-3-yl)carbonyl]-3-phenyl-8-[5-(trifluoromethyl)-1,2,4-oxadiazol-3-yl]-3,5-dihydro-2H-1,4-benzoxazepine, first eluting isomer (I-3): MS (ESI): mass calcd. for C₂₅H₂₄F₃N₃O₄, 487.17, m/z found 488.2 [M+H]⁺. ¹H NMR (400 MHz, DMSO-d₆) δ 7.66-7.30 (m, 8H), 5.89-5.85 (m, 1H), 5.13-5.10 (m, 1H), 4.78-4.52 (m, 3H), 3.66-3.64 (m, 1H), 3.49-3.47 (m, 1H), 3.31-3.28 (m, 2H), 1.74-1.57 (m, 2H), 1.35-1.19 (m, 5H).

(3S)-4-[(3-methyloxan-3-yl)carbonyl]-3-phenyl-8-[5-(trifluoromethyl)-1,2,4-oxadiazol-3-yl]-3,5-dihydro-2H-1,4-benzoxazepine, second eluting isomer (I-14): MS (ESI): mass calcd. for C₂₅H₂₄F₃N₃O₄, 487.17, m/z found 488.2 [M+H]⁺. ¹H NMR (400 MHz, DMSO-d₆) δ 7.66-7.29 (m, 8H), 5.90-5.86 (m, 1H), 5.14-5.10 (m, 1H), 4.78-4.57 (m, 3H), 3.67-3.64 (m, 1H), 3.56-3.54 (m, 1H), 3.27-3.24 (m, 2H), 1.97-1.81 (m, 2H), 1.57-1.50 (m, 2H), 1.12 (s, 3H).

### Example 1.15. Synthesis of (S)-1-(6-fluoro-3-phenyl-8-(5-(trifluoromethyl)-1,2,4-oxadiazol-3-yl)-2,3-dihydrobenzo[f][1,4]oxazepin-4(5H)-yl)-2,2-dimethylpropan-1-one (I-15)

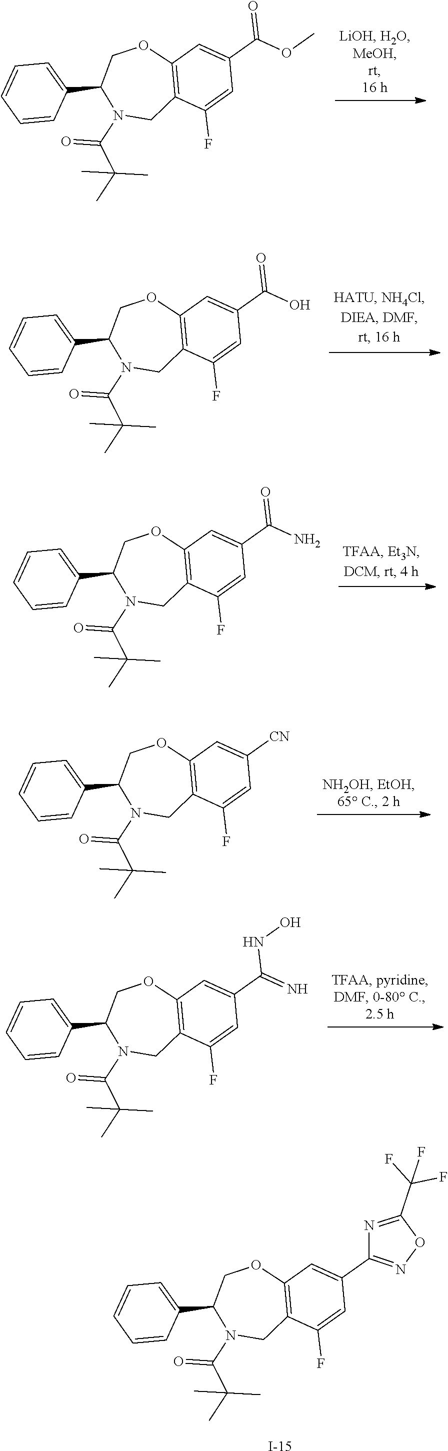

(S)-6-fluoro-3-phenyl-4-pivaloyl-2,3,4,5-tetrahydrobenzo[f][1,4]oxazepine-8-carboxylic acid. To a mixture of methyl (3S)-4-(2,2-dimethylpropanoyl)-6-fluoro-3-phenyl-3,5-dihydro-2H-1,4-benzoxazepine-8-carboxylate (800 mg, 2.08 mmol) in MeOH/H₂O (5/1, 20 mL) was added LiOH·H₂O (868 mg, 20.75 mmol). The reaction mixture was stirred at 25° C. for 16 h. The mixture was adjusted to pH=5-6 with 1M HCl (aq.). The mixture was extracted with EA (20 mL×3). The combined organic layer was washed with brine (20 mL×2), dried over Na₂SO₄ and concentrated under reduced pressure to afford crude (S)-6-fluoro-3-phenyl-4-pivaloyl-2,3,4,5-tetrahydrobenzo[f][1,4]oxazepine-8-carboxylic acid (800 mg, 90%) as a white solid. MS (ESI): mass calcd. for C₂₁H₂₂FNO₄, 371.15, m/z found 372.1 [M+H]⁺.

(S)-6-fluoro-3-phenyl-4-pivaloyl-2,3,4,5-tetrahydrobenzo[f][1,4]oxazepine-8-carboxamide. To a mixture of (3S)-4-(2,2-dimethylpropanoyl)-6-fluoro-3-phenyl-3,5-dihydro-2H-1,4-benzoxazepine-8-carboxylic acid (290 mg, 0.78 mmol) and N,N,N′,N′-Tetramethyl-O-(7-azabenzotriazol-1-yl)uronium (445 mg, 1.17 mmol) in DMF (5 mL) was added N,N-diisopropylethylamine (302 mg, 2.34 mmol) and ammonium chloride (63 mg, 1.17 mmol). The reaction mixture was stirred at 25° C. for 16 h under N₂. The mixture was quenched with water (20 mL) and extracted with EA (20 mL×3). The combined organic layer was washed with brine (20 mL×2), dried over Na₂SO₄ and concentrated under reduced pressure to afford a crude product. The crude product was purified by silica gel column chromatography eluted with DCM/MeOH (10:1) to afford (280 mg, 91%) as a white solid. MS (ESI): mass calcd. for C₂₁H₂₃FN₂O₃, 370.17, m/z found 371.1 [M+H]⁺.

(3S)-4-(2,2-dimethylpropanoyl)-6-fluoro-3-phenyl-3,5-dihydro-2H-1,4-benzoxazepine-8-carbonitrile. To a mixture of (3 S)-4-(2,2-dimethylpropanoyl)-6-fluoro-3-phenyl-3,5-dihydro-2H-1,4-benzoxazepine-8-carboxamide (390 mg, 1.05 mmol) and triethylamine (639 mg, 6.32 mmol) in DCM (10 mL) was added TFAA (663 mg, 3.16 mmol). The reaction mixture was stirred at 25° C. for additional 4 h under N₂. The mixture was quenched with water (20 mL) and extracted with EA (20 mL×3). The combined organic layer was washed with brine (20 mL×2), dried over Na₂SO₄ and concentrated under reduced pressure to afford a crude product. The crude product was purified by silica gel column chromatography eluted with DCM/MeOH (10:1) to afford (300 mg, 69%) as a white solid. MS (ESI): mass calcd. for C₂₁H₂₁FN₂O₂, 352.16, m/z found 353.1 [M+H]⁺.

(3S)-4-(2,2-dimethylpropanoyl)-6-fluoro-N-hydroxy-3-phenyl-3,5-dihydro-2H-1,4-benzoxazepine-8-carboximidamide. To a mixture of (3 S)-4-(2,2-dimethylpropanoyl)-6-fluoro-3-phenyl-3,5-dihydro-2H-1,4-benzoxazepine-8-carbonitrile (400 mg, 1.13 mmol) in EtOH (3 mL) was added NH₂OH/H₂O (225 mg, 3.41 mmol). The reaction mixture was stirred at 25° C. for additional 2 h under N₂. The mixture was quenched with ice water (20 mL). The mixture was extracted with EA (20 mL×3). The combined organic layer was washed with brine (20 mL×2), dried over Na₂SO₄ and concentrated under reduced pressure to afford a crude product which was purified by silica gel column chromatography eluted with PE/EA (3:1) to afford (400 mg, 95%) as colorless oil. MS (ESI): mass calcd. for C₂₁H₂₄FN₃O₃, 385.18, m/z found 386.1 [M+H]⁺.

(S)-1-(6-fluoro-3-phenyl-8-(5-(trifluoromethyl)-1,2,4-oxadiazol-3-yl)-2,3-dihydrobenzo[f][1,4]oxazepin-4(5H)-yl)-2,2-dimethylpropan-1-one. To a mixture of (3S)-4-(2,2-dimethylpropanoyl)-6-fluoro-N-hydroxy-3-phenyl-3, 5-dihydro-2H-1,4-benzoxazepine-8-carboximidamide (450 mg, 1.17 mmol) and pyridine (277 mg, 3.50 mmol) in DMF (5 mL) at 0° C. under N₂ was added TFAA (367 mg, 1.75 mmol). The reaction mixture was stirred at 80° C. for additional 3 h under N₂. The mixture was quenched with ice water (20 mL) and extracted with EA (20 mL×3). The combined organic layer was washed with brine (20 mL×2), dried over Na₂SO₄ and concentrated under reduced pressure to afford a crude product. The crude product was purified by RP-C18 column eluted with H₂O (0.5% FA/CH₃CN (100:0-50:50) to afford (300 mg, 69%) as a white solid. MS (ESI): mass calcd. for C₂₃H₂₁F₄N₃O₃, 463.15, m/z found 464.1 [M+H]⁺. ¹H NMR (400 MHz, DMSO) δ 7.58-7.11 (m, 7H), 5.93-5.91 (m, 1H), 5.37 (d, J=16.8 Hz, 1H), 4.87 (s, 1H), 4.58-4.44 (m, 2H), 1.13 (s, 9H).

### Example 1.16. Synthesis of (3S)-3-methyl-4-[(4-methyloxan-4-yl)carbonyl]-8-[5-(trifluoromethyl)-1,2,4-oxadiazol-3-yl]-3,5-dihydro-2H-1,4-benzoxazepine (I-16)

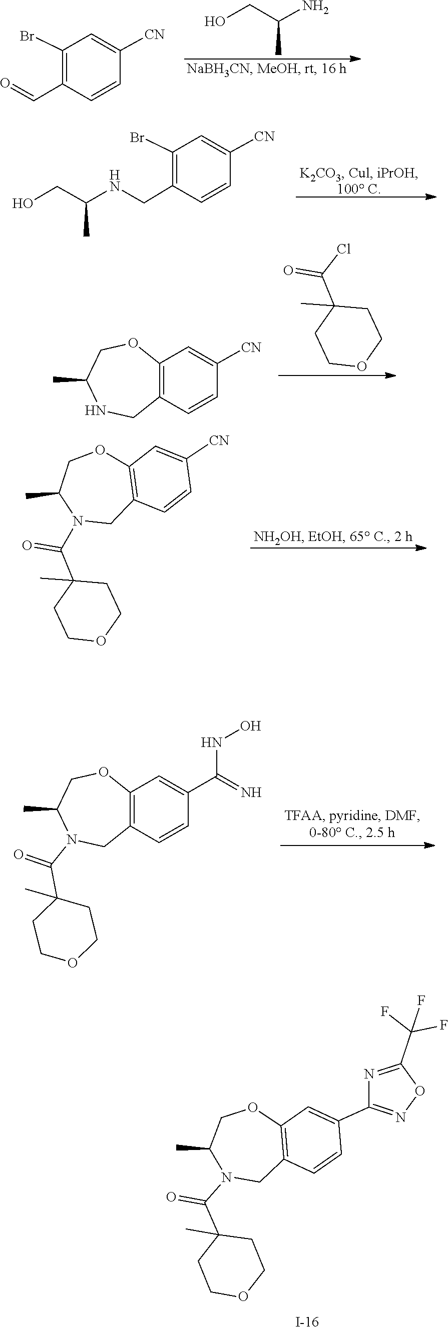

3-bromo-4-({[(2S)-1-hydroxypropan-2-yl]amino}methyl)benzonitrile. Sodium cyanoborohydride (2.51 g, 40 mmol) was added to the mixture of 3-bromo-4-formylbenzonitrile (4.2 g, 20 mmol) and (2S)-2-aminopropan-1-ol (1.65 g, 22 mmol) in MeOH (40 mL) at 0° C. The mixture was stirred at room temperature for 16 hours. The crude was diluted with water and extracted with EA (10 mL×2). The organic phase was washed with brine (10 mL×3), dried over sodium sulphate, filtered and concentrated in vacuo. The crude was triturated by EA/PE (1:10) for 6 hours to give 3-bromo-4-({[(2S)-1-hydroxypropan-2-yl]amino}methyl)benzonitrile (3.6 g, 66%) as a white solid. MS (ESI): mass calcd. for C₁₁H₁₃BrN₂O, 268.02, m/z found 269.0 [M+H]⁺. ¹H NMR (400 MHz, CDCl_{3-d}) δ 7.84 (s, 1H), 7.62-7.57 (m, 2H), 4.02-3.99 (m, 1H), 3.89-3.85 (m, 1H), 3.66-3.63 (m, 1H), 3.36-3.32 (m, 1H), 2.90-2.81 (m, 1H), 1.11 (d, J=6.4 Hz, 3H).

(3S)-3-methyl-2,3,4,5-tetrahydro-1,4-benzoxazepine-8-carbonitrile. Copper(I) iodide (420 mg, 2.2 mmol) was added to the mixture of 3-bromo-4-({[(2S)-1-hydroxypropan-2-yl]amino}methyl)benzonitrile (3 g, 11.1 mmol) and Potassium carbonate (3.07 g, 22.2 mmol) in iPrOH (15 mL). The reaction mixture was stirred at 100° C. for 16 hours. The crude was diluted with water and extracted with EA (10 mL×2). The organic phase was washed with brine (10 mL×3), dried over sodium sulphate, filtered and concentrated in vacuo. The residue was purified by chromatography column on silica gel (40 g) eluting with PE/EA (5%) to give (3S)-3-methyl-2,3,4,5-tetrahydro-1,4-benzoxazepine-8-carbonitrile (1.6 g, 69%) as a white solid. MS (ESI): mass calcd. for C₁₁H₁₂N₂O, 188.09, m/z found 189.1 [M+H]⁺.

(3S)-3-methyl-4-[(4-methyloxan-4-yl)carbonyl]-3,5-dihydro-2H-1,4-benzoxazepine-8-carbonitrile. 4-methyloxane-4-carbonyl chloride (1.12 g, 6.9 mmol) was added to the mixture of (3S)-3-methyl-2,3,4,5-tetrahydro-1,4-benzoxazepine-8-carbonitrile (1.3 g, 6.9 mmol), triethylamine (2.79 g, 27.6 mmol) and 4-DMAP (80 mg, 0.6 mmol) in dry DCM (10 mL) at 0° C. The mixture was stirred at room temperature for 16 hours. The residue was diluted with water and extracted with DCM (20 mL×2). The organic phase was washed with brine (10 mL×3), dried over sodium sulphate, filtered and concentrated in vacuo. The residue was purified by chromatography column on silica gel (40 g) eluting with PE/EA to give (3S)-3-methyl-4-[(4-methyloxan-4-yl)carbonyl]-3,5-dihydro-2H-1,4-benzoxazepine-8-carbonitrile (1.8 g, 75%) as a white solid. MS (ESI): mass calcd. for C₁₈H₂₂N₂O₃, 314.16, m/z found 315.1 [M+H]⁺.

(3S)—N-hydroxy-3-methyl-4-[(4-methyloxan-4-yl)carbonyl]-3,5-dihydro-2H-1,4-benzoxazepine-8-carboximidamide. NH₂OH/H₂O (50%, 12.6 g) was added to the mixture of (3S)-3-methyl-4-[(4-methyloxan-4-yl)carbonyl]-3, 5-dihydro-2H-1,4-benzoxazepine-8-carbonitrile (600 mg, 1.9 mmol) in EtOH (10 mL). The mixture was stirred at 65° C. for 2 hours. The crude was diluted with water and extracted with DCM (10 mL×2). The organic phase was washed with brine (10 mL×3), dried over sodium sulphate, filtered and concentrated in vacuo. The residue was purified by chromatography column on silica gel (25 g) eluting with PE/EA to give (3S)—N-hydroxy-3-methyl-4-[(4-methyloxan-4-yl)carbonyl]-3,5-dihydro-2H-1,4-benzoxazepine-8-carboximidamide (600 mg, 90%) as a white solid. MS (ESI): mass calcd. for C₁₈H₂₅N₃O₄, 347.18, m/z found 348.1 [M+H]⁺.

(3S)-3-methyl-4-[(4-methyloxan-4-yl)carbonyl]-8-[5-(trifluoromethyl)-1,2,4-oxadiazol-3-yl]-3,5-dihydro-2H-1,4-benzoxazepine. 2,2,2-trifluoroacetyl 2,2,2-trifluoroacetate (181 mg, 0.86 mmol) was added to the mixture of (3S)—N-hydroxy-3-methyl-4-[(4-methyloxan-4-yl)carbonyl]-3,5-dihydro-2H-1,4-benzoxazepine-8-carboximidamide (200 mg, 0.57 mmol) and pyridine (136 mg, 1.72 mmol) in DMF (4 mL) at 0° C. The mixture was stirred at 80° C. for 3 hours. The crude was diluted with water and extracted with EA (10 mL×2). The organic phase was washed with brine (10 mL×3), dried over sodium sulphate, filtered and concentrated in vacuo. The residue was purified by prep-HPLC (Instrument: Waters MS triggered Prep-LC with Acquity QDA detector, Column: Welch 10 u C18 250×21.2 mm, A water (0.1% FA), B Acetonitrile 45-75% B in 9 min, hold at 100% B for 2.0 mins, back to 45% B with 0.5 min, stop at 15 min, flow rate: 25 mL/min, wavelength: 214/254 nm, injection: 4) to give (3S)-3-methyl-4-[(4-methyloxan-4-yl)carbonyl]-8-[5-(trifluoromethyl)-1,2,4-oxadiazol-3-yl]-3, 5-dihydro-2H-1,4-benzoxazepine (89.2 mg, 33%) as a white solid. MS (ESI): mass calcd. for C₂₀H₂₂F₃N₃O₄, 425.16, m/z found 426.1 [M+H]⁺. ¹H NMR (400 MHz, CDCl₃-d) δ 7.73-7.71 (m, 1H), 7.65-7.63 (m, 1H), 7.38-7.34 (m, 1H), 5.04-4.69 (m, 2H), 4.46 (s, 1H), 4.25-4.09 (m, 2H), 3.77-3.37 (m, 4H), 2.21-2.07 (m, 2H), 1.69-1.60 (m, 1H), 1.50-1.44 (m, 1H), 1.35-1.23 (m, 6H).

### Example 1.17. Synthesis of 2,2-dimethyl-1-[(3S)-3-methyl-8-[5-(trifluoromethyl)-1,2,4-oxadiazol-3-yl]-3,5-dihydro-2H-1,4-benzoxazepin-4-yl]propan-1-one (I-17)

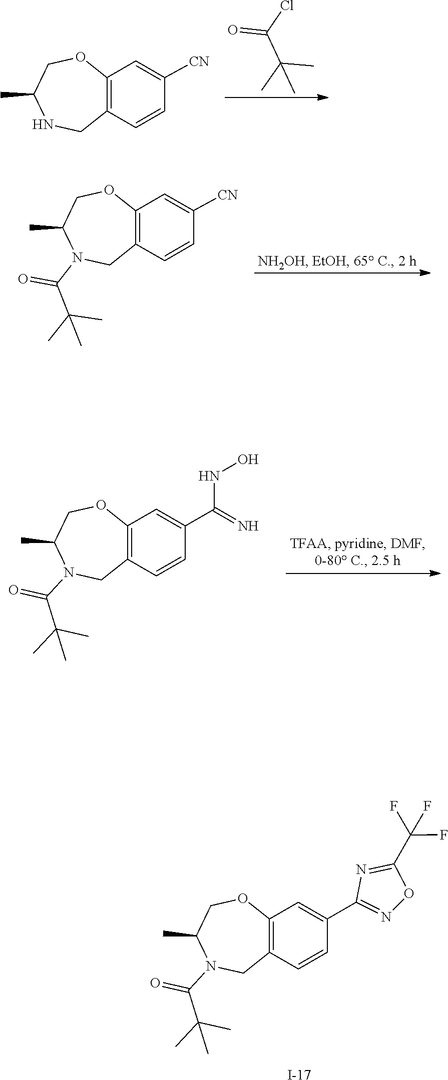

(3S)-4-(2,2-dimethylpropanoyl)-3-methyl-3,5-dihydro-2H-1,4-benzoxazepine-8-carbonitrile. 4-DMAP (39 mg, 0.32 mmol) was added to the mixture of (3S)-3-methyl-2,3,4,5-tetrahydro-1,4-benzoxazepine-8-carbonitrile (600 mg, 3.2 mmol), 2,2-dimethylpropanoyl chloride (961 mg, 8.0 mmol) and triethylamine (1.29 g, 12.7 mmol) in DCM (10 mL) at 0° C. The mixture was stirred at room temperature for 3 hours. The crude was diluted with water and extracted with DCM (10 mL×2). The organic phase was washed with brine (10 mL×3), dried over sodium sulphate, filtered and concentrated in vacuo. The residue was purified by chromatography column on silica gel (40 g) eluting with PE/EA (5%) to give (3S)-4-(2,2-dimethylpropanoyl)-3-methyl-3,5-dihydro-2H-1,4-benzoxazepine-8-carbonitrile (860 mg, 89%) as red oil. MS (ESI): mass calcd. for C₁₆H₂₀N₂O₂, 272.15, m/z found 273.1 [M+H]⁺.

(3S)-4-(2,2-dimethylpropanoyl)-N-hydroxy-3-methyl-3,5-dihydro-2H-1,4-benzoxazepine-8-carboximidamide. NH₂OH/H₂O (50%, 9.7 g) was added to the solution of (3S)-4-(2,2-dimethylpropanoyl)-3-methyl-3,5-dihydro-2H-1,4-benzoxazepine-8-carbonitrile (400 mg, 1.5 mmol) in EtOH (10 mL). The mixture was stirred at 65° C. for 2 hours. The crude was concentrated in vacuo, diluted with water and extracted with DCM (10 mL×2). The organic phase was washed with brine (10 mL×3), dried over sodium sulphate, filtered and concentrated in vacuo. The residue was purified by chromatography column on silica gel (25 g) eluting with PE/EA (20%-50%) to give (3S)-4-(2,2-dimethylpropanoyl)-N-hydroxy-3-methyl-3,5-dihydro-2H-1,4-benzoxazepine-8-carboximidamide (440 mg, 93%) as a white solid. MS (ESI): mass calcd. for C₁₆H₂₃N₃O₃, 305.17, m/z found 306.1 [M+H]⁺. ¹H NMR (400 MHz, DMSO-d) δ 9.59 (s, 1H), 7.24-7.23 (m, 2H), 7.11 (s, 1H), 5.76-5.74 (m, 2H), 4.81-4.49 (m, 3H), 4.25-3.95 (m, 3H), 1.15 (s, 9H).

2,2-dimethyl-1-[(3S)-3-methyl-8-[5-(trifluoromethyl)-1,2,4-oxadiazol-3-yl]-3,5-dihydro-2H-1,4-benzoxazepin-4-yl]propan-1-one. TFAA (206 mg, 0.98 mmol) was added to the mixture of (3S)-4-(2,2-dimethylpropanoyl)-N-hydroxy-3-methyl-3,5-dihydro-2H-1,4-benzoxazepine-8-carboximidamide (200 mg, 0.65 mmol) and pyridine (155 mg, 1.9 mmol) in DMF (2 mL) at 0° C. The mixture was stirred at 80° C. for 2.5 hours. The residue was extracted with EA (10 mL×2). The organic phase was washed with brine (10 mL×3), dried over sodium sulphate, filtered and concentrated in vacuo. The residue was purified by prep-HPLC (Instrument: Waters MS triggered Prep-LC with Acquity QDA detector, Column: Welch 10 u C18 250×21.2 mm, A water (0.1% FA), B Acetonitrile 55-85% B in 9 min, hold at 100% B for 2.0 mins, back to 55% B with 0.5 min, stop at 15 min, flow rate: 25 mL/min, wavelength: 214/254 nm, injection: 4) to give 2,2-dimethyl-1-[(3S)-3-methyl-8-[5-(trifluoromethyl)-1,2,4-oxadiazol-3-yl]-3,5-dihydro-2H-1,4-benzoxazepin-4-yl]propan-1-one (39.3 mg, 15%) as a white solid. MS (ESI): mass calcd. for C₁₈H₂₀F₃N₃O₃, 383.15, m/z found 384.1 [M+H]⁺. ¹H NMR (400 MHz, DMSO-d₆) δ 7.64-7.61 (m, 1H), 7.52-7.50 (m, 1H), 7.42 (d, J=1.6 Hz, 1H), 4.89-4.57 (m, 3H), 4.31-4.19 (m, 2H), 1.18-1.14 (m, 12H).

### Example 1.18. Synthesis of (3S)-6-fluoro-3-methyl-4-[(4-methyloxan-4-yl)carbonyl]-8-[5-(trifluoromethyl)-1,2,4-oxadiazol-3-yl]-3,5-dihydro-2H-1,4-benzoxazepine (I-18)

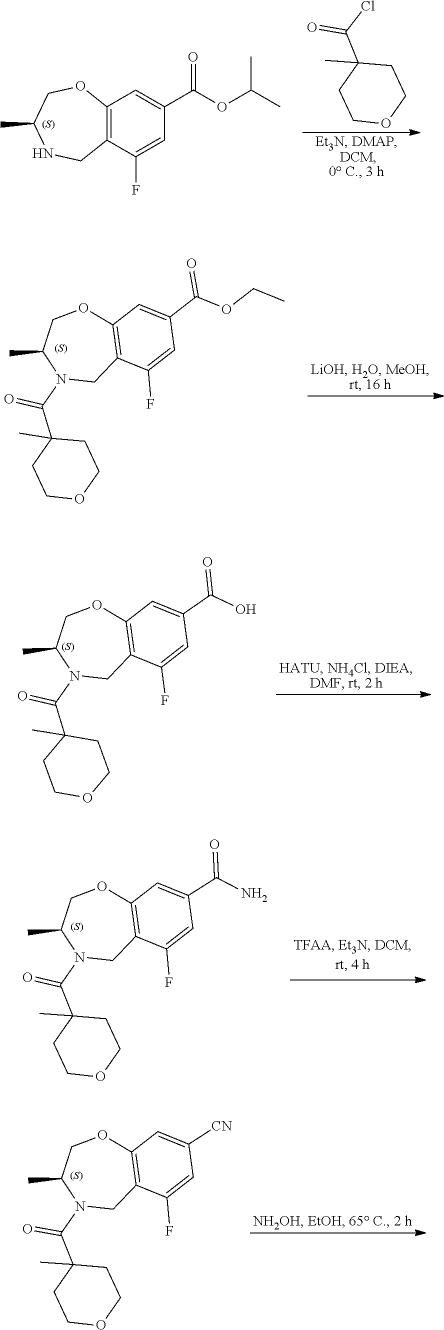

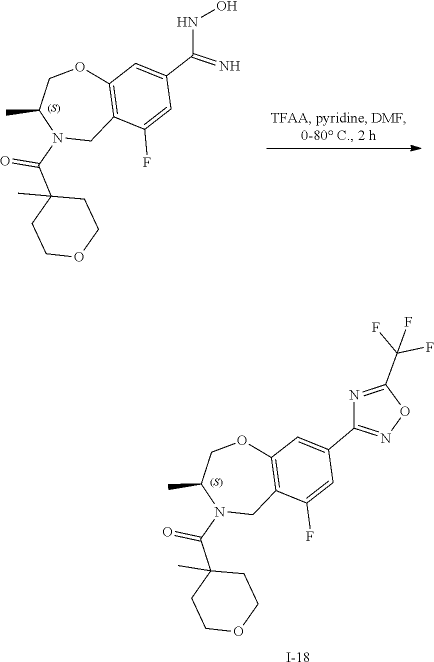

Isopropyl (3S)-6-fluoro-3-methyl-4-[(4-methyloxan-4-yl)carbonyl]-3,5-dihydro-2H-1,4-benzoxazepine-8-carboxylate. To a solution of isopropyl (3S)-6-fluoro-3-methyl-2,3,4,5-tetrahydro-1,4-benzoxazepine-8-carboxylate (1000 mg, 3.74 mmol), triethylamine (1136 mg, 11.22 mmol) and 4-dimethylaminopyridine (46 mg, 0.37 mmol) in DCM (15 mL) was added 4-methyloxane-4-carbonyl chloride (1217 mg, 7.48 mmol) at 0° C. The reaction mixture was stirred at 0° C. under N₂ for 3 h. The mixture was quenched with water (15 mL) and extracted with DCM (3×10 mL). The combined organic layer was dried over Na₂SO₄ and concentrated under reduced pressure to get a residue which was purified by column chromatography on silica gel (25 g) eluting with MeOH in DCM (0-2%) to afford isopropyl (3S)-6-fluoro-3-methyl-4-[(4-methyloxan-4-yl)carbonyl]-3,5-dihydro-2H-1,4-benzoxazepine-8-carboxylate (1.1 g, 75%) as a white solid. MS (ESI): mass calcd. for C₂₁H₂₈FNO₅, 393.2, m/z found 394.1 [M+H]⁺.

(3S)-6-fluoro-3-methyl-4-[(4-methyloxan-4-yl)carbonyl]-3,5-dihydro-2H-1,4-benzoxazepine-8-carboxylic acid. To a solution of isopropyl (3S)-6-fluoro-3-methyl-4-[(4-methyloxan-4-yl)carbonyl]-3,5-dihydro-2H-1,4-benzoxazepine-8-carboxylate (1.1 g, 2.8 mmol) in MeOH (8 mL) and H₂O (8 mL) was added lithium hydroxide (0.34 g, 14.0 mmol). The reaction mixture was stirred at room temperature for 16 h. The mixture was adjusted to pH=2-3 with 1M HCl and filtered. The filter cake was washed with H₂O (20 mL) and dried under reduced pressure to afford (3S)-6-fluoro-3-methyl-4-[(4-methyloxan-4-yl)carbonyl]-3,5-dihydro-2H-1,4-benzoxazepine-8-carboxylic acid (0.85 g, 86%) as a white solid. MS (ESI): mass calcd. for C₁₈H₂₂FNO₅, 351.1, m/z found 352.1 [M+H]⁺.

(3S)-6-fluoro-3-methyl-4-[(4-methyloxan-4-yl)carbonyl]-3,5-dihydro-2H-1,4-benzoxazepine-8-carboxamide. To a solution of (3S)-6-fluoro-3-methyl-4-[(4-methyloxan-4-yl)carbonyl]-3,5-dihydro-2H-1,4-benzoxazepine-8-carboxylic acid (500 mg, 1.42 mmol), amine hydrochloride (228 mg, 4.27 mmol) and HATU (649 mg, 1.71 mmol) in DMF (10 mL) was added DIEA (552 mg, 4.27 mmol). The reaction mixture was stirred at room temperature for 2 h. The mixture was diluted with water (20 mL) and extracted with EA (10 mL×3). The combined organic layer was washed with brine (20 mL×3) and concentrated to get a residue which was purified by column chromatography on silica gel (25 g) eluting with MeOH in DCM (0-5%) to afford (3S)-6-fluoro-3-methyl-4-[(4-methyloxan-4-yl)carbonyl]-3,5-dihydro-2H-1,4-benzoxazepine-8-carboxamide (430 mg, 87%) as a yellow solid. MS (ESI): mass calcd. for C₁₈H₂₃FN₂O₄, 350.2, m/z found 351.1 [M+H]⁺.

(3S)-6-fluoro-3-methyl-4-[(4-methyloxan-4-yl)carbonyl]-3,5-dihydro-2H-1,4-benzoxazepine-8-carbonitrile. To a solution of (3S)-6-fluoro-3-methyl-4-[(4-methyloxan-4-yl)carbonyl]-3,5-dihydro-2H-1,4-benzoxazepine-8-carboxamide (400 mg, 1.14 mmol) and trimethylamine (693 mg, 6.85 mmol) in DCM (10 mL) was added TFAA (719 mg, 3.42 mmol). The reaction mixture was stirred at room temperature for 4 h. The mixture was quenched with water (10 mL) and extracted with DCM (3×10 mL). The combined organic layer was dried over Na₂SO₄ and concentrated under reduced pressure to get a residue which was purified by column chromatography on silica gel (12 g) eluting with EA in PE (0-40%) to afford (3S)-6-fluoro-3-methyl-4-[(4-methyloxan-4-yl)carbonyl]-3,5-dihydro-2H-1,4-benzoxazepine-8-carbonitrile (320 mg, 84%) as a yellow solid. MS (ESI): mass calcd. for C₁₈H₂₁FN₂O₃, 332.2, m/z found 333.1 [M+H]⁺.

(3S)-6-fluoro-N-hydroxy-3-methyl-4-[(4-methyloxan-4-yl)carbonyl]-3,5-dihydro-2H-1,4-benzoxazepine-8-carboximidamide. To a solution of (3S)-6-fluoro-3-methyl-4-[(4-methyloxan-4-yl)carbonyl]-3,5-dihydro-2H-1,4-benzoxazepine-8-carbonitrile (290 mg, 0.87 mmol) in EtOH (5 mL) was added NH₂OH/H₂O (5764 mg, 87.25 mmol, 50%). The reaction mixture was stirred at 65° C. for 2 h. The mixture was cooled to room temperature and extracted with DCM (10 mL×3). The combined organic layer was dried over Na₂SO₄ and concentrated under reduced pressure to get a residue which was purified by column chromatography on silica gel (12 g) eluting with MeOH in DCM (0-5%) to afford (3S)-6-fluoro-N-hydroxy-3-methyl-4-[(4-methyloxan-4-yl)carbonyl]-3,5-dihydro-2H-1,4-benzoxazepine-8-carboximidamide (310 mg, 97%) as a white solid. MS (ESI): mass calcd. for C₁₈H₂₄FN₃O₄, 365.2, m/z found 366.0 [M+H]

(3S)-6-fluoro-3-methyl-4-[(4-methyloxan-4-yl)carbonyl]-8-[5-(trifluoromethyl)-1,2,4-oxadiazol-3-yl]-3,5-dihydro-2H-1,4-benzoxazepine. To a solution of (3S)-6-fluoro-N-hydroxy-3-methyl-4-[(4-methyloxan-4-yl)carbonyl]-3,5-dihydro-2H-1,4-benzoxazepine-8-carboximidamide (200 mg, 0.55 mmol) and pyridine (130 mg, 1.64 mmol) in DMF (3 mL) was added TFAA (230 mg, 1.09 mmol) at 0° C. The reaction mixture was stirred at 80° C. for 2 h. The mixture was cooled to room temperature and purified by Pre-HPLC (Instrument: Waters Prep-HPLC, Column: X-bridge Prep C18 5 um, A water (0.1% FA), B Acetonitrile 60-90% B in 9 min, hold at 100% B for 1 min, back to 60% B within 1.5 min, stop at 15 min, flow rate: 25 mL/min, wavelength: 214 nm, injection: 7) to afford (3S)-6-fluoro-3-methyl-4-[(4-methyloxan-4-yl)carbonyl]-8-[5-(trifluoromethyl)-1,2,4-oxadiazol-3-yl]-3,5-dihydro-2H-1,4-benzoxazepine (151.8 mg, 63%) as a white solid. MS (ESI): mass calcd. for C₂₀H₂₁F₄N₃O₄, 443.1, m/z found 444.1 [M+H]⁺. ¹H NMR (400 MHz, DMSO-d₆) δ 7.46 (d, J=9.2 Hz, 1H), 7.29 (s, 1H), 5.05 (d, J=16.8 Hz, 1H), 4.78 (s, 1H), 4.57-4.19 (m, 3H), 3.64-3.51 (m, 2H), 3.41-3.33 (m, 2H), 1.95 (d, J=10.8 Hz, 2H), 1.51 (s, 1H), 1.44-1.32 (m, 1H), 1.23 (s, 3H), 1.15 (d, J=3.6 Hz, 3H).

### Example 1.19. Synthesis of 1-[(3S)-6-fluoro-3-methyl-8-[5-(trifluoromethyl)-1,2,4-oxadiazol-3-yl]-3,5-dihydro-2H-1,4-benzoxazepin-4-yl]-2,2-dimethylpropan-1-one (I-19)

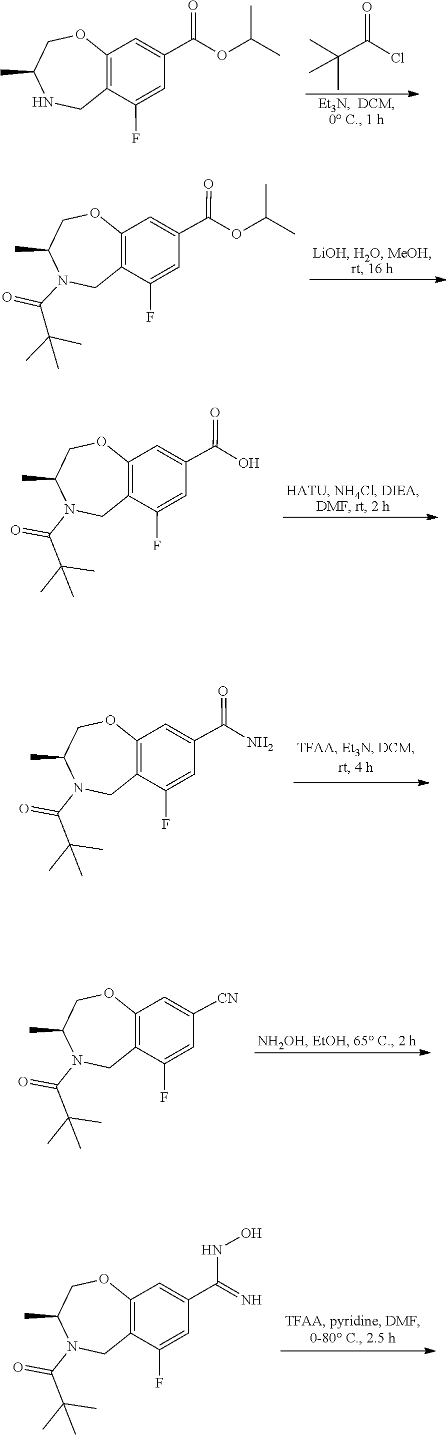

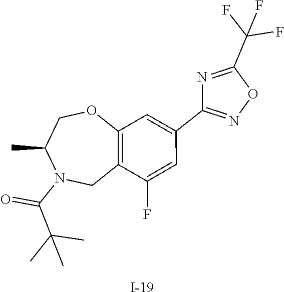

Isopropyl (3S)-4-(2,2-dimethylpropanoyl)-6-fluoro-3-methyl-3,5-dihydro-2H-1,4-benzoxazepine-8-carboxylate. To a solution of isopropyl (3S)-6-fluoro-3-methyl-2,3,4,5-tetrahydro-1,4-benzoxazepine-8-carboxylate (1.1 g, 4.1 mmol) and trimethylamine (1.24 g, 12.3 mmol) in DCM (15 mL) was added 2,2-dimethylpropanoyl chloride (1.48 g, 12.3 mmol) at 0° C. The reaction mixture was stirred at 0° C. under N₂ for 2 h. The mixture was quenched with water (15 mL) and extracted with DCM (3×10 mL). The combined organic layer was dried over Na₂SO₄ and concentrated under reduced pressure to get a residue which was purified by column chromatography on silica gel (25 g) eluting with MeOH in DCM (0-2%) to afford isopropyl (3S)-4-(2,2-dimethylpropanoyl)-6-fluoro-3-methyl-3,5-dihydro-2H-1,4-benzoxazepine-8-carboxylate (1.2 g, 82%) as a white solid. MS (ESI): mass calcd. for C₁₉H₂₆FNO₄, 351.2, m/z found 352.2 [M+H]⁺.

(3S)-4-(2,2-dimethylpropanoyl)-6-fluoro-3-methyl-3,5-dihydro-2H-1,4-benzoxazepine-8-carboxylic acid. To a solution of isopropyl (3S)-4-(2,2-dimethylpropanoyl)-6-fluoro-3-methyl-3,5-dihydro-2H-1,4-benzoxazepine-8-carboxylate (1.15 g, 3.3 mmol) in MeOH (8 mL) and H₂O (8 mL) was added Lithium hydroxide (0.4 g, 16.5 mmol). The reaction mixture was stirred at room temperature for 16 h. The mixture was adjusted to pH=2-3 with 1M HCl and filtered. The filter cake was washed with H₂O (20 mL) and dried under reduced pressure to afford (3S)-4-(2,2-dimethylpropanoyl)-6-fluoro-3-methyl-3,5-dihydro-2H-1,4-benzoxazepine-8-carboxylic acid (0.86 g, 83%) as a white solid. MS (ESI): mass calcd. for C₁₆H₂₀FNO₄, 309.1, m/z found 310.1 [M+H]⁺.

(3S)-4-(2,2-dimethylpropanoyl)-6-fluoro-3-methyl-3,5-dihydro-2H-1,4-benzoxazepine-8-carboxamide. To a solution of 4-(2,2-dimethylpropanoyl)-6-fluoro-3,5-dihydro-2H-1,4-benzoxazepine-8-carboxylic acid (500 mg, 1.62 mmol), amine hydrochloride (259 mg, 4.85 mmol) and HATU (738 mg, 1.94 mmol) in DMF (10 mL) was added DIEA (627 mg, 4.85 mmol). The reaction mixture was stirred at room temperature for 2 h. The mixture was diluted with water (20 mL) and extracted with EA (10 mL×3). The combined organic layer was washed with brine (20 mL×3) and concentrated to get a residue which was purified by column chromatography on silica gel (25 g) eluting with EA in PE (0-70%) to afford (3S)-4-(2,2-dimethylpropanoyl)-6-fluoro-3-methyl-3,5-dihydro-2H-1,4-benzoxazepine-8-carboxamide (450 mg, 90%) as a white solid. MS (ESI): mass calcd. for C₁₈H₂₃FN₂O₄, 308.2, m/z found 309.1 [M+H]⁺.

(3S)-4-(2,2-dimethylpropanoyl)-6-fluoro-3-methyl-3,5-dihydro-2H-1,4-benzoxazepine-8-carbonitrile. To a solution of (3S)-4-(2,2-dimethylpropanoyl)-6-fluoro-3-methyl-3,5-dihydro-2H-1,4-benzoxazepine-8-carboxamide (420 mg, 1.36 mmol) and trimethylamine (827 mg, 8.17 mmol) in DCM (10 mL) was added TFAA (858 mg, 4.08 mmol). The reaction mixture was stirred at room temperature for 4 h. The mixture was quenched with water (10 mL) and extracted with DCM (3×10 mL). The combined organic layer was dried over Na₂SO₄ and concentrated under reduced pressure to get a residue which was purified by column chromatography on silica gel (12 g) eluting with EA in PE (0-33%) to afford (3S)-4-(2,2-dimethylpropanoyl)-6-fluoro-3-methyl-3,5-dihydro-2H-1,4-benzoxazepine-8-carbonitrile (380 mg, 96%) as a yellow solid. MS (ESI): mass calcd. for C₁₆H₁₉FN₂O₂, 290.1, m/z found 291.1 [M+H]⁺.

(3S)-4-(2,2-dimethylpropanoyl)-6-fluoro-N-hydroxy-3-methyl-3,5-dihydro-2H-1,4-benzoxazepine-8-carboximidamide. To a solution of (3S)-4-(2,2-dimethylpropanoyl)-6-fluoro-3-methyl-3,5-dihydro-2H-1,4-benzoxazepine-8-carbonitrile (350 mg, 1.03 mmol) in EtOH (5 mL) was added NH₂OH/H₂O (6826 mg, 103.33 mmol). The reaction mixture was stirred at 65° C. for 2 h. The mixture was cooled to room temperature and extracted with DCM (10 mL×3). The combined organic layer was dried over Na₂SO₄ and concentrated under reduced pressure to get a residue which was purified by column chromatography on silica gel (12 g) eluting with MeOH in DCM (0-5%) to afford (3 S)-4-(2,2-dimethylpropanoyl)-6-fluoro-N-hydroxy-3-methyl-3,5-dihydro-2H-1,4-benzoxazepine-8-carboximidamide (370 mg, 95%) as a white solid. MS (ESI): mass calcd. for C₁₆H₂₂FN₃O₃, 323.2, m/z found 324.1 [M+H]⁺.

1-[(3S)-6-fluoro-3-methyl-8-[5-(trifluoromethyl)-1,2,4-oxadiazol-3-yl]-3,5-dihydro-2H-1,4-benzoxazepin-4-yl]-2,2-dimethylpropan-1-one. To a solution of (3 S)-4-(2,2-dimethylpropanoyl)-6-fluoro-N-hydroxy-3-methyl-3, 5-dihydro-2H-1,4-benzoxazepine-8-carboximidamide (200 mg, 0.62 mmol) and pyridine (147 mg, 1.86 mmol) in DMF (3 mL) was added TFAA (260 mg, 1.24 mmol) at 0° C. The reaction mixture was stirred at 80° C. for 2 h. The mixture was cooled to room temperature and purified by Pre-HPLC (Instrument: Waters Prep-HPLC, Column: X-bridge Prep C18 5 um, A water (0.1% FA), B Acetonitrile 70-95% B in 9 min, hold at 100% B for 1 min, back to 70% B within 1.5 min, stop at 15 min, flow rate: 25 mL/min, wavelength: 214 nm, injection: 8) to afford 1-[(3S)-6-fluoro-3-methyl-8-[5-(trifluoromethyl)-1,2,4-oxadiazol-3-yl]-3, 5-dihydro-2H-1,4-benzoxazepin-4-yl]-2,2-dimethylpropan-1-one (40.2 mg, 16%) as a white solid. MS (ESI): mass calcd. for C₁₈H₁₉F₄N₃O₃, 401.1, m/z found 402.2 [M+H]⁺. ¹H NMR (400 MHz, DMSO-d₆) δ 7.45 (d, J=9.6 Hz, 1H), 7.29 (s, 1H), 5.05 (d, J=16.8 Hz, 1H), 4.82-4.72 (m, 1H), 4.59-4.18 (m, 3H), 1.17 (s, 12H).

### Example 1.20. Synthesis of (S)-1-(8-(5-(difluoromethyl)-1,3,4-oxadiazol-2-yl)-3-methyl-2,3-dihydrobenzo[f][1,4]oxazepin-4(5H)-yl)-2,2-dimethylpropan-1-one (I-20)

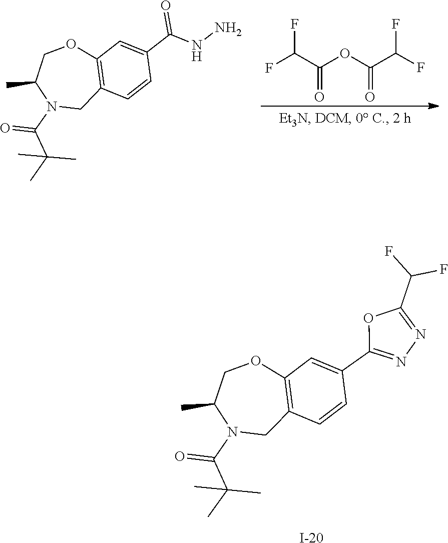

To a solution of (3S)-4-(2,2-dimethylpropanoyl)-3-methyl-3,5-dihydro-2H-1,4-benzoxazepine-8-carbohydrazide (250 mg, 0.82 mmol) and TEA (414 mg, 4.09 mmol) in DCM (10 mL) cooled to 0° C. was added 2,2-difluoroacetyl 2,2-difluoroacetate (427 mg, 2.46 mmol) under N₂, the reaction mixture was stirred at 25° C. for 4 h. The reaction mixture was quenched with water (30 mL) and extracted with DCM (50 mL×3), the combined organic layer washed with brine (80 mL), dried over Na₂SO₄, concentrated to get the residue. The residue was purified by prep-HPLC (Chromatographic columns:-Xbridge-C18 150×19 mm, 5 um Mobile Phase: CAN —H₂O (0.1% FA), B (Acetonitrile), flow rate: 20 mL/min, wavelength: 214/254 nm, injection: 10) to get (S)-1-(8-(5-(difluoromethyl)-1,3,4-oxadiazol-2-yl)-3-methyl-2,3-dihydrobenzo[f][1,4]oxazepin-4(5H)-yl)-2,2-dimethylpropan-1-one (148.1 mg, 49%) as a white solid. MS (ESI): mass calcd. for C₁₈H₂₀F₂N₃O₃ 365.16, m/z found 366.1 [M+H]⁺. ¹H NMR (400 MHz, DMSO-d₆) δ 7.75-7.28 (m, 4H), 4.90-4.46 (m, 3H), 4.29-4.14 (m, 2H), 1.13 (s, 12H).

### Example 1.21. Synthesis of (S)-2,2-dimethyl-1-(3-phenyl-8-(5-(trifluoromethyl)-1,3,4-oxadiazol-2-yl)-2,3-dihydrobenzo[f][1,4]oxazepin-4(5H)-yl)propan-1-one (I-21)

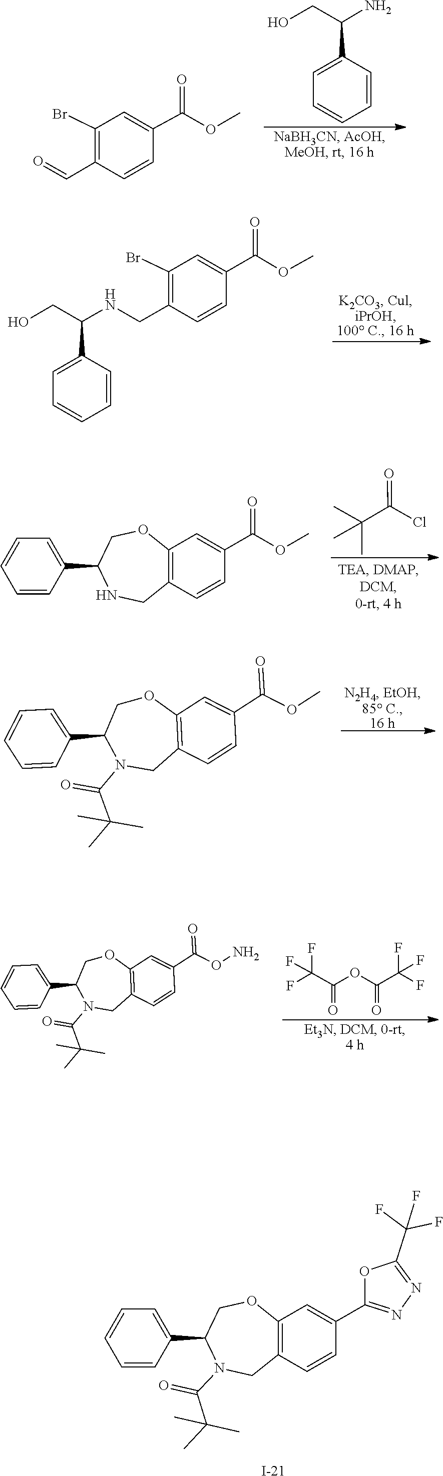

Methyl 3-bromo-4-({[(1S)-2-hydroxy-1-phenylethyl]amino}methyl) benzoate. To a solution of methyl 4-bromo-5-formylthiophene-2-carboxylate (15.0 g, 0.0617 mol) in MeOH (100 mL) was added (2S)-2-amino-2-phenylethanol (10.2 g, 0.0740 mol), AcOH (1.1 g, 0.0185 mol), NaBH₃CN (7.7 g, 0.1234 mol). The reaction mixture was stirred at 25° C. for 16 h. The mixture was quenched by H₂O (100 mL), extracted with EA (2*150 mL). The organic layer was washed with brine (150 mL), dried over anhydrous Na₂SO₄, concentrated. The residue was applied onto silica gel column, this resulted product (9.0 g, 38%) as a white solid. MS (ESI): mass calcd. for C₁₇H₁₈BrNO₃, 363.05, m/z found 364.0 [M+H]⁺.

(3S)-3-phenyl-2,3,4,5-tetrahydro-1,4-benzoxazepine-8-carboxylate. To a solution of methyl 3-bromo-4-({[(1S)-2-hydroxy-1-phenylethyl]amino}methyl) benzoate (8.5 g, 0.0233 mol) in IPA (100 mL) was added Copper(I) iodide (1.3 g, 0.0069 mmol), K₂CO₃ (6.4 g, 0.0466 mol). The reaction mixture was stirred at 100° C. for 16 h. The mixture was quenched by H₂O (100 mL), extracted with EA (2*200 mL). The organic layer was washed with brine (200 mL), dried over anhydrous Na₂SO₄, concentrated. The residue was applied onto silica gel column, this resulted product (4.0 g, 57%) as a white solid. MS (ESI): mass calcd. for C₁₇H₁₇NO₃, 283.12, m/z found 284.1 [M+H]⁺.

Methyl (3S)-4-(2,2-dimethylpropanoyl)-3-phenyl-3,5-dihydro-2H-1,4-benzoxazepine-8-carboxylate. To a solution of methyl (3S)-3-phenyl-2,3,4,5-tetrahydro-1,4-benzoxazepine-8-carboxylate (500 mg, 1.76 mmol) in DCM (10 mL) was added TEA (714 mg, 7.06 mmol), 4-DMAP (22 mg, 0.17 mmol), 2,2-dimethylpropanoyl chloride (426 mg, 3.53 mmol). The reaction mixture was stirred at 25° C. for 4 h. The mixture was quenched by H₂O (30 mL), extracted with DCM (2*30 mL). The organic layer was washed with brine (30 mL), dried over anhydrous Na₂SO₄, concentrated. The residue was applied onto silica gel column, this resulted product (550 mg, 80%) as a white solid. MS (ESI): mass calcd. for C₂₂H₂₅NO₄, 367.18, m/z found 368.2 [M+H]⁺.

(3S)-4-(2,2-dimethylpropanoyl)-3-phenyl-3,5-dihydro-2H-1,4-benzoxazepine-8-carbohydrazide. To a solution of methyl (3S)-4-(2,2-dimethylpropanoyl)-3-phenyl-3,5-dihydro-2H-1,4-benzoxazepine-8-carboxylate (550 mg, 1.50 mmol) in EtOH (10 mL) was added NH₂NH₂/H₂O (1515 mg, 29.94 mmol). The reaction mixture was stirred at 85° C. for 16 h. The mixture was concentrated under vacuum, this resulted product (600 mg, 98%) as a white solid. MS (ESI): mass calcd. for C₂₁H₂₅N₃O₃, 367.19, m/z found 368.2 [M+H]⁺.

(S)-2,2-dimethyl-1-(3-phenyl-8-(5-(trifluoromethyl)-1,3,4-oxadiazol-2-yl)-2,3-dihydrobenzo[f][1,4]oxazepin-4(5H)-yl)propan-1-one. To a solution of (3S)-4-(2,2-dimethylpropanoyl)-3-phenyl-3,5-dihydro-2H-1,4-benzoxazepine-8-carbohydrazide (250 mg, 0.68 mmol) in DCM (6 mL) was added TEA (550 mg, 5.44 mmol), 2,2,2-trifluoroacetyl 2,2,2-trifluoroacetate (572 mg, 2.72 mmol). The reaction mixture was stirred at 25° C. for 4 h. The mixture was quenched by H₂O (15 mL), extracted with DCM (2*20 mL). The organic layer was washed with brine (20 mL), dried over anhydrous Na₂SO₄, concentrated under vacuum. The residue was applied onto silica gel column, this resulted product (130 mg), then purified by Pre-HPLC, (Instrument: Waters MS-triggered Prep-LC with QDA detector, Column: Xbridge 5 u C18 150×19 mm, A water (0.1% FA), B Acetonitrile 55-85% B in 8 min, hold at 100% B for 2 min, back to 55% B with 0.5 min, stop at 13 min, flow rate: 20 mL/min, wavelength: 214/254 nm, injection: 4) to afford the target compound (59.5 mg, 19%) as a white solid. MS (ESI): mass calcd. for C₂₃H₂₂F₃N₃O₃, 445.16, m/z found 446.1[M+H]⁺. ¹H NMR (400 MHz, DMSO-d₆): δ 7.70-7.24 (m, 8H), 5.95-5.80 (m, 1H), 5.25-5.15 (m, 1H), 4.92-4.32 (m, 3H), 1.14 (s, 9H).

### Example 1.22. Synthesis of (S)-1-(8-(5-(difluoromethyl)-1,3,4-oxadiazol-2-yl)-3-phenyl-2,3-dihydrobenzo[f][1,4]oxazepin-4(5H)-yl)-2,2-dimethylpropan-1-one (I-22)

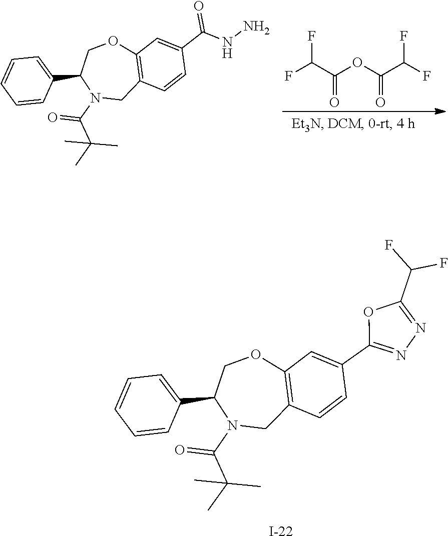

To a solution of (3S)-4-(2,2-dimethylpropanoyl)-3-phenyl-3,5-dihydro-2H-1,4-benzoxazepine-8-carbohydrazide (250 mg, 0.68 mmol) in DCM (6 mL) was added TEA (550 mg, 5.44 mmol), 2,2-difluoroacetyl 2,2-difluoroacetate (474 mg, 2.72 mmol). The reaction mixture was stirred at 25° C. for 4 h. The mixture was quenched by H₂O (15 mL), extracted with DCM (2*20 mL). The organic layer was washed with brine (20 mL), dried over anhydrous Na₂SO₄, concentrated under vacuum. The residue was applied onto silica gel column, this resulted product (130 mg), then purified by Pre-HPLC, (Instrument: Waters MS-triggered Prep-LC with QDA detector, Column: Xbridge 5 u C18 150×19 mm, A water (0.1% FA), B Acetonitrile 55-85% B in 8 min, hold at 100% B for 2 min, back to 55% B with 0.5 min, stop at 13 min, flow rate: 20 mL/min, wavelength: 214/254 nm, injection: 4) to afford the target compound (41.9 mg, 14%) as a white solid. MS (ESI): mass calcd. for C₂₃H₂₃F₂N₃O₃, 427.17, m/z found 428.2[M+H]⁺. ¹H NMR (400 MHz, DMSO-d₆): δ 7.70-7.25 (m, 9H), 5.95-5.80 (m, 1H), 5.25-5.15 (m, 1H), 4.92-4.32 (m, 3H), 1.14 (s, 9H).

### Example 1.23. Synthesis of 2,2-dimethyl-1-[(3S)-3-phenyl-8-[5-(trifluoromethyl)-1,2,4-oxadiazol-3-yl]-3,5-dihydro-2H-1,4-benzoxazepin-4-yl]propan-1-one (I-23)

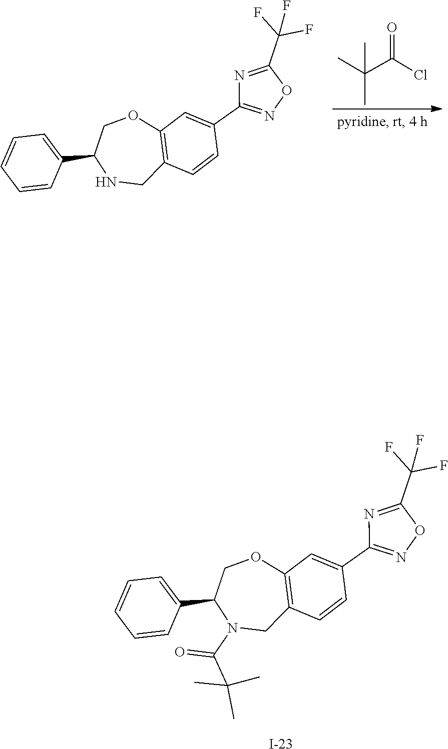

A mixture of (3S)-3-phenyl-8-[5-(trifluoromethyl)-1,2,4-oxadiazol-3-yl]-2,3,4,5-tetrahydro-1,4-benzoxazepine (100 mg, 0.276 mmol) and pivaloyl chloride (50 mg, 0.415 mmol) in dry pyridine (5 mL) was stirred at 25° C. for 4 h. The mixture was diluted with water (20 mL) and extracted with EtOAc (20 mL×3). The organic layer was dried over anhydrous Na₂SO₄, filtered and concentrated under reduced pressure to give the product 2,2-dimethyl-1-[(3S)-3-phenyl-8-[5-(trifluoromethyl)-1,2,4-oxadiazol-3-yl]-3,5-dihydro-2H-1,4-benzoxazepin-4-yl]propan-1-one (78 mg, 62%) as white solid. MS (ESI): mass calcd. for C₂₃H₂₂F₃N₃O₃, 445.16, m/z found 446.1 [M+H]⁺. ¹H NMR (400 MHz, DMSO-d₆) δ 7.65-7.63 (m, 1H), 7.54 (s, 1H), 7.46-7.29 (m, 6H), 5.89 (dd, J=5.6, 10.4 Hz, 1H), 5.17-5.13 (m, 1H), 4.77-4.52 (m, 3H), 1.14 (s, 9H).

### Example 1.24. Synthesis of (S)-2,2,2-trifluoro-1-(6-fluoro-3-methyl-8-(5-(trifluoromethyl)-1,2,4-oxadiazol-3-yl)-2,3-dihydrobenzo[f][1,4]oxazepin-4(5H)-yl)ethan-1-one (I-24)

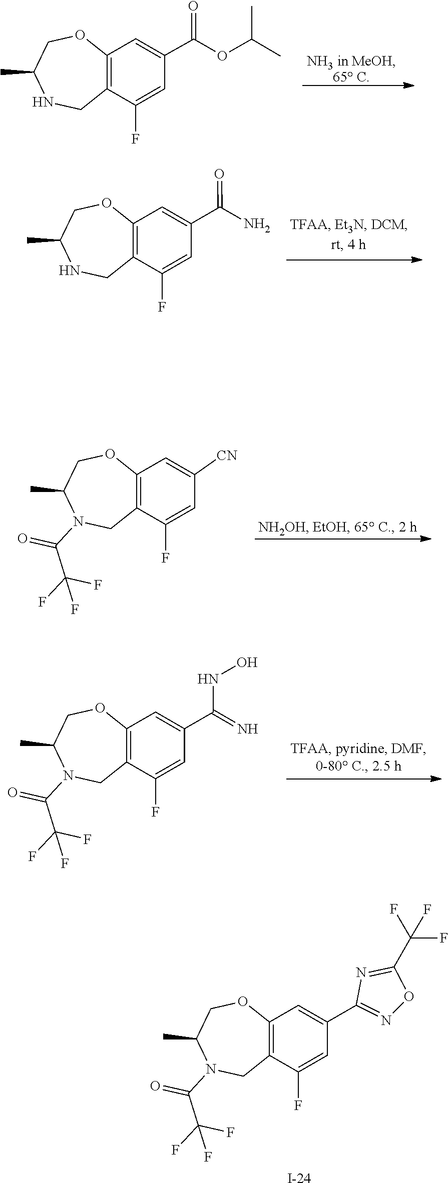

(S)-6-fluoro-3-methyl-2,3,4,5-tetrahydrobenzo[f][1,4]oxazepine-8-carboxamide. isopropyl (3S)-6-fluoro-3-methyl-2,3,4,5-tetrahydro-1,4-benzoxazepine-8-carboxylate (700 mg, 2.62 mmol) was added to NH₃ in MeOH (15 mL) under N₂, the reaction mixture was stirred at 70° C. for 16 h. LCMS showed that about 60% was product. The reaction mixture was directly concentrated to get (S)-6-fluoro-3-methyl-2,3,4,5-tetrahydrobenzo[f][1,4]oxazepine-8-carboxamide (620 mg, crude) as a yellow solid. MS (ESI): mass calcd. for C₁₁H₁₃FN₂O₂ 224.10, m/z found 225.1 [M+H]⁺.

(S)-6-fluoro-3-methyl-4-(2,2,2-trifluoroacetyl)-2,3,4,5-tetrahydrobenzo[f][1,4]oxazepine-8-carbonitrile. To a solution of (3S)-6-fluoro-3-methyl-2,3,4,5-tetrahydro-1,4-benzoxazepine-8-carboxamide (600 mg, 2.68 mmol) and trimethylamine (812 mg, 8.03 mmol) in DCM (15 mL) cooled to 0° C. was added TFAA (1686 mg, 8.03 mmol) under N₂, the reaction mixture was stirred at 25° C. for 2 h. The reaction mixture was quenched with water (20 mL) and extracted with DCM (30 mL×3), the combined organic layer washed with brine (50 mL), dried over Na₂SO₄, concentrated to get the residue. The residue was purified by column chromatography on silica gel (25 g) eluting with EtOAc in petroleum ether (25%), fraction with MS signal of desired product was collected and concentrated to get (S)-6-fluoro-3-methyl-4-(2,2,2-trifluoroacetyl)-2,3,4,5-tetrahydrobenzo[f][1,4]oxazepine-8-carbonitrile (550 mg, 68%) as a yellow oil. MS (ESI): mass calcd. for C₁₃H₁₀F₄N₂O₂ 302.07, m/z found 303.1 [M+H]⁺.

(S)-6-fluoro-N-hydroxy-3-methyl-4-(2,2,2-trifluoroacetyl)-2,3,4,5-tetrahydrobenzo[f][1,4]oxazepine-8-carboximidamide. To a solution of (3S)-6-fluoro-3-methyl-4-(2,2,2-trifluoroacetyl)-3,5-dihydro-2H-1,4-benzoxazepine-8-carbonitrile (500 mg, 1.65 mmol) in EtOH (20 mL) was added NH₂OH in water (20 mL, 50% ww, 165.4 mmol) under N₂, the reaction mixture was stirred at 65° C. for 1 h. The reaction mixture was directly concentrated to get (S)-6-fluoro-N-hydroxy-3-methyl-4-(2,2,2-trifluoroacetyl)-2,3,4,5-tetrahydrobenzo[f][1,4]oxazepine-8-carboximidamide (500 mg, 82%) as a white solid. MS (ESI): mass calcd. for C₁₃H₁₃F₄N₃O₃ 335.09, m/z found 336.1[M+H]⁺.

(S)-2,2,2-trifluoro-1-(6-fluoro-3-methyl-8-(5-(trifluoromethyl)-1,2,4-oxadiazol-3-yl)-2,3-dihydrobenzo[f][1,4]oxazepin-4(5H)-yl)ethan-1-one. To a solution of (S)-6-fluoro-N-hydroxy-3-methyl-4-(2,2,2-trifluoroacetyl)-3, 5-dihydro-2H-1,4-benzoxazepine-8-carboximidamide (400 mg, 1.19 mmol) and pyridine (944 mg, 11.93 mmol) in DMF (15 mL) cooled to 0° C. was added TFAA (1002 mg, 4.77 mmol) under N₂, the reaction mixture was stirred at 85° C. for 3 h. The reaction mixture was quenched with water (20 mL) and extracted with EtOAc (30 mL×3), the combined organic layer washed with brine (30 mL), dried over Na₂SO₄, concentrated to get the residue. The residue was purified by column chromatography on silica gel (25 g) eluting with EtOAc in petroleum ether (15%), fraction with MS signal of desired product was collected and concentrated to get (S)-2,2,2-trifluoro-1-(6-fluoro-3-methyl-8-(5-(trifluoromethyl)-1,2,4-oxadiazol-3-yl)-2,3-dihydrobenzo[f][1,4]oxazepin-4(5H)-yl)ethan-1-one (129 mg, 26%) as a white solid. MS (ESI): mass calcd. for C₁₅H₁₀F₇N₃O₃ 413.06, m/z found 414.0 [M+H]⁺. ¹H NMR (400 MHz, CD₃OD) δ 7.58-7.35 (m, 2H), 5.30-5.05 (m, 1H), 5.00-4.93 (m, 1H), 4.65-4.57 (m, 1H), 4.53-4.34 (m, 2H), 1.38-1.23 (m, 3H).

### Example 1.25. Synthesis of 1-[(3S)-6-fluoro-3-methyl-8-[5-(trifluoromethyl)-1,2,4-oxadiazol-3-yl]-3,5-dihydro-2H-1,4-benzoxazepin-4-yl]ethanone (I-25)

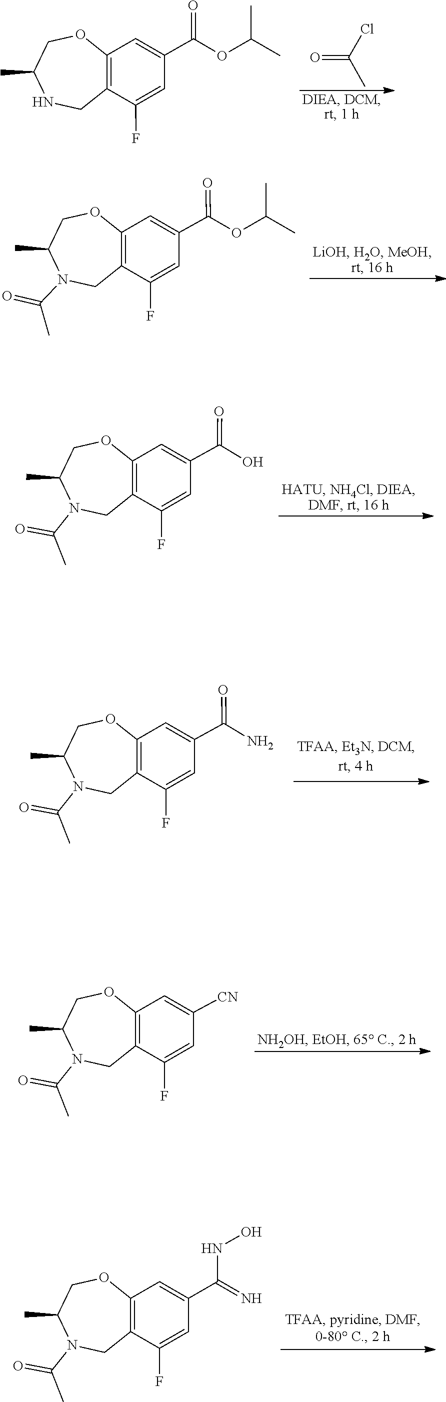

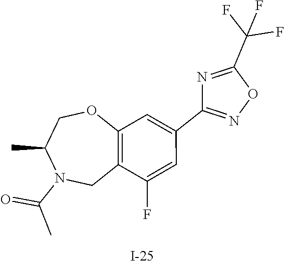

Isopropyl (3S)-4-acetyl-6-fluoro-3-methyl-3,5-dihydro-2H-1,4-benzoxazepine-8-carboxylate. To a solution of isopropyl (3S)-3-cyclopropyl-6-fluoro-2,3,4,5-tetrahydro-1,4-benzoxazepine-8-carboxylate (800 mg, 3.0 mmol) and NEt₃ (909 mg, 8.98 mmol) in DCM (10 mL) stirred under nitrogen atmosphere at 0° C. was added acetyl chloride (353 mg, 4.49 mmol). The reaction mixture was stirred at room temperature for 3 h under nitrogen atmosphere. The mixture was quenched with water (15 mL) and extracted with DCM (3×10 mL). The combined organic layer was dried over Na₂SO₄ and concentrated under reduced pressure to get a residue which was purified by column chromatography on silica gel (25 g) eluting with EA in PE (0-30%) to afford isopropyl (3S)-4-acetyl-6-fluoro-3-methyl-3,5-dihydro-2H-1,4-benzoxazepine-8-carboxylate (950 mg, 92%) as yellow oil. MS (ESI): mass calcd. for C₁₆H₂₀FNO₄, 309.14, m/z found 310.2 [M+H]⁺.

(3S)-4-acetyl-6-fluoro-3-methyl-3,5-dihydro-2H-1,4-benzoxazepine-8-carboxylic acid. To a solution of isopropyl (3S)-4-acetyl-6-fluoro-3-methyl-3,5-dihydro-2H-1,4-benzoxazepine-8-carboxylate (950 mg, 3.07 mmol) in THE (10 mL) and H₂O (10 mL) was added Lithium hydroxide (221 mg, 6.31 mmol). The reaction mixture was stirred at room temperature for 2 h. The mixture was adjusted to pH=2-3 with 1M HCl and extracted with DCM (3×10 mL). The combined organic layer was dried over Na₂SO₄ and concentrated under reduced pressure to get the required product (3S)-4-acetyl-6-fluoro-3-methyl-3,5-dihydro-2H-1,4-benzoxazepine-8-carboxylic acid (450 mg, 49%) as yellow oil. MS (ESI): mass calcd. for C₁₃H₁₄FNO₄, 267.09, m/z found 268.0 [M+H]⁺.

(3S)-4-acetyl-6-fluoro-3-methyl-3,5-dihydro-2H-1,4-benzoxazepine-8-carboxamide. To a solution of (3S)-4-acetyl-6-fluoro-3-methyl-3,5-dihydro-2H-1,4-benzoxazepine-8-carboxylic acid (450 mg, 1.68 mmol) and Ammonium chloride (270 mg, 5.05 mmol) in DMF (15 mL) was added DIEA (653 mg, 5.05 mmol), HATU (1280 mg, 3.37 mmol) at room temperature. The reaction mixture stirred at room temperature for 12 h under nitrogen atmosphere. The mixture was diluted with water (20 mL) and extracted with EA (10 mL×3). The combined organic layer was washed with brine (20 mL×3) and concentrated to get the required product (3S)-4-acetyl-6-fluoro-3-methyl-3,5-dihydro-2H-1,4-benzoxazepine-8-carboxamide (400 mg, 54%) as yellow oil. MS (ESI): mass calcd. for C₁₃H₁₅FN₂O₃, 266.11, m/z found 267.1 [M+H]⁺.

(3S)-4-acetyl-6-fluoro-3-methyl-3,5-dihydro-2H-1,4-benzoxazepine-8-carbonitrile. To a solution of (3S)-4-acetyl-6-fluoro-3-methyl-3,5-dihydro-2H-1,4-benzoxazepine-8-carboxamide (400 mg, 1.50 mmol) and NEt₃ (947 mg, 4.50 mmol) in DCM (10 mL) stirred under nitrogen atmosphere at 0° C. was added TFAA (456 mg, 4.50 mmol). The reaction mixture was stirred at room temperature for 2 h under nitrogen atmosphere. The mixture was quenched with water (10 mL) and extracted with DCM (3×10 mL). The combined organic layer was dried over Na₂SO₄ and concentrated under reduced pressure to get a residue which was purified by column chromatography on silica gel (25 g) eluting with EA in PE (0-20%) to afford (3S)-4-acetyl-6-fluoro-3-methyl-3,5-dihydro-2H-1,4-benzoxazepine-8-carbonitrile (348 mg, 84%) as yellow oil. MS (ESI): mass calcd. for C₁₃H₁₃FN₂O₂, 248.10, m/z found 249.1 [M+H]⁺.

(3S)-4-acetyl-6-fluoro-N-hydroxy-3-methyl-3,5-dihydro-2H-1,4-benzoxazepine-8-carboximidamide. To a solution of (3S)-4-acetyl-6-fluoro-3-methyl-3,5-dihydro-2H-1,4-benzoxazepine-8-carbonitrile (348 mg, 1.40 mmol) in EtOH (15 mL) was added NH₂OH/H₂O (4.6 g, 140 mmol, 50%). The reaction mixture was stirred at 65° C. for 2 h under nitrogen atmosphere. The mixture was cooled to room temperature and concentrated under reduced pressure to get the required product (3S)-4-acetyl-6-fluoro-N-hydroxy-3-methyl-3,5-dihydro-2H-1,4-benzoxazepine-8-carboximidamide (300 mg, 68%) as a white solid. MS (ESI): mass calcd. for C₁₃H₁₆FN₃O₃, 281.12, m/z found 282.1[M+H]⁺.

1-[(3S)-6-fluoro-3-methyl-8-[5-(trifluoromethyl)-1,2,4-oxadiazol-3-yl]-3,5-dihydro-2H-1,4-benzoxazepin-4-yl]ethanone. To a solution of (3S)-4-acetyl-6-fluoro-N-hydroxy-3-methyl-3,5-dihydro-2H-1,4-benzoxazepine-8-carboximidamide (300 mg, 1.07 mmol) and NEt₃ (324 mg, 3.20 mmol) in DMF (20 mL) was added TFAA (448 mg, 2.13 mmol) at 0° C. under nitrogen atmosphere. The reaction mixture was stirred at 80° C. for 2 h under nitrogen atmosphere. The mixture was cooled to room temperature and purified by Pre-HPLC (Instrument: Waters MS-triggered Prep-LC with QDA detector, Xbridge 5 u C18 150×19 mm, A water (0.1% TFA), B Acetonitrile 50-60% B in 8 min, hold at 100% B for 2 min, back to 50% B within 0.5 min, stop at 13 min, flow rate: 20 mL/min, wavelength: 214 nm, injection: to afford 1-[(3S)-6-fluoro-3-methyl-8-[5-(trifluoromethyl)-1,2,4-oxadiazol-3-yl]-3,5-dihydro-2H-1,4-benzoxazepin-4-yl]ethanone (27.4 mg, 7%) as a white solid. MS (ESI): mass calcd. for C₁₅H₁₃F₄N₃O₃, 359.09, m/z found 360.1 [M+H]⁺. ¹H NMR (400 MHz, MeOD-d₄) δ 7.54-7.38 (m, 2H), 5.57-4.21 (m, 5H), 2.21-2.04 (m, 3H), 1.34-1.15 (m, 3H).

### Example 1.26. Synthesis of (3S)-3-(methoxymethyl)-4-[(4-methyloxan-4-yl)carbonyl]-8-[5-(trifluoromethyl)-1,2,4-oxadiazol-3-yl]-3,5-dihydro-2H-1,4-benzoxazepine (I-26)

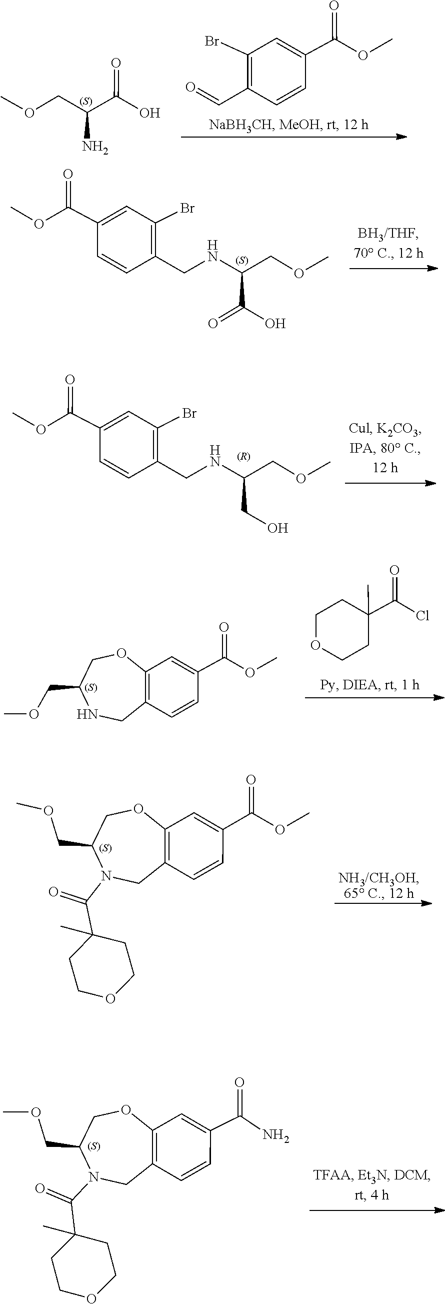

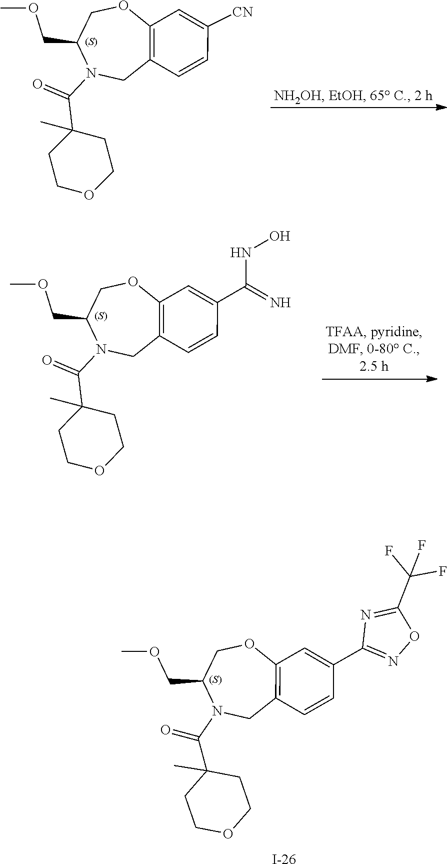

(2S)-2-({[2-bromo-4-(methoxycarbonyl)phenyl]methyl}amino)-3-methoxypropanoic acid. To a mixture of (2S)-2-amino-3-methoxypropanoic acid (4.0 g, 33.58 mmol) and methyl 3-bromo-4-formylbenzoate (8.2 g, 33.58 mmol) in MeOH (50 mL) was added NaBH₃CN (6.3 g, 100.73 mmol). The reaction mixture was stirred at 25° C. for 16 hours. The mixture was diluted with water (50 mL) and extracted with EA (100 mL×3). The mixture was concentrated under reduced pressure to give a residue which was purified by reversed phase column (40% A in B; A: CH₃CN, B: 0.1% FA in water) to afford (2S)-2-({[2-bromo-4-(methoxycarbonyl)phenyl]methyl}amino)-3-methoxypropanoic acid (4.5 g, 35%) as yellow oil. MS (ESI): mass calcd. for C₁₃H₁₆BrNO₅, 345.0, m/z found 346.0 [M+H]⁺.

Methyl 3-bromo-4-({[(2R)-1-hydroxy-3-methoxypropan-2-yl]amino}methyl)benzoate. To a solution of (2S)-2-({[2-bromo-4-(methoxycarbonyl)phenyl]methyl}amino)-3-methoxypropanoic acid (4.5 g, 13.00 mmol) in THE (20 mL) was added BH₃ (65 mL). The reaction mixture was stirred at 25° C. for 16 hours. The mixture was quenched with MeOH (20 mL). The mixture was diluted with water (50 mL) and extracted with EA (100 mL×3). The organic layer was concentrated and the residue was purified by flash chromatograph on silica gel column (PE:EA=1:1) to afford methyl 3-bromo-4-({[(2R)-1-hydroxy-3-methoxypropan-2-yl]amino}methyl)benzoate (3.0 g, 62%) as white oil. MS (ESI): mass calcd. for C₁₃H₁₈BrNO₄, 331.0, m/z found 332.0 [M+H]⁺.

Methyl (3S)-3-(methoxymethyl)-2,3,4,5-tetrahydro-1,4-benzoxazepine-8-carboxylate. To a solution of methyl 3-bromo-4-({[(2R)-1-hydroxy-3-methoxypropan-2-yl]amino}methyl)benzoate (3.0 g, 9.03 mmol) in IPA (20 mL) were added Copper(I) iodide (344 mg, 1.81 mmol) and K₂CO₃ (3.7 g, 27.09 mmol). The reaction mixture was stirred at 80° C. for 16 hours. After cooling to room temperature, the reaction mixture was concentrated under reduced pressure. The organic layer was concentrated and the residue was purified by flash chromatograph on silica gel column (PE:EA=3:1) to afford methyl (3S)-3-(methoxymethyl)-2,3,4,5-tetrahydro-1,4-benzoxazepine-8-carboxylate (2.0 g, 71%) as yellow oil. MS (ESI): mass calcd. for C₁₃H₁₇NO₄, 251.1, m/z found 252.1 [M+H]⁺.

Methyl (3S)-3-(methoxymethyl)-4-[(4-methyloxan-4-yl)carbonyl]-3,5-dihydro-2H-1,4-benzoxazepine-8-carboxylate. To a solution of methyl (3S)-3-(methoxymethyl)-2,3,4,5-tetrahydro-1,4-benzoxazepine-8-carboxylate (500 mg, 1.99 mmol) in DCM (10 mL) was added 4-methyloxane-4-carbonyl chloride (647 mg, 3.98 mmol) and TEA (604 mg, 5.97 mmol). The reaction mixture was stirred at 25° C. for 16 hours. The reaction mixture was concentrated under reduced pressure. The organic layer was concentrated and the residue was purified by flash chromatograph on silica gel column (PE:EA=1:1) to afford methyl (3S)-3-(methoxymethyl)-4-[(4-methyloxan-4-yl)carbonyl]-3,5-dihydro-2H-1,4-benzoxazepine-8-carboxylate (500 mg, 60%) as yellow oil. MS (ESI): mass calcd. for C₂₀H₂₇NO₆, 377.1, m/z found 378.1 [M+H]⁺.

(3S)-3-(methoxymethyl)-4-[(4-methyloxan-4-yl)carbonyl]-3,5-dihydro-2H-1,4-benzoxazepine-8-carboxamide. To a solution of methyl (3S)-3-(methoxymethyl)-4-[(4-methyloxan-4-yl)carbonyl]-3,5-dihydro-2H-1,4-benzoxazepine-8-carboxylate (500 mg, 1.32 mmol) was added NH₃ in MeOH (20 mL). The reaction mixture was stirred at 75° C. for 16 hours. After cooling to room temperature, the reaction mixture was concentrated under reduced pressure to afford (3S)-3-(methoxymethyl)-4-[(4-methyloxan-4-yl)carbonyl]-3,5-dihydro-2H-1,4-benzoxazepine-8-carboxamide (300 mg, 56%) as yellow oil. MS (ESI): mass calcd. for C₁₉H₂₆N₂O₅, 362.1, m/z found 363.1 [M+H]⁺.

(3S)-3-(methoxymethyl)-4-[(4-methyloxan-4-yl)carbonyl]-3,5-dihydro-2H-1,4-benzoxazepine-8-carbonitrile. To a solution of (3S)-3-(methoxymethyl)-4-[(4-methyloxan-4-yl)carbonyl]-3,5-dihydro-2H-1,4-benzoxazepine-8-carboxamide (300 mg, 0.83 mmol) in DCM (10 mL) was added TEA (1.3 g, 12.42 mmol) and TFAA (522 mg, 2.48 mmol). The reaction mixture was stirred at 25° C. for 3 hours. The reaction mixture was concentrated under reduced pressure. The organic layer was concentrated and the residue was purified by flash chromatograph on silica gel column (PE:EA=3:1) to afford (3S)-3-(methoxymethyl)-4-[(4-methyloxan-4-yl)carbonyl]-3,5-dihydro-2H-1,4-benzoxazepine-8-carbonitrile (200 mg, 63%) as yellow oil. MS (ESI): mass calcd. for C₁₉H₂₄N₂O₄, 344.1, m/z found 345.1 [M+H]⁺.

(3S)—N-hydroxy-3-(methoxymethyl)-4-[(4-methyloxan-4-yl)carbonyl]-3,5-dihydro-2H-1,4-benzoxazepine-8-carboximidamide. To a solution of (3S)-3-(methoxymethyl)-4-[(4-methyloxan-4-yl)carbonyl]-3,5-dihydro-2H-1,4-benzoxazepine-8-carbonitrile (200 mg, 0.58 mmol) in EtOH (5 mL) was added NH₂OH/H₂O (58 mg, 1.74 mmol). The reaction mixture was stirred at 65° C. for 3 hours. The reaction mixture was concentrated under reduced pressure to afford (3S)—N-hydroxy-3-(methoxymethyl)-4-[(4-methyloxan-4-yl)carbonyl]-3,5-dihydro-2H-1,4-benzoxazepine-8-carboximidamide (150 mg, 62%) as a yellow solid. MS (ESI): mass calcd. for C₁₉H₂₇N₃O₅, 377.2, m/z found 378.1 [M+H]⁺.

(3S)-3-(methoxymethyl)-4-[(4-methyloxan-4-yl)carbonyl]-8-[5-(trifluoromethyl)-1,2,4-oxadiazol-3-yl]-3,5-dihydro-2H-1,4-benzoxazepine. To a mixture of (3S)—N-hydroxy-3-(methoxymethyl)-4-[(4-methyloxan-4-yl)carbonyl]-3,5-dihydro-2H-1,4-benzoxazepine-8-carboximidamide (150 mg, 0.40 mmol) and pyridine (94 mg, 1.19 mmol) in DMF (5 mL) was added TFAA (417 mg, 1.99 mmol). The reaction mixture was stirred at 80° C. for 3 hours. The mixture was concentrated under reduced pressure to give a residue which was purified by reversed phase column (20% A in B; A: CH₃CN, B: 0.1% FA in water) to afford (3S)-3-(methoxymethyl)-4-[(4-methyloxan-4-yl)carbonyl]-8-[5-(trifluoromethyl)-1,2,4-oxadiazol-3-yl]-3,5-dihydro-2H-1,4-benzoxazepine (54.7 mg, 29%) as yellow oil. MS (ESI): mass calcd. for C₂₁H₂₄F₃N₃O₅, 455.1, m/z found 456.1 [M+H]⁺. ¹H NMR (400 MHz, CDCl₃) δ 7.83-7.56 (m, 2H), 7.29 (d, J=15.2 Hz, 1H), 5.22-4.60 (m, 3H), 4.53-4.25 (m, 2H), 3.85-3.19 (m, 9H), 2.24-2.04 (m, 2H), 1.63-1.42 (m, 2H), 1.30 (s, 3H).

### Example 1.27. Synthesis of 1-[(3S)-3-(methoxymethyl)-8-[5-(trifluoromethyl)-1,2,4-oxadiazol-3-yl]-3,5-dihydro-2H-1,4-benzoxazepin-4-yl]-2,2-dimethylpropan-1-one (I-27)

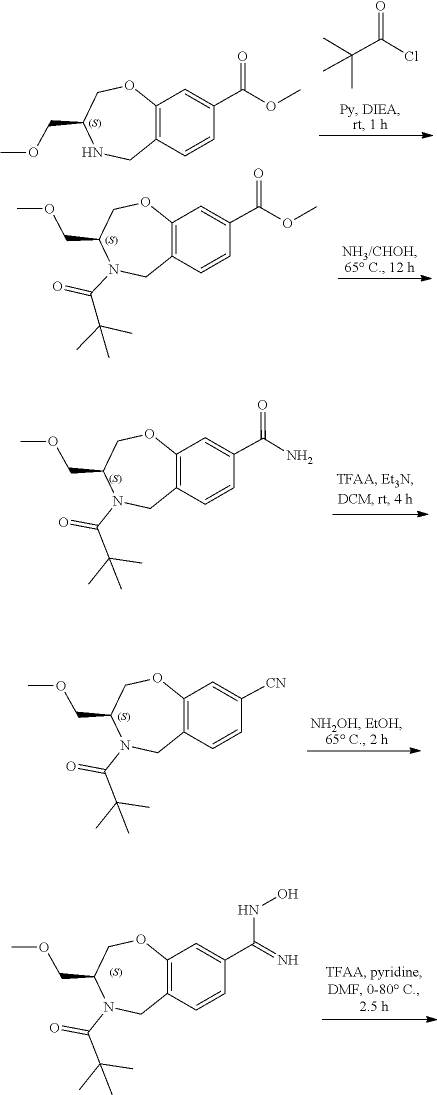

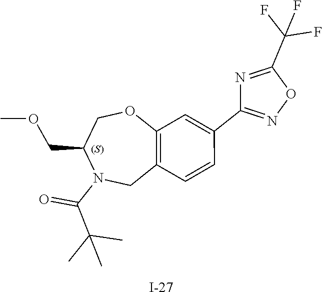

Methyl (3S)-4-(2,2-dimethylpropanoyl)-3-(methoxymethyl)-3,5-dihydro-2H-1,4-benzoxazepine-8-carboxylate. To a solution of methyl (3S)-3-(methoxymethyl)-2,3,4,5-tetrahydro-1,4-benzoxazepine-8-carboxylate (500 mg, 1.99 mmol) in DCM (20 mL) was added 2,2-dimethylpropanoyl chloride (480 mg, 3.98 mmol) and TEA (604 mg, 5.97 mmol). The reaction mixture was stirred at 25° C. for 3 hours. The reaction mixture was concentrated under reduced pressure. The mixture was diluted with water (20 mL) and extracted with DCM (10 mL×3). The organic layer was concentrated, the residue was purified by flash chromatograph on silica gel column (PE:EA=1:1) to afford methyl (3S)-4-(2,2-dimethylpropanoyl)-3-(methoxymethyl)-3,5-dihydro-2H-1,4-benzoxazepine-8-carboxylate (500 mg, 67%) as yellow oil. MS (ESI): mass calcd. for C₁₈H₂₅NO₅, 335.1, m/z found 336.2 [M+H]⁺.

(3S)-4-(2,2-dimethylpropanoyl)-3-(methoxymethyl)-3,5-dihydro-2H-1,4-benzoxazepine-8-carboxamide. To a solution of methyl (3S)-4-(2,2-dimethylpropanoyl)-3-(methoxymethyl)-3,5-dihydro-2H-1,4-benzoxazepine-8-carboxylate (500 mg, 1.49 mmol) was added NH₃ in MeOH (20 mL). The reaction mixture was stirred at 75° C. for 16 hours. After cooling to room temperature, the reaction mixture was concentrated under reduced pressure to afford (3S)-4-(2,2-dimethylpropanoyl)-3-(methoxymethyl)-3,5-dihydro-2H-1,4-benzoxazepine-8-carboxamide (300 mg, 57%) as yellow oil. MS (ESI): mass calcd. for C₁₇H₂₄N₂O₄, 320.0, m/z found 321.1 [M+H]⁺.

(3S)-4-(2,2-dimethylpropanoyl)-3-(methoxymethyl)-3,5-dihydro-2H-1,4-benzoxazepine-8-carbonitrile. To a solution of (3S)-4-(2,2-dimethylpropanoyl)-3-(methoxymethyl)-3,5-dihydro-2H-1,4-benzoxazepine-8-carboxamide (300 mg, 0.94 mmol) in DCM (10 mL) was added TEA (1.4 g, 14.05 mmol) and TFAA (590 mg, 2.81 mmol). The reaction mixture was stirred at 25° C. for 3 hours. The reaction mixture was concentrated under reduced pressure. The organic layer was concentrated, the residue was purified by flash chromatograph on silica gel column (PE:EA=3:1) to afford (3S)-4-(2,2-dimethylpropanoyl)-3-(methoxymethyl)-3,5-dihydro-2H-1,4-benzoxazepine-8-carbonitrile (200 mg, 64%) as yellow oil. MS (ESI): mass calcd. for C₁₇H₂₂N₂O₃, 302.1, m/z found 303.1 [M+H]>.

(3S)-4-(2,2-dimethylpropanoyl)-N-hydroxy-3-(methoxymethyl)-3,5-dihydro-2H-1,4-benzoxazepine-8-carboximidamide. To a solution of (3S)-4-(2,2-dimethylpropanoyl)-3-(methoxymethyl)-3,5-dihydro-2H-1,4-benzoxazepine-8-carbonitrile (200 mg, 0.66 mmol) in EtOH (5 mL) was added NH₂OH/H₂O (44 mg, 1.32 mmol). The reaction mixture was stirred at 65° C. for 3 hours. The reaction mixture was concentrated under reduced pressure to afford (3S)-4-(2,2-dimethylpropanoyl)-N-hydroxy-3-(methoxymethyl)-3,5-dihydro-2H-1,4-benzoxazepine-8-carboximidamide (150 mg, 61%) as a yellow solid. MS (ESI): mass calcd. for C₁₇H₂₅N₃O₄, 335.1, m/z found 336.1 [M+H]⁺.

1-[(3S)-3-(methoxymethyl)-8-[5-(trifluoromethyl)-1,2,4-oxadiazol-3-yl]-3,5-dihydro-2H-1,4-benzoxazepin-4-yl]-2,2-dimethylpropan-1-one. To a mixture of (3 S)-4-(2,2-dimethylpropanoyl)-N-hydroxy-3-(methoxymethyl)-3,5-dihydro-2H-1,4-benzoxazepine-8-carboximidamide (150 mg, 0.45 mmol) and pyridine (106 mg, 1.34 mmol) in DMF (5 mL) was added TFAA (470 mg, 2.24 mmol). The reaction mixture was stirred at 80° C. for 3 hours. The mixture was concentrated under reduced pressure to give a residue which was purified by reversed phase column (20% A in B; A: CH₃CN, B: 0.1% FA in water) to afford 1-[(3S)-3-(methoxymethyl)-8-[5-(trifluoromethyl)-1,2,4-oxadiazol-3-yl]-3,5-dihydro-2H-1,4-benzoxazepin-4-yl]-2,2-dimethylpropan-1-one (46.9 mg, 25%) as yellow oil. MS (ESI): mass calcd. for C₁₉H₂₂F₃N₃O₄, 413.1, m/z found 414.1 [M+H]⁺. ¹H NMR (400 MHz, CDCl₃) δ 7.74-7.64 (m, 2H), 7.32 (s, 1H), 5.00 (d, J=16.0 Hz, 1H), 4.81 (s, 1H), 4.56-4.47 (m, 1H), 4.40-4.20 (m, 1H), 3.86-3.72 (m, 1H), 3.70-3.62 (m, 1H), 3.39 (s, 3H), 1.88 (s, 1H), 1.26 (s, 9H).

### Example 1.28. Synthesis of 6-fluoro-4-[(4-methyloxan-4-yl) carbonyl]-8-[5-(trifluoromethyl)-1,2,4-oxadiazol-3-yl]-3,5-dihydro-2H-1,4-benzoxazepine (I-28)

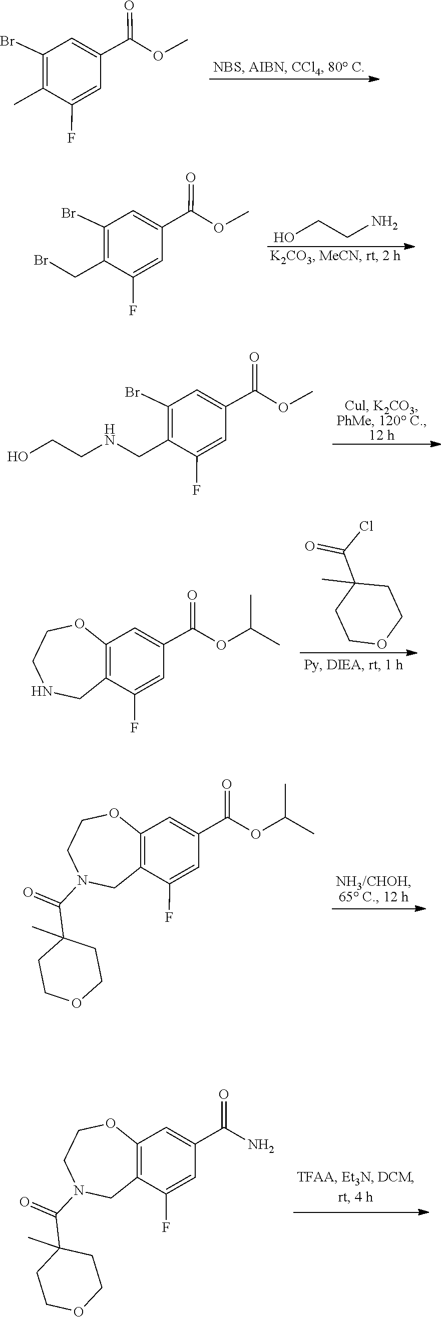

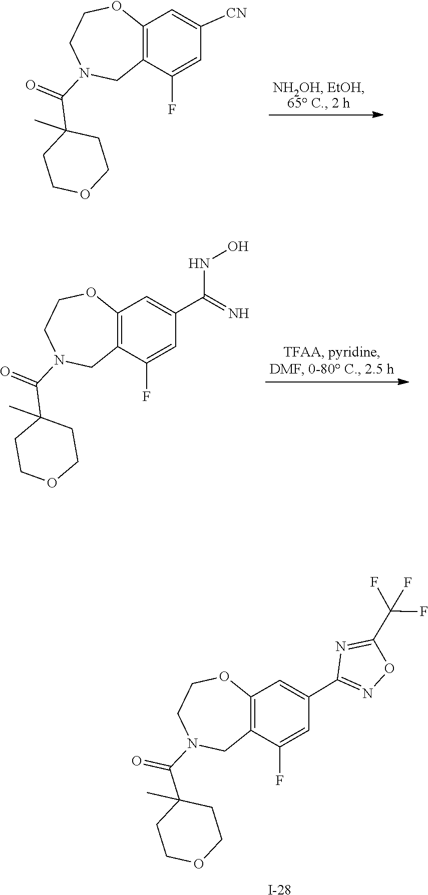

Methyl 3-bromo-4-(bromomethyl)-5-fluorobenzoate. AIBN (1.05 g, 6.4 mmol) was added to the mixture of methyl 3-bromo-5-fluoro-4-methylbenzoate (15.8 g, 64 mmol) and N-bromosuccinimide (12.53 g, 70.4 mmol) in CCl₄ (20 mL). The mixture was stirred at 80° C. for 8 hours. The mixture was diluted with water (50 mL) and extracted with DCM (20 mL×3). The organic layer was dried over sodium sulphate, filtered and concentrated in vacuo. The residue was purified by chromatography column on silica gel (80 g) eluting with PE (100%) to give product methyl 3-bromo-4-(bromomethyl)-5-fluorobenzoate (18 g, 82%) as colorless oil. ¹H NMR (400 MHz, DMSO-d₆) δ 8.00 (s, 1H), 7.79 (d, J=10.0 Hz, 1H), 4.72 (d, J=2.0 Hz, 2H), 3.89 (s, 3H).

Methyl 3-bromo-5-fluoro-4-{[(2-hydroxyethyl)amino]methyl}benzoate. K₂CO₃ (3.40 g, 24.6 mmol) was added to the mixture of methyl 3-bromo-4-(bromomethyl)-5-fluorobenzoate (4 g, 12.3 mmol) and 2-aminoethanol (2.63 g, 43.0 mmol) in MeCN (20 mL). The mixture was stirred at room temperature for 2 hours. The residue was diluted with water (50 mL) and extracted with DCM (20 mL×2). The organic phase was washed with brine (30 mL×3), dried over sodium sulphate, filtered and concentrated in vacuo. The residue was purified by chromatography column on silica gel (80 g) eluting with DCM/MeOH (3%) to give product methyl 3-bromo-5-fluoro-4-{[(2-hydroxyethyl)amino]methyl}benzoate (2.9 g, 73%) as a colorless oil. MS (ESI): mass calcd. for C₁₁H₁₃BrFNO₃, 305.01, m/z found 306.0 [M+H]⁺.

Isopropyl 6-fluoro-2,3,4,5-tetrahydro-1,4-benzoxazepine-8-carboxylate. Potassium carbonate (2.71 g, 19.6 mmol) was added to the mixture of methyl 3-bromo-5-fluoro-4-{[(2-hydroxyethyl) amino]methyl}benzoate (3 g, 9.8 mmol) and Copper(I) iodide (370 mg, 1.9 mmol) in iPrOH (20 mL). The mixture was stirred at 100° C. for 16 hours. The residue was diluted with water (50 mL) and extracted with DCM (20 mL×2). The organic layer was washed with brine (30 mL×3), dried over sodium sulphate, filtered and concentrated in vacuo. The residue was purified by chromatography column on silica gel (80 g) eluting with PE/EA (50%) to give product isopropyl 6-fluoro-2,3,4,5-tetrahydro-1,4-benzoxazepine-8-carboxylate (2.04 g, 78%) as a red oil. MS (ESI): mass calcd. for C₁₃H₁₆FNO₃, 253.11, m/z found 254.1[M+H]⁺.

Isopropyl 6-fluoro-4-[(4-methyloxan-4-yl)carbonyl]-3,5-dihydro-2H-1,4-benzoxazepine-8-carboxylate. 4-methyloxane-4-carbonyl chloride (1.7 g, 10.5 mmol) was added to the mixture of isopropyl 6-fluoro-2,3,4,5-tetrahydro-1,4-benzoxazepine-8-carboxylate (890 mg, 3.5 mmol), triethylamine (1.8 g, 17.6 mmol) and 4-DMAP (43 mg, 0.35 mmol) in DCM (10 mL) at 0° C. The mixture was stirred at room temperature for 2 hours. The crude was diluted with water (50 mL) and extracted with DCM (10 mL×2). The organic layer was washed with brine (10 mL×3), dried over sodium sulphate, filtered and concentrated in vacuo. The residue was purified by chromatography column on silica gel (40 g) eluting with PE/EA (10%) to give product isopropyl 6-fluoro-4-[(4-methyloxan-4-yl)carbonyl]-3,5-dihydro-2H-1,4-benzoxazepine-8-carboxylate (1.3 g, 88%) as white solid. MS (ESI): mass calcd. for C₂₀H₂₆FNO₅, 379.18, m/z found 380.2 [M+H]⁺.

6-fluoro-4-[(4-methyloxan-4-yl) carbonyl]-3,5-dihydro-2H-1,4-benzoxazepine-8-carboxamide. The mixture of NH₃ in MeOH (7 M, 20.7 mL) and isopropyl 6-fluoro-4-[(4-methyloxan-4-yl) carbonyl]-3,5-dihydro-2H-1,4-benzoxazepine-8-carboxylate (1.1 g, 2.9 mmol) was stirred at 70° C. for 20 hours. The mixture was concentrated in vacuo to afford the crude 6-fluoro-4-[(4-methyloxan-4-yl) carbonyl]-3,5-dihydro-2H-1,4-benzoxazepine-8-carboxamide (800 mg, 66%) as a white solid. MS (ESI): mass calcd. for C₁₇H₂₁FN₂O₄, 336.15, m/z found 337.1 [M+H]⁺.

6-fluoro-4-[(4-methyloxan-4-yl) carbonyl]-3,5-dihydro-2H-1,4-benzoxazepine-8-carbonitrile. TFAA (1.75 g, 8.32 mmol) was added to the solution of 6-fluoro-4-[(4-methyloxan-4-yl) carbonyl]-3,5-dihydro-2H-1,4-benzoxazepine-8-carboxamide (300 mg, 0.8919 mmol) and triethylamine (3.16 g, 31.22 mmol) in DCM (20 mL) at 0° C. The mixture was stirred at 25° C. for 2 hours. The mixture was diluted with water (50 mL) and extracted with DCM (10 mL×2). The organic layer was dried over sodium sulphate, filtered and concentrated in vacuo. The residue was purified by chromatography column on silica gel (40 g) eluting with PE/EA (10%) to give product 6-fluoro-4-[(4-methyloxan-4-yl) carbonyl]-3,5-dihydro-2H-1,4-benzoxazepine-8-carbonitrile (550 mg, 75%) as a white solid. MS (ESI): mass calcd. for C₁₇H₁₉FN₂O₃, 318.14, m/z found 319.1 [M+H]⁺.

6-fluoro-N-hydroxy-4-[(4-methyloxan-4-yl) carbonyl]-3,5-dihydro-2H-1,4-benzoxazepine-8-carboximidamide. NH₂OH/H₂O (50%, 9.34 g, 141.4 mmol) was added to the solution of 6-fluoro-4-[(4-methyloxan-4-yl) carbonyl]-3,5-dihydro-2H-1,4-benzoxazepine-8-carbonitrile (450 mg, 1.4 mmol) in EtOH (5 mL). The mixture was stirred at 65° C. for 2 hours. The residue was concentrated in vacuo to afford 6-fluoro-N-hydroxy-4-[(4-methyloxan-4-yl) carbonyl]-3,5-dihydro-2H-1,4-benzoxazepine-8-carboximidamide (500 mg, 99%) as white solid. MS (ESI): mass calcd. for C₁₇H₂₂FN₃O₄, 351.16, m/z found 352.2 [M+H]⁺.

6-fluoro-4-[(4-methyloxan-4-yl) carbonyl]-8-[5-(trifluoromethyl)-1,2,4-oxadiazol-3-yl]-3,5-dihydro-2H-1,4-benzoxazepine. Trifluoroacetic anhydride (628 mg, 3.0 mmol) was added to the mixture of 6-fluoro-N-hydroxy-4-[(4-methyloxan-4-yl) carbonyl]-3,5-dihydro-2H-1,4-benzoxazepine-8-carboximidamide (350 mg, 1.0 mmol) and pyridine (788 mg, 10.0 mmol) in DMF (5 mL) at 0° C. The mixture was stirred at 80° C. for 3 hours. The mixture was concentrated and the residue was purified by chromatography column on silica gel (40 g) eluting with PE/EA to give 6-fluoro-4-[(4-methyloxan-4-yl) carbonyl]-8-[5-(trifluoromethyl)-1,2,4-oxadiazol-3-yl]-3,5-dihydro-2H-1,4-benzoxazepine (84 mg, 19%) as a white solid. MS (ESI): mass calcd. for C₁₉H₁₉F₄N₃O₄, 429.13, m/z found 430.1 [M+H]⁺. ¹H NMR (400 MHz, CDCl₃-d) δ 7.58-7.53 (m, 2H), 4.81 (s, 2H), 4.34-4.27 (m, 2H), 4.11-4.04 (m, 2H), 3.74-3.69 (m, 2H), 3.60-3.54 (m, 2H), 2.17-2.10 (m, 2H), 1.60-1.51 (m, 2H), 1.32 (s, 3H).

### Example 1.29. Synthesis of 1-{6-fluoro-8-[5-(trifluoromethyl)-1,2,4-oxadiazol-3-yl]-3,5-dihydro-2H-1,4-benzoxazepin-4-yl}-2,2-dimethylpropan-1-one (I-29)

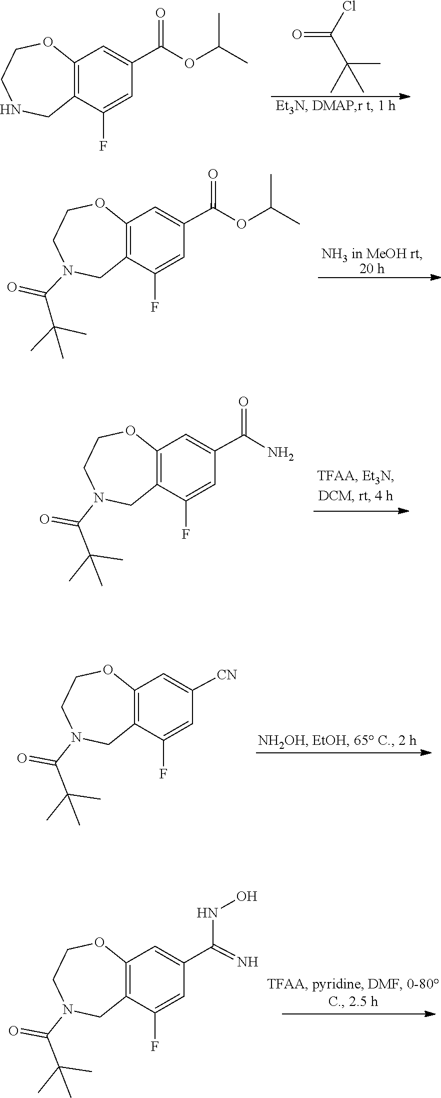

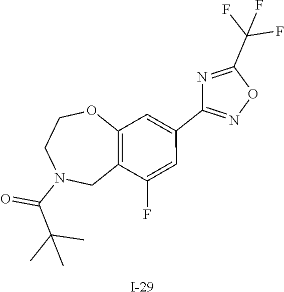

Isopropyl 4-(2,2-dimethylpropanoyl)-6-fluoro-3,5-dihydro-2H-1,4-benzoxazepine-8-carboxylate. 2,2-dimethylpropanoyl chloride (1.41 g, 11.7 mmol) was added to the mixture of isopropyl 6-fluoro-2,3,4,5-tetrahydro-1,4-benzoxazepine-8-carboxylate (1 g, 3.9 mmol), triethylamine (1.58 g, 15.6 mmol) and 4-DMAP (50 mg, 0.4 mmol) in DCM (5 mL) at 0° C. The mixture was stirred at room temperature for 1 hour. The mixture was diluted with water (50 mL) and extracted with DCM (10 mL×2). The organic layer was washed with brine (10 mL×3), dried over sodium sulphate, filtered and concentrated in vacuo. The residue was purified by chromatography column on silica gel (40 g) eluting with PE/EA (10%) to give product isopropyl 4-(2,2-dimethylpropanoyl)-6-fluoro-3,5-dihydro-2H-1,4-benzoxazepine-8-carboxylate (1.27 g, 95%) as white solid. MS (ESI): mass calcd. for C₁₈H₂₄FNO₄, 337.17, m/z found 338.2 [M+H]⁺.

4-(2,2-dimethylpropanoyl)-6-fluoro-3,5-dihydro-2H-1,4-benzoxazepine-8-carboxamide. The mixture of NH₃ in MeOH (7 M, 20 mL) and isopropyl 4-(2,2-dimethylpropanoyl)-6-fluoro-3,5-dihydro-2H-1,4-benzoxazepine-8-carboxylate (950 mg, 2.82 mmol) was stirred at 70° C. for 20 hours. The mixture was concentrated in vacuo to afford the crude 4-(2,2-dimethylpropanoyl)-6-fluoro-3,5-dihydro-2H-1,4-benzoxazepine-8-carboxamide (600 mg, 65%) as a white solid. MS (ESI): mass calcd. for C₁₅H₁₉FN₂O₃, 294.14, m/z found 295.1 [M+H]⁺.

4-(2,2-dimethylpropanoyl)-6-fluoro-3,5-dihydro-2H-1,4-benzoxazepine-8-carbonitrile. Trifluoroacetic anhydride (855 mg, 4.08 mmol) was added to the mixture of 4-(2,2-dimethylpropanoyl)-6-fluoro-3,5-dihydro-2H-1,4-benzoxazepine-8-carboxamide (300 mg, 1.02 mmol) and triethylamine (1.55 g, 15.30 mmol) in DCM (30 mL) at 0° C. The mixture was stirred at room temperature for 2 hours. The mixture was diluted with water (50 mL) and extracted with DCM (10 mL×2). The organic layer was washed with brine (10 mL×3), dried over sodium sulphate, filtered and concentrated in vacuo. The residue was purified by chromatography column on silica gel (40 g) eluting with PE/EA (10%) to give product 4-(2,2-dimethylpropanoyl)-6-fluoro-3,5-dihydro-2H-1,4-benzoxazepine-8-carbonitrile (220 mg, 78%) as a white solid. MS (ESI): mass calcd. for C₁₅H₁₇FN₂O₂, 276.13, m/z found 277.1 [M+H]⁺.

4-(2,2-dimethylpropanoyl)-6-fluoro-N-hydroxy-3,5-dihydro-2H-1,4-benzoxazepine-8-carboximidamide. NH₂OH/H₂O (2.39 g, 36.2 mmol) was added to the mixture of 4-(2,2-dimethylpropanoyl)-6-fluoro-3,5-dihydro-2H-1,4-benzoxazepine-8-carbonitrile (200 mg, 0.72 mmol) in EtOH (10 mL). The mixture was stirred at 65° C. The mixture was stirred at room temperature for 4 hours. The mixture was concentrated in vacuo to afford 4-(2,2-dimethylpropanoyl)-6-fluoro-N-hydroxy-3, 5-dihydro-2H-1,4-benzoxazepine-8-carboximidamide (220 mg, 97%) as white solid. MS (ESI): mass calcd. for C₁₅H₂₀FN₃O₃, 309.15, m/z found 310.2 [M+H]⁺.

1-{6-fluoro-8-[5-(trifluoromethyl)-1,2,4-oxadiazol-3-yl]-3,5-dihydro-2H-1,4-benzoxazepin-4-yl}-2,2-dimethylpropan-1-one. TFAA (407 mg, 1.94 mmol) and pyridine (511 mg, 6.47 mmol) were added to the 4-(2,2-dimethylpropanoyl)-6-fluoro-N-hydroxy-3,5-dihydro-2H-1,4-benzoxazepine-8-carboximidamide (200 mg, 0.65 mmol) in DMF (10 mL). The mixture was stirred at 80° C. for 3 hours. The mixture was concentrated and the residue was purified by chromatography column on silica gel (40 g) eluting with PE/EA (5%) to give 1-{6-fluoro-8-[5-(trifluoromethyl)-1,2,4-oxadiazol-3-yl]-3, 5-dihydro-2H-1,4-benzoxazepin-4-yl}-2,2-dimethylpropan-1-one (220 mg, 87%) as white solid. MS (ESI): mass calcd. for C₁₇H₁₇F₄N₃O₃, 387.12, m/z found 388.1 [M+H]⁺. ¹H NMR (400 MHz, CDCl₃-d) δ 7.58-7.52 (m, 2H), 4.81 (s, 2H), 4.31-4.26 (m, 2H), 4.10-4.04 (m, 2H), 1.28 (s, 9H).

### Example 1.30. Synthesis of (3S)-6-fluoro-4-{[3-(methoxymethyl)oxetan-3-yl]carbonyl}-3-methyl-8-[5-(trifluoromethyl)-1,2,4-oxadiazol-3-yl]-3,5-dihydro-2H-1,4-benzoxazepine (I-30)

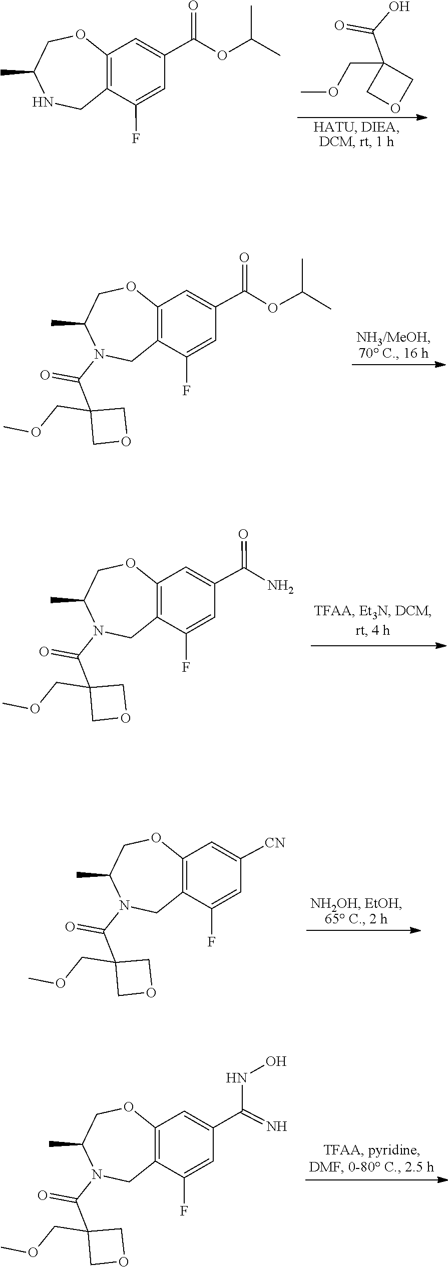

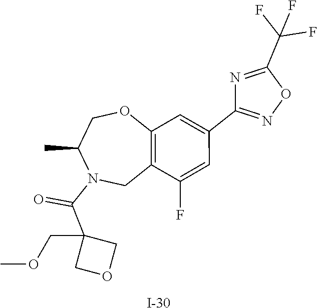

Isopropyl (3S)-6-fluoro-4-{[3-(methoxymethyl)oxetan-3-yl]carbonyl}-3-methyl-3,5-dihydro-2H-1,4-benzoxazepine-8-carboxylate. To a solution of isopropyl (3S)-6-fluoro-3-methyl-2,3,4,5-tetrahydro-1,4-benzoxazepine-8-carboxylate (549 mg, 2.05 mmol) and 3-(methoxymethyl)oxetane-3-carboxylic acid (300 mg, 2.05 mmol) in DMF (20 mL) was added NEt₃ (623 mg, 6.16 mmol), HBTU (934 mg, 2.46 mmol). The reaction mixture was stirred at room temperature for 2 h under nitrogen atmosphere. The mixture was quenched with water (20 mL) and extracted with EA (15 mL×3). The combined organic layer was dried over Na₂SO₄ and concentrated under reduced pressure to get a residue which was purified by column chromatography on silica gel (25 g) eluting with EA in PE (0-25%) to afford isopropyl (3S)-6-fluoro-4-{[3-(methoxymethyl)oxetan-3-yl]carbonyl}-3-methyl-3, 5-dihydro-2H-1,4-benzoxazepine-8-carboxylate (444 mg, 46%) as yellow oil. MS (ESI): mass calcd. for C₂₀H₂₆FNO₆, 395.17, m/z found 396.1 [M+H]⁺.

(3S)-6-fluoro-4-{[3-(methoxymethyl)oxetan-3-yl]carbonyl}-3-methyl-3,5-dihydro-2H-1,4-benzoxazepine-8-carboxamide. To the oil of isopropyl (3S)-6-fluoro-4-{[3-(methoxymethyl)oxetan-3-yl]carbonyl}-3-methyl-3, 5-dihydro-2H-1,4-benzoxazepine-8-carboxylate (444 mg, 1.12 mmol) was added NH₃ in MeOH (1.0 mL, 5.6 mmol) at room temperature. The reaction mixture was stirred at 70° C. for 12 h under nitrogen atmosphere. The mixture was concentrated under reduced pressure to get the required product (3S)-6-fluoro-4-{[3-(methoxymethyl)oxetan-3-yl]carbonyl}-3-methyl-3, 5-dihydro-2H-1,4-benzoxazepine-8-carboxamide (368 mg, 74%) as yellow oil. MS (ESI): mass calcd. for C₁₇H₂₁FN₂O₅, 352.14, m/z found 353.1 [M+H]⁺.

(3S)-6-fluoro-4-{[3-(methoxymethyl)oxetan-3-yl]carbonyl}-3-methyl-3,5-dihydro-2H-1,4-benzoxazepine-8-carbonitrile. To a solution of (3S)-6-fluoro-4-{[3-(methoxymethyl)oxetan-3-yl]carbonyl}-3-methyl-3, 5-dihydro-2H-1,4-benzoxazepine-8-carboxamide (368 mg, 1.04 mmol) and NEt₃ (317 mg, 3.13 mmol) in DCM (10 mL) at 0° C. was added TFAA (658 mg, 3.13 mmol). The reaction mixture was stirred at room temperature for 2 h. The mixture was quenched with water (20 mL) and extracted with DCM (10 mL×3). The combined organic layer was washed with brine (10 mL×3) and concentrated under reduced pressure to get a residue which was purified by column chromatography on silica gel (12 g) eluting with EA in PE (0-40%) to give (3S)-6-fluoro-4-{[3-(methoxymethyl)oxetan-3-yl]carbonyl}-3-methyl-3,5-dihydro-2H-1,4-benzoxazepine-8-carbonitrile (326 mg, 75%) as yellow oil. MS (ESI): mass calcd. for C₁₇H₁₉FN₂O₄, 334.13, m/z found 319.1 [M+H]⁺.

(3S)-6-fluoro-N-hydroxy-4-{[3-(methoxymethyl)oxetan-3-yl]carbonyl}-3-methyl-3,5-dihydro-2H-1,4-benzoxazepine-8-carboximidamide. To a solution of (3S)-6-fluoro-4-{[3-(methoxymethyl)oxetan-3-yl]carbonyl}-3-methyl-3, 5-dihydro-2H-1,4-benzoxazepine-8-carbonitrile (326 mg, 0.98 mmol) in EtOH (10 mL) was added NH₂OH/H₂O (3.2 g, 97.5 mmol). The reaction mixture was stirred at 65° C. for 2 h under nitrogen atmosphere. The mixture was concentrated under reduced pressure to get the required product (3S)-6-fluoro-N-hydroxy-4-{[3-(methoxymethyl)oxetan-3-yl]carbonyl}-3-methyl-3, 5-dihydro-2H-1,4-benzoxazepine-8-carboximidamide (220 mg, 55%) as a white solid. MS (ESI): mass calcd. for C₁₇H₂₂FN₃O₅, 367.15, m/z found 368.2 [M+H]⁺.

(3S)-6-fluoro-4-{[3-(methoxymethyl)oxetan-3-yl]carbonyl}-3-methyl-8-[5-(trifluoromethyl)-1,2,4-oxadiazol-3-yl]-3,5-dihydro-2H-1,4-benzoxazepine. To a solution of (3S)-6-fluoro-N-hydroxy-4-{[3-(methoxymethyl)oxetan-3-yl]carbonyl}-3-methyl-3,5-dihydro-2H-1,4-benzoxazepine-8-carboximidamide (220 mg, 0.60 mmol) and pyridine (142 mg, 1.80 mmol) in DMF (20 mL) was added TFAA (378 mg, 1.80 mmol) at 0° C. under nitrogen atmosphere. The reaction mixture was stirred at 80° C. for 2 h under nitrogen atmosphere. The mixture was cooled to room temperature and concentrated under reduced pressure to get a residue which was purified by column chromatography on silica gel (12 g) eluting with EA in PE (0-40%) to give (3S)-6-fluoro-4-{[3-(methoxymethyl)oxetan-3-yl]carbonyl}-3-methyl-8-[5-(trifluoromethyl)-1,2,4-oxadiazol-3-yl]-3,5-dihydro-2H-1,4-benzoxazepine (102.4 mg, 36%) as a white solid. MS (ESI): mass calcd. for C₁₉H₁₉F₄N₃O₅, 445.13, m/z found 446.1 [M+H]⁺. ¹H NMR (400 MHz, CDCl₃) δ 7.55-7.43 (m, 2H), 5.53-3.61 (m, 11H), 3.34 (s, 3H), 1.38 (d, J=6.8 Hz, 1H), 1.16 (d, J=6.4 Hz, 2H).

### Example 1.31. Synthesis of (3S)-6-fluoro-4-{[1-(methoxymethyl)cyclopropyl]carbonyl}-3-methyl-8-[5-(trifluoromethyl)-1,2,4-oxadiazol-3-yl]-3,5-dihydro-2H-1,4-benzoxazepine (I-31)

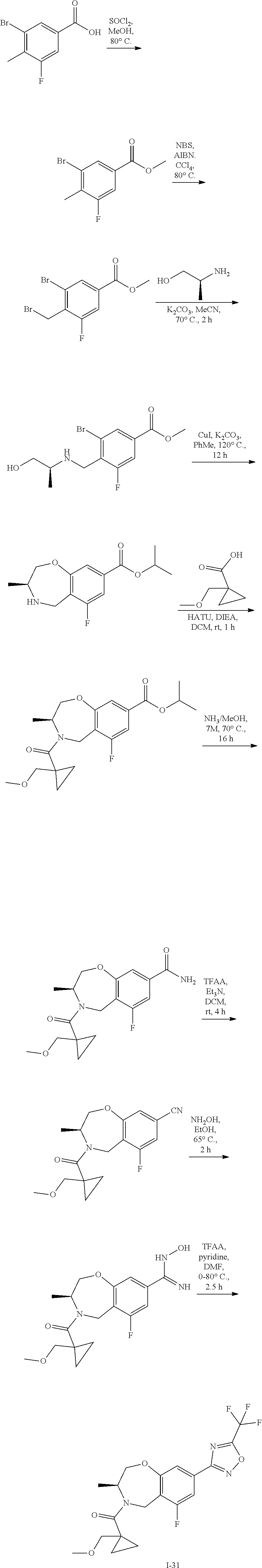

Methyl 3-bromo-5-fluoro-4-methylbenzoate. To a solution of 3-bromo-5-fluoro-4-methylbenzoic acid (25.0 g, 107.3 mmol) in MeOH (300 mL) was added SOCl₂ (10 mL) at 0° C. under nitrogen atmosphere. The reaction mixture was stirred at 80° C. for 16 h under nitrogen atmosphere. The mixture was cooled to room temperature and concentrated under reduced pressure to get a residue which was purified by column chromatography on silica gel (120 g) eluting with EA in PE (0-5%) to afford methyl 3-bromo-5-fluoro-4-methylbenzoate (26.0 g, 88%) as yellow oil. MS (ESI): mass calcd. for C₉H₈BrFO₂, 246.0, m/z found 247.0 [M+H]⁺.

Methyl 3-bromo-4-(bromomethyl)-5-fluorobenzoate. To a solution of methyl 3-bromo-5-fluoro-4-methylbenzoate (26.0 g, 105.2 mmol) in CCl₄ (300 mL) were added NBS (20.6 g, 115.7 mmol) and AIBN (1.7 g, 10.5 mmol) at room temperature under nitrogen atmosphere. The reaction mixture was stirred at 80° C. for 16 h under nitrogen atmosphere. The mixture was quenched with water (100 mL) and concentrated under reduced pressure to remove CCl₄. The mixture was extracted with EA (100 mL×3). The combine organic layer was dried over Na₂SO₄ and concentrated under reduced pressure to get a residue which was purified by column chromatography on silica gel (120 g) eluting with EA in PE (0-10%) to afford methyl 3-bromo-4-(bromomethyl)-5-fluorobenzoate (30.0 g, 79%) as yellow oil. MS (ESI): mass calcd. for C₉H₇Br₂FO₂, 323.9, m/z found 324.9 [M+H]⁺.

Methyl 3-bromo-5-fluoro-4-{[(2-hydroxyethyl)amino]methyl}benzoate. To a solution of methyl 3-bromo-4-(bromomethyl)-5-fluorobenzoate (10.0 g, 30.7 mmol) in ACN (100 mL) were added (2S)-2-aminopropan-1-ol (6.9 g, 92.1 mmol) and K₂CO₃ (12.7 g, 92.1 mmol) at room temperature under nitrogen atmosphere. The reaction mixture was stirred at room temperature for 3 h under nitrogen atmosphere. The mixture was concentrated under reduced pressure and extracted with EA (100 mL×3). The combine organic layer was dried over Na₂SO₄ and concentrated under reduced pressure to get a residue which was purified by column chromatography on silica gel (120 g) eluting with MeOH in DCM (0-5%) to afford methyl 3-bromo-5-fluoro-4-{[(2-hydroxyethyl)amino]methyl}benzoate (8.0 g, 77%) as a white solid. MS (ESI): mass calcd. for C₁₂H₁₅BrFNO₃, 319.0, m/z found 320.0 [M+H]⁺.

Isopropyl (3S)-6-fluoro-3-methyl-2,3,4,5-tetrahydro-1,4-benzoxazepine-8-carboxylate. To a solution of methyl 3-bromo-5-fluoro-4-({[(2S)-1-hydroxypropan-2-yl]amino}methyl)benzoate (7.0 g, 21.90 mmol) in IPA (100 mL) were added Copper(I) iodide (0.4 g, 2.19 mmol) and K₂CO₃ (9.1 g, 65.70 mol) at room temperature under nitrogen atmosphere. The reaction mixture was stirred at 80° C. for 16 hours under nitrogen atmosphere. The mixture was cooled to room temperature and filtered. The filtrate was concentrated to get a residue which was purified by column chromatography on silica gel (80 g) eluting with EA in PE (0-50%) to afford isopropyl (3S)-6-fluoro-3-methyl-2,3,4,5-tetrahydro-1,4-benzoxazepine-8-carboxylate (3.8 g, 62%) as yellow oil. MS (ESI): mass calcd. for C₁₂H₁₄FNO₃, 239.10, m/z found 240.1 [M+H]⁺.

Isopropyl (3S)-6-fluoro-4-{[1-(methoxymethyl)cyclopropyl]carbonyl}-3-methyl-3,5-dihydro-2H-1,4-benzoxazepine-8-carboxylate. To a solution of isopropyl (3S)-6-fluoro-3-methyl-2,3,4,5-tetrahydro-1,4-benzoxazepine-8-carboxylate (534 mg, 2.0 mmol) and 1-(methoxymethyl)cyclopropane-1-carboxylic acid (260 mg, 2.0 mmol) in DMF (25 mL) was added NEt₃ (404 mg, 4.0 mmol), HBTU (1.2 g, 3.0 mmol) at room temperature under nitrogen atmosphere. The reaction mixture was stirred at room temperature for 2 h under nitrogen atmosphere. The mixture was quenched with water (20 mL) and extracted with DCM (3×15 mL). The combined organic layer was dried over Na₂SO₄ and concentrated under reduced pressure to get a residue which was purified by column chromatography on silica gel (25 g) eluting with EA in PE (0-25%) to afford isopropyl (3S)-6-fluoro-4-{[1-(methoxymethyl)cyclopropyl]carbonyl}-3-methyl-3, 5-dihydro-2H-1,4-benzoxazepine-8-carboxylate (720 mg, 85%) as transparent viscous liquid. MS (ESI): mass calcd. for C₂₀H₂₆FNO₅, 379.18, m/z found 380.1 [M+H]⁺.

(3S)-6-fluoro-4-{[1-(methoxymethyl)cyclopropyl]carbonyl}-3-methyl-3,5-dihydro-2H-1,4-benzoxazepine-8-carboxamide. To the liquid of isopropyl (3S)-6-fluoro-4-{[1-(methoxymethyl)cyclopropyl]carbonyl}-3-methyl-3, 5-dihydro-2H-1,4-benzoxazepine-8-carboxylate (500 mg, 1.32 mmol) was added NH₃ in MeOH (20 mL, 131.8 mmol) at room temperature. The reaction mixture was stirred at 70° C. for 12 h under nitrogen atmosphere. The mixture was concentrated under reduced pressure to get the required product (3S)-6-fluoro-4-{[1-(methoxymethyl)cyclopropyl]carbonyl}-3-methyl-3, 5-dihydro-2H-1,4-benzoxazepine-8-carboxamide (414 mg, 79%) as yellow oil. MS (ESI): mass calcd. for C₁₇H₂₁FN₂O₄, 336.15, m/z found 337.1 [M+H]⁺.

(3S)-6-fluoro-4-{[1-(methoxymethyl)cyclopropyl]carbonyl}-3-methyl-3,5-dihydro-2H-1,4-benzoxazepine-8-carbonitrile. To a solution of (3S)-6-fluoro-4-{[1-(methoxymethyl)cyclopropyl]carbonyl}-3-methyl-3, 5-dihydro-2H-1,4-benzoxazepine-8-carboxamide (441 mg, 1.23 mmol) and trimethylamine (374 mg, 3.69 mmol) in DCM (10 mL) was added TFAA (776 mg, 3.69 mmol) under nitrogen atmosphere. The reaction mixture was stirred at room temperature for 4 h under nitrogen atmosphere. The mixture was quenched with water (20 mL) and extracted with DCM (10 mL×3). The combined organic layer was washed with brine (10 mL×3) and concentrated under reduced pressure to get a residue which was purified by column chromatography on silica gel (25 g) eluting with EA in PE (0-40%) to afford (3S)-6-fluoro-4-{[1-(methoxymethyl)cyclopropyl]carbonyl}-3-methyl-3,5-dihydro-2H-1,4-benzoxazepine-8-carbonitrile (222 mg, 48%) as transparent viscous liquid. MS (ESI): mass calcd. for C₁₇H₁₉FN₂O₃, 318.14, m/z found 319.1 [M+H]⁺.

(3S)-6-fluoro-N-hydroxy-4-{[1-(methoxymethyl)cyclopropyl]carbonyl}-3-methyl-3,5-dihydro-2H-1,4-benzoxazepine-8-carboximidamide. To a solution of (3S)-6-fluoro-4-{[1-(methoxymethyl)cyclopropyl]carbonyl}-3-methyl-3, 5-dihydro-2H-1,4-benzoxazepine-8-carbonitrile (222 mg, 0.70 mmol) in EtOH (20 mL) was added NH₂OH/H₂O (2.3 g, 69.7 mmol, 50%) under nitrogen atmosphere at room temperature. The reaction mixture was stirred at 65° C. for 2 h under nitrogen atmosphere. The mixture was concentrated under reduced pressure to get the required product (3S)-6-fluoro-N-hydroxy-4-{[1-(methoxymethyl)cyclopropyl]carbonyl}-3-methyl-3,5-dihydro-2H-1,4-benzoxazepine-8-carboximidamide (300 mg, 83%) as a white solid. MS (ESI): mass calcd. for C₁₇H₂₂FN₃O₄, 351.16, m/z found 352.1 [M+H]⁺.

(3S)-6-fluoro-4-{[1-(methoxymethyl)cyclopropyl]carbonyl}-3-methyl-8-[5-(trifluoromethyl)-1,2,4-oxadiazol-3-yl]-3,5-dihydro-2H-1,4-benzoxazepine. To a solution of (3S)-6-fluoro-N-hydroxy-4-{[1-(methoxymethyl)cyclopropyl]carbonyl}-3-methyl-3, 5-dihydro-2H-1,4-benzoxazepine-8-carboximidamide (300 mg, 0.85 mmol) and pyridine (68 mg, 2.55 mmol) in DMF (10 mL) was added TFAA (535 mg, 2.55 mmol) at 0° C. under nitrogen atmosphere. The reaction mixture was stirred at 80° C. for 6 h under nitrogen atmosphere. The mixture was cooled to room temperature and concentrated under reduced pressure to get a residue which was purified by column chromatography on silica gel (25 g) eluting with EA in PE (0-40%) to give (3S)-6-fluoro-4-{[1-(methoxymethyl)cyclopropyl]carbonyl}-3-methyl-8-[5-(trifluoromethyl)-1,2,4-oxadiazol-3-yl]-3,5-dihydro-2H-1,4-benzoxazepine (35.0 mg, 10%) as white oil. MS (ESI): mass calcd. for C₁₉H₁₉F₄N₃O₄, 429.13, m/z found 430.1 [M+H]⁺. ¹H NMR (400 MHz, MeOD-d₄) δ 7.59-7.36 (m, 2H), 5.86-5.03 (m, 2H), 4.71-4.20 (m, 3H), 3.64 (d, J=10.4 Hz, 1H), 3.29-2.96 (m, 4H), 1.44-1.11 (m, 3H), 0.93-0.55 (m, 4H).

### Example 1.32. Synthesis of (3S)-3-cyclopropyl-4-[(4-methyloxan-4-yl) carbonyl]-8-[5-(trifluoromethyl)-1,2,4-oxadiazol-3-yl]-3,5-dihydro-2H-1,4-benzoxazepine (I-32)

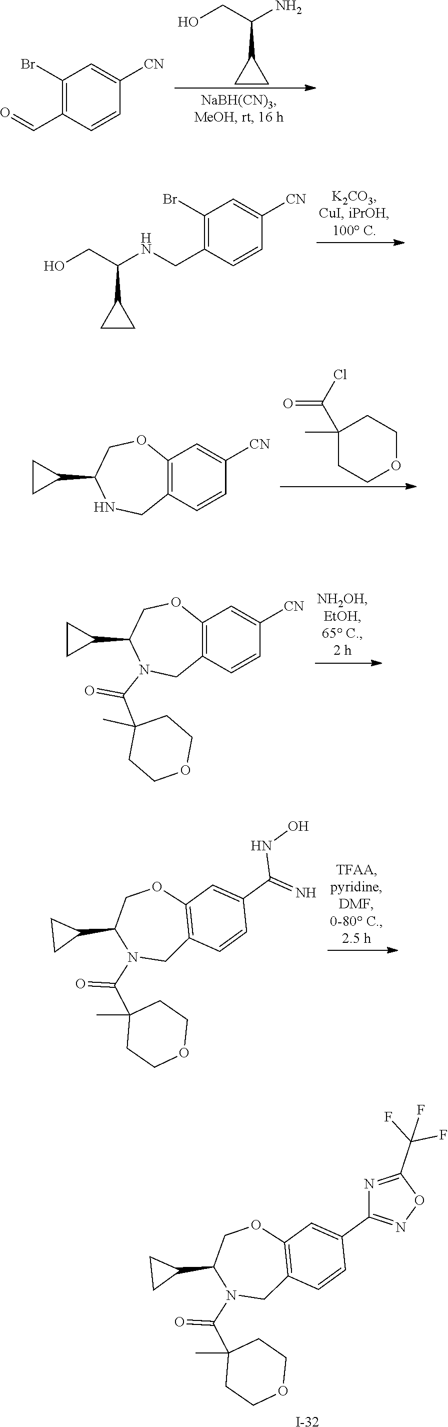

3-bromo-4-({[(1S)-1-cyclopropyl-2-hydroxyethyl]amino}methyl) benzonitrile. To a solution of 3-bromo-4-formylbenzonitrile (4 g, 0.019 mol), (2S)-2-amino-2-cyclopropylethanol (2.31 g, 0.0228 mol) in MeOH (60 ml) stirred under nitrogen at 25° C. for 12 h. NaBH₃CN (3.58 g, 0.0057 mol) was added. The reaction mixture was stirred at 0° C. for 0.5 h. The mixture was quenched by NH₄Cl (aq, 50 mL) and extracted with EtOAc (80 mL×3). The organic layer was dried over anhydrous Na₂SO₄, filtered and concentrated under reduced pressure to give the crude product. The residue was purified by Flash Chromatography to give 3-bromo-4-({[(1S)-1-cyclopropyl-2-hydroxyethyl]amino}methyl) benzonitrile (1.45 g, 22%) as an orange solid. MS (ESI): mass calcd. for C₁₃H₁₅BrN₂O, 294.04, m/z found 295.1 [M+H]⁺.

(3S)-3-cyclopropyl-2,3,4,5-tetrahydro-1,4-benzoxazepine-8-carbonitrile. A mixture of 3-bromo-4-({[(1S)-1-cyclopropyl-2-hydroxyethyl]amino}methyl) benzonitrile (1.45 g, 0.0049 mol), Potassium carbonate (1.35 g, 0.0098 mol) and Copper (I) iodide (0.47 g, 0.0024 mol) in i-PrOH (60 ml) was stirred at 100° C. for 12 h. The reaction mixture was cooled to room temperature. The mixture was filtered and concentrated under reduced pressure to give the crude product. The mixture was diluted with water and extracted with EtOAc (50 mL×3). The organic layer was dried over anhydrous Na₂SO₄, filtered and concentrated under reduced pressure to give the crude product. The residue was purified by flash chromatography to give (3S)-3-cyclopropyl-2,3,4,5-tetrahydro-1,4-benzoxazepine-8-carbonitrile (430 mg, 32%) as an orange oil. MS (ESI): mass calcd. for C₁₃H₁₄N₂O, 214.11, m/z found 215.2 [M+H]⁺.

(3S)-3-cyclopropyl-4-[(4-methyloxan-4-yl) carbonyl]-3,5-dihydro-2H-1,4-benzoxazepine-8-carbonitrile. To a solution of (3S)-3-cyclopropyl-2,3,4,5-tetrahydro-1,4-benzoxazepine-8-carbonitrile (350 mg, 1.6335 mmol) in pyridine (4 ml) stirred under nitrogen at 0° C. 4-methyloxane-4-carbonyl chloride (664 mg, 4.0837 mmol) was added. The reaction mixture was stirred at 25° C. for 24 h. The mixture was concentrated to afford the crude product. The crude product was purified by RP-C18 column eluted with H₂O (0.5% FA)/CH₃CN (100:0→50:50) to afford (3S)-3-cyclopropyl-4-[(4-methyloxan-4-yl) carbonyl]-3,5-dihydro-2H-1,4-benzoxazepine-8-carbonitrile (750 mg, 76%) as an orange oil. MS (ESI): mass calcd. for C₂₀H₂₄N₂O₃, 340.18, m/z found 341.1 [M+H]⁺.

(3S)-3-cyclopropyl-N-hydroxy-4-[(4-methyloxan-4-yl) carbonyl]-3,5-dihydro-2H-1,4-benzoxazepine-8-carboximidamide. A mixture of (3S)-3-cyclopropyl-4-[(4-methyloxan-4-yl) carbonyl]-3,5-dihydro-2H-1,4-benzoxazepine-8-carbonitrile (770 mg, 2.2619 mmol) and NH₂OH/H₂O (224 mg, 6.7857 mmol) in EtOH (10 ml) was stirred at 65° C. for 2.5 h. The reaction mixture was cooled to room temperature. The mixture was diluted with water and extracted with EtOAc (30 mL×3). The organic layer was dried over anhydrous Na₂SO₄, filtered and concentrated under reduced pressure to give (3S)-3-cyclopropyl-N-hydroxy-4-[(4-methyloxan-4-yl) carbonyl]-3,5-dihydro-2H-1,4-benzoxazepine-8-carboximidamide (670 mg, 68%) as orange solid. MS (ESI): mass calcd. for C₂₀H₂₇N₃O₄, 373.20, m/z found 374.2 [M+H]⁺.

(3S)-3-cyclopropyl-4-[(4-methyloxan-4-yl) carbonyl]-8-[5-(trifluoromethyl)-1,2,4-oxadiazol-3-yl]-3,5-dihydro-2H-1,4-benzoxazepine. To a solution of (3S)-3-cyclopropyl-N-hydroxy-4-[(4-methyloxan-4-yl) carbonyl]-3,5-dihydro-2H-1,4-benzoxazepine-8-carboximidamide (630 mg, 1.687 mmol), pyridine (400 mg, 5.061 mmol) in DMF (10 ml) stirred under nitrogen at 0° C. TFAA (531 mg, 2.53 mmol) was added. The reaction mixture was stirred at 80° C. for 12 h. The reaction mixture was cooled to room temperature. The mixture was diluted with water and extracted with EtOAc (30 mL×3). The organic layer was dried over anhydrous Na₂SO₄, filtered and concentrated under reduced pressure to give the crude product. The crude product was purified by RP-C18 column eluted with H₂O (0.5% FA)/CH₃CN (100:0→50:50) to afford (3S)-3-cyclopropyl-4-[(4-methyloxan-4-yl) carbonyl]-8-[5-(trifluoromethyl)-1,2,4-oxadiazol-3-yl]-3,5-dihydro-2H-1,4-benzoxazepine (270 mg, 34%) as a white solid. MS (ESI): mass calcd. for C₂₂H₂₄F₃N₃O₄, 451.17, m/z found 452.1 [M+H]⁺. ¹H NMR (400 MHz, CD₃Cl) δ 7.74-7.70 (m, 2H), 7.33 (s, 1H), 4.96-4.78 (m, 2H), 4.27 (s, 3H), 3.74-3.70 (m, 1H), 3.60-3.54 (m, 2H), 3.37 (m, 1H), 2.20-2.16 (m, 1H), 2.06-2.01 (m, 1H), 1.74 (s, 1H), 1.54-1.48 (m, 2H), 1.27 (s, 3H), 0.72-0.50 (m, 4H).

### Example 1.33. Synthesis of (S)-3-methyl-4-(methylsulfonyl)-8-(5-(trifluoromethyl)-1,2,4-oxadiazol-3-yl)-2,3,4,5-tetrahydrobenzo[f][1,4]oxazepine (I-33)

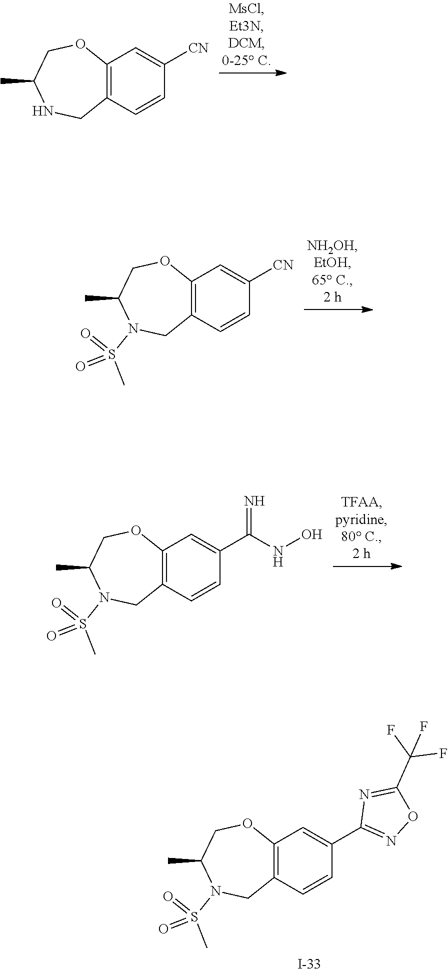

(S)-3-methyl-4-(methylsulfonyl)-2,3,4,5-tetrahydrobenzo[f][1,4]oxazepine-8-carbonitrile. To the mixture of (S)-3-methyl-2,3,4,5-tetrahydrobenzo[f][1,4]oxazepine-8-carbonitrile (200 mg, 1.06 mmol), triethylamine (323 mg, 3.19 mmol) in DCM (5 mL) stirred under nitrogen at 0° C. was added a solution of MsCl (183 mg, 1.59 mmol) in DCM (1 mL) dropwise. The reaction mixture was stirred at 25° C. for 1 h. After completion, the reaction was quenched with with ice water (10 mL) and extracted with DCM (20 mL×3). The combined organic layers were dried (Na₂SO₄), filtered, and concentrated under reduced pressure to afford (S)-3-methyl-4-(methylsulfonyl)-2,3,4,5-tetrahydrobenzo[f][1,4]oxazepine-8-carbonitrile (280 mg, 99%) as a white solid. MS (ESI): mass calcd. for C₁₂H₁₄N₂O₃S, 266.1 (exact mass), m/z found 289.0 [M+Na]⁺.

(S)—N-hydroxy-3-methyl-4-(methylsulfonyl)-2,3,4,5-tetrahydrobenzo[f][1,4]oxazepine-8-carboximidamide. To the mixture of (S)-3-methyl-4-(methylsulfonyl)-2,3,4,5-tetrahydrobenzo[f][1,4]oxazepine-8-carbonitrile (250 mg, 0.94 mmol) in EtOH (5 mL) was added NH₂OH/H₂O (96 mg, 1.88 mmol). The reaction mixture was stirred at 65° C. for 2 h. After completion, reaction mixture was concentrated under reduced pressure. The residue was extracted with EtOAc (20 mL×3). The combined organic layers were dried (Na₂SO₄), filtered, and concentrated to afford (S)—N-hydroxy-3-methyl-4-(methylsulfonyl)-2,3,4,5-tetrahydrobenzo[f][1,4]oxazepine-8-carboximidamide (280 mg, 99%) as a yellow solid. MS (ESI): mass calcd. for C₁₂H₁₇N₃O₄S, 299.1 (exact mass), m/z found 300.1 [M+H]⁺.

(S)-3-methyl-4-(methylsulfonyl)-8-(5-(trifluoromethyl)-1,2,4-oxadiazol-3-yl)-2,3,4,5-tetrahydrobenzo[f][1,4]oxazepine. To the mixture of (S)—N-hydroxy-3-methyl-4-(methylsulfonyl)-2,3,4,5-tetrahydrobenzo[f][1,4]oxazepine-8-carboximidamide (230 mg, 0.77 mmol) and pyridine (182 mg, 2.30 mmol) in DMF (4 mL) was added TFAA (323 mg, 1.54 mmol). The reaction mixture was stirred at 80° C. for 2 h. After completion, reaction mixture was concentrated under reduced pressure. The residue was purified by reverse phase (60% MeCN in A, A: 0.1% TFA in water) to afford (S)-3-methyl-4-(methylsulfonyl)-8-(5-(trifluoromethyl)-1,2,4-oxadiazol-3-yl)-2,3,4,5-tetrahydrobenzo[f][1,4]oxazepine (85.3 mg, 26%) as a white solid. MS (ESI): mass calcd. for C₁₄H₁₄F₃N₃O₄S, 377.1 (exact mass), m/z found 378.0 [M+H]⁺. ¹H NMR (400 MHz, DMSO-d₆) δ 7.67 (dd, J=8.0, 1.2 Hz, 1H), 7.53-7.42 (m, 2H), 4.93 (d, J=17.6 Hz, 1H), 4.56 (d, J=18.0 Hz, 1H), 4.35-4.24 (m, 3H), 2.68 (s, 3H), 1.20 (d, J=5.6 Hz, 3H).

### Example 1.34. Synthesis of (3S)-3-methyl-4-(2-methylpropane-2-sulfonyl)-8-[5-(trifluoromethyl)-1,2,4-oxadiazol-3-yl]-3,5-dihydro-2H-1,4-benzoxazepine (I-34)

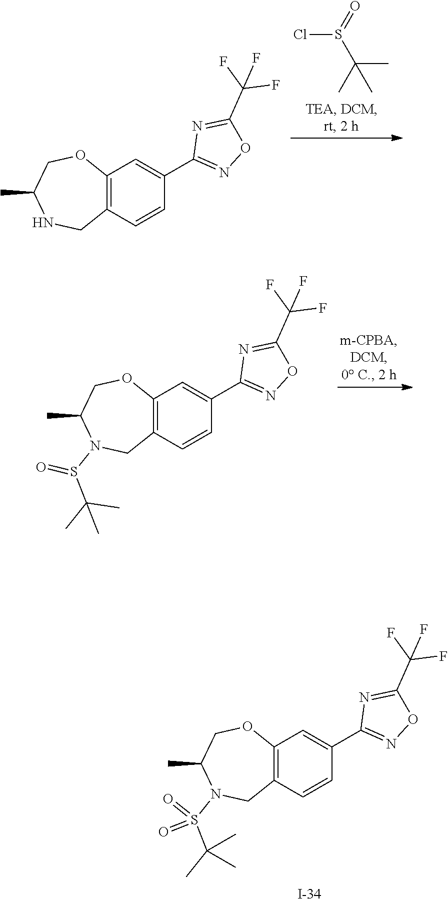

(3S)-3-methyl-4-(2-methylpropane-2-sulfinyl)-8-[5-(trifluoromethyl)-1,2,4-oxadiazol-3-yl]-3,5-dihydro-2H-1,4-benzoxazepine. To a mixture of (3S)-3-methyl-8-[5-(trifluoromethyl)-1,2,4-oxadiazol-3-yl]-2,3,4,5-tetrahydro-1,4-benzoxazepine (100 mg, 0.33 mmol) in DCM (5 mL) was added TEA (101 mg, 1.00 mmol) and 2-methylpropane-2-sulfinyl chloride (71 mg, 0.50 mmol). The reaction mixture was stirred at 25° C. for 2 hours. The reaction mixture was concentrated under reduced pressure. The residue which was purified with prep-TLC (EA/PE=1/1, Rf=0.4) to afford (3S)-3-methyl-4-(2-methylpropane-2-sulfinyl)-8-[5-(trifluoromethyl)-1,2,4-oxadiazol-3-yl]-3,5-dihydro-2H-1,4-benzoxazepine (80 mg, 53%) as white solid. MS (ESI): mass calcd. for C₁₇H₂₀F₃N₃O₃S, 403.1, m/z found 404.0 [M+H]⁺.

(3S)-3-methyl-4-(2-methylpropane-2-sulfonyl)-8-[5-(trifluoromethyl)-1,2,4-oxadiazol-3-yl]-3,5-dihydro-2H-1,4-benzoxazepine. To a solution of (3S)-3-methyl-4-(2-methylpropane-2-sulfinyl)-8-[5-(trifluoromethyl)-1,2,4-oxadiazol-3-yl]-3,5-dihydro-2H-1,4-benzoxazepine (60 mg, 0.15 mmol) in DCM (3 mL) was added mCPBA (51 mg, 0.30 mmol). The reaction mixture was stirred at 25° C. for 3 hours. The mixture was concentrated and the residue was purified by Prep-HPLC (Instrument: Waters Prep-HPLC, Column: Xbridge prep c18 5 um OBD 19*150 mm, A water (0.1% FA), B Acetonitrile 10-20% B in 8 min, hold at 100% B at for 2 min, back to 5% B with 0.5 min, stop at 13 min, flow rate: 20 ml/min, wavelength: 214/254 nm, injection: 7) to afford (3S)-3-methyl-4-(2-methylpropane-2-sulfonyl)-8-[5-(trifluoromethyl)-1,2,4-oxadiazol-3-yl]-3,5-dihydro-2H-1,4-benzoxazepine (43.4 mg, 69%) as a white solid. MS (ESI): mass calcd. for C₁₇H₂₀F₃N₃O₄S, 419.1, m/z found 420.0 [M+H]⁺. ¹H NMR (400 MHz, MeOD) δ 7.69-7.61 (m, 1H), 7.57 (s, 1H), 7.27 (d, J=8.0 Hz, 1H), 4.75-7.62 (m, 1H), 4.57-4.41 (m, 1H), 4.31-3.99 (m, 3H), 1.30 (d, J=5.6 Hz, 3H), 1.16 (s, 9H).

### Example 1.35. Synthesis of (S)-3,4-dimethyl-8-(5-(trifluoromethyl)-1,2,4-oxadiazol-3-yl)-2,3,4,5-tetrahydrobenzo[f][1,4]oxazepine (I-35)

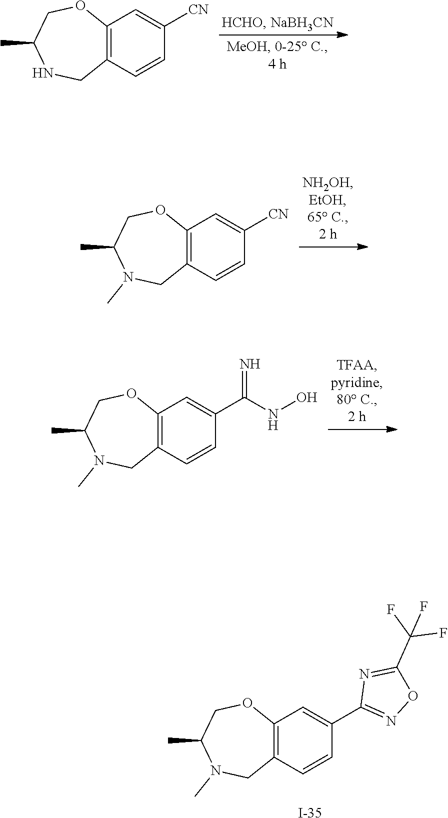

(S)-3,4-dimethyl-2,3,4,5-tetrahydrobenzo[f][1,4]oxazepine-8-carbonitrile. To the mixture of (S)-3-methyl-2,3,4,5-tetrahydrobenzo[f][1,4]oxazepine-8-carbonitrile (400 mg, 2.114 mmol), NaBH₃CN (199 mg, 3.171 mmol) and AcOH (13 mg, 0.212 mmol) in MeOH (10 mL) was added formaldehyde aqueous solution (190 mg, 6.342 mmol). The reaction mixture was stirred at room temperature for 3 h. The mixture was evaporated to afford a crude. The crude was extracted with EA (50 mL×3). The combined organic layers were washed with brine (30 mL×2), dried over Na₂SO₄ and concentrated under reduced pressure to afford crude product. The crude mixture was purified by Flash Chromatography with MeCN/0.1% TFA (35%) to afford (S)-3,4-dimethyl-2,3,4,5-tetrahydrobenzo[f][1,4]oxazepine-8-carbonitrile (350 mg, 81%) as a colourless oil. MS (ESI): mass calcd. for C₁₂H₁₄N₂O. 202.11, m/z found 203.2 [M+H]⁺.

(S)—N-hydroxy-3,4-dimethyl-2,3,4,5-tetrahydrobenzo[f][1,4]oxazepine-8-carboximidamide. To the mixture of (S)-3,4-dimethyl-2,3,4,5-tetrahydrobenzo[f][1,4]oxazepine-8-carbonitrile (350 mg, 1.722 mmol) and NH₂OH/H₂O (171 mg, 5.166 mmol) in EtOH (10 mL) at room temperature. The reaction mixture was stirred at 65° C. for 2 h. The mixture was evaporated to afford a crude. The crude was extracted with EA (50 mL×3). The combined organic layers were washed with brine (30 mL×2), dried over Na₂SO₄ and concentrated under reduced pressure to afford (S)—N-hydroxy-3,4-dimethyl-2,3,4,5-tetrahydrobenzo[f][1,4]oxazepine-8-carboximidamide (350 mg, 86%) as a yellow solid. MS (ESI): mass calcd. for C₁₂H₁₇N₃O₂. 235.13, m/z found 236.1 [M+H]⁺.

(S)-3,4-dimethyl-8-(5-(trifluoromethyl)-1,2,4-oxadiazol-3-yl)-2,3,4,5-tetrahydrobenzo[f][1,4]oxazepine. To the mixture of (S)—N-hydroxy-3,4-dimethyl-2,3,4,5-tetrahydrobenzo[f][1,4]oxazepine-8-carboximidamide (350 mg, 1.488 mmol) in DMF (dry, 10 mL) was added TFAA (937 mg, 4.463 mmol) and pyridine (353 mg, 4.463 mmol). The reaction mixture was stirred at 70° C. for 2 h under N₂. The mixture was cooled to the room temperature. The mixture was purified by Flash Chromatography with MeCN/0.1% TFA (35%) to afford (S)-3,4-dimethyl-8-(5-(trifluoromethyl)-1,2,4-oxadiazol-3-yl)-2,3,4,5-tetrahydrobenzo[f][1,4]oxazepine (350 mg, 75%) as a yellow oil. MS (ESI): mass calcd. for C₁₄H₁₄F₃N₃O₂. 313.10, m/z found 314.1 [M+H]⁺. ¹H NMR (400 MHz, DMSO-d₆) δ 7.97-7.57 (m, 3H), 4.57 (m, 3H), 4.16 (m, 1H), 3.79 (m, 1H), 2.78 (m, 3H), 1.37 (m, 3H).

### Example 1.36. Synthesis of (S)-3-methyl-4-(2,2,2-trifluoroethyl)-8-(5-(trifluoromethyl)-1,2,4-oxadiazol-3-yl)-2,3,4,5-tetrahydrobenzo[f][1,4]oxazepine (I-36)

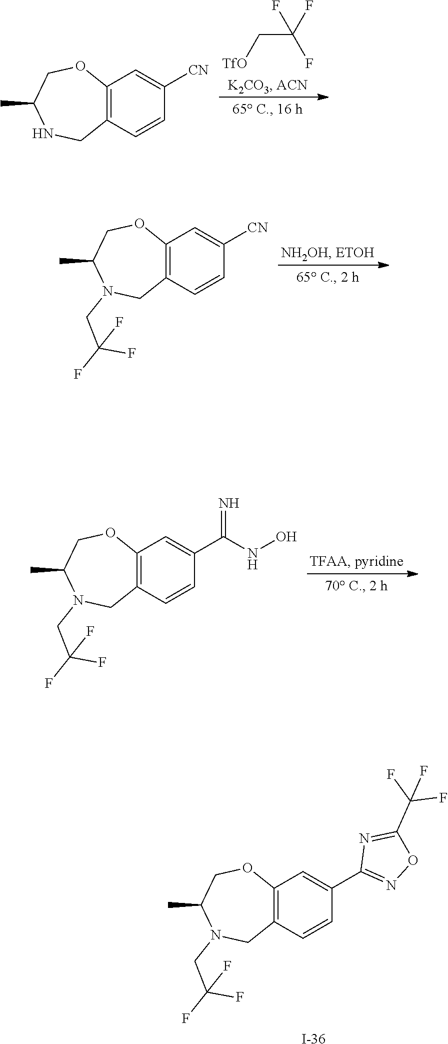

(S)-3-methyl-4-(2,2,2-trifluoroethyl)-2,3,4,5-tetrahydrobenzo[f][1,4]oxazepine-8-carbonitrile. To the mixture of (S)-3-methyl-2,3,4,5-tetrahydrobenzo[f][1,4]oxazepine-8-carbonitrile (400 mg, 2.13 mmol), K₂CO₃ (881 mg, 6.37 mmol) in ACN (15 mL) was added 2,2,2-trifluoroethyl trifluoromethanesulfonate (986 mg, 4.25 mmol). The mixture was stirred at 65° C. for 16 h. The mixture was quenched with water (40 mL) and extracted with EA (50 mL×3). The combined organic layers were washed with brine (30 mL×2), dried over Na₂SO₄ and concentrated under reduced pressure to afford crude product. The crude product was purified by Flash Chromatography with PE/EtOAC (0-50%) to afford (S)-3-methyl-4-(2,2,2-trifluoroethyl)-2,3,4,5-tetrahydrobenzo[f][1,4]oxazepine-8-carbonitrile (250 mg, 45%) as a white solid. MS (ESI): mass calcd. for C₁₃H₁₃F₃N₂O. 270.3, m/z found 271.1 [M+H]⁺.

(S)—N-hydroxy-3-methyl-4-(2,2,2-trifluoroethyl)-2,3,4,5-tetrahydrobenzo[f][1,4]oxazepine-8-carboximidamide. To the mixture of (S)-3-methyl-4-(2,2,2-trifluoroethyl)-2,3,4,5-tetrahydrobenzo[f][1,4]oxazepine-8-carbonitrile (250 mg, 0.93 mmol) in EtOH (10 mL) was added NH₂OH/H₂O (61 mg, 1.85 mmol) at room temperature. The reaction mixture was stirred at 65° C. for 2 h. The mixture was quenched with water (40 mL) and extracted with EA (50 mL×3). The combined organic layers were washed with brine (30 mL×2), dried over Na₂SO₄ and concentrated under reduced pressure to afford (S)—N-hydroxy-3-methyl-4-(2,2,2-trifluoroethyl)-2,3,4,5-tetrahydrobenzo[f][1,4]oxazepine-8-carboximidamide (200 mg, 71%) as a white solid. S (ESI): mass calcd. for C₁₃H₁₆F₃N₃O₂. 303.3, m/z found 304.1 [M+H]⁺.

(S)-3-methyl-4-(2,2,2-trifluoroethyl)-8-(5-(trifluoromethyl)-1,2,4-oxadiazol-3-yl)-2,3,4,5-tetrahydrobenzo[f][1,4]oxazepine. To the mixture of (S)—N-hydroxy-3-methyl-4-(2,2,2-trifluoroethyl)-2,3,4,5-tetrahydrobenzo[f][1,4]oxazepine-8-carboximidamide (170 mg, 0.56 mmol) in DMF (dry, 10 mL) was added TFAA (353 mg, 1.68 mmol) and pyridine (133 mg, 1.68 mmol). The reaction mixture was stirred at 70° C. for 2 h under N₂. The mixture was cooled to room temperature and concentrated under reduced pressure. The residue was purified by Flash Chromatography with MeCN/0.1% TFA (35%) to afford (S)-3-methyl-4-(2,2,2-trifluoroethyl)-8-(5-(trifluoromethyl)-1,2,4-oxadiazol-3-yl)-2,3,4,5-tetrahydrobenzo[f][1,4]oxazepine (100 mg, 50%) as a white solid. MS (ESI): mass calcd. for C₁₅H₁₃F₆N₃O₂. 381.3, m/z found 382.1 [M+H]. ¹H NMR (400 MHz, DMSO-d₆) δ 7.70-7.68 (m, 1H), 7.44-7.42 (m, 1H), 4.35-4.31 (m, 1H), 4.16-4.12 (m, 1H), 3.99-3.89 (m, 2H), 3.31-3.19 (m, 3H), 1.14-1.13 (m, 3H).

### Example 1.37. Synthesis of 6-fluoro-4-[(3-methyloxetan-3-yl)carbonyl]-8-[5-(trifluoromethyl)-1,2,4-oxadiazol-3-yl]-3,5-dihydro-2H-1,4-benzoxazepine (I-37)

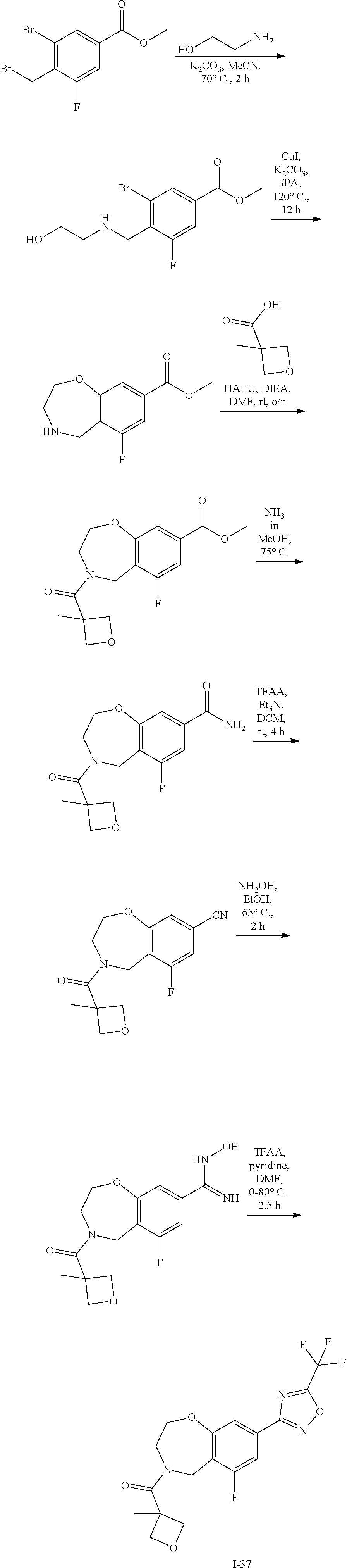

Methyl 3-bromo-5-fluoro-4-{[(2-hydroxyethyl)amino]methyl}benzoate. To a solution of methyl 3-bromo-4-(bromomethyl)-5-fluorobenzoate (1.0 g, 3.07 mmol) in ACN (10 mL) was added 2-aminoethanol (375 mg, 6.14 mmol) and K₂CO₃ (1.3 g, 9.20 mmol). The reaction mixture was stirred at 25° C. for 3 hours. The reaction mixture was filtered and the filtrate was concentrated. The residue was purified by flash chromatograph on silica gel column (PE:EA=3:1) to afford methyl 3-bromo-5-fluoro-4-{[(2-hydroxyethyl)amino]methyl}benzoate (800 mg, 77%) as yellow solid. MS (ESI): mass calcd. for C₁₁H₁₃BrFNO₃, 305.0, m/z found 306.0 [M+H]⁺.

Methyl 6-fluoro-2,3,4,5-tetrahydro-1,4-benzoxazepine-8-carboxylate. To a solution of methyl 3-bromo-5-fluoro-4-{[(2-hydroxyethyl)amino]methyl}benzoate (750 mg, 2.45 mmol) in i-PrOH (10 mL) was added Copper(I) iodide (47 mg, 0.25 mmol) and K₂CO₃ (1.0 g, 7.35 mmol). The reaction mixture was stirred at 80° C. for 16 hours. After cooling to room temperature, the reaction mixture was concentrated under reduced pressure. The residue was diluted with water (30 mL) and extracted with EA (50 mL×3). The organic layer was concentrated and the residue was purified by flash chromatograph on silica gel column (DCM:MeOH=10:1) to afford methyl 6-fluoro-2,3,4,5-tetrahydro-1,4-benzoxazepine-8-carboxylate (500 mg, 82%) as yellow solid. MS (ESI): mass calcd. for C₁₁H₁₂FNO₃, 225.1, m/z found 226.1[M+H]⁺.

Methyl 6-fluoro-4-[(3-methyloxetan-3-yl)carbonyl]-3,5-dihydro-2H-1,4-benzoxazepine-8-carboxylate. To a solution of methyl 6-fluoro-2,3,4,5-tetrahydro-1,4-benzoxazepine-8-carboxylate (450 mg, 2.00 mmol) and 3-methyloxetane-3-carboxylic acid (464 mg, 4.00 mmol) in DCM (10 mL) was added HATU (1.5 g, 4.00 mmol) and DIEA (775 mg, 5.99 mmol). The reaction mixture was stirred at 25° C. for 16 hours. The reaction mixture was concentrated under reduced pressure. The residue was purified by flash chromatograph on silica gel column (PE:EA=3:1) to afford methyl 6-fluoro-4-[(3-methyloxetan-3-yl)carbonyl]-3,5-dihydro-2H-1,4-benzoxazepine-8-carboxylate (300 mg, 42%) as yellow solid. MS (ESI): mass calcd. for C₁₆H₁₈FNO₅, 323.1, m/z found 324.0[M+H]⁺.

6-fluoro-4-[(3-methyloxetan-3-yl)carbonyl]-3,5-dihydro-2H-1,4-benzoxazepine-8-carboxamide. To a mixture of methyl 6-fluoro-4-[(3-methyloxetan-3-yl)carbonyl]-3,5-dihydro-2H-1,4-benzoxazepine-8-carboxylate (300 mg, 0.93 mmol) was added NH₃ in MeOH (10 mL). The reaction mixture was stirred at 75° C. for 16 hours. The reaction mixture was concentrated under reduced pressure to afford 6-fluoro-4-[(3-methyloxetan-3-yl)carbonyl]-3,5-dihydro-2H-1,4-benzoxazepine-8-carboxamide (240 mg, 76%) as yellow solid. MS (ESI): mass calcd. for C₁₅H₁₇FN₂O₄, 308.1, m/z found 309.1 [M+H]⁺.

6-fluoro-4-[(3-methyloxetan-3-yl)carbonyl]-3,5-dihydro-2H-1,4-benzoxazepine-8-carbonitrile. To a solution of 6-fluoro-4-[(3-methyloxetan-3-yl)carbonyl]-3,5-dihydro-2H-1,4-benzoxazepine-8-carboxamide (220 mg, 0.71 mmol) in DCM (10 mL) were added TFAA (450 mg, 2.14 mmol) and TEA (361 mg, 3.57 mmol). The reaction mixture was stirred at 25° C. for 3 hours. The mixture was concentrated and the residue was purified by flash chromatograph on silica gel column (PE:EA=3:1) to afford 6-fluoro-4-[(3-methyloxetan-3-yl)carbonyl]-3,5-dihydro-2H-1,4-benzoxazepine-8-carbonitrile (150 mg, 65%) as yellow solid. MS(ESI): mass calcd. for C₁₅H₁₅FN₂O₃, 290.1, m/z found 291.1 [M+H]⁺.

6-fluoro-N-hydroxy-4-[(3-methyloxetan-3-yl)carbonyl]-3,5-dihydro-2H-1,4-benzoxazepine-8-carboximidamide. To a solution of 6-fluoro-4-[(3-methyloxetan-3-yl)carbonyl]-3,5-dihydro-2H-1,4-benzoxazepine-8-carbonitrile (130 mg, 0.45 mmol) in EtOH (5 mL) were added NH₂OH/H₂O (44 mg, 1.34 mmol). The reaction mixture was stirred at 65° C. for 3 hours. After cooling to room temperature, the reaction mixture was concentrated under reduced pressure to afford 6-fluoro-N-hydroxy-4-[(3-methyloxetan-3-yl)carbonyl]-3,5-dihydro-2H-1,4-benzoxazepine-8-carboximidamide (100 mg, 62%) as yellow solid. MS (ESI): mass calcd. for C₁₅H₁₈FN₃O₄, 323.1, m/z found 324.1 [M+H]⁺.

6-fluoro-4-[(3-methyloxetan-3-yl)carbonyl]-8-[5-(trifluoromethyl)-1,2,4-oxadiazol-3-yl]-3,5-dihydro-2H-1,4-benzoxazepine. To a solution of 6-fluoro-N-hydroxy-4-[(3-methyloxetan-3-yl)carbonyl]-3,5-dihydro-2H-1,4-benzoxazepine-8-carboximidamide (200 mg, 0.62 mmol) in DMF (5 mL) was added TFAA (390 mg, 1.86 mmol) and pyridine (245 mg, 3.09 mmol). The reaction mixture was stirred at 80° C. for 3 hours. The mixture was concentrated and the residue was purified by Prep-HPLC (Instrument: Waters Prep-HPLC, Column: Xbridge prep c18 5 um OBD 19*150 mm, A water (0.1% FA), B Acetonitrile 10-20% B in 8 min, hold at 100% B at for 2 min, back to 5% B with 0.5 min, stop at 13 min, flow rate: 20 ml/min, wavelength: 214/254 nm, injection: 12) to afford 6-fluoro-4-[(3-methyloxetan-3-yl)carbonyl]-8-[5-(trifluoromethyl)-1,2,4-oxadiazol-3-yl]-3,5-dihydro-2H-1,4-benzoxazepine (70.4 mg, 28%) as a yellow oil. MS (ESI): mass calcd. for C₁₇H₁₅F₄N₃O₄, 401.1, m/z found 402.1 [M+H]⁺. ¹H NMR (400 MHz, CDCl₃) δ 7.66-7.50 (m, 2H), 5.01-4.92 (m, 2H), 4.83 (s, 1H), 4.50-4.20 (m, 5H), 4.05 (s, 1H), 3.50-3.43 (m, 1H), 1.67 (d, J=5.2 Hz, 3H).

### Example 1.38. Synthesis of 1-[(3S)-6-fluoro-3-methyl-8-[5-(trifluoromethyl)-1,2,4-oxadiazol-3-yl]-3,5-dihydro-2H-1,4-benzoxazepin-4-yl]-2-methylpropan-1-one (I-38)

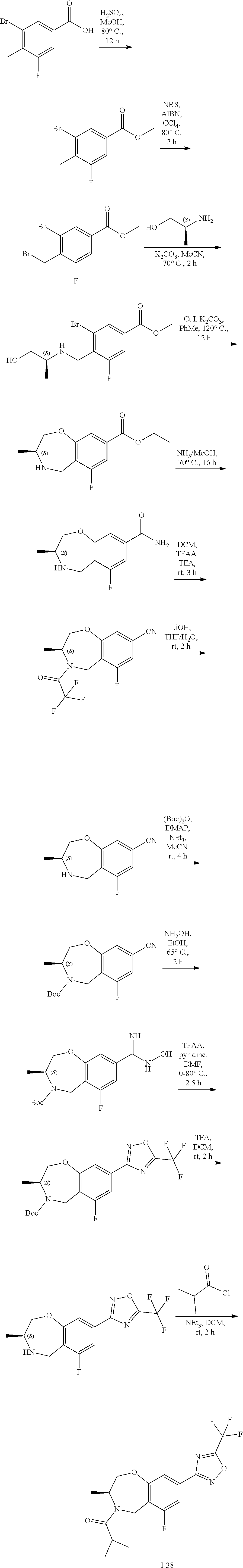

Methyl 3-bromo-5-fluoro-4-methylbenzoate. To a solution of 3-bromo-5-fluoro-4-methylbenzoic acid (50.0 g, 214.60 mmol) in MeOH (500 mL) was added H₂SO₄ (20 mL). The reaction mixture was stirred at 80° C. for 16 h under nitrogen atmosphere. The mixture was cooled to room temperature and concentrated under reduced pressure to get a crude which was purified by column chromatography on silica gel (330 g) eluting with EA in PE (0-5%) to afford methyl 3-bromo-5-fluoro-4-methylbenzoate (52.0 g, 88%) as yellow oil. MS (ESI): mass calcd. for C₉H₈BrFO₂, 246.0, m/z found 247.0 [M+H]⁺.

Methyl 3-bromo-4-(bromomethyl)-5-fluorobenzoate. To a solution of methyl 3-bromo-5-fluoro-4-methylbenzoate (52.0 g, 210.40 mmol) in CCl₄ (600 mL) was added NBS (41.2 g, 231.44 mmol) and AIBN (3.4 g, 21.14 mmol) at room temperature under nitrogen atmosphere. The reaction mixture was stirred at 80° C. for 16 h under nitrogen atmosphere. The mixture was quenched with water (200 mL) and concentrated under reduced pressure to remove CCl₄. The mixture was extracted with DCM (200 mL×3). The combine organic layer was dried over Na₂SO₄ and concentrated under reduced pressure to afford a crude. The crude was purified by column chromatography on silica gel (120 g) eluting with EA in PE (0-10%) to afford methyl 3-bromo-4-(bromomethyl)-5-fluorobenzoate (50.0 g, 66%) as yellow oil. MS (ESI): mass calcd. for C₉H₇Br₂FO₂, 323.9, m/z found 324.9 [M+H]⁺.

Methyl 3-bromo-5-fluoro-4-{[(2-hydroxyethyl)amino]methyl}benzoate. To a solution of methyl 3-bromo-4-(bromomethyl)-5-fluorobenzoate (24.0 g, 73.6 mmol) in ACN (300 mL) was added (2S)-2-aminopropan-1-ol (11.1 g, 147.25 mmol) and K₂CO₃ (30.5 g, 220.88 mmol) at room temperature under nitrogen atmosphere. The reaction mixture was stirred at room temperature for 3 h under nitrogen atmosphere. The mixture was concentrated under reduced pressure and extracted with EA (300 mL×3). The combine organic layer was dried over Na₂SO₄ and concentrated under reduced pressure to afford a crude. The crude was purified by column chromatography on silica gel (330 g) eluting with MeOH in DCM (0-5%) to afford methyl 3-bromo-5-fluoro-4-{[(2-hydroxyethyl)amino]methyl}benzoate (19.8 g, 82%) as a white solid. MS (ESI): mass calcd. for C₁₂H₁₅BrFNO₃, 319.0, m/z found 320.0 [M+H]⁺.

Isopropyl (3S)-6-fluoro-3-methyl-2,3,4,5-tetrahydro-1,4-benzoxazepine-8-carboxylate. To a solution of methyl 3-bromo-5-fluoro-4-({[(2S)-1-hydroxypropan-2-yl]amino}methyl)benzoate (19.8 g, 62.03 mmol) in ⁱ—PrOH (300 mL) was added Copper(I) iodide (1.2 g, 6.20 mmol) and K₂CO₃ (25.7 g, 186.10 mmol) at room temperature under nitrogen atmosphere. The reaction mixture was stirred at 80° C. for 16 h under nitrogen atmosphere. The mixture was cooled to room temperature and filtered. The filtrate was concentrated to get a residue which was purified by column chromatography on silica gel (330 g) eluting with EA in PE (0-50%) to afford isopropyl (3S)-6-fluoro-3-methyl-2,3,4,5-tetrahydro-1,4-benzoxazepine-8-carboxylate (12.0 g, 81%) as yellow oil. MS (ESI): mass calcd. for C₁₂H₁₄FNO₃, 239.10, m/z found 240.1 [M+H]⁺.

(3S)-6-fluoro-3-methyl-2,3,4,5-tetrahydro-1,4-benzoxazepine-8-carboxamide. To the liquid of isopropyl (3S)-6-fluoro-3-methyl-2,3,4,5-tetrahydro-1,4-benzoxazepine-8-carboxylate (12.0 g, 44.9 mmol) was added NH₃ in MeOH (32 mL, 225.0 mmol, 7M) at room temperature under nitrogen atmosphere. The reaction mixture was stirred at 70° C. for 24 h under nitrogen atmosphere. The mixture was concentrated under reduced pressure to get the required product (3S)-6-fluoro-3-methyl-2,3,4,5-tetrahydro-1,4-benzoxazepine-8-carboxamide (10.8 g, 91%) as green oil. MS (ESI): mass calcd. for C₁₁H₁₃FN₂O₂, 224.10, m/z found 225.1 [M+H]⁺.

(3S)-6-fluoro-3-methyl-4-(2,2,2-trifluoroacetyl)-3,5-dihydro-2H-1,4-benzoxazepine-8-carbonitrile. To a solution of (3S)-6-fluoro-3-methyl-2,3,4,5-tetrahydro-1,4-benzoxazepine-8-carboxamide (10.8 g, 48.2 mmol) and NEt₃ (14.6 g, 144.6 mmol) in DCM (100 mL) at 0° C. was added TFAA (30.3 g, 144.6 mmol). The reaction mixture was stirred at room temperature for 4 h under nitrogen atmosphere. The mixture was quenched with water (100 mL) and extracted with DCM (3×50 mL). The combined organic layer was dried over Na₂SO₄ and concentrated under reduced pressure to get the required product (3S)-6-fluoro-3-methyl-4-(2,2,2-trifluoroacetyl)-3,5-dihydro-2H-1,4-benzoxazepine-8-carbonitrile (9.9 g, 68%) as yellow oil. MS (ESI): mass calcd. for C₁₃H₁₀F₄N₂O₂, 302.07, m/z found 303.0 [M+H]⁺.

(3S)-6-fluoro-3-methyl-2,3,4,5-tetrahydro-1,4-benzoxazepine-8-carbonitrile. To a solution of (3S)-6-fluoro-3-methyl-4-(2,2,2-trifluoroacetyl)-3,5-dihydro-2H-1,4-benzoxazepine-8-carbonitrile (9.9 g, 32.8 mmol) in THF (40 mL) and H₂O (40 mL) was added Lithium hydroxide (3.2 g, 131.2 mmol). The reaction mixture was stirred at room temperature for 2 h. The mixture was adjusted to pH=2-3 with 1M HCl and extracted with EA (3×30 mL). The combined organic layer was dried over Na₂SO₄ and concentrated under reduced pressure to get the required product (3S)-6-fluoro-3-methyl-2,3,4,5-tetrahydro-1,4-benzoxazepine-8-carbonitrile (12.3 mg, 83%) as a white solid. MS (ESI): mass calcd. for C₁₁H₁₁FN₂O, 206.09, m/z found 207.1 [M+H]⁺.

Tert-butyl (3S)-8-cyano-6-fluoro-3-methyl-3,5-dihydro-2H-1,4-benzoxazepine-4-carboxylate. To a solution of (3S)-6-fluoro-3-methyl-2,3,4,5-tetrahydro-1,4-benzoxazepine-8-carbonitrile (3.0 g, 14.5 mmol), NEt₃ (4.4 g, 43.5 mmol) and Di-tert-butyl dicarbonate (6.33 g, 29.0 mmol) in MeCN (30 mL) stirred under nitrogen atmosphere at room temperature was added 4-Dimethylaminopyridine (0.18 g, 1.4 mmol). The reaction mixture was stirred at room temperature for 2 h under nitrogen atmosphere. The mixture was quenched with water (20 mL) and extracted with EA (20 mL×3). The combined organic layer was washed with brine (20 mL×3) and concentrated to get a residue which was purified by column chromatography on silica gel (40 g) eluting with EA in PE (0-50%) to tert-butyl (3S)-8-cyano-6-fluoro-3-methyl-3,5-dihydro-2H-1,4-benzoxazepine-4-carboxylate (750 mg, 15%) as a white solid. MS (ESI): mass calcd. for C₁₆H₁₉FN₂O₃, 306.14, m/z found 329.1 [M+Na]⁺.

Tert-butyl (S)-6-fluoro-8-(N-hydroxycarbamimidoyl)-3-methyl-2,3-dihydrobenzo[f][1,4]oxazepine-4(5H)-carboxylate. To a solution of tert-butyl (3S)-8-cyano-6-fluoro-3-methyl-3,5-dihydro-2H-1,4-benzoxazepin-4-yl formate (750 mg, 2.44 mmol) in EtOH (20 mL) was added NH₂OH/H₂O (1.6 g, 24.4 mmol) under nitrogen atmosphere. The reaction mixture was stirred at 65° C. for 2 h under nitrogen atmosphere. The mixture was cooled to room temperature and concentrated under reduced pressure to get the required product tert-butyl (S)-6-fluoro-8-(N-hydroxycarbamimidoyl)-3-methyl-2,3-dihydrobenzo[f][1,4]oxazepine-4(5H)-carboxylate (890 mg, 96%) as a white solid. MS (ESI): mass calcd. for C₁₆H₂₂FN₃O₄, 339.16, m/z found 340.2 [M+H]⁺.

Tert-butyl (S)-6-fluoro-3-methyl-8-(5-(trifluoromethyl)-1,2,4-oxadiazol-3-yl)-2,3-dihydrobenzo[f][1,4]oxazepine-4(5H)-carboxylate. To a solution of tert-butyl (3S)-6-fluoro-8-(N-hydroxycarbamimidoyl)-3-methyl-3,5-dihydro-2H-1,4-benzoxazepin-4-yl formate (0.89 g, 2.6 mmol) and pyridine (0.69 g, 8.7 mmol) in DMF (20 mL) was added TFAA (1.83 g, 8.7 mmol) at 0° C. under nitrogen atmosphere. The reaction mixture was stirred at 80° C. for 2 h under nitrogen atmosphere. The mixture was cooled to room temperature and concentrated under reduced pressure to get a residue which was purified by column chromatography on silica gel (25 g) eluting with EA in PE (0-30%) to tert-butyl (S)-6-fluoro-3-methyl-8-(5-(trifluoromethyl)-1,2,4-oxadiazol-3-yl)-2,3-dihydrobenzo[f][1,4]oxazepine-4(5H)-carboxylate (735 mg, 68%) as colourless oil. MS (ESI): mass calcd. for C₁₈H₁₉F₄N₃O₄, 417.13, m/z found 440.1 [M+Na]⁺.

(3S)-6-fluoro-3-methyl-8-[5-(trifluoromethyl)-1,2,4-oxadiazol-3-yl]-2,3,4,5-tetrahydro-1,4-benzoxazepine. The mixture of tert-butyl (3S)-6-fluoro-3-methyl-8-[5-(trifluoromethyl)-1,2,4-oxadiazol-3-yl]-3,5-dihydro-2H-1,4-benzoxazepin-4-yl formate (200 mg, 0.48 mmol) in HCl (5 mL, 4 M in EA) was stirred at room temperature for 2 h under nitrogen atmosphere. The mixture was quenched with water (10 mL) and extracted with EA (5 mL×3). The combined organic layer was washed with brine (10 mL×3) and concentrated to get the required product (3S)-6-fluoro-3-methyl-8-[5-(trifluoromethyl)-1,2,4-oxadiazol-3-yl]-2,3,4,5-tetrahydro-1,4-benzoxazepine (123 mg, 73%) as a white solid. MS (ESI): mass calcd. for C₁₃H₁₁F₄N₃O₂, 317.08, m/z found 318.1 [M+H]⁺.

1-[(3S)-6-fluoro-3-methyl-8-[5-(trifluoromethyl)-1,2,4-oxadiazol-3-yl]-3,5-dihydro-2H-1,4-benzoxazepin-4-yl]-2-methylpropan-1-one. To a solution of (3S)-6-fluoro-3-methyl-8-[5-(trifluoromethyl)-1,2,4-oxadiazol-3-yl]-2,3,4,5-tetrahydro-1,4-benzoxazepine (100 mg, 0.32 mmol) and NEt₃ (96 mg, 0.95 mmol) in DCM (10 mL) was added 2-methylpropanoyl chloride (51 mg, 0.47 mmol) at room temperature. The reaction mixture stirred at room temperature for 2 h under nitrogen atmosphere. The mixture was quenched with water (10 mL) and extracted with DCM (10 mL×3). The combined organic layer was washed with brine (10 mL×3) and concentrated to get a residue which was purified by column chromatography on silica gel (12 g) eluting with EA in PE (0-30%) to the required product 1-[(3S)-6-fluoro-3-methyl-8-[5-(trifluoromethyl)-1,2,4-oxadiazol-3-yl]-3,5-dihydro-2H-1,4-benzoxazepin-4-yl]-2-methylpropan-1-one (30.5 mg, 25%) as white oil. MS (ESI): mass calcd. for C₁₇H₁₇F₄N₃O₃, 387.12, m/z found 388.1 [M+H]⁺. ¹H NMR (400 MHz, CDCl₃) δ 7.55-7.42 (m, 2H), 5.75-3.93 (m, 5H), 2.90-2.66 (m, 1H), 1.32 (d, J=6.8 Hz, 1H), 1.25-1.16 (m, 5H), 1.13 (d, J=6.8 Hz, 1H), 0.91 (d, J=6.8 Hz, 2H).

### Example 1.39. Synthesis of (3S)-6-fluoro-3-methyl-4-[(oxan-4-yl)carbonyl]-8-[5-(trifluoromethyl)-1,2,4-oxadiazol-3-yl]-3,5-dihydro-2H-1,4-benzoxazepine (I-39)

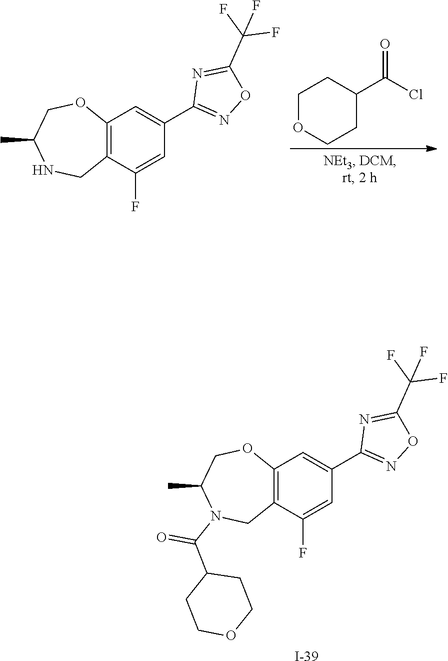

To a solution of (3S)-6-fluoro-3-methyl-8-[5-(trifluoromethyl)-1,2,4-oxadiazol-3-yl]-2,3,4,5-tetrahydro-1,4-benzoxazepine (140 mg, 0.44 mmol) and NEt₃ (90 mg, 0.88 mmol) in DCM (10 mL) was added oxane-4-carbonyl chloride (85 mg, 0.57 mmol) at 0° C. under nitrogen atmosphere. The reaction mixture was stirred at room temperature for 2 h under nitrogen atmosphere. The mixture was quenched with water (20 mL) and extracted with DCM (10 mL×3). The combined organic layer was dried over Na₂SO₄ and concentrated under reduced pressure to get a residue which was purified by column chromatography on silica gel (12 g) eluting with EA in PE (0-30%) to afford (3S)-6-fluoro-3-methyl-4-[(oxan-4-yl)carbonyl]-8-[5-(trifluoromethyl)-1,2,4-oxadiazol-3-yl]-3,5-dihydro-2H-1,4-benzoxazepine (69.8 mg, 37%) as a white solid. MS (ESI): mass calcd. for C₁₉H₁₉F₄N₃O₄, 429.13, m/z found 430.1 [M+H]⁺. ¹H NMR (400 MHz, CDCl₃) δ 7.59-7.31 (m, 2H), 5.63-3.85 (m, 7H), 3.52-3.26 (m, 2H), 2.80-2.53 (m, 1H), 2.10-1.57 (m, 4H), 1.32 (d, J=6.4 Hz, 1H), 1.22 (d, J=6.8 Hz, 2H).

### Example 1.40. Synthesis of 1-[(3S)-6-fluoro-3-methyl-8-[5-(trifluoromethyl)-1,2,4-oxadiazol-3-yl]-3,5-dihydro-2H-1,4-benzoxazepin-4-yl]-2-hydroxyethanone (I-40)

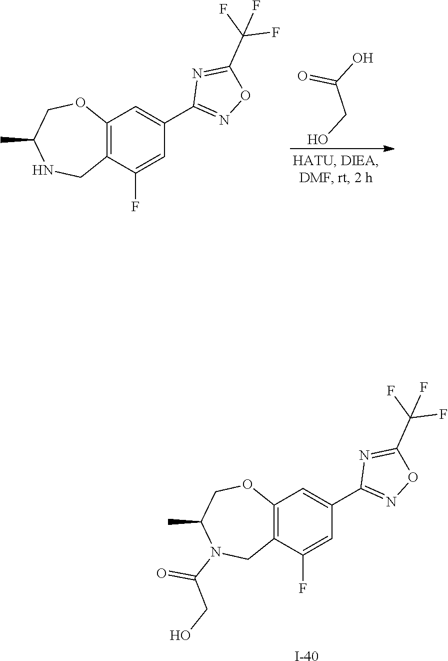

To a solution of (3S)-6-fluoro-3-methyl-8-[5-(trifluoromethyl)-1,2,4-oxadiazol-3-yl]-2,3,4,5-tetrahydro-1,4-benzoxazepine (100 mg, 0.32 mmol) and hydroxyacetic acid (29 mg, 0.0.38 mmol) in DMF (5 mL) was added DIEA (122 mg, 0.95 mmol) and HATU (240 mg, 0.63 mmol) at room temperature. The reaction mixture stirred at room temperature for 4 h under nitrogen atmosphere. The mixture was purified via prep-HPLC (Instrument: Waters MS-triggered Prep-LC with QDA detector, Column: WELCH Xtimate C18 21.2*250 mm 10 um, A H₂O (0.1% FA), B Acetonitrile 52-62% B in 9 min, hold at 100% B for 1 min, back to 52% B with 1.5 min, stop at 15 min, flow rate: 25 mL/min, wavelength: 214/254 nm, injection: 7) to give 1-[(3S)-6-fluoro-3-methyl-8-[5-(trifluoromethyl)-1,2,4-oxadiazol-3-yl]-3,5-dihydro-2H-1,4-benzoxazepin-4-yl]-2-hydroxyethanone (22.8 mg, 19%) as colourless oil. MS (ESI): mass calcd. for C₁₅H₁₃F₄N₃O₄, 375.08, m/z found 376.0 [M+H]⁺. ¹H NMR (400 MHz, CDCl₃) δ 7.56-7.42 (m, 2H), 5.88-3.90 (m, 7H), 2.40 (s, 1H), 1.36-1.24 (m, 3H).

### Example 1.41. Synthesis of 1-[(3S)-6-fluoro-3-methyl-8-[5-(trifluoromethyl)-1,2,4-oxadiazol-3-yl]-3,5-dihydro-2H-1,4-benzoxazepin-4-yl]-2-methoxyethanone (I-41)

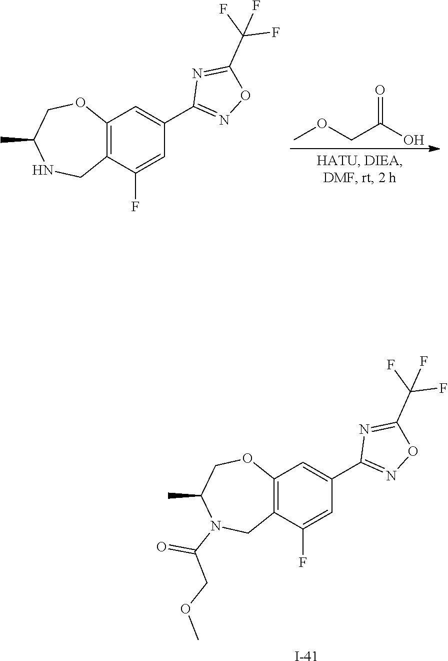

To a solution of (3S)-6-fluoro-3-methyl-8-[5-(trifluoromethyl)-1,2,4-oxadiazol-3-yl]-2,3,4,5-tetrahydro-1,4-benzoxazepine (120 mg, 0.38 mmol) and methoxyacetic acid (45 mg, 0.49 mmol) in DMF (10 mL) was added DIEA (147 mg, 1.13 mmol) and HATU (216 mg, 0.57 mmol) at room temperature. The reaction mixture stirred at room temperature for 2 h under nitrogen atmosphere.

The mixture was quenched with water (20 mL) and extracted with EA (15 mL×3). The combined organic layer was dried over Na₂SO₄ and concentrated under reduced pressure to get a residue which was purified by column chromatography on silica gel (12 g) eluting with EA in PE (0-30%) to afford 1-[(3S)-6-fluoro-3-methyl-8-[5-(trifluoromethyl)-1,2,4-oxadiazol-3-yl]-3,5-dihydro-2H-1,4-benzoxazepin-4-yl]-2-methoxyethanone (83.8 mg, 57%) as a white solid. MS (ESI): mass calcd. for C₁₆H₁₅F₄N₃O₄, 389.10, m/z found 390.1 [M+H]⁺. ¹H NMR (400 MHz, CDCl₃) δ 7.52-7.41 (m, 2H), 5.64-4.02 (m, 7H), 3.40 (s, 1H), 3.23 (s, 2H), 1.32 (d, J=6.4 Hz, 1H), 1.24 (d, J=6.8 Hz, 2H).

### Example 1.42. Synthesis of (S)-3-(dimethylamino)-1-(6-fluoro-3-methyl-8-(5-(trifluoromethyl)-1,2,4-oxadiazol-3-yl)-2,3-dihydrobenzo[f][1,4]oxazepin-4(5H)-yl)propan-1-one (I-42)

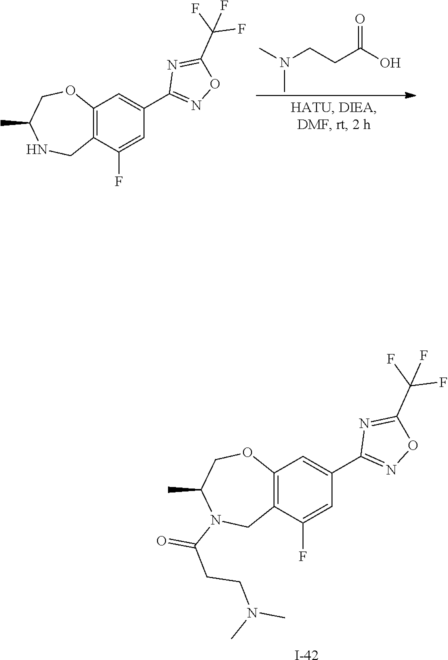

To a solution of (3S)-6-fluoro-3-methyl-8-[5-(trifluoromethyl)-1,2,4-oxadiazol-3-yl]-2,3,4,5-tetrahydro-1,4-benzoxazepine (150 mg, 0.47 mmol) and 3-(dimethylamino)propanoic acid (72 mg, 0.61 mmol) in DMF (3 mL) was added N,N,N′,N′-Tetramethyl-O-(7-azabenzotriazol-1-yl)uranium (360 mg, 0.95 mmol) and N,N-Diisopropylethylamine (183 mg, 1.42 mmol) under N₂. The reaction mixture was stirred at 25° C. for 1 h. The reaction mixture was quenched with water (10 mL) and extracted with EtOAc (15 mL×3). The combined organic layer was washed with brine (20 mL), dried over Na₂SO₄, concentrated to get the residue. The residue was purified by prep-TLC (MeOH/DCM=1/12, rf=0.6), fraction with MS signal of desired product was collected and concentrated to get the crude. The crude was purified by prep-HPLC (Chromatographic columns: Xbridge 5 u-C18 150×19 mm, 5 um Mobile Phase A: ACN-—H₂O, B (Acetonitrile), flow rate: 20 mL/min, wavelength: 214/254 nm, injection: 6) to get (S)-3-(dimethylamino)-1-(6-fluoro-3-methyl-8-(5-(trifluoromethyl)-1,2,4-oxadiazol-3-yl)-2,3-dihydrobenzo[f][1,4]oxazepin-4(5H)-yl)propan-1-one (17.5 mg, 9%) as a yellow oil. MS (ESI): mass calcd. for C₁₈H₂₀NF4N₄O₃ 416.15, m/z found 417.1 [M+H]⁺. ¹H NMR (400 MHz, MeOD) δ 7.60-7.29 (m, 2H), 5.54-4.28 (m, 5H), 3.12-2.89 (m, 3H), 2.85-2.35 (m, 7H), 1.34-1.18 (m, 3H).

### Example 1.43. Synthesis of (S)-2-(dimethylamino)-1-(6-fluoro-3-methyl-8-(5-(trifluoromethyl)-1,2,4-oxadiazol-3-yl)-2,3-dihydrobenzo[f][1,4]oxazepin-4(5H)-yl)ethan-1-one (I-43)

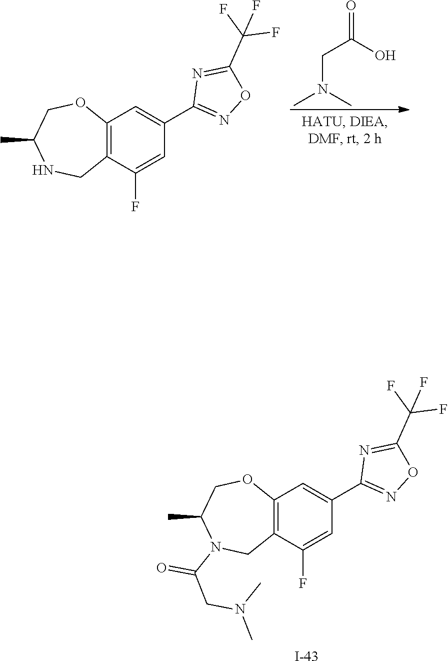

To a solution of (3S)-6-fluoro-3-methyl-8-[5-(trifluoromethyl)-1,2,4-oxadiazol-3-yl]-2,3,4,5-tetrahydro-1,4-benzoxazepine (150 mg, 0.47 mmol) and (dimethylamino)acetic acid (63 mg, 0.61 mmol) in DMF (3 mL) was added N,N,N′,N′-Tetramethyl-O-(7-azabenzotriazol-1-yl)uranium (360 mg, 0.95 mmol) and N,N-Diisopropylethylamine (183 mg, 1.42 mmol) under N₂. The reaction mixture was stirred at 25° C. for 1 h. The reaction mixture was quenched with water (10 mL) and extracted with EtOAc (15 mL×3). The combined organic layer was washed with brine (20 mL), dried over Na₂SO₄, concentrated to get the residue. The residue was purified by prep-TLC (MeOH/DCM=1/12, rf=0.6), fraction with MS signal of desired product was collected and concentrated to get (S)-2-(dimethylamino)-1-(6-fluoro-3-methyl-8-(5-(trifluoromethyl)-1,2,4-oxadiazol-3-yl)-2,3-dihydrobenzo[f][1,4]oxazepin-4(5H)-yl)ethan-1-one (37.6 mg, 20%) as a yellow oil. MS (ESI): mass calcd. for C₁₇H₁₈F₄N₄O₃ 402.35, m/z found 403.1 [M+H]⁺. ¹H NMR (400 MHz, CD₃OD) δ 7.39-7.27 (m, 2H), 5.39-4.17 (m, 5H), 3.55-3.10 (m, 1H), 3.03 (s, 1H), 2.20-1.95 (m, 6H), 1.22-1.04 (m, 3H).

### Example 1.44. Synthesis of (3S)-4-benzoyl-6-fluoro-3-methyl-8-[5-(trifluoromethyl)-1,2,4-oxadiazol-3-yl]-3,5-dihydro-2H-1,4-benzoxazepine (I-44)

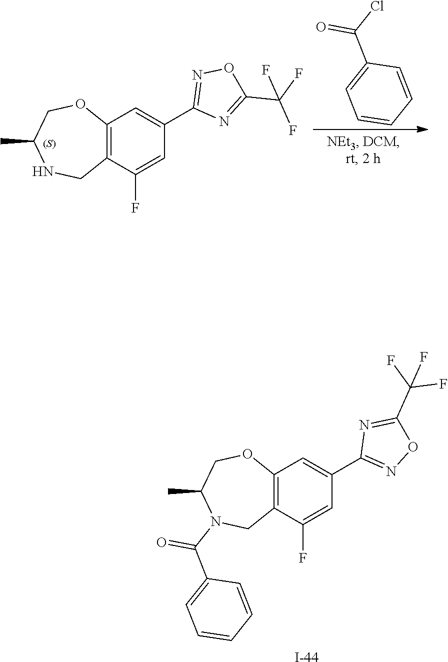

Benzoyl chloride (88 mg, 0.64 mmol) and NEt₃ (192 mg, 1.9 mmol) were added to the (3S)-6-fluoro-3-methyl-8-[5-(trifluoromethyl)-1,2,4-oxadiazol-3-yl]-2,3,4,5-tetrahydro-1,4-benzoxazepine (100 mg, 0.32 mmol) in DCM (10 mL). The mixture was stirred at 25° C. for 2 hours. The mixture was quenched with water (30 mL) and extracted with EA (20 mL×3). The combined organic layers were washed with brine (20 mL×2), dried over Na₂SO₄, concentrated under reduced pressure to afford crude product. The crude product was purified by silica gel column chromatography eluted with PE/EA to afford product is (3S)-4-benzoyl-6-fluoro-3-methyl-8-[5-(trifluoromethyl)-1,2,4-oxadiazol-3-yl]-3,5-dihydro-2H-1,4-benzoxazepine (60 mg, 45%) as a white solid. MS (ESI): mass calcd. for C₂₀H₁₅F₄N₃O₃, 421.4, m/z found 422.1 [M+H]⁺. ¹H NMR (400 MHz, CDCl₃) δ 7.55-7.16 (m, 7H), 5.66-4.23 (m, 5H), 1.33-1.25 (m, 3H).

### Example 1.45. Synthesis of (3S)-6-fluoro-3-methyl-4-[(pyridin-3-yl)carbonyl]-8-[5-(trifluoromethyl)-1,2,4-oxadiazol-3-yl]-3,5-dihydro-2H-1,4-benzoxazepine (I-45)

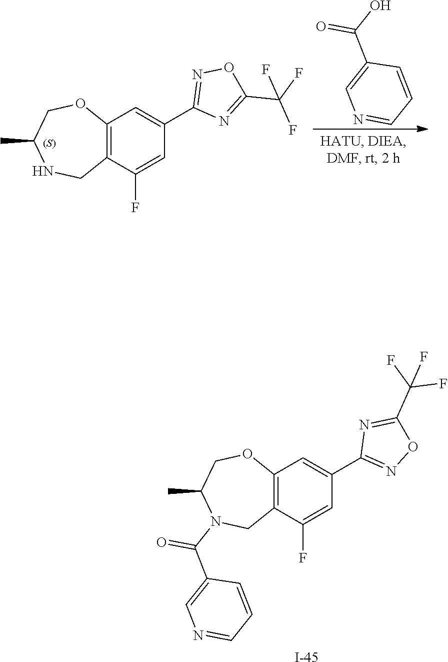

N,N,N′,N′-Tetramethyl-O-(7-azabenzotriazol-1-yl)uronium (60 mg, 0.16 mmol) and N,N-Diisopropylethylamine (102 mg, 0.79 mmol) and pyridine-4-carboxylic acid (39 mg, 0.32 mmol) were added to the mixture of (3S)-6-fluoro-3-methyl-8-[5-(trifluoromethyl)-1,2,4-oxadiazol-3-yl]-2,3,4,5-tetrahydro-1,4-benzoxazepine (50 mg, 0.16 mmol) in DMF (10 mL). The mixture was stirred at 25° C. for 2 hours. The mixture was quenched with water (30 mL) and extracted with EA (20 mL×3). The combined organic layers were washed with brine (20 mL×2), dried over Na₂SO₄, concentrated under reduced pressure to afford crude product. The crude product was purified by silica gel column chromatography eluted with PE/EA to afford product is (3S)-6-fluoro-3-methyl-4-[(pyridin-3-yl)carbonyl]-8-[5-(trifluoromethyl)-1,2,4-oxadiazol-3-yl]-3,5-dihydro-2H-1,4-benzoxazepine (60 mg, 45%) as a white solid. MS (ESI): mass calcd. for C₁₉H₁₄F₄N₄O₃, 422.3, m/z found 423.0 [M+H]⁺. ¹H NMR (400 MHz, CDCl₃) δ 8.65-8.48 (m, 2H), 7.51-7.19 (m, 4H), 5.51-4.20 (m, 5H), 1.28-1.18 (m, 3H).

### Example 1.46. Synthesis of 1-[(3S)-6-fluoro-3-methyl-8-[5-(trifluoromethyl)-1,2,4-oxadiazol-3-yl]-3,5-dihydro-2H-1,4-benzoxazepin-4-yl]-2-(pyridin-4-yl) ethenone (I-46)

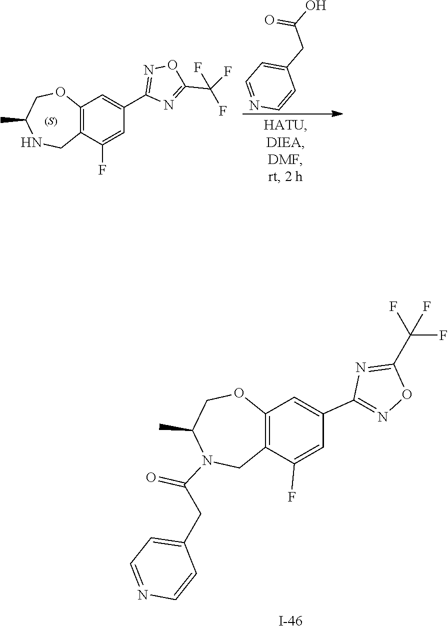

N,N-Diisopropylethylamine (204 mg, 1.58 mmol) and N,N,N′,N′-Tetramethyl-O-(7-azabenzotriazol-1-yl)uronium (120 mg, 0.32 mmol) and pyridin-4-ylacetic acid (86 mg, 0.63 mmol) were added to the mixture of (3S)-6-fluoro-3-methyl-8-[5-(trifluoromethyl)-1,2,4-oxadiazol-3-yl]-2,3,4,5-tetrahydro-1,4-benzoxazepine (100 mg, 0.32 mmol) in DMF (10 mL). The mixture was stirred at 25° C. for 2 hours. The mixture was quenched with water (30 mL) and extracted with EA (20 mL×3). The combined organic layers were washed with brine (20 mL×2), dried over Na₂SO₄, concentrated under reduced pressure to afford crude product. The crude product was purified by silica gel column chromatography eluted with PE/EA to afford product is 1-[(3S)-6-fluoro-3-methyl-8-[5-(trifluoromethyl)-1,2,4-oxadiazol-3-yl]-3,5-dihydro-2H-1,4-benzoxazepin-4-yl]-2-(pyridin-4-yl) ethenone (91 mg, 66%) as a white solid. MS (ESI): mass calcd. for C₂₀H₁₆F₄N₄O₃, 436.4, m/z found 437.1 [M+H]⁺. ¹H NMR (400 MHz, MeOD) δ 8.45 (d, J=8.0 Hz, 1H), 8.28 (d, J=4.0 Hz, 1H), 7.49-7.31 (m, 3H), 7.18 (d, J=8.0 Hz, 1H), 5.53-3.83 (m, 7H), 1.32-1.22 (m, 3H).

### Example 1.47. Synthesis of (3-methyloxetan-3-yl)(8-(5-(trifluoromethyl)-1,2,4-oxadiazol-3-yl)-2H-spiro[benzo[f][1,4]oxazepine-3,1′-cyclopropan]-4(5H)-yl)methanone (I-47)

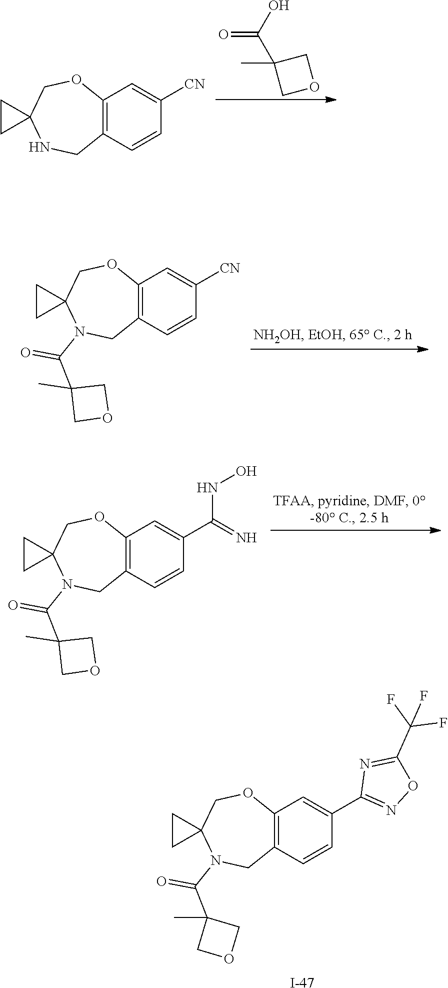

4-(3-methyloxetane-3-carbonyl)-4,5-dihydro-2H-spiro[benzo[f][1,4]oxazepine-3,1′-cyclopropane]-8-carbonitrile. To a solution of 4,5-dihydro-2H-spiro[benzo[f][1,4]oxazepine-3,1′-cyclopropane]-8-carbonitrile (100 mg, 0.49 mmol) and 3-methyloxetane-3-carboxylic acid (86 mg, 0.74 mmol) in DMF (10 mL) was added DMTMM (293 mg, 0.99 mmol). The reaction mixture was stirred at 25° C. for 18 h. The reaction mixture was concentrated in vacuo, purified by anti-phase using CH₃CN: H₂O to obtain 4-(3-methyloxetane-3-carbonyl)-4,5-dihydro-2H-spiro[benzo[f][1,4]oxazepine-3,1′-cyclopropane]-8-carbonitrile (51 mg, 30%) as a yellow solid. MS (ESI): mass calcd. for C₁₇H₁₈N₂O₃, 298.13, m/z found 299.1 [M+H]⁺

N-hydroxy-4-(3-methyloxetane-3-carbonyl)-4,5-dihydro-2H-spiro[benzo[f][1,4]oxazepine-3,1′-cyclopropane]-8-carboximidamide. To a solution of 4-(3-methyloxetane-3-carbonyl)-4,5-dihydro-2H-spiro[benzo[f][1,4]oxazepine-3,1′-cyclopropane]-8-carbonitrile (50 mg, 0.16 mmol) in EtOH (3 mL) was added hydroxylamine (16 mg, 0.50 mmol). The reaction mixture was stirred at 65° C. for 2 h. The reaction mixture was concentrated in vacuo to obtain the crude product N-hydroxy-4-(3-methyloxetane-3-carbonyl)-4,5-dihydro-2H-spiro[benzo[f][1,4]oxazepine-3,1′-cyclopropane]-8-carboximidamide (50 mg, crude) as a white solid. MS (ESI): mass calcd. for C₁₇H₂₁N₃O₄, 331.15, m/z found 332.2 [M+H]⁺

To a solution of N-hydroxy-4-(3-methyloxetane-3-carbonyl)-4,5-dihydro-2H-spiro[benzo[f][1,4]oxazepine-3,1′-cyclopropane]-8-carboximidamide (50 mg, crude), pyridine (47 mg, 0.60 mmol) in DMF (11 mL) stirred under nitrogen at 0° C. was added 2,2,2-trifluoroacetyl 2,2,2-trifluoroacetate (95 mg, 0.45 mmol). The reaction mixture was stirred at 80° C. for 2 h. The solution was concentrated in vacuo, purified by C₁₈-column using CH₃CN: H₂O to obtain (3-methyloxetan-3-yl)(8-(5-(trifluoromethyl)-1,2,4-oxadiazol-3-yl)-2H-spiro[benzo[f][1,4]oxazepine-3,1′-cyclopropan]-4(5H)-yl)methanone (3.84 mg, 6%) as a white solid. MS (ESI): mass calcd. for C₁₉H₁₈F₃N₃O₄, 409.12, m/z found 410.0 [M+H]⁺. ¹H NMR (400 MHz, MeOD) δ 7.75-7.73 (m, 1H), 7.66 (s, 1H), 7.42-7.29 (m, 1H), 4.85-4.80 (m, 2H), 4.29 (s, 4H), 3.93 (s, 2H), 1.54 (s, 3H), 0.99 (d, J=7.2 Hz, 4H).

### Example 1.48. Synthesis of (S)-1-(4-(6-fluoro-3-methyl-8-(5-(trifluoromethyl)-1,2,4-oxadiazol-3-yl)-2,3,4,5-tetrahydrobenzo[f][1,4]oxazepine-4-carbonyl)-4-methylpiperidin-1-yl)ethan-1-one (I-48)

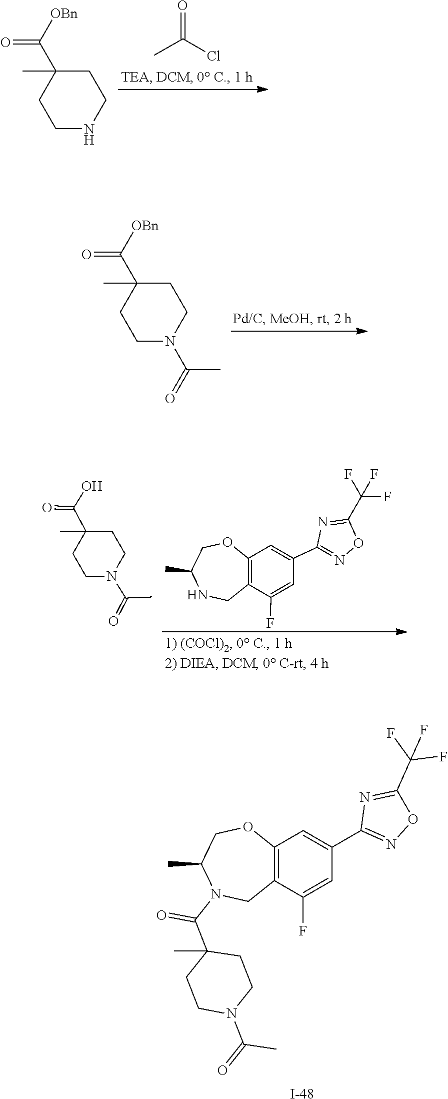

Benzyl 1-acetyl-4-methylpiperidine-4-carboxylate. To a solution of benzyl 4-methylpiperidine-4-carboxylate (3.0 g, 12.9 mmol) in DCM (30 mL) was added TEA (3.9 g, 38.7 mmol), acetyl chloride (1.5 g, 19.3 mmol) at ice bath. The reaction mixture was stirred at 25° C. for 2 h. The reaction mixture was quenched by H₂O (50 mL), extracted with DCM (50 mL*2). The organic layer was washed with brine (50 mL), dried over anhydrous Na₂SO₄, concentrated. The residue was purified by silica gel column chromatography eluted with PE/EA (3:1) to afford benzyl 1-acetyl-4-methylpiperidine-4-carboxylate (2.0 g, 53%) as a white solid. MS (ESI): mass calcd. for C₁₆H₂₁NO₃, 275.15, m/z found 276.1 [M+H]⁺.

1-acetyl-4-methylpiperidine-4-carboxylic acid. To a solution of benzyl 1-acetyl-4-methylpiperidine-4-carboxylate (1.0 g, 3.6 mmol) in MeOH (10 mL) was added Pd/C (190 mg, 1.8 mmol). The reaction mixture was stirred at 25° C. for 2 h under H₂ atmosphere. The mixture was filtered through celite, and the filtrate was concentrated to afford 1-acetyl-4-methylpiperidine-4-carboxylic acid (600 mg, 86%) as a white solid. MS (ESI): mass calcd. for C₉H₁₅NO₃, 185.11, m/z found 186.1 [M+H]⁺.

(S)-1-(4-(6-fluoro-3-methyl-8-(5-(trifluoromethyl)-1,2,4-oxadiazol-3-yl)-2,3,4,5-tetrahydrobenzo[f][1,4]oxazepine-4-carbonyl)-4-methylpiperidin-1-yl)ethan-1-one. To a solution of 1-acetyl-4-methylpiperidine-4-carboxylic acid (233 mg, 1.26 mmol) in DCM (10 mL) was added (COCl)₂ (160 mg, 1.26 mmol), DMF (18 mg, 0.25 mmol) at ice bath under N₂ atmosphere. The reaction mixture was stirred for 1 h at 0° C., then concentrated. The residue was dissolved in DCM (10 mL), added (3S)-6-fluoro-3-methyl-8-[5-(trifluoromethyl)-1,2,4-oxadiazol-3-yl]-2,3,4,5-tetrahydro-1,4-benzoxazepine (80 mg, 0.25 mmol), DIEA (326 mg, 2.52 mmol) at ice bath. The reaction mixture was stirred at 25° C. for 15 h. The reaction mixture was quenched with H₂O (20 mL) and extracted with DCM (20 mL*2). The organic layer was washed with brine (30 mL), dried over Na₂SO₄, filtered, the filtrate was concentrated to give a residue which was purified by silica gel column chromatography eluted with DCM:EA (2:1) to afford (S)-1-(4-(6-fluoro-3-methyl-8-(5-(trifluoromethyl)-1,2,4-oxadiazol-3-yl)-2,3,4,5-tetrahydrobenzo[f][1,4]oxazepine-4-carbonyl)-4-methylpiperidin-1-yl)ethan-1-one (16.8 mg, 13%) as a white solid. MS (ESI): mass calcd. for C₂₂H₂₄F₄N₄O₄, 484.17, m/z found 485.1 [M+H]⁺. ¹H NMR (400 MHz, dmso) δ 7.46 (d, J=9.6 Hz, 1H), 7.29 (s, 1H), 5.08-4.98 (m, 1H), 4.81-4.72 (m, 1H), 4.48-4.25 (m, 3H), 2.47-2.42 (m, 1H), 2.30-1.85 (m, 8H), 1.62-1.49 (m, 1H), 1.44-1.36 (m, 1H), 1.22-1.10 (m, 6H).

### Example 1.49. Synthesis of (3S)-6-fluoro-3-methyl-4-[(pyridin-4-yl)carbonyl]-8-[5-(trifluoromethyl)-1,2,4-oxadiazol-3-yl]-3,5-dihydro-2H-1,4-benzoxazepine (I-49)

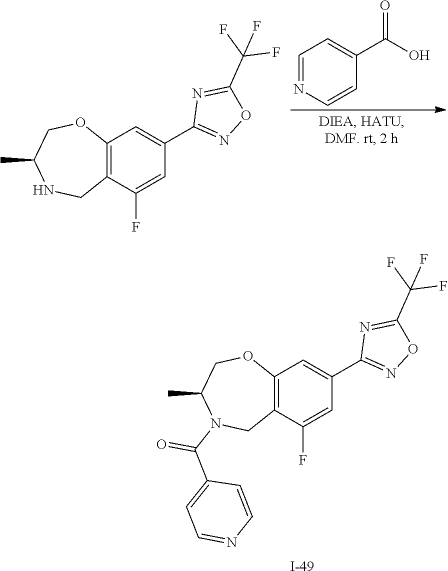

To a solution of (3S)-6-fluoro-3-methyl-8-[5-(trifluoromethyl)-1,2,4-oxadiazol-3-yl]-2,3,4,5-tetrahydro-1,4-benzoxazepine (150 mg, 0.473 mmol) and pyridine-4-carboxylic acid (88 mg, 0.709 mmol) in DMF (5 mL) were added N,N,N′,N′-Tetramethyl-O-(7-azabenzotriazol-1-yl)uranium (360 mg, 0.946 mmol) and N,N-Diisopropylethylamine (184 mg, 1.418 mmol). The mixture was stirred at 20° C. for 2 h. The mixture was diluting with water (15 mL) and extracted with DCM (5 mL×3), dried over Na₂SO₄ and filtered. The filtrate was concentrated in reduced pressure vacuum to give a residue which was purified by flash column with PE:EA (4:1) to afford (3S)-6-fluoro-3-methyl-4-[(pyridin-4-yl)carbonyl]-8-[5-(trifluoromethyl)-1,2,4-oxadiazol-3-yl]-3,5-dihydro-2H-1,4-benzoxazepine (56 mg, 28%) as a white solid. MS (ESI): mass calcd. for C₁₉H₁₄F₄N₄O₃, 422.10, m/z found 423 [M+H]⁺. ¹H NMR (400 MHz, CDCl₃) δ 8.79-8.62 (m, 2H), 7.62-7.38 (m, 3H), 7.19 (d, J=4.8 Hz, 1H), 5.75-5.13 (m, 1H), 4.54 (q, J=17.2 Hz, 1H), 4.42-4.05 (m, 3H), 1.39-1.25 (m, 3H).

### Example 1.50. Synthesis of 2-methyl-8-[5-(trifluoromethyl)-1,2,4-oxadiazol-3-yl]-4,5-dihydro-2H-1,4-benzoxazepin-3-one (I-50)

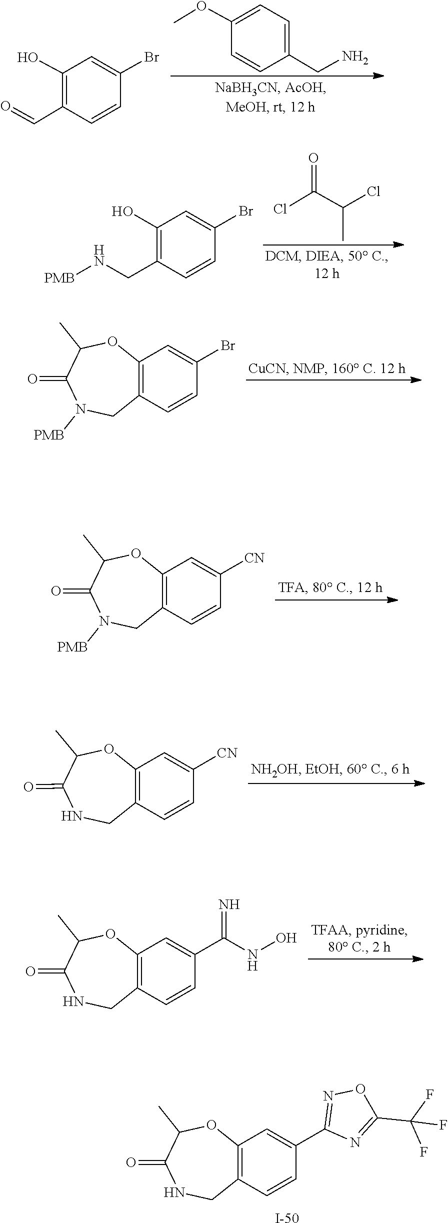

5-bromo-2-{[(2-methoxy-5-methylphenyl)amino]methyl}phenol. To a solution of 4-bromo-2-hydroxybenzaldehyde (10 g, 0.0497 mmol) in MeOH (20 mL) was added (4-ethylphenyl)methanamine (6.72 g, 0.0497 mmol), AcOH (10 mL, 0.00497) and NaBH₃CN (12.49 g, 0.1988 mmol). The mixture was stirred at 50° C. for 12 h. The mixture was quenched with ice water (40 mL) and extracted with EA (3×20 mL). The combine organic layer was dried over Na₂SO₄ and concentrated under reduced pressure to afford crude which was purified by silica gel column chromatography eluted with PE/EA (5:1) to afford 5-bromo-2-{[(2-methoxy-5-methylphenyl)amino]methyl}phenol (5 g, 29%) as a yellow solid. MS (ESI): mass calcd. for C₁₅H₁₆BrNO₂, 322.2, m/z found 323.2 [M+H]⁺.

8-bromo-4-(2-methoxy-5-methylphenyl)-2-methyl-2,5-dihydro-1,4-benzoxazepin-3-one. To a solution of 5-bromo-2-{[(2-methoxy-5-methylphenyl)amino]methyl}phenol (5 g, 0.0155 mmol) in DCM (100 mL) were added DIEA (6.01 g, 0.0465 mmol) and 2-chloropropanoyl chloride (1.97 g, 0.0155 mmol). The mixture was stirred at 25° C. for 12 h. The mixture was quenched with ice water (100 mL) and extracted with DCM (3×50 mL). The combine organic layer was dried over Na₂SO₄ and concentrated under reduced pressure to afford crude which was purified by silica gel column chromatography eluted with PE/EA (5:1) to afford 8-bromo-4-(2-methoxy-5-methylphenyl)-2-methyl-2,5-dihydro-1,4-benzoxazepin-3-one (5 g, 77%) as a yellow solid. MS (ESI): mass calcd. for C₁₈H₁₈BrNO₃, 376.2, m/z found 377.2 [M+H]

4-(2-methoxy-5-methylphenyl)-2-methyl-3-oxo-2,5-dihydro-1,4-benzoxazepine-8-carbonitrile. To a solution of 8-bromo-4-(2-methoxy-5-methylphenyl)-2-methyl-2,5-dihydro-1,4-benzoxazepin-3-one (1 g, 2.7 mmol) in N-Methylpyrrolidone (8 mL) were added CuCN (1.45 g, 16.2 mmol). The mixture was stirred at 160° C. for 12 h. The mixture was quenched with ice water (20 mL) and extracted with EA (3×20 mL). The combine organic layer was dried over Na₂SO₄ and concentrated under reduced pressure to afford crude which was purified by silica gel column chromatography eluted with PE/EA (1:1) to afford 4-(2-methoxy-5-methylphenyl)-2-methyl-3-oxo-2,5-dihydro-1,4-benzoxazepine-8-carbonitrile (700 mg, 74%) as a yellow solid. MS (ESI): mass calcd. for C₁₉H₁₈N₂O₃, 322.4, m/z found 323.4 [M+H]⁺.

2-methyl-3-oxo-4,5-dihydro-2H-1,4-benzoxazepine-8-carbonitrile. A solution of 4-(2-methoxy-5-methylphenyl)-2-methyl-3-oxo-2,5-dihydro-1,4-benzoxazepine-8-carbonitrile (300 mg, 0.93 mmol) in TFA (5 mL) was stirred at 80° C. for 12 h. Upon completion, the solvent was evaporated under reduced pressure to afford 2-methyl-3-oxo-4,5-dihydro-2H-1,4-benzoxazepine-8-carbonitrile (150 mg, 80%) as a yellow solid. MS (ESI): mass calcd. for C₁₁H₁₀N₂O₂, 220.2, m/z found 221.2 [M+H]⁺.

N-hydroxy-2-methyl-3-oxo-4,5-dihydro-2H-1,4-benzoxazepine-8-carboximidamide. To a solution of 2-methyl-3-oxo-4,5-dihydro-2H-1,4-benzoxazepine-8-carbonitrile (150 mg, 0.74 mmol) in EtOH (5 mL) was added NH₂OH/H₂O (74 mg, 2.23 mmol). The reaction mixture was stirred at 60° C. for 6 h. Upon completion, the solvent was evaporated under reduced pressure to afford N-hydroxy-2-methyl-3-oxo-4,5-dihydro-2H-1,4-benzoxazepine-8-carboximidamide (150 mg, 85%) as a white solid. MS (ESI): mass calcd. for C₁₁H₁₃N₃O₃, 235.3, m/z found 236.3 [M+H]⁺.

2-methyl-8-[5-(trifluoromethyl)-1,2,4-oxadiazol-3-yl]-4,5-dihydro-2H-1,4-benzoxazepin-3-one. To a solution of N-hydroxy-2-methyl-3-oxo-4,5-dihydro-2H-1,4-benzoxazepine-8-carboximidamide (200 mg, 0.85 mmol) in DMF (5 mL) was added TFAA (1 mL) and pyridine (269 mg, 3.4 mmol). The mixture was stirred at 80° C. for 2 h. The solvent was removed to afford the crude product. The crude product was purified by RP-C18 column eluted with H₂O (0.5% FA)/CH₃CN (100:0→50:50) to afford 2-methyl-8-[5-(trifluoromethyl)-1,2,4-oxadiazol-3-yl]-4,5-dihydro-2H-1,4-benzoxazepin-3-one (88 mg, 33%) as a white solid. MS (ESI): mass calcd. for C₁₃H₁₀F₃N₃O₃, 313.2, m/z found 314.2 [M+H]⁺. ¹H NMR (400 MHz, DMSO) δ 8.35 (m, 1H), 7.62 (m, 1H), 7.46 (m, 2H), 5.32 (m, 1H), 4.89 (m, 1H), 4.09 (m, 1H), 1.39 (d, J=8.0 Hz, 3H).

### Example 1.51. Synthesis of 4-(1H-benzo[d]imidazol-2-yl)-8-(5-(trifluoromethyl)-1,2,4-oxadiazol-3-yl)-2,3,4,5-tetrahydrobenzo[f][1,4]oxazepine (I-51)

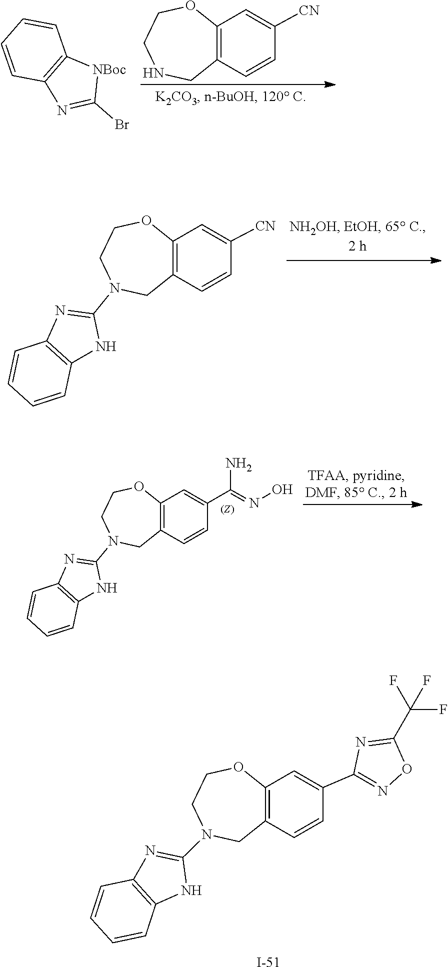

4-(1H-benzo[d]imidazol-2-yl)-2,3,4,5-tetrahydrobenzo[f][1,4]oxazepine-8-carbonitrile. To a solution of tert-butyl 2-bromo-1,3-benzodiazole-1-carboxylate (150 mg, 0.50 mmol) and 2,3,4,5-tetrahydro-1,4-benzoxazepine-8-carbonitrile (88 mg, 0.50 mmol) in n-BuOH (3 mL) was added sodium carbonate (161 mg, 1.51 mmol) under N₂. The reaction mixture was stirred at 120° C. for 16 h. The reaction mixture was quenched with water (10 mL) and extracted with EtOAc (20 mL×3). The combined organic layer washed with brine (20 mL), dried over Na₂SO₄, concentrated to get the residue. The residue was purified by C18-column (phase A: water; phase B: ACN, 5˜35% B) to get 4-(1H-benzo[d]imidazol-2-yl)-2,3,4,5-tetrahydrobenzo[f][1,4]oxazepine-8-carbonitrile (45 mg, 31%) as a white solid. MS (ESI): mass calcd. for C₁₇H₁₄N₄O 290.12, m/z found 291.1 [M+H]⁺.

(Z)-4-(1H-benzo[d]imidazol-2-yl)-N′-hydroxy-2,3,4,5-tetrahydrobenzo[f][1,4]oxazepine-8-carboximidamide. 4-(1H-1,3-benzodiazol-2-yl)-3,5-dihydro-2H-1,4-benzoxazepine-8-carbonitrile (45 mg, 0.16 mmol) was added to NH₂OH/EtOH=1/1 (4 mL). The reaction mixture was stirred at 65° C. for 2 h. The reaction mixture was concentrated to get (Z)-4-(1H-benzo[d]imidazol-2-yl)-N′-hydroxy-2,3,4,5-tetrahydrobenzo[f][1,4]oxazepine-8-carboximidamide (45 mg, 90%) as a white solid. MS (ESI): mass calcd. for C₁₇H₁₇N₅O₂ 323.14, m/z found 324.1 [M+H]⁺.

4-(1H-benzo[d]imidazol-2-yl)-8-(5-(trifluoromethyl)-1,2,4-oxadiazol-3-yl)-2,3,4,5-tetrahydrobenzo[f][1,4]oxazepine. To a solution of 4-(1H-1,3-benzodiazol-2-yl)-N′-hydroxy-3,5-dihydro-2H-1,4-benzoxazepine-8-carboximidamide (45 mg, 0.14 mmol) in DMF (3 mL) was added pyridine (110 mg, 1.39 mmol) and TFAA (117 mg, 0.56 mmol) under N₂. The reaction mixture was stirred at 85° C. for 2 h. The reaction mixture was quenched with water (10 mL) and extracted with DCM (15 mL×3). The combined organic layer was washed with brine (20 mL), dried over Na₂SO₄, concentrated to get the residue. The residue was purified by prep-HPLC (Chromatographic columns: -Xbridge-C18 150×19 mm, 5 μm Mobile Phase: ACN-—H₂O (0.1% FA), B (Acetonitrile), flow rate: 20 mL/min, wavelength: 214/254 nm, injection: 8) to get 4-(1H-benzo[d]imidazol-2-yl)-8-(5-(trifluoromethyl)-1,2,4-oxadiazol-3-yl)-2,3,4,5-tetrahydrobenzo[f][1,4]oxazepine (11.3 mg, 20%) as a white solid. MS (ESI): mass calcd. for C₁₉H₁₄F₃N₅O₂ 401.11, m/z found 402.1[M+H]⁺. ¹H NMR (400 MHz, CD₃OD) δ 7.74-7.66 (m, 1H), 7.65-7.52 (m, 2H), 7.17-7.12 (m, 2H), 6.98-6.92 (m, 2H), 4.78 (s, 2H), 4.31-4.18 (m, 2H), 4.05-3.92 (m, 2H).

### Example 1.52. Synthesis of 4-[(3-methyloxetan-3-yl) carbonyl]-8-[5-(trifluoromethyl)-1,2,4-oxadiazol-3-yl]-3,5-dihydro-2H-1,4-benzoxazepine (I-52)

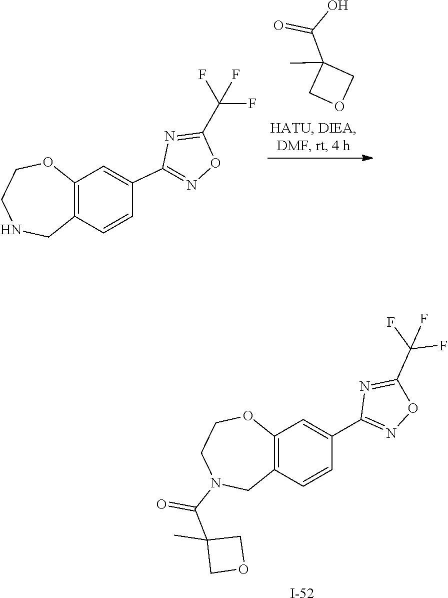

To a solution of 3-methyloxetane-3-carboxylic acid (37 mg, 0.319 mmol) and HATU (140 mg, 0.368 mmol) in DMF (5 mL) stirred under nitrogen at 25° C. for 0.5 h were added 8-[5-(trifluoromethyl)-1,2,4-oxadiazol-3-yl]-2,3,4,5-tetrahydro-1,4-benzoxazepine (70 mg, 0.245 mmol) and DIEA (95 mg, 0.736 mmol). The reaction mixture was stirred at 25° C. for 11.5 h. The mixture was diluted with water (20 mL) and extracted with EtOAc (20 mL×3). The organic layer was dried over anhydrous Na₂SO₄, filtered and concentrated under reduced pressure to give the crude product. The residue was purified by Prep-HPLC to give 4-[(3-methyloxetan-3-yl) carbonyl]-8-[5-(trifluoromethyl)-1,2,4-oxadiazol-3-yl]-3,5-dihydro-2H-1,4-benzoxazepine (70.0 mg, 73%) as a white solid. MS (ESI): mass calcd. for C₁₇H₁₆F₃N₃O₄, 383.11, m/z found 384.1 [M+H]⁺. ¹H NMR (400 MHz, DMSO) δ 7.72-7.52 (m, 3H), 4.77-4.24 (m, 8H), 3.87-3.44 (m, 2H), 1.56-1.52 (m, 3H).

### Example 1.53. Synthesis of 2-methyl-1-{8-[5-(trifluoromethyl)-1,2,4-oxadiazol-3-yl]-3,5-dihydro-2H-1,4-benzoxazepin-4-yl}propan-1-one (I-53)

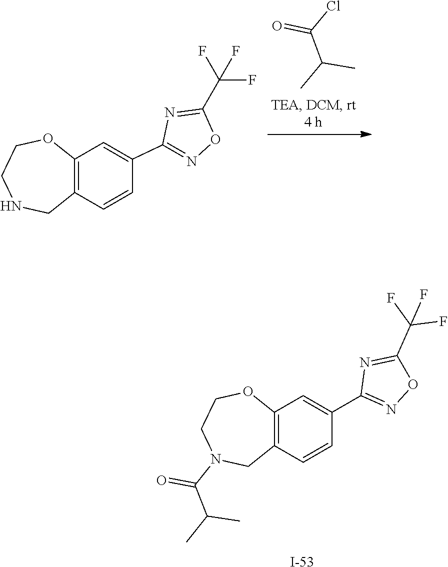

To a solution of 8-[5-(trifluoromethyl)-1,2,4-oxadiazol-3-yl]-2,3,4,5-tetrahydro-1,4-benzoxazepine (61 mg, 0.2139 mmol) and TEA (43 mg, 0.4278 mmol) in DCM (3 mL) stirred under nitrogen at 0° C. was added isobutyryl chloride (27 mg, 0.256 mmol). The reaction mixture was stirred at 25° C. for 4 h. The mixture was diluted with water (20 mL) and extracted with EtOAc (20 mL×3). The organic layer was dried over anhydrous Na₂SO₄, filtered and concentrated under reduced pressure to give the crude product. The residue was purified by Prep-HPLC to give 2-methyl-1-{8-[5-(trifluoromethyl)-1,2,4-oxadiazol-3-yl]-3,5-dihydro-2H-1,4-benzoxazepin-4-yl}propan-1-one (44.1 mg, 57%) as a colorless oil. MS (ESI): mass calcd. for C₁₆H₁₆F₃N₃O₃, 355.11, m/z found 356.1 [M+H]⁺. ¹H NMR (400 MHz, DMSO) δ 7.74-7.48 (m, 3H), 4.81 (s, 1H), 4.66 (s, 1H), 4.26-4.18 (m, 2H), 3.95-3.87 (m, 2H), 3.04-2.83 (m, 1H), 0.97-0.89 (m, 6H).

### Example 1.54. Synthesis of 4-[(oxan-4-yl) carbonyl]-8-[5-(trifluoromethyl)-1,2,4-oxadiazol-3-yl]-3,5-dihydro-2H-1,4-benzoxazepine (I-54)

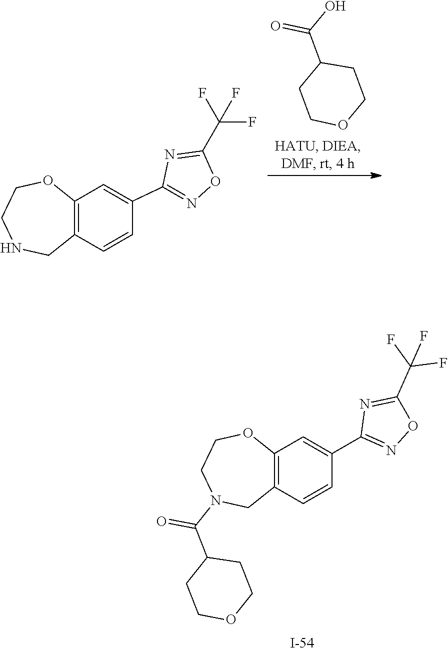

To a solution of oxane-4-carboxylic acid (41 mg, 0.319 mmol) and HATU (139 mg, 0.3681 mmol) in DMF (4 mL) stirred under nitrogen at 25° C. for 0.5 h were added 8-[5-(trifluoromethyl)-1,2,4-oxadiazol-3-yl]-2,3,4,5-tetrahydro-1,4-benzoxazepine (70 mg, 0.245 mmol) and DIEA (95 mg, 0.736 mmol). The reaction mixture was stirred at 25° C. for 11.5 h. The mixture was diluted with water (20 mL) and extracted with EtOAc (20 mL×3). The organic layer was dried over anhydrous Na₂SO₄, filtered and concentrated under reduced pressure to give the crude product. The residue was purified by Prep-HPLC to give 4-[(oxan-4-yl) carbonyl]-8-[5-(trifluoromethyl)-1,2,4-oxadiazol-3-yl]-3,5-dihydro-2H-1,4-benzoxazepine (52 mg, 52%) as a white solid. MS (ESI): mass calcd. for C₁₈H₁₈F₃N₃O₄, 397.12, m/z found 398.1 [M+H]⁺. ¹H NMR (400 MHz, DMSO) δ 7.72-7.49 (m, 3H), 4.86 (s, 1H), 4.67 (s, 1H), 4.25-4.18 (m, 2H), 3.98-3.77 (m, 4H), 3.41-3.35 (m, 2H), 3.05-2.85 (m, 1H), 1.56-1.28 (m, 4H).

### Example 1.55. Synthesis of (S)-(1-ethyl-4-methylpiperidin-4-yl)(6-fluoro-3-methyl-8-(5-(trifluoromethyl)-1,2,4-oxadiazol-3-yl)-2,3-dihydrobenzo[f][1,4]oxazepin-4(5H)-yl)methanone (I-55)

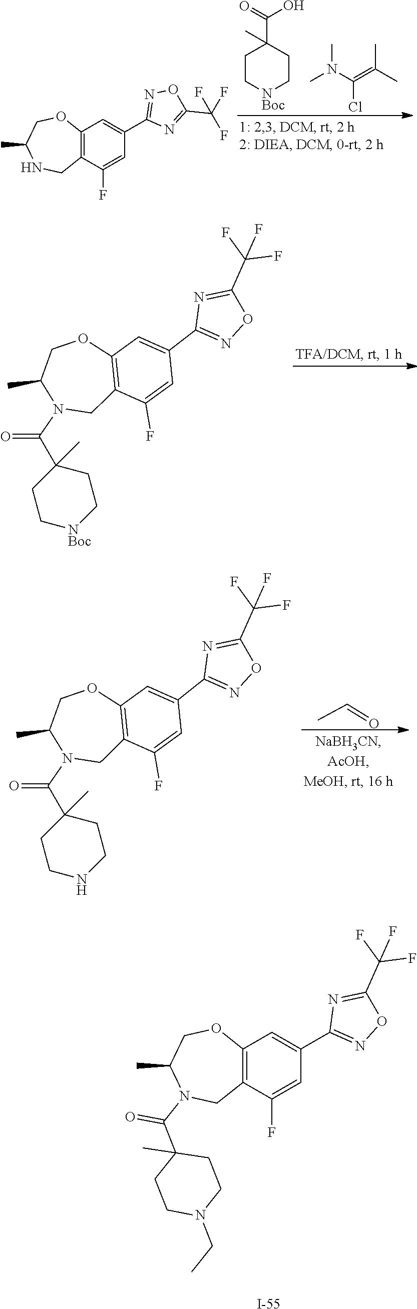

Tert-butyl 4-{[(3S)-6-fluoro-3-methyl-8-[5-(trifluoromethyl)-1,2,4-oxadiazol-3-yl]-3,5-dihydro-2H-1,4-benzoxazepin-4-yl]carbonyl}-4-methylpiperidine-1-carboxylate. To a solution of 1-[(tert-butoxy) carbonyl]-4-methylpiperidine-4-carboxylic acid (614 mg, 2.52 mmol) in DCM (10 mL) was added 1-chloro-N, N, 2-trimethylpropenylamine (337 mg, 2.52 mmol). The reaction mixture was stirred at 25° C. for 2 h under N₂ atmosphere, then concentrated. The residue was dissolved in DCM (10 mL), added (3S)-6-fluoro-3-methyl-8-[5-(trifluoromethyl)-1,2,4-oxadiazol-3-yl]-2,3,4,5-tetrahydro-1,4-benzoxazepine (400 mg, 1.26 mmol) and DIEA (652 mg, 5.04 mmol). The reaction mixture was stirred at 25° C. for 1 h. The reaction mixture was quenched by H₂O (30 mL), extracted with DCM (30 mL×2). The organic layer was washed with brine (30 mL), dried over anhydrous Na₂SO₄, concentrated. The residue was purified by silica gel column chromatography eluted with PE/EA (3:1) to afford tert-butyl 4-{[(3S)-6-fluoro-3-methyl-8-[5-(trifluoromethyl)-1,2,4-oxadiazol-3-yl]-3,5-dihydro-2H-1,4-benzoxazepin-4-yl]carbonyl}-4-methylpiperidine-1-carboxylate (300 mg, 42%) as a white solid. MS (ESI): mass calcd. for C₂₅H₃₀F₄N₄O₅, 542.22, m/z found 565.1 [M+Na]⁺.

(3S)-6-fluoro-3-methyl-4-[(4-methylpiperidin-4-yl) carbonyl]-8-[5-(trifluoromethyl)-1,2,4-oxadiazol-3-yl]-3,5-dihydro-2H-1,4-benzoxazepine. To a mixture of tert-butyl 4-{[(3S)-6-fluoro-3-methyl-8-[5-(trifluoromethyl)-1,2,4-oxadiazol-3-yl]-3,5-dihydro-2H-1,4-benzoxazepin-4-yl]carbonyl}-4-methylpiperidine-1-carboxylate (300 mg, 0.54 mmol) in DCM (10 mL) was added TFA (3 mL). The mixture was stirred at 25° C. for 1 hour. The mixture was concentrated to afford (3S)-6-fluoro-3-methyl-4-[(4-methylpiperidin-4-yl) carbonyl]-8-[5-(trifluoromethyl)-1,2,4-oxadiazol-3-yl]-3,5-dihydro-2H-1,4-benzoxazepine (220 mg, 90%) as a white solid. MS (ESI): mass calcd. for C₂₀H₂₂F₄N₄O₃, 442.16, m/z found 443.2 [M+H]⁺.

(S)-(1-ethyl-4-methylpiperidin-4-yl)(6-fluoro-3-methyl-8-(5-(trifluoromethyl)-1,2,4-oxadiazol-3-yl)-2,3-dihydrobenzo[f][1,4]oxazepin-4(5H)-yl)methanone. To a mixture of (3S)-6-fluoro-3-methyl-4-[(4-methylpiperidin-4-yl) carbonyl]-8-[5-(trifluoromethyl)-1,2,4-oxadiazol-3-yl]-3,5-dihydro-2H-1,4-benzoxazepine (100 mg, 0.22 mmol) in MeOH (6 mL) were added acetaldehyde (20 mg, 0.45 mmol), AcOH (1.5 mg, 0.02 mmol) and NaBH₃CN (21 mg, 0.33 mmol). The mixture was stirred at 25° C. for 16 hours. The reaction mixture was poured into H₂O (20 mL), extracted with EtOAc (20 mL*3), the combine organic layer was washed with brine (20 mL), dried over Na₂SO₄, filtered, concentrated in vacuo. The residue was purified by Pre-HPLC (Instrument: Waters MS-triggered Prep-LC with QDA detector, Column: Xbridge 5 u C18 150×19 mm, A water (0.1% FA), B Acetonitrile 50-80% B in 8 min, hold at 100% B for 2 min, back to 50% B with 0.5 min, stop at 13 min, flow rate: 20 mL/min, wavelength: 214/254 nm, injection: 7) to afford (S)-(1-ethyl-4-methylpiperidin-4-yl)(6-fluoro-3-methyl-8-(5-(trifluoromethyl)-1,2,4-oxadiazol-3-yl)-2,3-dihydrobenzo[f][1,4]oxazepin-4(5H)-yl)methanone (43 mg, 43%) as a white solid. MS (ESI): mass calcd. for C₂₂H₂₆F₄N₄O₃, 470.19, m/z found 471.1[M+H]⁺. ¹H NMR (400 MHz, DMSO-d₆) δ 7.46 (d, J=9.6 Hz, 1H), 7.29 (s, 1H), 5.10-4.99 (m, 1H), 4.85-4.70 (m, 1H), 4.53-4.25 (m, 3H), 2.81-2.65 (m, 2H), 2.49-2.29 (m, 4H), 2.21-1.95 (m, 2H), 1.67-1.40 (m, 2H), 1.27-1.13 (m, 6H), 1.06-0.98 (m, 3H).

### Example 1.56. Synthesis of (S)-(6-fluoro-3-methyl-8-(5-(trifluoromethyl)-1,2,4-oxadiazol-3-yl)-2,3-dihydrobenzo[f][1,4]oxazepin-4(5H)-yl)(1-isopropyl-4-methylpiperidin-4-yl)methanone (I-56)

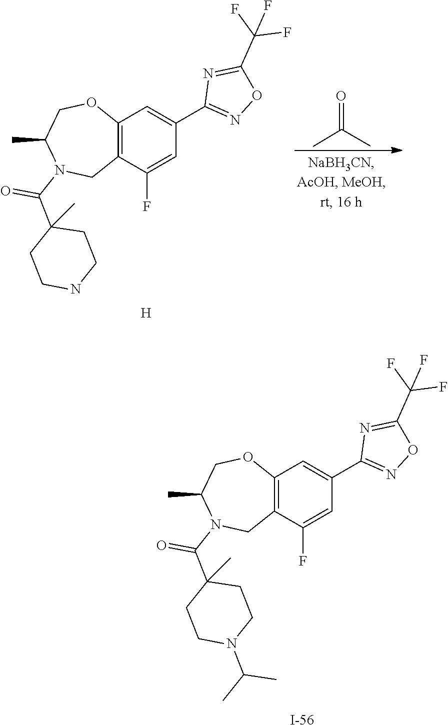

To a solution of ((3S)-6-fluoro-3-methyl-4-[(4-methylpiperidin-4-yl) carbonyl]-8-[5-(trifluoromethyl)-1,2,4-oxadiazol-3-yl]-3,5-dihydro-2H-1,4-benzoxazepine (100 mg, 0.23 mmol) in MeOH (5 mL) was added acetone (262 mg, 4.5 mmol), AcOH (14 mg, 0.23 mmol) and NaBH₃CN (21 mg, 0.34 mmol). The reaction mixture was stirred at 25° C. for 16 h. The mixture was quenched by H₂O (15 mL), extracted with EA (20 mL×2). The organic layer was washed with brine (40 mL), dried over anhydrous Na₂SO₄, concentrated under vacuum. The residue was purified by Pre-HPLC, (Instrument: Waters MS-triggered Prep-LC with QDA detector, Column: Xbridge 5 u C18 150×19 mm, A water (0.1% FA), B Acetonitrile 40-70% B in 8 min, hold at 100% B for 2 min, back to 40% B with 0.5 min, stop at 13 min, flow rate: 20 mL/min, wavelength: 214/254 nm, injection: 8) to afford (S)-(6-fluoro-3-methyl-8-(5-(trifluoromethyl)-1,2,4-oxadiazol-3-yl)-2,3-dihydrobenzo[f][1,4]oxazepin-4(5H)-yl)(1-isopropyl-4-methylpiperidin-4-yl)methanone (26.3 mg) as a white solid. MS (ESI): mass calcd. For C₂₃H₂₈F₄N₄O₃, 484.21, m/z found 485.3 [M+H]⁺. ¹H NMR (400 MHz, DMSO-d₆) δ 7.46 (d, J=9.6 Hz, 1H), 7.29 (s, 1H), 5.15-4.95 (m, 1H), 4.85-4.67 (m, 1H), 4.59-4.23 (m, 3H), 3.10-2.76 (m, 3H), 2.71-2.52 (m, 2H), 2.28-2.05 (m, 2H), 1.75-1.45 (m, 2H), 1.29-1.14 (m, 6H), 1.04 (d, J=6.0 Hz, 6H).

### Example 1.57. Synthesis of (S)-(4-(2-methoxyethyl)-1-methylpiperidin-4-yl)(3-methyl-8-(5-(trifluoromethyl)-1,2,4-oxadiazol-3-yl)-2,3-dihydrobenzo[f][1,4]oxazepin-4(5H)-yl)methanone (I-57)

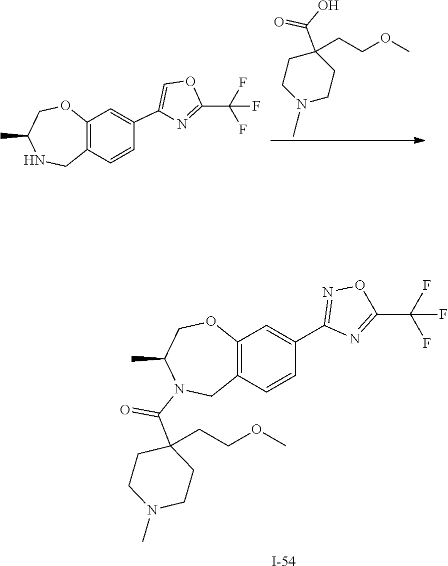

To a solution of (S)-3-methyl-8-(5-(trifluoromethyl)-1,2,4-oxadiazol-3-yl)-2,3,4,5-tetrahydrobenzo[f][1,4]oxazepine (109 mg, 0.36 mmol), 4-(2-methoxyethyl)-1-methylpiperidine-4-carboxylic acid (60 mg, 0.30 mmol) and DIEA (310 mg, 2.40 mmol) in DCM (5 mL) was added Phosphorus oxychloride (124 mg, 0.81 mmol) at 0° C. The reaction mixture was stirred under nitrogen at 25° C. for 16 h. To the solution was added NaHCO₃ aq. (10 mL) and DCM (15 mL). The mixture was extracted with DCM (10 mL×2). The organic layer was concentrated, purified by Prep-HPLC to obtain (S)-(4-(2-methoxyethyl)-1-methylpiperidin-4-yl)(3-methyl-8-(5-(trifluoromethyl)-1,2,4-oxadiazol-3-yl)-2,3-dihydrobenzo[f][1,4]oxazepin-4(5H)-yl)methanone (20.17 mg, 14%) as a white solid. MS (ESI): mass calcd. for C₂₃H₂₉F₃N₄O₄, 482.21, m/z found 483.2 [M+H]⁺. ¹H NMR (400 MHz, DMSO) δ 8.29 (s, 1H), 7.64-7.62 (m, 1H), 7.42 (s, 1H), 4.85-4.75 (m, 3H), 4.31-4.19 (m, 2H), 3.12-3.03 (m, 7H), 2.08-2.05 (m, 7H), 1.69-1.66 (m, 2H), 1.37-1.11 (m, 5H).

Examples 1.58-1.60. Synthesis of (S)-(3-(4H-1,2,4-triazol-4-yl)cyclobutyl)(6-fluoro-3-methyl-8-(5-(trifluoromethyl)-1,2,4-oxadiazol-3-yl)-2,3-dihydrobenzo[f][1,4]oxazepin-4(5H)-yl)methanone; racemate (I-58), first and second eluting isomers (I-59 and I-60)

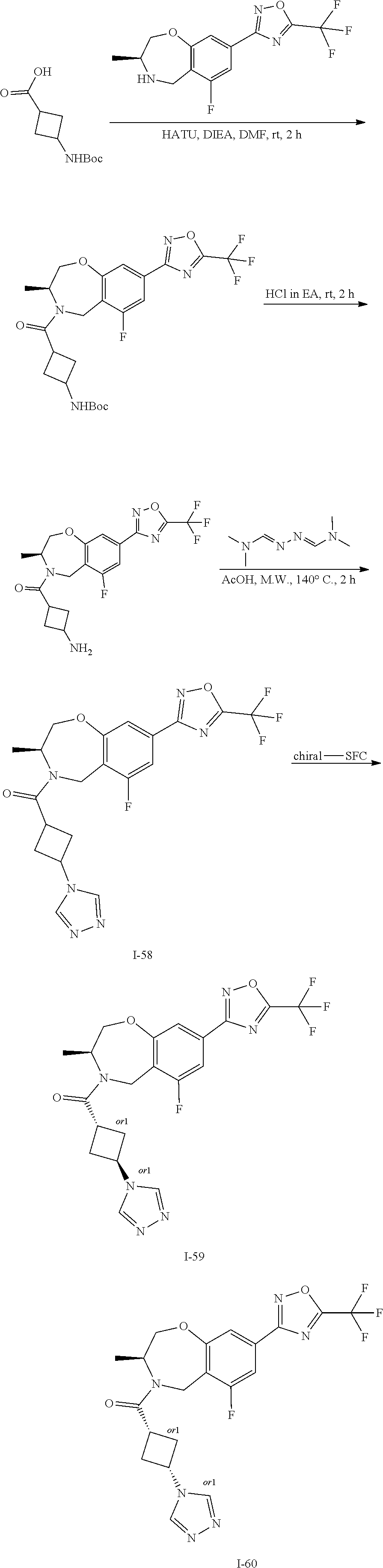

Tert-butyl (S)-(3-(6-fluoro-3-methyl-8-(5-(trifluoromethyl)-1,2,4-oxadiazol-3-yl)-2,3,4,5-tetrahydrobenzo[f][1,4]oxazepine-4-carbonyl) cyclobutyl) carbamate. DIEA (733 mg, 5.67 mmol) was added to the mixture of 3-{[(tert-butoxy) carbonyl]amino}cyclobutane-1-carboxylic acid (407 mg, 1.89 mmol), (3S)-6-fluoro-3-methyl-8-[5-(trifluoromethyl)-1,2,4-oxadiazol-3-yl]-2,3,4,5-tetrahydro-1,4-benzoxazepine (600 mg, 1.89 mmol) and HATU (863 mg, 2.27 mmol) in DMF (10 mL). The mixture was stirred at 25° C. for 2 hours. The reaction mixture was quenched with water (20 mL) and extracted with EtOAc (20 mL×3). The combined organic layer was washed with brine (30 mL), dried over Na₂SO₄, concentrated to get the residue. The residue was purified by column chromatography on silica gel (12 g) eluting with MeOH in DCM (3%), fraction with MS signal of desired product was collected and concentrated to get tert-butyl (S)-(3-(6-fluoro-3-methyl-8-(5-(trifluoromethyl)-1,2,4-oxadiazol-3-yl)-2,3,4,5-tetrahydrobenzo[f][1,4]oxazepine-4-carbonyl) cyclobutyl) carbamate (680 mg, 63%) as yellow oil. MS (ESI): mass calcd. for C₂₃H₂₆F₄N₄O₅, 514.18, m/z found 515.2 [M+H]⁺.

(S)-(3-aminocyclobutyl) (6-fluoro-3-methyl-8-(5-(trifluoromethyl)-1,2,4-oxadiazol-3-yl)-2,3-dihydrobenzo[f][1,4]oxazepin-4(5H)-yl) methanone. Tert-butyl N-(3-{[(3S)-6-fluoro-3-methyl-8-[5-(trifluoromethyl)-1,2,4-oxadiazol-3-yl]-3,5-dihydro-2H-1,4-benzoxazepin-4-yl]carbonyl}cyclobutyl) carbamate (500 mg, 0.97 mmol) was added to HCl in EA (10 mL). The reaction mixture was stirred at 25° C. for 2 hours. The reaction mixture was concentrated to get (S)-(3-aminocyclobutyl) (6-fluoro-3-methyl-8-(5-(trifluoromethyl)-1,2,4-oxadiazol-3-yl)-2,3-dihydrobenzo[f][1,4]oxazepin-4(5H)-yl) methanone (400 mg, 95%) as yellow oil. MS (ESI): mass calcd. for C₁₈H₁₈F₄N₄O₃, 414.13, m/z found 415.1 [M+H]⁺.

(S)-(3-(4H-1,2,4-triazol-4-yl)cyclobutyl)(6-fluoro-3-methyl-8-(5-(trifluoromethyl)-1,2,4-oxadiazol-3-yl)-2,3-dihydrobenzo[f][1,4]oxazepin-4(5H)-yl)methanone. N′—[(E)-N′—[(N,N-dimethylamino)methylidene]amino]-N,N-dimethylmethanimidamide (515 mg, 3.62 mmol) was added to the mixture of 3-{[(3S)-6-fluoro-3-methyl-8-[5-(trifluoromethyl)-1,2,4-oxadiazol-3-yl]-3,5-dihydro-2H-1,4-benzoxazepin-4-yl]carbonyl}cyclobutan-1-amine (500 mg, 1.21 mmol) in AcOH (15 mL). The mixture was stirred at 140° C. in microwave under N₂ protected for 2 hours. The reaction mixture was concentrated to get the residue. The residue was purified by prep-HPLC (phase A: H₂O, phase B: ACN 5˜42%), fraction with MS signal of desired product was collected and concentrated to get the (S)-(3-(4H-1,2,4-triazol-4-yl)cyclobutyl)(6-fluoro-3-methyl-8-(5-(trifluoromethyl)-1,2,4-oxadiazol-3-yl)-2,3-dihydrobenzo[f][1,4]oxazepin-4(5H)-yl)methanone (I-57) (220 mg, 39%) as a white solid. MS (ESI): mass calcd. for C₂₀H₁₈F₄N₆O₃, 466.14, m/z found 467.1 [M+H]⁺.

(S)-(3-(4H-1,2,4-triazol-4-yl)cyclobutyl)(6-fluoro-3-methyl-8-(5-(trifluoromethyl)-1,2,4-oxadiazol-3-yl)-2,3-dihydrobenzo[f][1,4]oxazepin-4(5H)-yl)methanone (220 mg, 0.47 mmol) was split via prep-SFC (Daicel CHIRALPAK AD_3 3×150 mm, 3 μm mobile phase A/B: CO₂/MeOH (0.1% EDA))=70/30, flow rate: 2.0 mL/min, column temp: 37° C. to give a first eluting isomer (23.1 mg, 21%) as a white solid, and a second eluting isomer (79 mg, 72%) as a white solid.

(S)-(3-(4H-1,2,4-triazol-4-yl)cyclobutyl)(6-fluoro-3-methyl-8-(5-(trifluoromethyl)-1,2,4-oxadiazol-3-yl)-2,3-dihydrobenzo[f][1,4]oxazepin-4(5H)-yl)methanone, first eluting isomer (I-58), MS (ESI): mass calcd. for C₂₀H₁₈F₄N₆O₃, 466.14, m/z found 467.1 [M+H]⁺. ¹H NMR (400 MHz, MeOD) δ 8.66-8.62 (m, 2H), 7.51-7.41 (m, 2H), 5.48 (d, J=16.0 Hz, 1H), 5.10-4.74 (m, 2H), 4.44-4.31 (m, 3H), 3.67-3.45 (m, 1H), 3.13-2.39 (m, 4H), 1.29-1.08 (m, 3H).

(S)-(3-(4H-1,2,4-triazol-4-yl)cyclobutyl)(6-fluoro-3-methyl-8-(5-(trifluoromethyl)-1,2,4-oxadiazol-3-yl)-2,3-dihydrobenzo[f][1,4]oxazepin-4(5H)-yl)methanone, second eluting isomer (I-59), MS (ESI): mass calcd. for C₂₀H₁₈F₄N₆O₃, 466.14, m/z found 467.1 [M+H]⁺. ¹H NMR (400 MHz, MeOD) δ 8.69-8.59 (m, 2H), 7.51-7.37 (m, 2H), 5.45 (d, J=16.0 Hz, 1H), 5.12-4.75 (m, 2H), 4.54-4.30 (m, 3H), 3.48-3.22 (m, 1H), 2.89-2.59 (m, 3H), 2.50-2.30 (m, 1H), 1.32-1.19 (m, 3H).

### Examples 1.61-1.63. Synthesis of ((S)-6-fluoro-3-methyl-8-(5-(trifluoromethyl)-1,2,4-oxadiazol-3-yl)-2,3-dihydrobenzo[f][1,4]oxazepin-4(5H)-yl)(1-methylpiperidin-3-yl)methanone; racemate (I-61), first and second eluting isomers (I-62 and I-63)

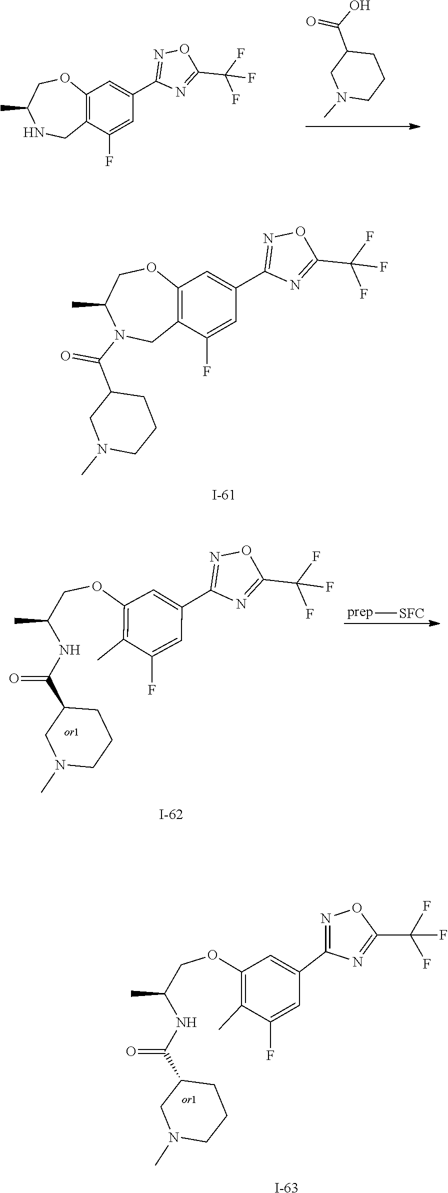

(3S)-6-fluoro-3-methyl-4-[(1-methylpiperidin-3-yl) carbonyl]-8-[5-(trifluoromethyl)-1,2,4-oxadiazol-3-yl]-3,5-dihydro-2H-1,4-benzoxazepine. DIEA (163 mg, 1.3 mmol) and N,N,N′,N′-Tetramethyl-O-(7-azabenzotriazol-1-yl)uronium (240 mg, 0.6 mmol) and 1-methylpiperidine-3-carboxylic acid (90 mg, 0.6 mmol) were added to the (3S)-6-fluoro-3-methyl-8-[5-(trifluoromethyl)-1,2,4-oxadiazol-3-yl]-2,3,4,5-tetrahydro-1,4-benzoxazepine (100 mg, 0.3 mmol) in DMF (10 mL). The mixture was stirred at 25° C. for 2 hours. The mixture was diluted with water (30 mL) and extracted with EA (30 mL×2). The organic phase was washed with brine (30 mL×3), dried over sodium sulphate, filtered, concentrated in vacuo. The residue was purified by chromatography column on silica gel eluting with PE/EA (1:1) to give product of (3S)-6-fluoro-3-methyl-4-[(1-methylpiperidin-3-yl) carbonyl]-8-[5-(trifluoromethyl)-1,2,4-oxadiazol-3-yl]-3,5-dihydro-2H-1,4-benzoxazepine (I-61) (80 mg, 57%) as a white solid. MS (ESI): mass calcd. for C₂₀H₂₂F₄N₄O₃, 442.16, m/z found 443.1 [M+H]⁺.

(3S)-6-fluoro-3-methyl-4-[(1-methylpiperidin-3-yl) carbonyl]-8-[5-(trifluoromethyl)-1,2,4-oxadiazol-3-yl]-3,5-dihydro-2H-1,4-benzoxazepine (80 mg) was separated by prep-SFC Apparatus: SFC 150, Column: Daicel CHIRALCEL IC, 250 mm, 30 mm ID, 10 m, Mobile phase: CO₂/MeOH [0.2% NH₃ (7M Solution in MeOH)]=60/40. Flow rate: 80 g/min, wavelength: UV 214 nm. Temperature: 35° C.) to give 8.8 mg of a first eluting isomer (I-62) as a white solid, and 22.6 mg of a second eluting isomers (I-63) as a white solid.

(3S)-6-fluoro-3-methyl-4-[(1-methylpiperidin-3-yl) carbonyl]-8-[5-(trifluoromethyl)-1,2,4-oxadiazol-3-yl]-3,5-dihydro-2H-1,4-benzoxazepine, first eluting isomer (I-62), MS (ESI): mass calcd. for C₂₀H₂₂F₄N₄O₃, 442.16, m/z found 443.1 [M+H]⁺. ¹H NMR (400 MHz, DMSO-d6) δ 7.48-7.39 (m, 1H), 7.28-7.25 (d, J=12.0 Hz, 1H), 5.20-4.75 (m, 2H), 4.50-4.23 (m, 3H), 2.79-2.63 (m, 3H), 2.11-2.06 (d, J=16.0 Hz, 3H), 1.88-1.32 (m, 6H), 1.18-1.00 (m, 3H).

(3S)-6-fluoro-3-methyl-4-[(1-methylpiperidin-3-yl) carbonyl]-8-[5-(trifluoromethyl)-1,2,4-oxadiazol-3-yl]-3,5-dihydro-2H-1,4-benzoxazepine, second eluting isomer (I-63), MS (ESI): mass calcd. for C₂₀H₂₂F₄N₄O₃, 442.16, m/z found 443.1 [M+H]⁺. ¹H NMR (400 MHz, DMSO-d6) δ 7.56-7.45 (m, 1H), 7.34-7.30 (d, J=12.0 Hz, 1H), 5.28-4.31 (m, 5H), 3.09-2.61 (m, 6H), 3.23-1.06 (m, 9H).

### Examples 1.64-1.66. Synthesis of (1,3-dimethylpyrrolidin-3-yl)((S)-6-fluoro-3-methyl-8-(5-(trifluoromethyl)-1,2,4-oxadiazol-3-yl)-2,3-dihydrobenzo[f][1,4]oxazepin-4(5H)-yl)methanone; racemate (I-64), first and second eluting isomers (I-65 and I-66)

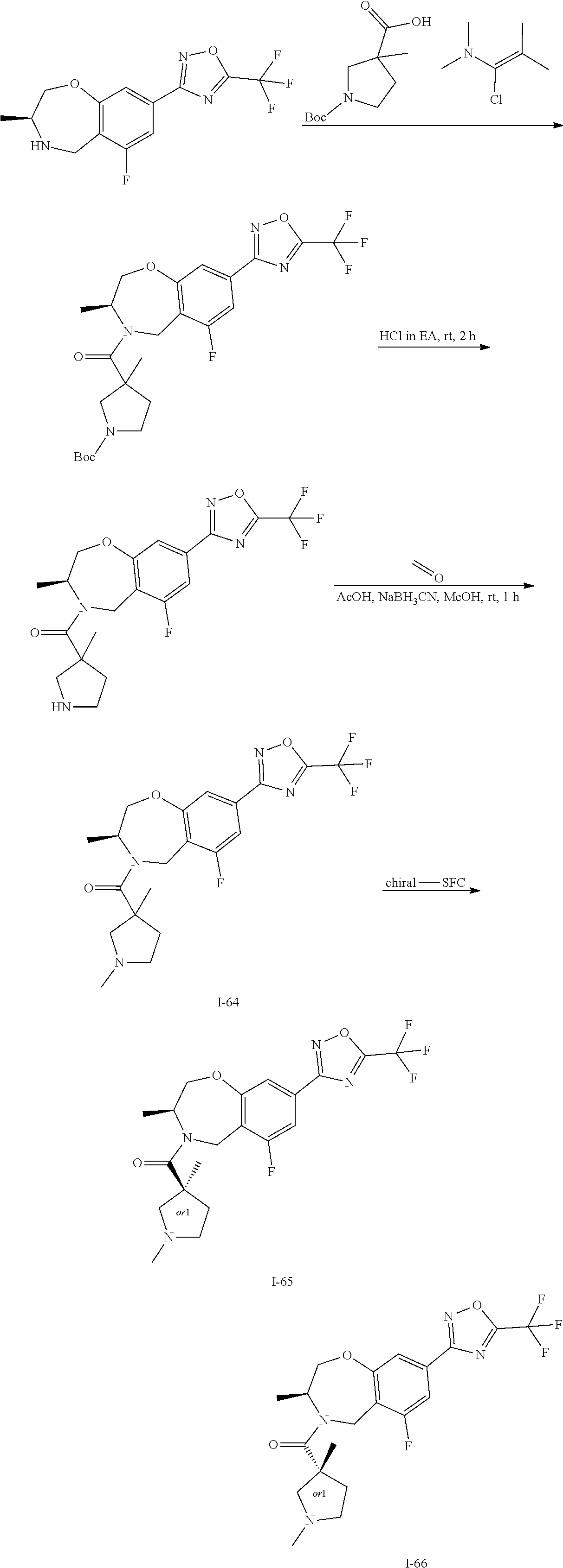

tert-butyl 3-((S)-6-fluoro-3-methyl-8-(5-(trifluoromethyl)-1,2,4-oxadiazol-3-yl)-2,3,4,5-tetrahydrobenzo[f][1,4]oxazepine-4-carbonyl)-3-methylpyrrolidine-1-carboxylate. A mixture of 1-(tert-butoxycarbonyl)-3-methylpyrrolidine-3-carboxylic acid (500 mg, 2.17 mmol) and 1-chloro-N,N,2-trimethylpropenylamine (580 mg, 4.34 mmol) in DCM (3 mL) was stirred at 25° C. for 2 h. The mixture was concentrated to afford tert-butyl[3-(carbonochloridoyl)-3-methylpyrrolidin-1-yl]formate (500 mg, 83%) as a yellow oil. The crude product was directly used to the next step. To a solution of (3S)-6-fluoro-3-methyl-8-[5-(trifluoromethyl)-1,2,4-oxadiazol-3-yl]-2,3,4,5-tetrahydro-1,4-benzoxazepine (300 mg, 0.95 mmol) in DCM (5 mL) was added DIEA (489 mg, 3.78 mmol) and tert-butyl[3-(carbonochloridoyl)-3-methylpyrrolidin-1-yl]formate (470 mg, 1.89 mmol). The mixture was stirred at 25° C. for 0.5 h under N₂. The mixture was concentrated and purified by silica gel column (PE/EA=5/1) to afford tert-butyl 3—((S)-6-fluoro-3-methyl-8-(5-(trifluoromethyl)-1,2,4-oxadiazol-3-yl)-2,3,4,5-tetrahydrobenzo[f][1,4]oxazepine-4-carbonyl)-3-methylpyrrolidine-1-carboxylate (360 mg, 68%) as yellow oil. MS (ESI): mass calcd. for C₂₄H₂₈F₄N₄O₅ 528.20, m/z found 551.2 [M+Na]⁺.

(3S)-6-fluoro-3-methyl-4-[(3-methylpyrrolidin-3-yl) carbonyl]-8-[5-(trifluoromethyl)-1,2,4-oxadiazol-3-yl]-3,5-dihydro-2H-1,4-benzoxazepine. A mixture of tert-butyl (3-{[(3S)-6-fluoro-3-methyl-8-[5-(trifluoromethyl)-1,2,4-oxadiazol-3-yl]-3,5-dihydro-2H-1,4-benzoxazepin-4-yl]carbonyl}-3-methylpyrrolidin-1-yl) formate (340 mg, 0.64 mmol) in HCl/EA (3 mL) was stirred at 25° C. for 2 h. The mixture was concentrated and purified by Prep-TLC (PE/EA=2/1) to afford (3S)-6-fluoro-3-methyl-4-[(3-methylpyrrolidin-3-yl) carbonyl]-8-[5-(trifluoromethyl)-1,2,4-oxadiazol-3-yl]-3,5-dihydro-2H-1,4-benzoxazepine (280 mg, 97%) as a white solid. MS (ESI): mass calcd. for C₁₉H₂₀F₄N₄O₃ 428.15, m/z found 429.1 [M+H]⁺.

(3S)-4-[(1,3-dimethylpyrrolidin-3-yl) carbonyl]-6-fluoro-3-methyl-8-[5-(trifluoromethyl)-1,2,4-oxadiazol-3-yl]-3,5-dihydro-2H-1,4-benzoxazepine. To a solution of (3S)-6-fluoro-3-methyl-4-[(3-methylpyrrolidin-3-yl) carbonyl]-8-[5-(trifluoromethyl)-1,2,4-oxadiazol-3-yl]-3,5-dihydro-2H-1,4-benzoxazepine (250 mg, 0.58 mmol) and HCHO (35 mg, 1.17 mmol) in MeOH (3 mL) was added AcOH (0.01 mL). After 15 minutes, NaBH₃CN (73 mg, 1.17 mmol) was added to the mixture. The mixture was stirred at 25° C. for 1 h. The reaction mixture was quenched with H₂O (50 mL) and extracted with EA (50 mL×3). The combined organic layers were dried over Na₂SO₄, concentrated. The residue was purified by Prep-TLC (PE/EA=2/1) to afford (3S)-4-[(1,3-dimethylpyrrolidin-3-yl) carbonyl]-6-fluoro-3-methyl-8-[5-(trifluoromethyl)-1,2,4-oxadiazol-3-yl]-3,5-dihydro-2H-1,4-benzoxazepine (I-64) (100 mg, 37%) as a white solid. MS (ESI): mass calcd. for C₂₀H₂₂F₄N₄O₃ 442.16, m/z found 443.2 [M+H]⁺.

(3S)-4-[(1,3-dimethylpyrrolidin-3-yl)carbonyl]-6-fluoro-3-methyl-8-[5-(trifluoromethyl)-1,2,4-oxadiazol-3-yl]-3,5-dihydro-2H-1,4-benzoxazepine (100 mg) was split via prep-SFC (mobile phase A/B: CO₂/MeOH (0.1% DEA)=60/40, flow rate: 1.5 mL/min, column temp: 37° C.) to give a first eluting isomer (I-65) (2.2 mg, 2%) as a white solid, and a second eluting isomer (I-66) (20.4 mg, 20%) as a white solid.

(3S)-4-[(1,3-dimethylpyrrolidin-3-yl)carbonyl]-6-fluoro-3-methyl-8-[5-(trifluoromethyl)-1,2,4-oxadiazol-3-yl]-3,5-dihydro-2H-1,4-benzoxazepine, first eluting isomer (I-65) MS (ESI): mass calcd. for C₂₀H₂₂F₄N₄O₃ 442.16, m/z found 443.2 [M+H]⁺. ¹H NMR (400 MHz, MeOD) δ 7.47-7.41 (m, 2H), 5.27 (d, J=16.0 Hz, 1H), 4.67-4.64 (m, 1H), 4.44-4.40 (m, 2H), 4.36-4.25 (m, 1H), 3.97 (d, J=8.0 Hz, 1H), 3.12-3.10 (m, 1H), 2.92-2.90 (m, 1H), 2.73 (s, 3H), 2.62-2.57 (m, 2H), 2.29-2.19 (m, 1H), 1.56 (s, 3H), 1.35 (d, J=8.0 Hz, 3H).

(3S)-4-[(1,3-dimethylpyrrolidin-3-yl)carbonyl]-6-fluoro-3-methyl-8-[5-(trifluoromethyl)-1,2,4-oxadiazol-3-yl]-3,5-dihydro-2H-1,4-benzoxazepine, second eluting isomer (I-66) MS (ESI): mass calcd. for C₂₀H₂₂F₄N₄O₃ 442.16, m/z found 443.2 [M+H]⁺. ¹H NMR (400 MHz, MeOD) δ 7.47-7.41 (m, 2H), 5.27 (d, J=16.0 Hz, 1H), 4.66-4.64 (m, 1H), 4.47-4.42 (m, 2H), 4.28-4.25 (m, 1H), 3.97 (d, J=12.0 Hz, 1H), 3.13-3.11 (m, 1H), 2.90 (m, 1H), 2.73 (s, 3H), 2.64-2.57 (m, 2H), 2.28-2.19 (m, 1H), 1.56 (s, 3H), 1.35 (d, J=4.0 Hz, 3H).

### Example 2.01. HDAC6 TR-FRET Displacement Assay

### Materials and Reagents

HDAC6 and Tb-anti-GST antibody were purchased from Signal Chem (cat #PC124) and Cisbio (cat #61GSTTAH), respectively. HEPES, pH 7.5 was purchased from Teknova (Cat #H1575). All other buffer components, NaCl (cat #S5150), KCl (cat #60121), Triton X-100 (cat #T9284), GSH (cat #G4251), BGG (cat #G5009), and BSA (cat #A2153) were purchased from SIGMA.

### Experimental Procedure

Displacement of fluorescein labeled tracer 2-(6-hydroxy-3-oxo-3H-xanthen-9-yl)-5-({[5-(4-{[7-(hydroxycarbamoyl)-2H,3H,4H,5H-thieno[2,3-f][1,4]oxazepine-4-carbonyl]amino}phenoxy)pentyl]carbamothioyl}amino)benzoic acid from HDAC6 was measured in vitro using a TR-FRET assay. The assays were performed in 1536-well plates in a total volume of 6 μL in assay buffer consisting of 50 mM HEPES pH 7.5, 50 mM NaCl, 50 mM KCl, 0.01% Triton X-100, 0.5 mM GSH, 0.03% BGG, 0.01% BSA.

For concentration response curves (IC₅₀ determination), Compounds were serially diluted 3-fold from 2 mM highest concentration to generate a 10-point concentration gradient. To perform the assay, 30 nL of compound gradient solutions were transferred to the assay plate using the ECHO acoustic dispenser to achieve a final concentration range of 10 μM to 0.000508 uM in 6 μL reaction. 3 μL of HDAC6 in assay buffer were added to the assay plates and pre-incubate at room temp. for 30 mins. After pre-incubation, 3 μL of the tracer and TB-anti-GST antibody mix were added to the assay plates. The final concentrations of HDAC6, tracer, and TB-anti-GST antibody for each assay are 2 nM, 1 nM, and 0.5 nM, respectively. Binding reactions were equilibrated at 25° C. for an additional 6 hours, then read in endpoint mode on the BMG PheraStar (BMG labtech) equipped with a 337-520-490 optical module. The ratio of the 520/490 nm emission was calculated to determine the relative amounts of HDAC6 and tracer complex residue in each well.

### Data Analysis

The percent inhibition of HDAC6 (IC₅₀) was calculated according to the following equation.

$$\% Inhibition = ( ( Median\_high - Response\_raw \_data ) / ( Median\_high - Median\_low ) ) * 10 \; 0 .$$

As set forth in Table 1, “A” indicates an HDAC6 IC₅₀ value of less than 200 nM; “B” indicates an HDAC6 IC₅₀ value of greater than or equal to 200 nM and less than 500 nM; and “C” indicates an HDAC6 IC₅₀ value of greater than or equal to 500 nM and less than 5 μM.

TABLE 1

| Compound | HDAC6 IC₅₀ |
|---|---|
| 1 | A |
| 3 | A |
| 4 | A |
| 5 | A |
| 6 | C |
| 7 | A |
| 8 | C |
| 13 | A |
| 14 | C |
| 15 | B |
| 16 | A |
| 17 | A |
| 18 | A |
| 19 | A |
| 20 | A |
| 22 | A |
| 23 | B |
| 24 | A |
| 25 | B |
| 26 | A |
| 27 | A |
| 28 | A |
| 29 | A |
| 30 | A |
| 31 | B |
| 32 | A |
| 33 | B |
| 34 | B |
| 35 | B |
| 36 | C |
| 37 | A |
| 38 | A |
| 39 | A |
| 40 | B |
| 41 | A |
| 42 | B |
| 43 | B |
| 44 | B |
| 45 | B |
| 46 | A |
| 47 | B |
| 48 | B |
| 49 | B |
| 50 | A |
| 51 | A |
| 52 | B |
| 53 | B |
| 54 | B |
| 55 | B |
| 56 | A |
| 71 | A |
| 72 | B |
| 73 | C |
| 74 | C |
| 75 | A |
| 76 | C |
| 77 | B |
| 78 | A |
| 80 | A |
| 81 | A |
| 82 | A |
| 83 | A |
| 84 | B |
| 85 | B |
| 86 | A |
| 88 | B |
| 89 | C |
| 90 | A |
| 91 | B |
| 93 | B |
| 94 | A |
| 95 | B |
| 96 | B |
| 97 | B |
| 98 | A |
| 99 | B |
| 100 | B |
| 103 | B |
| 104 | B |
| 105 | C |
| 107 | C |
| 109 | C |
| 111 | C |
| 112 | B |
| 113 | C |
| 114 | C |
| 115 | C |
| 121 | A |
| 125 | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than the specific embodiments that have been represented by way of examples.

1. A compound of formula I: 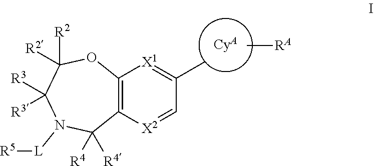
or a pharmaceutically acceptable salt thereof, wherein:
each of X¹ and X² is independently CR¹ or N;
each R¹ is independently hydrogen, halogen, —CN, or an optionally substituted C₁₋₆ aliphatic;
Cy^{A} is 1,2,4-oxadiazolyl or 1,3,4-oxadiazolyl;
R^{A} is methyl, optionally substituted with 1-3 fluoro;
each of R², R^{2′}, R³, R^{3′}, R⁴, R^{4′}, and R⁵ is independently hydrogen, halogen, —CN, —OR, —N(R)₂, —S(O)₂R, or an optionally substituted group selected from the group consisting of C₁₋₆ aliphatic, a 3- to 7-membered saturated or partially unsaturated monocyclic carbocycyl, a 3- to 7-membered saturated or partially unsaturated monocyclic heterocyclyl having 1-2 heteroatoms independently selected from oxygen, nitrogen, or sulfur, phenyl, a 5- to 6-membered monocyclic heteroaryl having 1-3 heteroatoms independently selected from oxygen, nitrogen, or sulfur, an 8- to 10-membered bicyclic aryl, a 7- to 12-member saturated or partially unsaturated bicyclic heterocyclyl having 1-4 heteroatoms independently selected from oxygen, nitrogen, or sulfur, and an 8- to 12-membered bicyclic heteroaryl having 1-4 heteroatoms independently selected from oxygen, nitrogen or sulfur; or
R³ and R^{3′}, together with their intervening atoms, form an optionally substituted 3- to 6-membered spirocyclic ring having 0-3 heteroatoms independently selected from oxygen, nitrogen, or sulfur; or
R³ and R^{3′} form ═O;
L is a covalent bond, or an optionally substituted C₁₋₃ hydrocarbon chain, wherein 1-3 methylene units are optionally and independently replaced with —O—, —C(O)—, —S(O)₂—, or —NR—; and
each R is independently hydrogen or optionally substituted C₁₋₆ aliphatic.

2. The compound of claim 1, wherein R^{2′} is hydrogen, and R² is an optionally substituted C₁₋aliphatic.

3. The compound of claim 1 or 2, wherein R^{2′} is hydrogen, and R² is hydrogen, methyl, or isopropyl.

4. The compound of any one of claims 1-3, wherein R⁴ and R^{4′} are hydrogen.

5. The compound of any one of claims 1-4, wherein the compound is of Formula II: 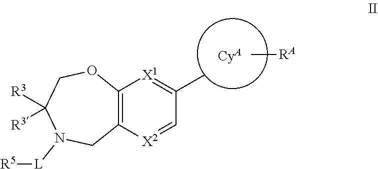
or a pharmaceutically acceptable salt thereof.

6. The compound of any one of claims 1-5, wherein X¹ and X² are CR¹.

7. The compound of any one of claims 1-5, wherein X¹ and X² are N.

8. The compound of any one of claims 1-5, wherein X¹ is N and X² is CR¹.

9. The compound of any one of claims 1-5, wherein X¹ is CR¹ and X² is N.

10. The compound of any one of claims 1-9, wherein Cy^{A} is

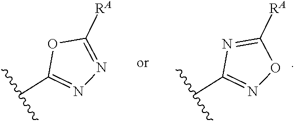

11. The compound of any one of claims 1-10, wherein the compound is of Formula III-a or III-b: 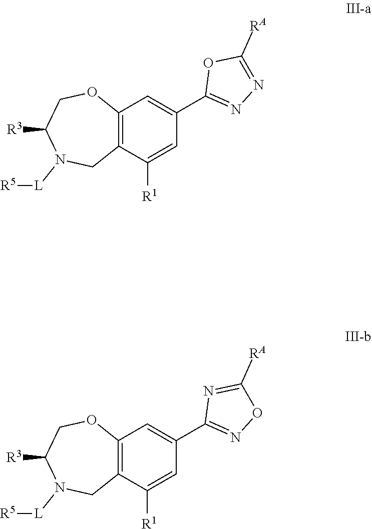
or a pharmaceutically acceptable salt thereof.

12. The compound of any one of claims 1-11, wherein R^{3′} is hydrogen, and R³ is an optionally substituted group selected from the group consisting of C₁₋aliphatic, a 3- to 7-membered saturated or partially unsaturated monocyclic carbocycyl, or phenyl.

13. The compound of any one of claims 1-12, wherein R^{3′} is hydrogen, and R³ is hydrogen, methyl, phenyl, cyclopropyl, or

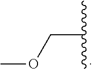 [SMILES: *COC]

14. The compound of any one of claims 1-13, wherein R^{3′} is hydrogen, and R³ is methyl or phenyl.

15. The compound of any one of claims 1-11, wherein R³ and R^{3′}, together with their intervening atoms, form an optionally substituted 3- to 6-membered spirocyclic ring having 0-3 heteroatoms independently selected from oxygen, nitrogen, or sulfur.

16. The compound of any one of claims 1-11 or 15, wherein R³ and R^{3′}, together with their intervening atoms, form a 3-membered spirocarbocyclic ring.

17. The compound of any one of claims 1-11, wherein R³ and R^{3′} form ═O.

18. The compound of any one of claims 1-14, wherein the compound is of Formula IV-a, IV-b, IV-c, or IV-d: 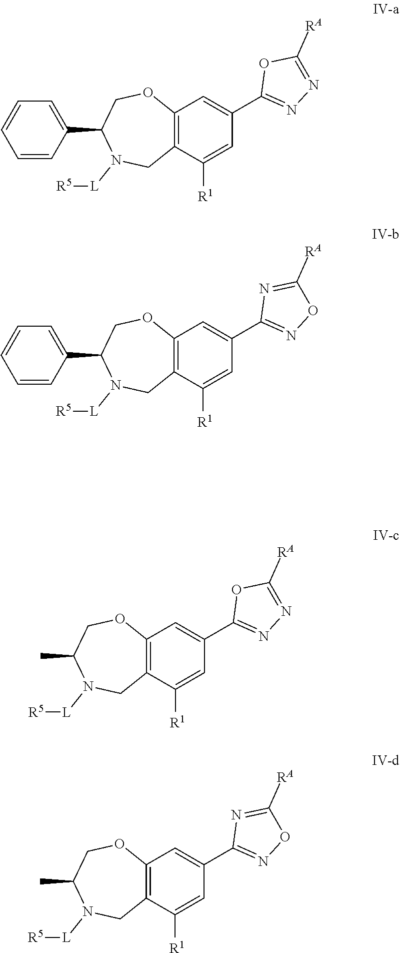
or a pharmaceutically acceptable salt thereof

19. The compound of any one of claims 1-18, wherein each R¹ is independently hydrogen or fluoro.

20. The compound of any one of claims 1-19, wherein R^{A} is —CF₂ or CF₃.

21. The compound of any one of claims 1-20, wherein R^{A} is —CF₃.

22. The compound of any one of claims 1-21, wherein L is a covalent bond.

23. The compound of any one of claims 1-21, wherein L is an optionally substituted C₁₋₃ hydrocarbon chain, wherein 1 methylene unit is replaced with —C(O)—.

24. The compound of any one of claims 1-21, wherein L is a covalent bond, —C(O)—, —C(O)CH₂—*, —C(O)CH₂CH₂—*, —C(O)O—*, —C(O)CH₂O—*, —CH₂—, —S(O)₂—, or —C(O)CH₂C(O)—, wherein * represents the point of attachment to R⁵.

25. The compound of any one of claims 1-24, wherein R⁵ is optionally substituted C₁₋₆ aliphatic.

26. The compound of any one of claims 1-24, wherein R⁵ is an optionally substituted 3- to 7-membered saturated or partially unsaturated monocyclic carbocycyl.

27. The compound of any one of claims 1-24 or 26, wherein R⁵ is an optionally substituted cyclopropyl.

28. The compound of any one of claims 1-24, wherein R⁵ is an optionally substituted 3- to 7-membered saturated or partially unsaturated monocyclic heterocyclyl having 1-2 heteroatoms independently selected from oxygen, nitrogen, or sulfur.

29. The compound of any one of claims 1-24 or 28, wherein R⁵ is optionally substituted oxetanyl, tetrahydropyranyl, or tetrahydrofuranyl.

30. The compound of any one of claims 1-24 or 28, wherein R⁵ is optionally substituted azetidinyl, pyrrolidinyl, or piperidinyl.

31. The compound of any one of claims 1-24, wherein R⁵ is optionally substituted 5- to 6-membered monocyclic heteroaryl having 1-3 heteroatoms independently selected from oxygen, nitrogen, or sulfur.

32. The compound of any one of claims 1-24 or 31, wherein R⁵ is optionally substituted furanyl, isoxazolyl, thiazolyl, thiadiazolyl, or pyridinyl.

33. The compound of any one of claims 1-24, wherein R⁵ is an optionally substituted 8- to 10-membered bicyclic aryl (e.g., benzo[b][1,4]oxazinonyl).

34. The compound of any one of claims 1-24, wherein R⁵ is an optionally substituted 7-12-membered saturated or partially unsaturated bicyclic heterocyclyl having 1-4 heteroatoms independently selected from oxygen, nitrogen, or sulfur (e.g., 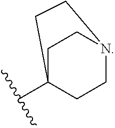 [SMILES: *C12CCN(CC1)CC2]
or triazolopyridinyl).

35. The compound of any one of claims 1-24, wherein R⁵ is an optionally substituted 8- to 12-membered bicyclic heteroaryl having 1-4 heteroatoms independently selected from oxygen, nitrogen or sulfur.

36. The compound of any one of claims 1-24 or 35, wherein R⁵ is an optionally substituted benzoimidazolyl, pyrazolopyridinyl, pyrrolooxazolyl, or pyrazolopyrimidinyl.

37. The compound of any one of claims 1-24, wherein R⁵ is hydrogen, —CN, —CH₃, —CF₃, —OH, —OMe, —C(CH₃)₂₀H, C(CH₃)₂NH₂, —C(CH₃)₂F, —NH₂, —NHCH₃, —N(CH₃)₂, isopropyl, t-butyl, —S(O)₂CH₃,

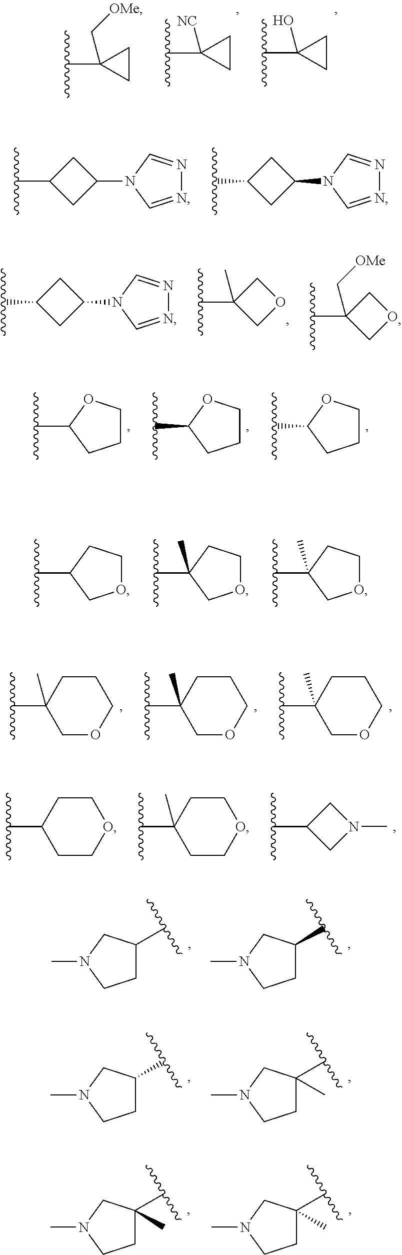

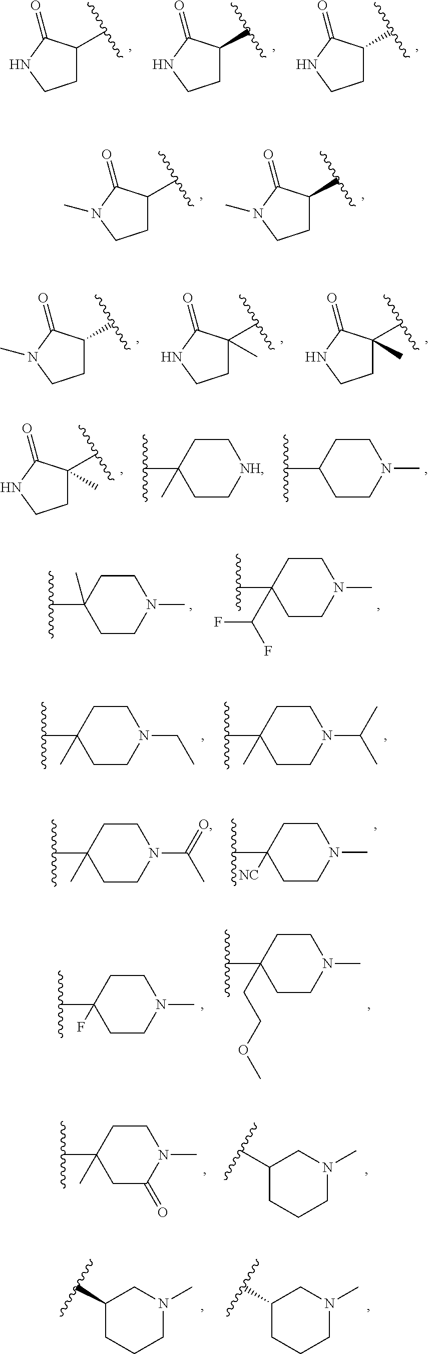

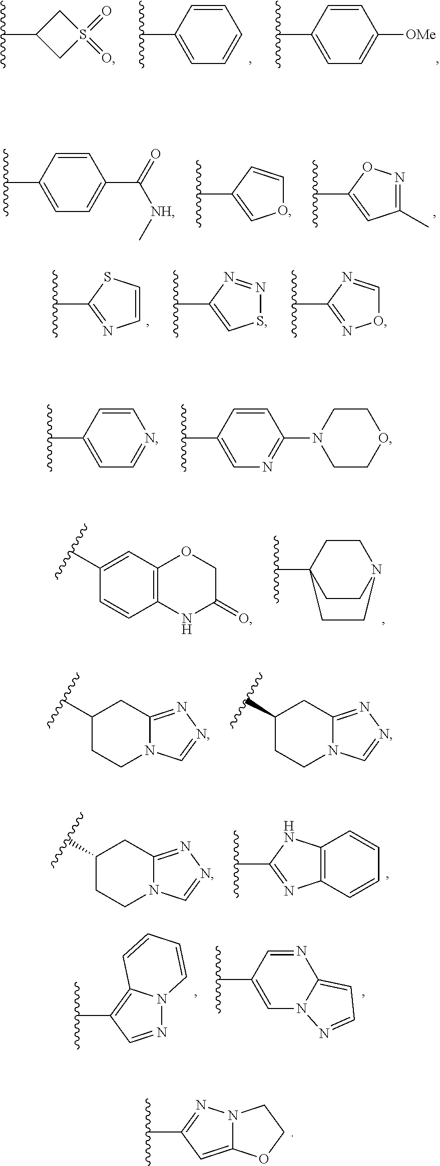

38. The compound of any one of claims 1-21, wherein -L-R⁵ is:

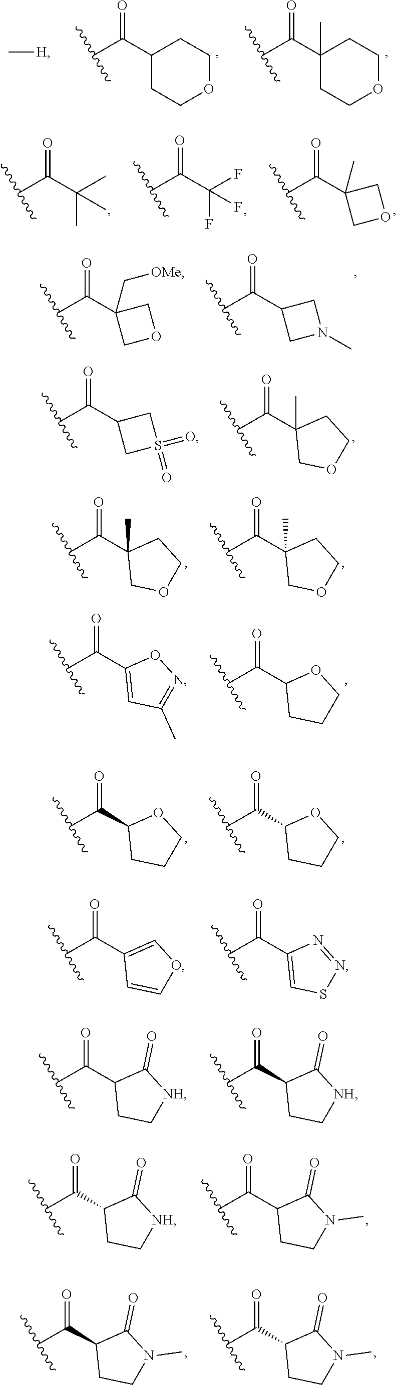

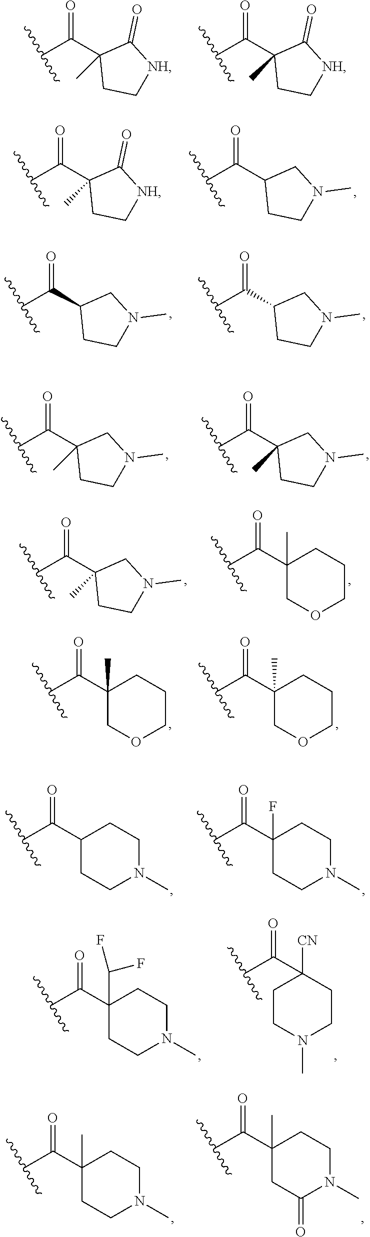

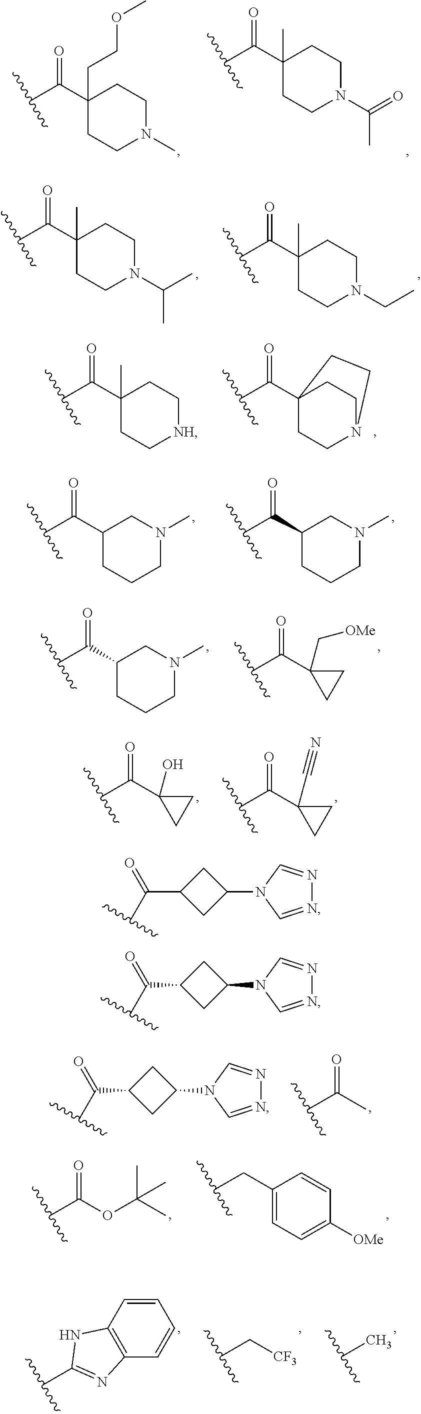

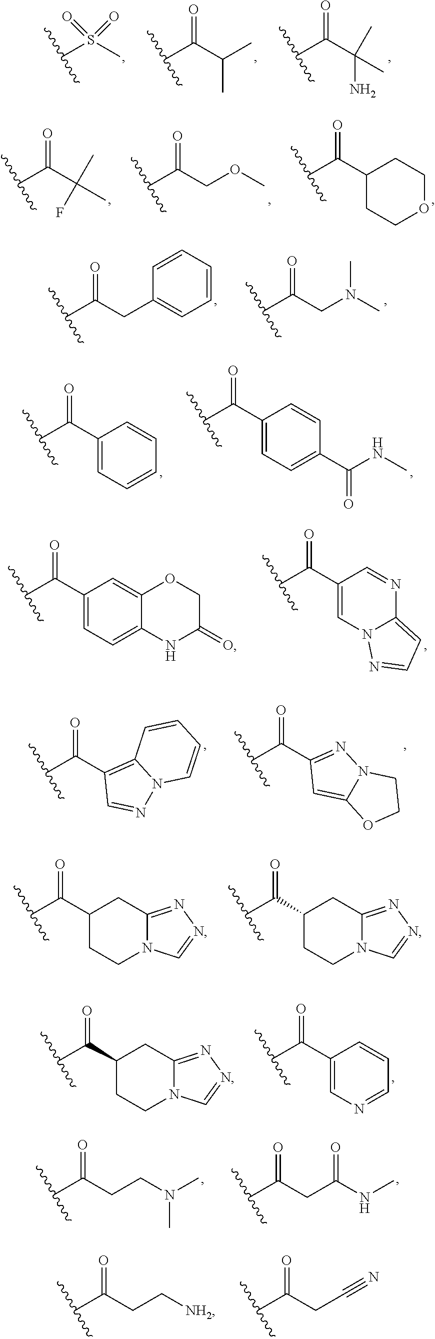

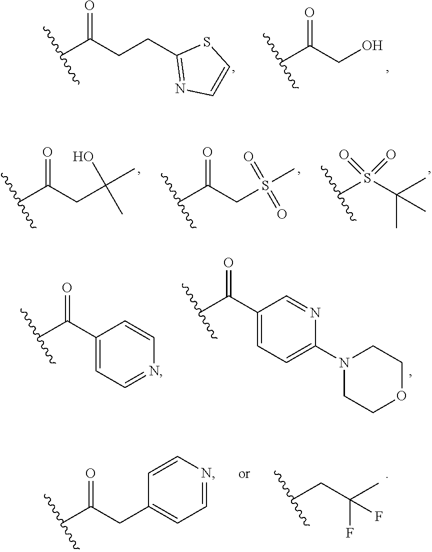

39. The compound of any one of claims 1-21, wherein the compound is of Formula V: 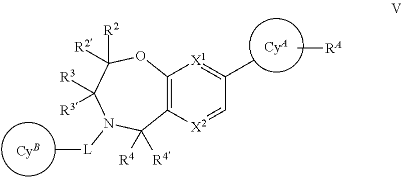
or a pharmaceutically acceptable salt thereof,
wherein:
Cy^{B} is an optionally substituted 3- to 7-membered saturated or partially unsaturated monocyclic heterocyclyl having 1-2 heteroatoms independently selected from oxygen, nitrogen, or sulfur, 5- to 6-membered monocyclic heteroaryl having 1-3 heteroatoms independently selected from oxygen, nitrogen, or sulfur, a 7- to 12-member saturated or partially unsaturated bicyclic heterocyclyl having 1-4 heteroatoms independently selected from oxygen, nitrogen, or sulfur, or an 8- to 12-membered bicyclic heteroaryl having 1-4 heteroatoms independently selected from oxygen, nitrogen or sulfur; and
wherein Cy^{B} comprises at least oxygen or nitrogen heteroatom.

40. The compound of any one of claims 1-21, wherein the compound is of Formula VI, VI-a, or VI-b: 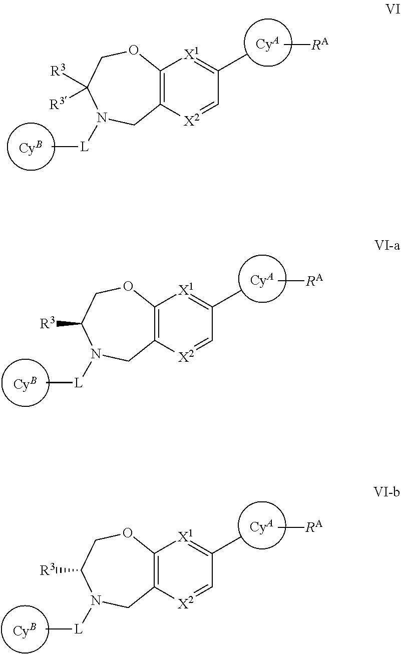
or a pharmaceutically acceptable salt thereof

41. The compound of any one of claims 1-21, wherein the compound is of Formula VII-a or VII-b: 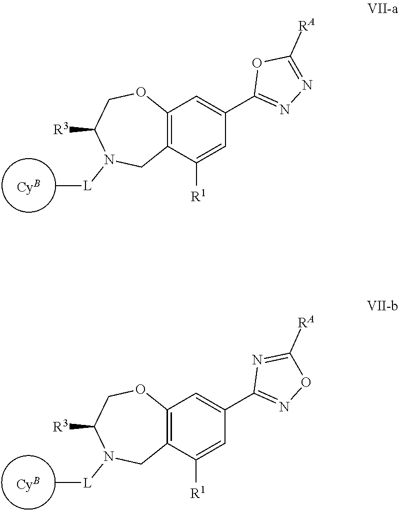
or a pharmaceutically acceptable salt thereof

42. The compound of any one of claims 1-21, wherein the compound is of Formula VIII-a, VIII-b, VIII-c, or VIII-d: 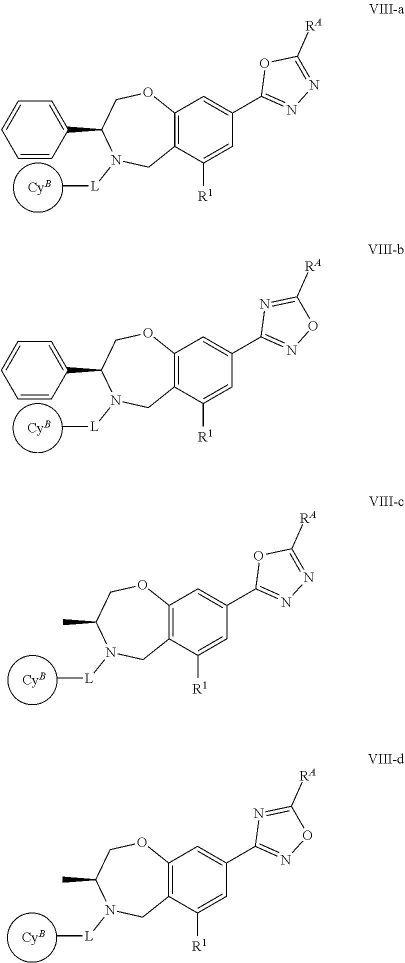
or a pharmaceutically acceptable salt thereof

43. The compound of any one of claims 39-42, wherein Cy^{B} comprises at least one oxygen heteroatom.

44. The compound of any one of claims 39-42, wherein Cy^{B} comprises at least one nitrogen heteroatom.

45. The compound of any one of claims 39-42 or 44, wherein Cy^{B} is 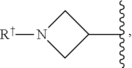
wherein R^{†} is C₁₋₄ aliphatic (e.g., methyl).

46. The compound of any one of claims 39-42, wherein Cy^{B} is

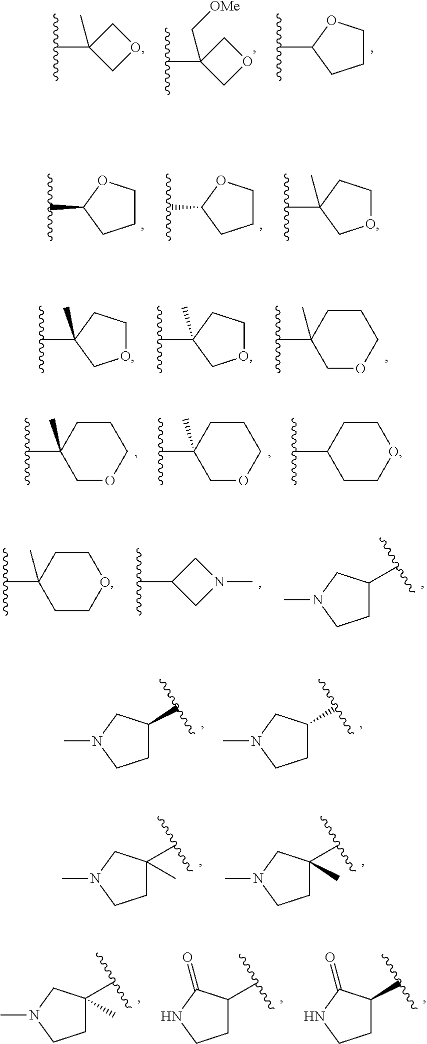

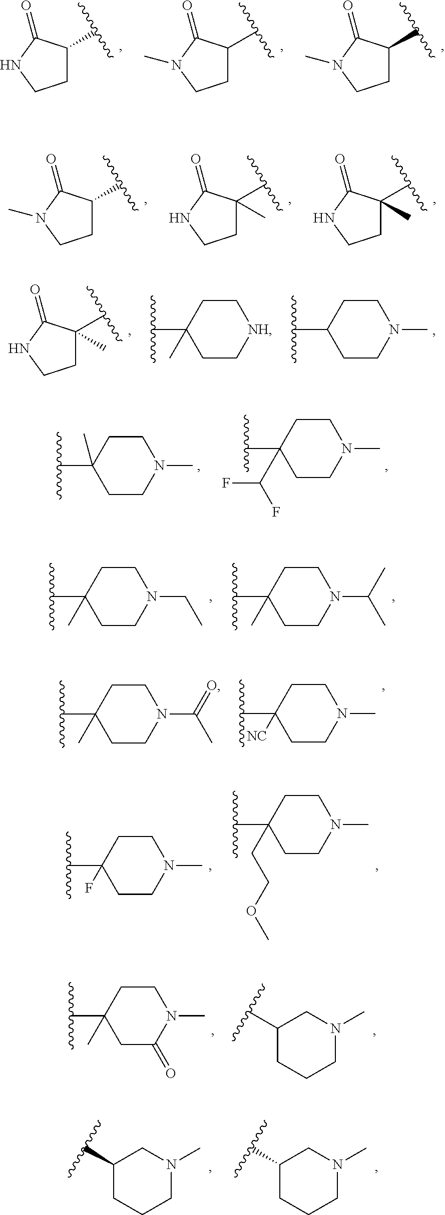

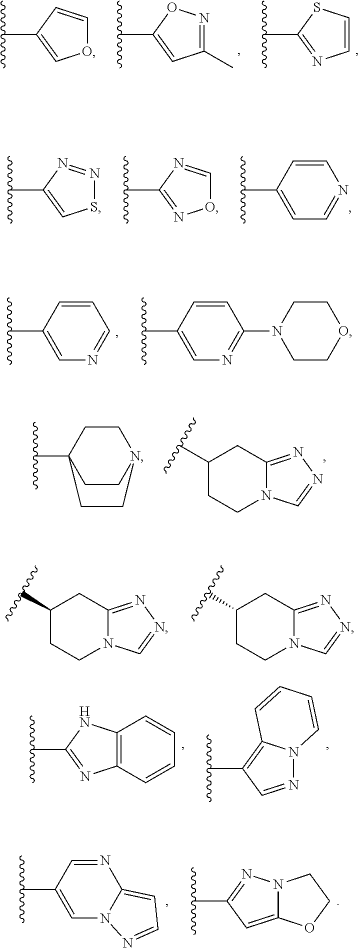

47. The compound of any one of claims 39-42, wherein L-Cy^{B} is:

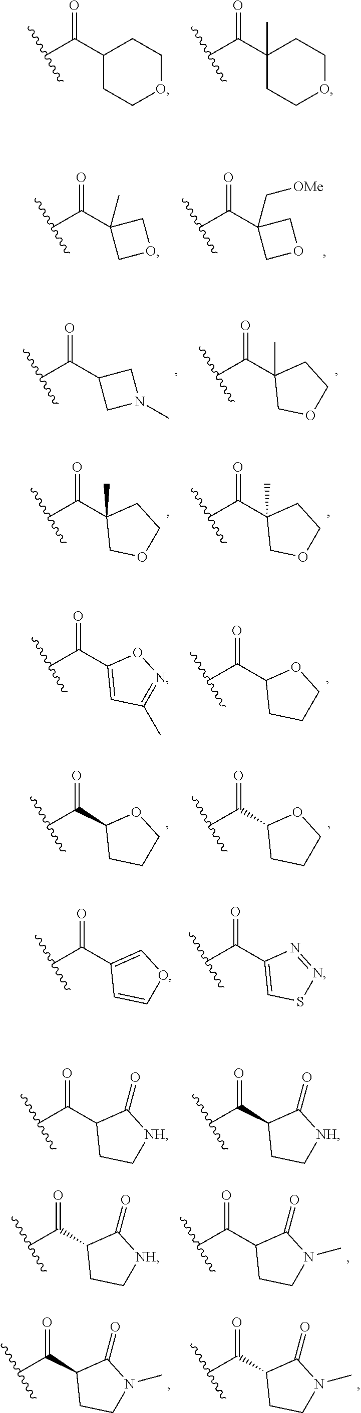

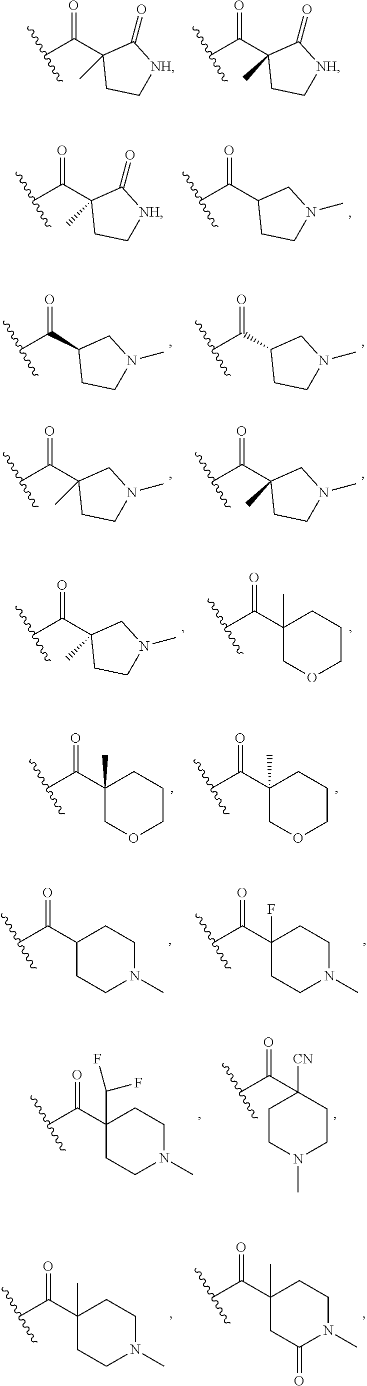

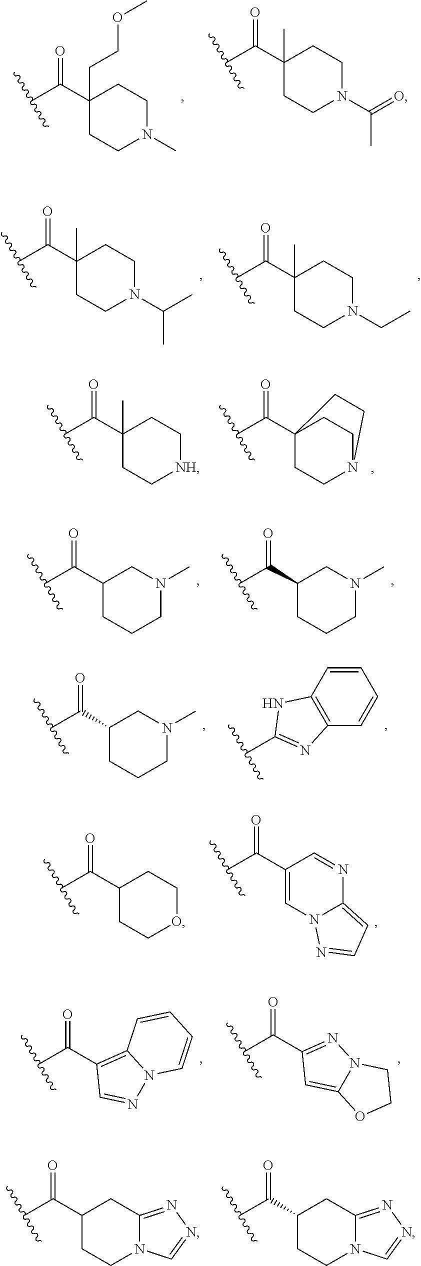

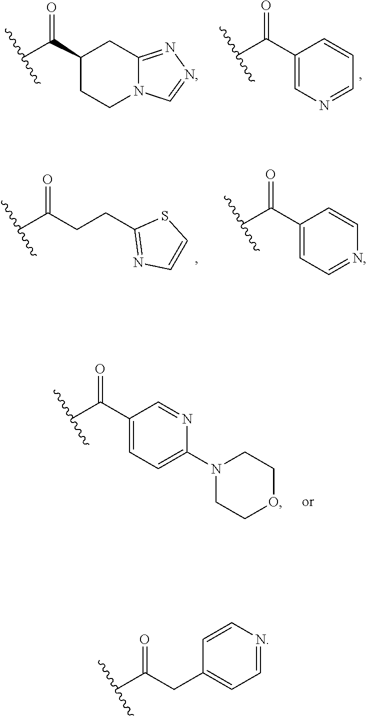 [SMILES: *C(=O)[C@@H]1CCn2cnnc2C1 ; *C(=O)c1cccnc1 ; *C(=O)CCc1nccs1 ; *C(=O)c1ccncc1 ; *C(=O)c1ccc(N2CCOCC2)nc1 ; *C(=O)Cc1ccncc1]

48. The compound of claim 1, wherein the compound is selected from compounds I-1 through I-132, or a pharmaceutically acceptable salt thereof.

49. A pharmaceutical composition comprising a compound of any one of claims 1-48, or a pharmaceutically acceptable salt thereof, and a pharmaceutically acceptable carrier, adjuvant, or vehicle.

50. A method of inhibiting activity of HDAC6, or a mutant thereof, in a biological sample or in a patient, comprising a step of contacting the biological sample or administering to a patient a compound according to any one of claims 1-48, or a pharmaceutically acceptable salt thereof, or a pharmaceutical composition according to claim 49.

51. A method of treating a disease or disorder associated with HDAC6, or a mutant thereof, the method comprising a step of administering to a patient in need thereof a compound according to any one of claims 1-48, or a pharmaceutically acceptable salt thereof, or a pharmaceutical composition according to claim 49.

52. A method of treating a cancer, the method comprising a step of administering to a patient in need thereof a compound according to any one of claims 1-48, or a pharmaceutically acceptable salt thereof, or a pharmaceutical composition according to claim 49.

53. The method of claim 52, wherein the cancer is multiple myeloma, colon cancer, lymphoma (e.g., histiocytic lymphoma, cutaneous T-cell lymphomas, and relapsed or refractory peripheral T-cell lymphomas), or glioblastoma.

54. The method of claim 52, wherein the cancer is an ARID1A-mutated cancer.

55. A method of treating a disease or disorder associated with the central nervous system, the method comprising a step of administering to a patient in need thereof a compound according to any one of claims 1-48, or a pharmaceutically acceptable salt thereof, or a pharmaceutical composition according to claim 49.

56. The method of claim 55, wherein the disease or disorder associated with the central nervous system is Amyotrophic Lateral Sclerosis (ALS), Alzheimer's disease, Parkinson's disease, Rett syndrome (RTT), Charcot-Marie-Tooth (CMT) disease, Fragile X Syndrome (FXS), Rubinstein-Taybi syndrome, depression, or schizophrenia

57. A method of treating a disease, disorder, or condition associated with the peripheral nervous system, the method comprising a step of administering to a patient in need thereof a compound according to any one of claims 1-48, or a pharmaceutically acceptable salt thereof, or a pharmaceutical composition according to claim 49.

58. The method of claim 57, wherein the disease, disorder, or condition associated with the peripheral nervous system is peripheral nerve injury or peripheral inflammation.

59. A method of treating chemotherapy-induced peripheral neuropathy, the method comprising a step of administering to a patient in need thereof a compound according to any one of claims 1-48, or a pharmaceutically acceptable salt thereof, or a pharmaceutical composition according to claim 49.

60. The method of claim 59, wherein the patient has received or is receiving taxol.

61. The method of claim 59, wherein the patient has received or is receiving cisplatin.
